(19) United States
(12) Patent Application Publication
(10) Pub. No.: US 2022/0402928 A1
(43) Pub. Date: Dec. 22, 2022

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR); Seoyeon KIM, Daejeon (KR); Da Jung LEE, Daejeon (KR)

(21) Appl. No.: 17/640,465

(22) PCT Filed: Apr. 14, 2021
(86) PCT No.: PCT/KR2021/004692

§ 371 Date: Mar. 4, 2022

(30) Foreign Application Priority Data
Apr. 14, 2020 (KR) 10-2020-0045515
Apr. 13, 2021 (KR) 10-2021-0048072

(51) Int. Cl. C07D 491/048; H01L 51/00

(52) U.S. Cl. CPC C07D 491/048; H01L 51/0052; H01L 51/0067; H01L 51/0071; H01L 51/0073; H01L 51/0074

(57) ABSTRACT

Provided is a compound of Chemical Formula 1:

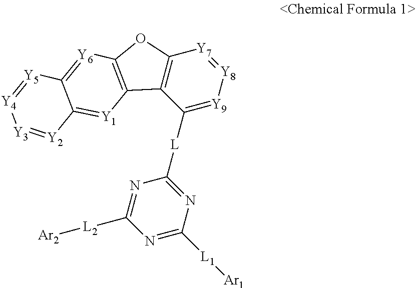

wherein:
Y₁ to Y₉ are each independently N, C—H, C-D, or C-L′-R, provided that at least one of Y₁ to Y₉ is N;
L′ is a single bond or a substituted or unsubstituted C₆₋₆₀ arylene;
R is a substituted or unsubstituted C₆₋₆₀ aryl or C₂₋₆₀ heteroaryl containing any one or more of N, O and S;
L is a single bond, a substituted or unsubstituted C₆₋₆₀ arylene, or a substituted or unsubstituted C₂₋₆₀ heteroarylene containing any one or more of N, O and S;
L₁ and L₂ are each independently a single bond or a substituted or unsubstituted C₆₋₆₀ arylene; and
Ar₁ and Ar₂ are each independently a substituted or unsubstituted C₆₋₆₀ aryl or C₂₋₆₀ heteroaryl containing any one or more of N, O and S;
   and an organic light emitting device including the same.

### CROSS-REFERENCE TO RELATED APPLICATION(S)

This application is a National Stage Application of International Application No. PCT/KR2021/004692 filed on Apr. 14, 2021, which claims the benefit of Korean Patent Application No. 10-2020-0045515 filed on Apr. 14, 2020 and Korean Patent Application No. 10-2021-0048072 filed on Apr. 13, 2021 in the Korean Intellectual Property Office, the disclosures of which are incorporated herein by reference in their entirety.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### BRIEF DESCRIPTION

### Technical Problem

It is an object of the present disclosure to provide a novel compound and an organic light emitting device comprising the same.

### Technical Solution

According to an aspect of the present disclosure, there is provided a compound of the following Chemical Formula 1:

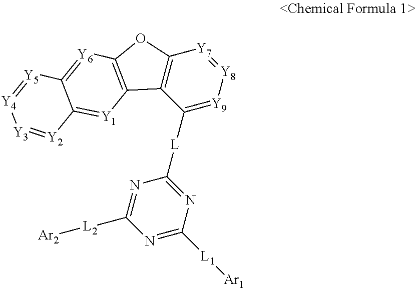

wherein, in Chemical Formula 1:

Y₁ to Y₉ are each independently N, C—H, C-D, or C-L′-R, with the proviso that at least one of Y₁ to Y₉ is N;

L′ is a single bond or a substituted or unsubstituted C₆₋₆₀ arylene;

R is a substituted or unsubstituted C₆₋₆₀ aryl or a substituted or unsubstituted C₂₋₆₀ heteroaryl containing any one or more heteroatoms selected from the group consisting of N, O and S;

L is a single bond, a substituted or unsubstituted C₆₋₆₀ arylene, or a substituted or unsubstituted C₂₋₆₀ heteroarylene containing any one or more heteroatoms selected from the group consisting of N, O and S;

L₁ and L₂ are each independently a single bond or a substituted or unsubstituted C₆₋₆₀ arylene; and

Ar₁ and Ar₂ are each independently a substituted or unsubstituted C₆₋₆₀ aryl or a substituted or unsubstituted C₂₋₆₀ heteroaryl containing any one or more heteroatoms selected from the group consisting of N, O and S.

According to another aspect of the present disclosure, there is provided an organic light emitting device comprising: a first electrode; a second electrode that is provided opposite to the first electrode; and a light emitting layer that is provided between the first electrode and the second electrode, wherein the light emitting layer comprises the compound of Chemical Formula 1.

### Advantageous Effects

The above-mentioned compound of Chemical Formula 1 is used as a material of an organic material layer in an organic light emitting device, and thus, can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION

Hereinafter, embodiments of the present disclosure will be described in more detail to facilitate understanding of the invention.

(Definition of Terms)

As used herein, the notation [symbol] and [symbol] mean a bond linked to another substituent group, and “D” means deuterium.

As used herein, the term “substituted or unsubstituted” means being unsubstituted or substituted with one or more substituents selected from the group consisting of deuterium, a halogen group, a cyano group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenyl group can be an aryl group, or it can also be interpreted as a substituent in which two phenyl groups are linked.

In the present disclosure, the carbon number of a carbonyl group is not particularly limited, but is preferably 1 to 40. Specifically, the carbonyl group can be a group having the following structural formulas, but is not limited thereto:

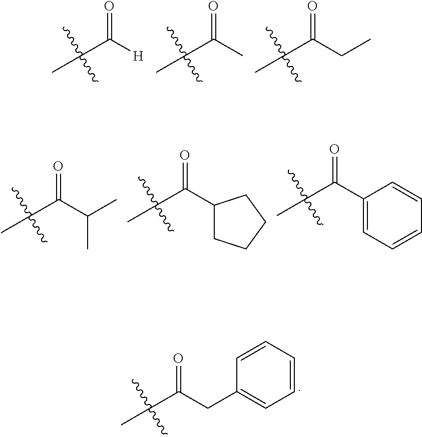

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a group having the following structural formulas, but is not limited thereto:

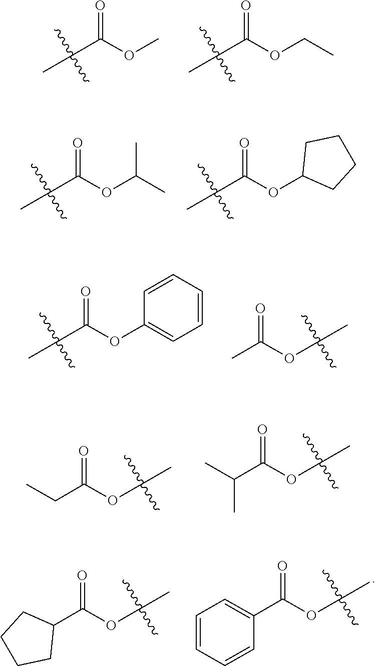

In the present disclosure, the carbon number of an imide group is not particularly limited, but is preferably 1 to 25. Specifically, the imide group can be a group having the following structural formulas, but is not limited thereto:

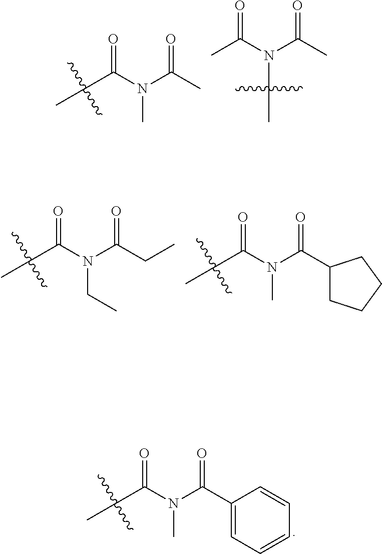

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a dimethylboron group, a triethylboron group, a t-butylmethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethyl-cyclopentyl, cyclohexyl, 3-methylcyclohexyl, 4-methylcyclohexyl, 2,3-dimethylcyclohexyl, 3,4,5-trimethylcyclohexyl, 4-tert-butyl-cyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenylyl group, a terphenylyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group can be substituted, and two substituents can be linked with each other to form a spiro structure. In the case where the fluorenyl group is substituted,

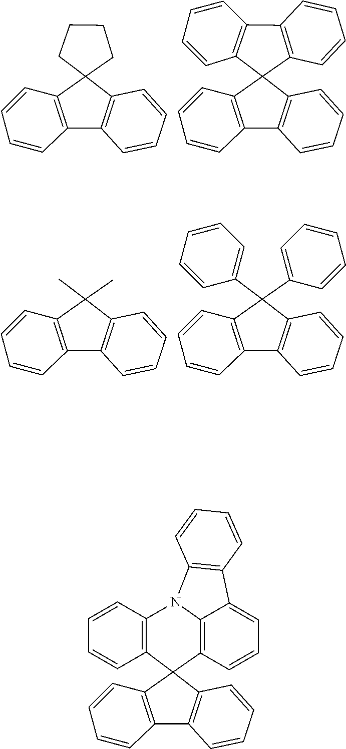

and the like can be formed.

In the present disclosure, a heteroaryl is a heteroaryl containing at least one of O, N, Si and S as a heteroatom, and the carbon number thereof is not particularly limited, but is preferably to 60. Examples of the heteroaryl include xanthene, thioxanthene, a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, the arylamine group and the arylsilyl group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine can be applied to the above-mentioned description of the heteroaryl. In the present disclosure, the alkenyl group in the aralkenyl group is the same as the above-mentioned examples of the alkenyl group. In the present disclosure, the above-mentioned description of the aryl group can be applied except that the arylene is a divalent group. In the present disclosure, the above-mentioned description of the heteroaryl can be applied except that the heteroarylene is a divalent group. In the present disclosure, the above-mentioned description of the aryl group or cycloalkyl group can be applied except that the hydrocarbon ring is not a monovalent group but formed by combining two substituent groups. In the present disclosure, the aforementioned description of the heteroaryl can be applied, except that the heterocyclic ring is not a monovalent group but formed by combining two substituent groups.

In the present disclosure, the term “deuterated or substituted with deuterium” means that at least one usable hydrogen in each chemical formula or substituent group is replaced by deuterium. In one example, being at least 10% deuterated in each formula means that at least 10% of the usable hydrogen is replaced by deuterium. In one example, each chemical formula can be at least 20%, at least 30%, at least 40%, at least 50%, at least 60%, at least 70%, at least 80%, at least 90%, or 100% deuterated.

(Compound)

The present disclosure provides the compound of Chemical Formula 1.

The compound of Chemical Formula 1 has a structure in which it has a triazinyl group at the 1-position of benzonaphthofuran, and at least one of the carbon atoms at the remaining positions is replaced with a “nitrogen atom”.

Such compounds can exhibit excellent energy transfer properties and stability as compared with a compound in which the triazinyl group is substituted at a position other than the 1-position and a compound not substituted by the triazinyl group. Therefore, the organic light emitting device employing the above compound can exhibit device characteristics in which luminous efficiency and lifetime are simultaneously improved, as compared with an organic light emitting device employing a compound in which the triazinyl group is substituted at a position other than the 1-position and a compound not substituted with a triazinyl group.

In one embodiment, one of Y₁ to Y₉ can be N.

Specifically, one of Y₁ to Y₉ is N, and the rest are each independently C—H or C-D; or

one of Y₁ to Y₉ can be N, and one of the rests can be C-L′-R, and the others can be each independently C—H or C-D.

More specifically,

one of Y₁ to Y₉ is N, and the rest are all C—H; or

one of Y₁ to Y₉ is N, and the rest are all C-D; or

one of Y₁ to Y₉ is N, one of the rests is C-L′-R, and the others are each independently C—H; or

one of Y is N, one of the rests is C-L′-R, and the others are each independently C-D.

For example,

Y₁ is N, and Y₂, Y₃, Y₄, Y₅, Y₆, Y₇, Y₈ and Y₉ are each independently C—H, C-D, or C-L′-R; or

Y₂ is N, and Y₁, Y₃, Y₄, Y₅, Y₆, Y₇, Y₈ and Y₉ are each independently C—H, C-D, or C-L′-R; or

Y₃ is N, and Y₁, Y₂, Y₄, Y₅, Y₆, Y₇, Y₈ and Y₉ are each independently C—H, C-D, or C-L′-R; or

Y₄ is N, and Y₁, Y₂, Y₃, Y₅, Y₆, Y₇, Y₈ and Y₉ are each independently C—H, C-D, or C-L′-R; or

Y₅ is N, and Y₁, Y₂, Y₃, Y₄, Y₆, Y₇, Y₈ and Y₉ are each independently C—H, C-D, or C-L′-R; or

Y₆ is N, and Y₁, Y₂, Y₃, Y₄, Y₅, Y₇, Y₈ and Y₉ are each independently C—H, C-D, or C-L′-R; or

is N, and Y₁ Y₂, Y₃, Y₄, Y₅, Y₆, Y₈ and Y₉ are each independently C—H, C-D, or C-L′-R; or

Y₈ is N, and Y₁ Y₂, Y₃, Y₄, Y₅, Y₆, Y₇ and Y₉ are each independently C—H, C-D, or C-L′-R; or

Y₉ is N, and Y₁ Y₂, Y₃, Y₄, Y₅, Y₆, Y₇ and Y₈ are each independently C—H, C-D, or C-L′-R.

Further, in Chemical Formula 1, L′ can be a single bond or a C₆₋₂₀ arylene that is unsubstituted or substituted with deuterium.

Specifically, L′ can be a single bond; phenylene that is unsubstituted or substituted with deuterium; or naphthylene that is unsubstituted or substituted with deuterium.

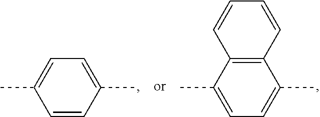

For example, L′ is a single bond, but is not limited thereto.

Further, in Chemical Formula 1, R is a C₆₋₆₀ aryl, or a C₂₋₂₀ heteroaryl containing any one heteroatom selected from the group consisting of N, O and S,

where R can be unsubstituted or substituted with one or more, for example, one or two substituents selected from the group consisting of deuterium, C₁₋₁₀ alkyl and C₆₋₂₀ aryl.

Specifically, R is any one selected from the group consisting of the following:

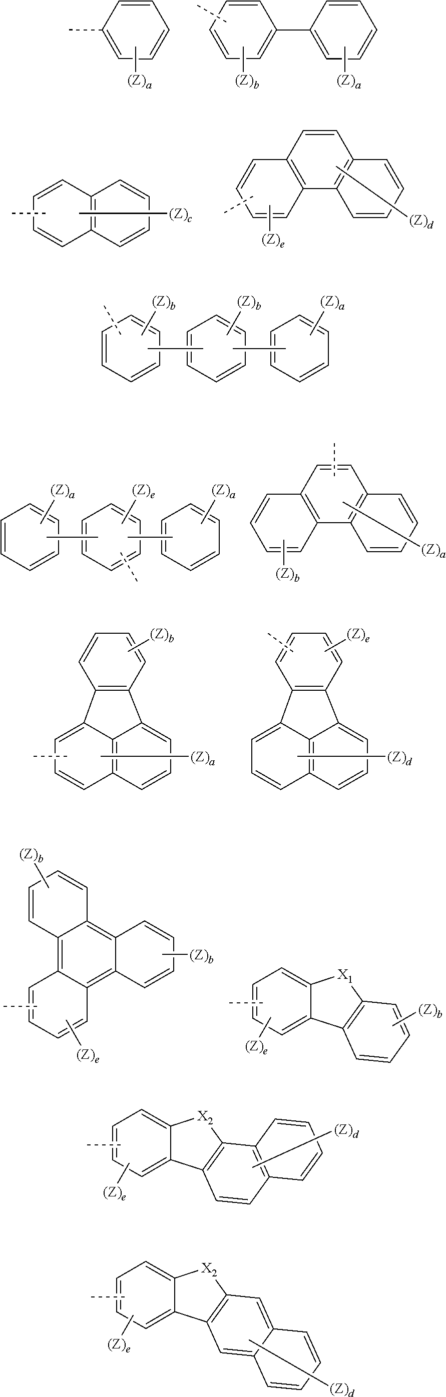

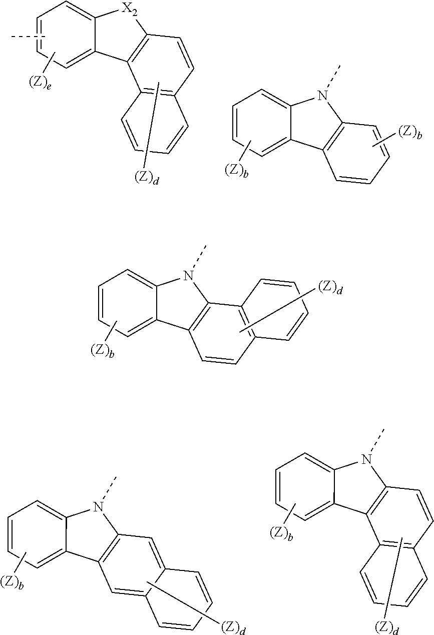

wherein:

X₁ and X₂ are each independently O, S, or N(phenyl);

each Z is independently deuterium (D), C₁₋₁₀ alkyl, or C₆₋₂₀ aryl;

each a is independently an integer of 0 to 5;

each b is independently an integer of 0 to 4;

each c is independently an integer of 0 to 7;

each d is independently an integer of 0 to 6; and

each e is independently an integer of 0 to 3.

For example, R can be phenyl, biphenylyl, naphthyl, phenanthryl, dibenzofuranyl, dibenzothiophenyl, or carbazolyl.

Further, for example, R can be any one selected from the group consisting of the following, but is not limited thereto:

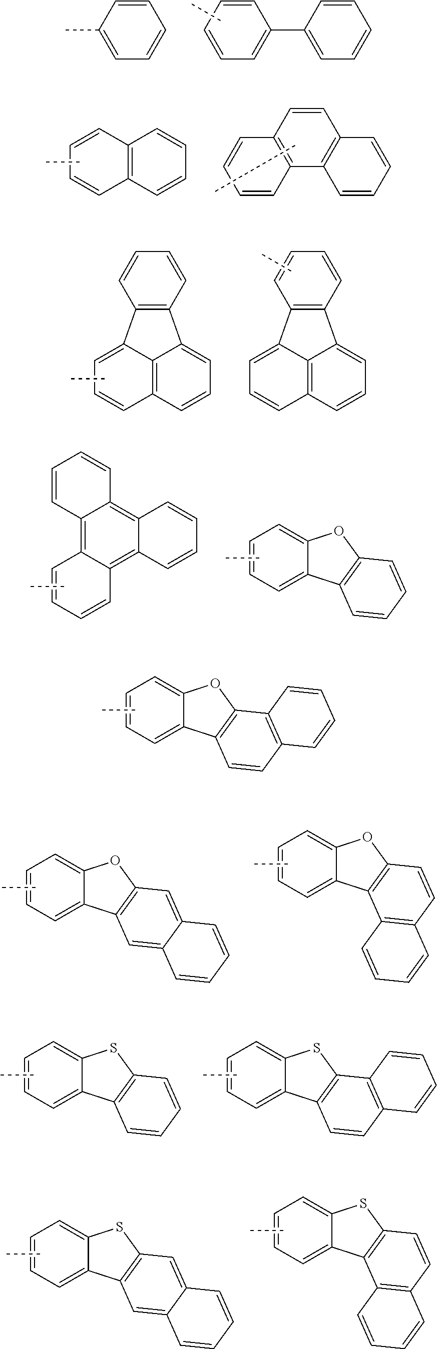

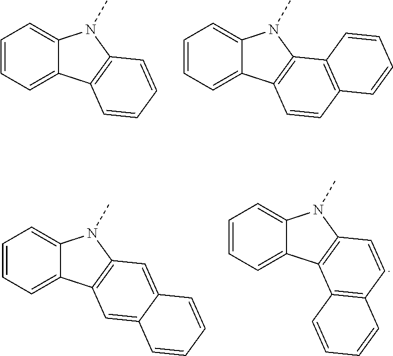

Further, in Chemical Formula 1, L can be a single bond or a C₆₋₂₀ arylene that is unsubstituted or substituted with deuterium.

Specifically, L can be a single bond; phenylene that is unsubstituted or substituted with deuterium; or naphthylene that is unsubstituted or substituted with deuterium.

More specifically, L can be a single bond or any one selected from the group consisting of the following:

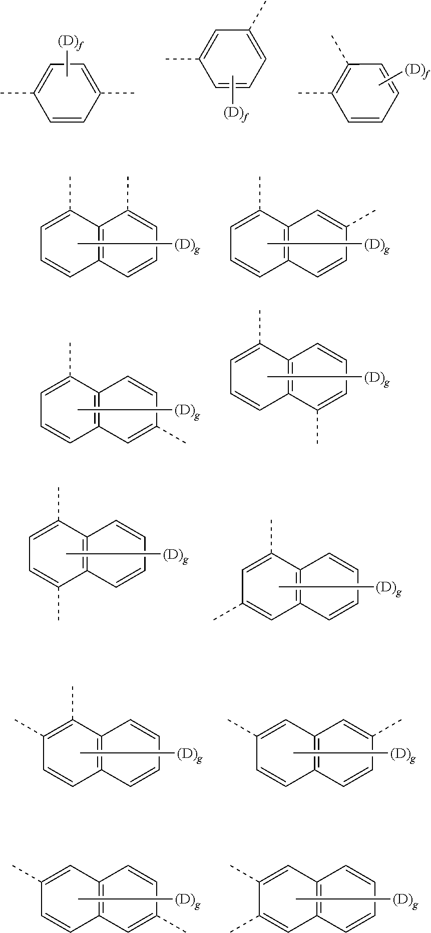

wherein:

D means deuterium;

each f is independently an integer of 0 to 4; and

each g is independently an integer of 0 to 6.

For example, L can be a single bond, or any one selected from the group consisting of the following:

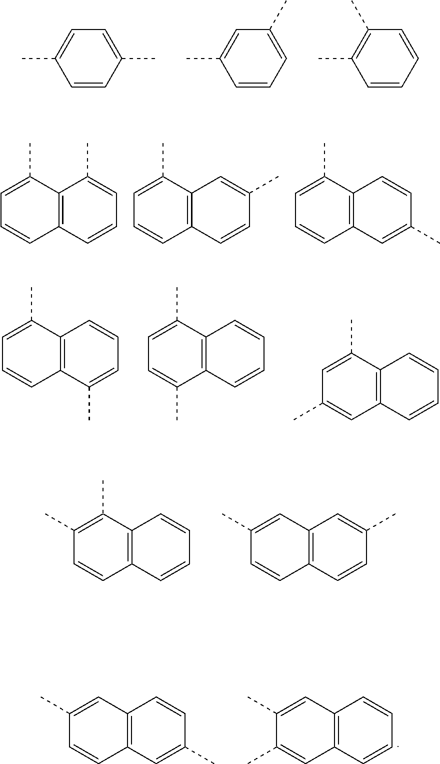

Further, in Chemical Formula 1, L₁ and L₂ can be each independently a single bond or a C₆₋₂₀ arylene that is unsubstituted or substituted with deuterium.

Specifically, L₁ and L₂ can be each independently a single bond; phenylene that is unsubstituted or substituted with deuterium; biphenyldiyl that is unsubstituted or substituted with deuterium; or naphthylene that is unsubstituted or substituted with deuterium.

And, one of L₁ and L₂ can be a single bond.

Further, in Chemical Formula 1, Ar₁ and Ar₂ are each independently a C₆₋₂₀ aryl or a C₂₋₂₀ heteroaryl containing one heteroatom selected from the group consisting of N, O and S,

where Ar₁ and Ar₂ can be unsubstituted or substituted with one or more substituents selected from the group consisting of deuterium, a C₁₋₁₀ alkyl and a C₆₋₂₀ aryl.

Specifically, Ar₁ and Ar₂ are each independently phenyl, biphenylyl, terphenylyl, naphthyl, phenanthryl, fluorenyl, dibenzofuranyl, dibenzothiophenyl, benzonaphthofuranyl, benzonaphthothiophenyl, carbazolyl, or benzocarbazolyl,

where Ar₁ and Ar₂ can be unsubstituted or substituted with one or more, for example, one or two substituents selected from the group consisting of deuterium, C₁₋₁₀ alkyl and C₆₋₂₀ aryl.

For example, Ar₁ and Ar₂ can be each independently any one selected from the group consisting of the following, but are not limited thereto:

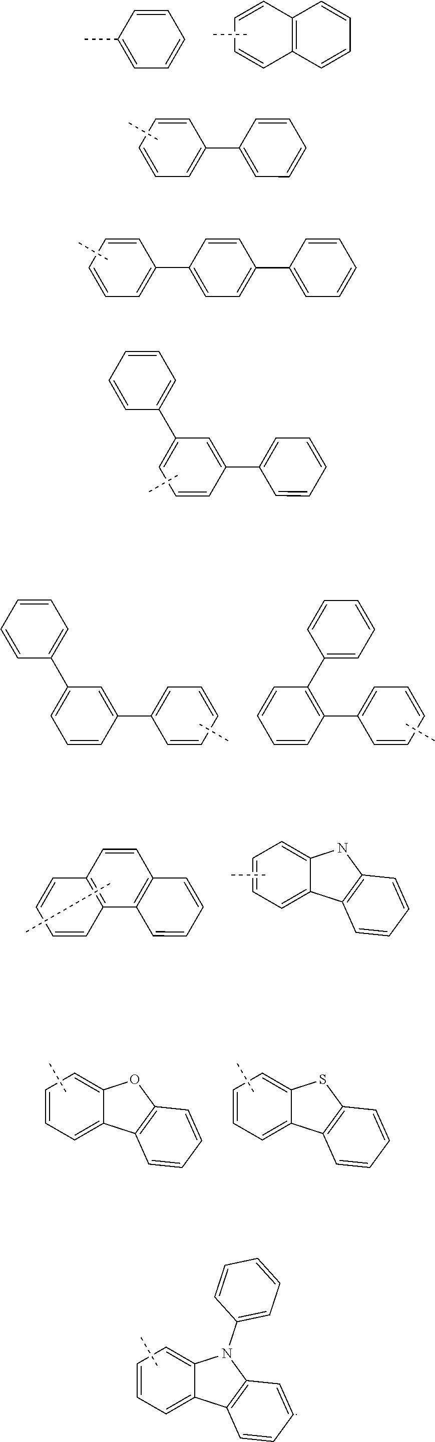

Further, in Chemical Formula 1, one of Ar₁ and Ar₂ can be phenyl that is unsubstituted or substituted with deuterium; biphenylyl that is unsubstituted or substituted with deuterium; or naphthyl that is unsubstituted or substituted with deuterium.

Specifically, one of Ar₁ and Ar₂ can be phenyl, biphenylyl, or naphthyl.

For example, one of Ar₁ and Ar₂ can be

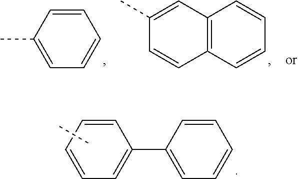

Also, in Chemical Formula 1, Ar₁ and Ar₂ can be identical to or different from each other.

When Ar₁ and Ar₂ are identical to each other, both Ar₁ and Ar₂ can be

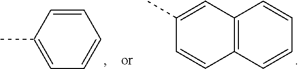

Further, in Chemical Formula 1,

L₁ is a single bond, and L₂ is

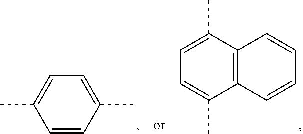

and

Ar₁ is phenyl, naphthyl, or biphenylyl;

Ar₂ can be any one selected from the group consisting of the following:

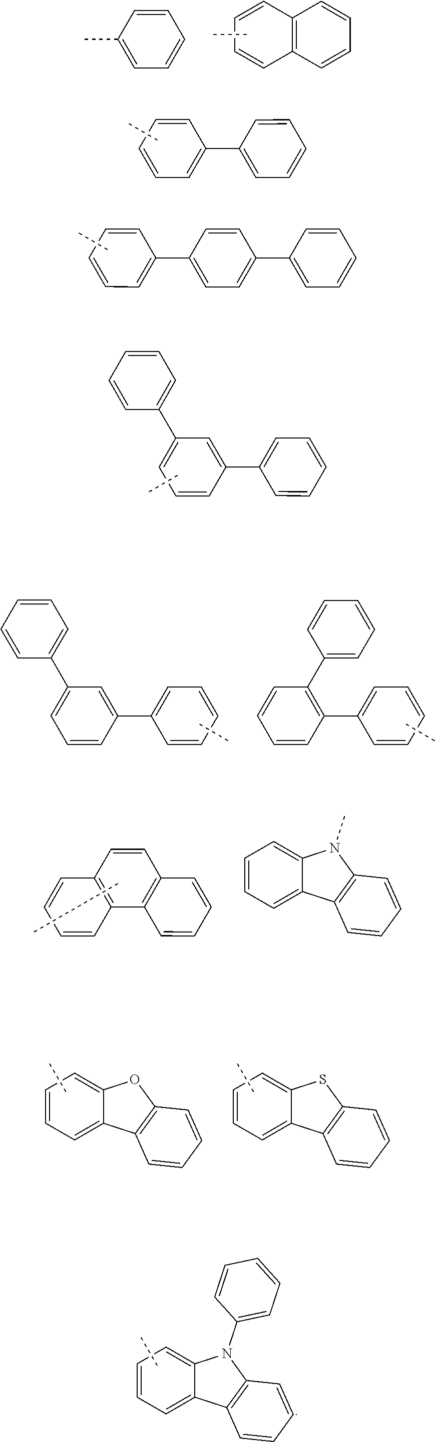

Further, the compound can be any one of the following Chemical Formulas 1-1 to 1-3:

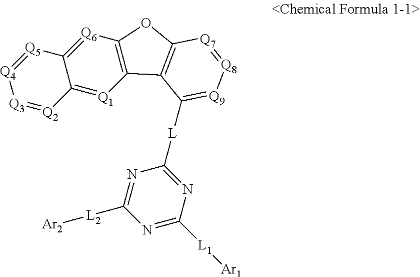

wherein, in Chemical Formula 1-1:

Q₁ to Q₉ are each independently N or C—H, with the proviso that one of Q₁ to Q₉ is N; and

L, L₁, L₂, Ar₁ and Ar₂ are as defined in Chemical Formula 1;

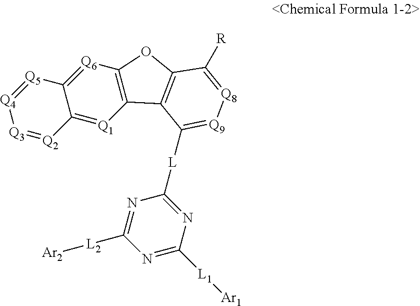

wherein, in Chemical Formula 1-2:

Q₁ to Q₆, Q₈ and Q₉ are each independently N or C—H, with the proviso that one of Q₁ to Q₆, Q₈ and Q₉ is N; and

R, L, L₁, L₂, Ar₁ and Ar₂ are as defined in Chemical Formula 1;

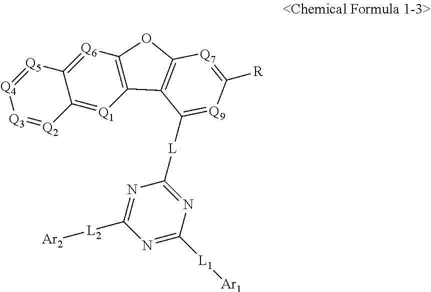

wherein, in Chemical Formulas 1-3,

Q₁ to Q₇ and Q₉ are each independently N or C—H, with the proviso that one of Q₁ to Q₇ and Q₉ is N, and

R, L, L₁, L₂, Ar₁ and Ar₂ are as defined in Chemical Formula 1. As an example, the compound is any one selected from the group consisting of the following compounds:

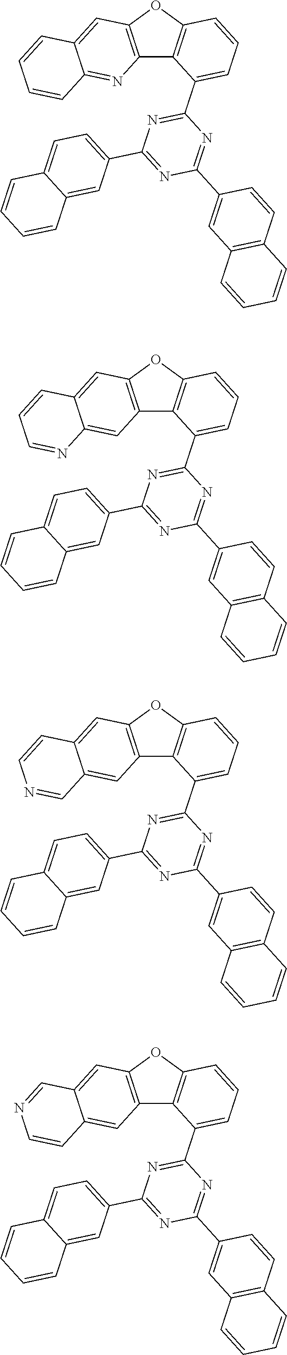 [SMILES: c1ccc2cc(-c3nc(-c4ccc5ccccc5c4)nc(-c4cccc5oc6cc7ccccc7nc6c45)n3)ccc2c1 ; c1ccc2cc(-c3nc(-c4ccc5ccccc5c4)nc(-c4cccc5oc6cc7cccnc7cc6c45)n3)ccc2c1 ; c1ccc2cc(-c3nc(-c4ccc5ccccc5c4)nc(-c4cccc5oc6cc7ccncc7cc6c45)n3)ccc2c1 ; c1ccc2cc(-c3nc(-c4ccc5ccccc5c4)nc(-c4cccc5oc6cc7cnccc7cc6c45)n3)ccc2c1]

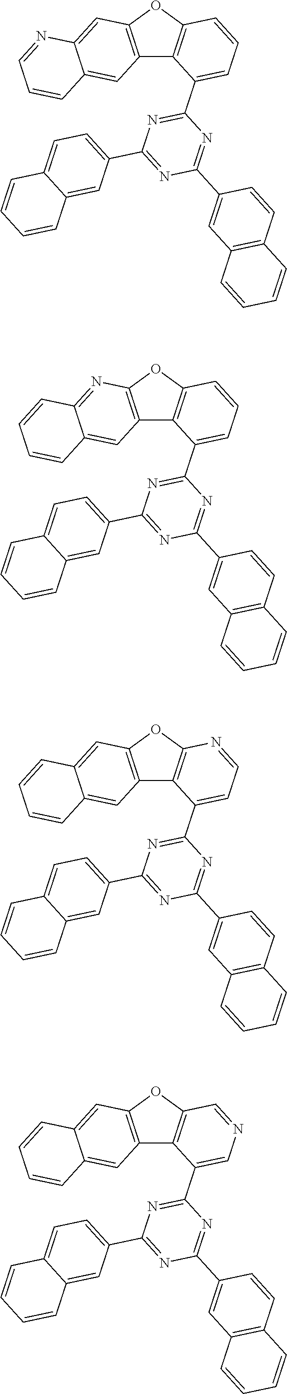 [SMILES: c1ccc2cc(-c3nc(-c4ccc5ccccc5c4)nc(-c4cccc5oc6cc7ncccc7cc6c45)n3)ccc2c1 ; c1ccc2cc(-c3nc(-c4ccc5ccccc5c4)nc(-c4cccc5oc6nc7ccccc7cc6c45)n3)ccc2c1 ; c1ccc2cc(-c3nc(-c4ccc5ccccc5c4)nc(-c4ccnc5oc6cc7ccccc7cc6c45)n3)ccc2c1 ; c1ccc2cc(-c3nc(-c4ccc5ccccc5c4)nc(-c4cncc5oc6cc7ccccc7cc6c45)n3)ccc2c1]

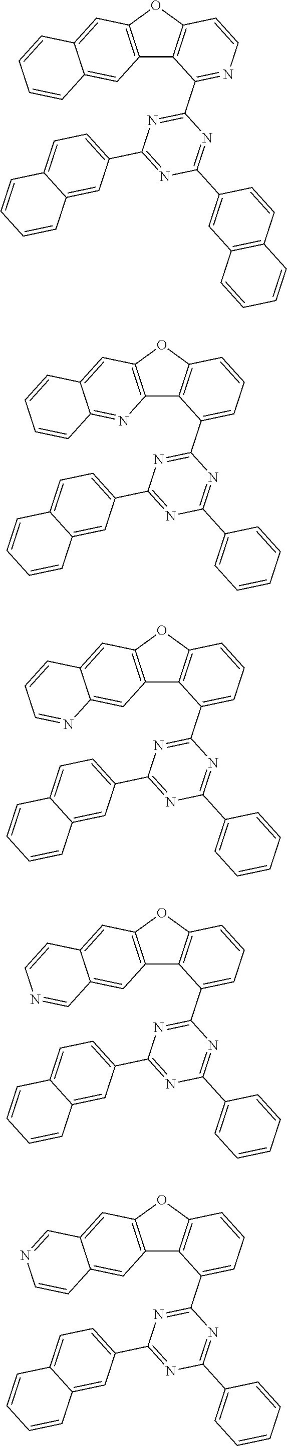 [SMILES: c1ccc2cc(-c3nc(-c4ccc5ccccc5c4)nc(-c4nccc5oc6cc7ccccc7cc6c45)n3)ccc2c1 ; c1ccc(-c2nc(-c3ccc4ccccc4c3)nc(-c3cccc4oc5cc6ccccc6nc5c34)n2)cc1 ; c1ccc(-c2nc(-c3ccc4ccccc4c3)nc(-c3cccc4oc5cc6cccnc6cc5c34)n2)cc1 ; c1ccc(-c2nc(-c3ccc4ccccc4c3)nc(-c3cccc4oc5cc6ccncc6cc5c34)n2)cc1 ; c1ccc(-c2nc(-c3ccc4ccccc4c3)nc(-c3cccc4oc5cc6cnccc6cc5c34)n2)cc1]

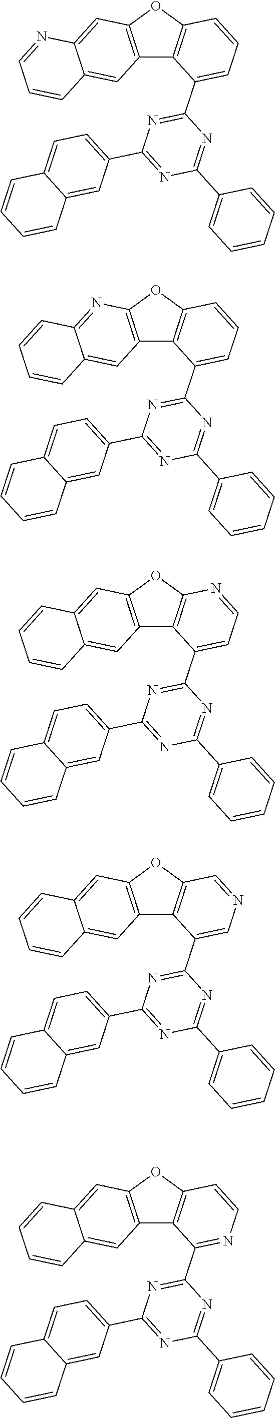 [SMILES: c1ccc(-c2nc(-c3ccc4ccccc4c3)nc(-c3cccc4oc5cc6ncccc6cc5c34)n2)cc1 ; c1ccc(-c2nc(-c3ccc4ccccc4c3)nc(-c3cccc4oc5nc6ccccc6cc5c34)n2)cc1 ; c1ccc(-c2nc(-c3ccc4ccccc4c3)nc(-c3ccnc4oc5cc6ccccc6cc5c34)n2)cc1 ; c1ccc(-c2nc(-c3ccc4ccccc4c3)nc(-c3cncc4oc5cc6ccccc6cc5c34)n2)cc1 ; c1ccc(-c2nc(-c3ccc4ccccc4c3)nc(-c3nccc4oc5cc6ccccc6cc5c34)n2)cc1]

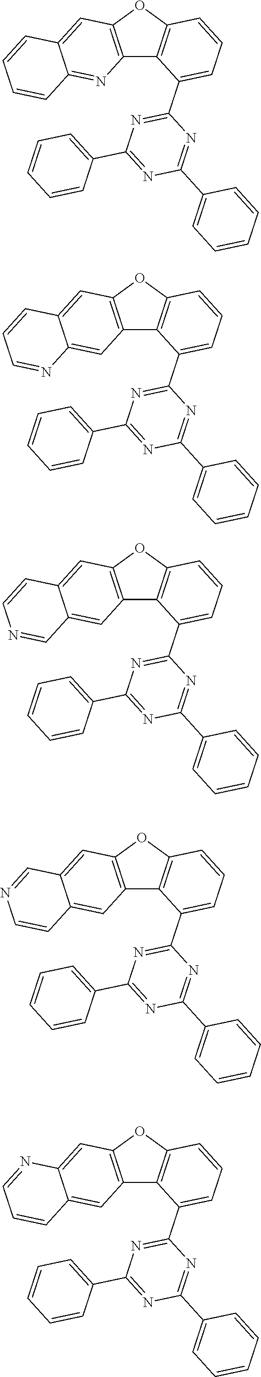

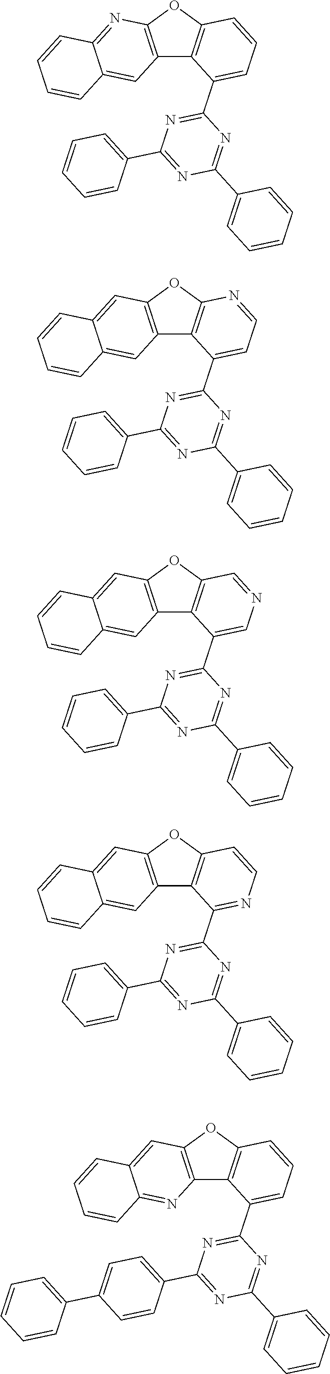

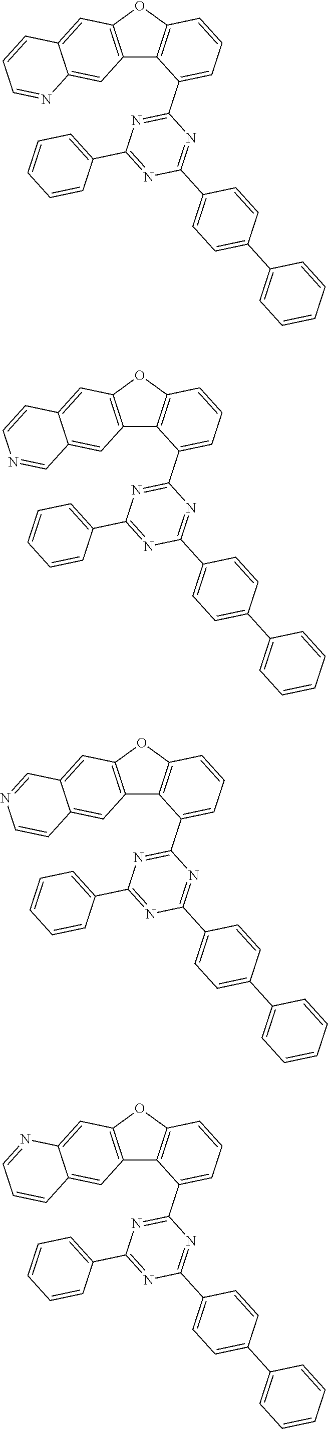 [SMILES: c1ccc(-c2ccc(-c3nc(-c4ccccc4)nc(-c4cccc5oc6cc7cccnc7cc6c45)n3)cc2)cc1 ; c1ccc(-c2ccc(-c3nc(-c4ccccc4)nc(-c4cccc5oc6cc7ccncc7cc6c45)n3)cc2)cc1 ; c1ccc(-c2ccc(-c3nc(-c4ccccc4)nc(-c4cccc5oc6cc7cnccc7cc6c45)n3)cc2)cc1 ; c1ccc(-c2ccc(-c3nc(-c4ccccc4)nc(-c4cccc5oc6cc7ncccc7cc6c45)n3)cc2)cc1]

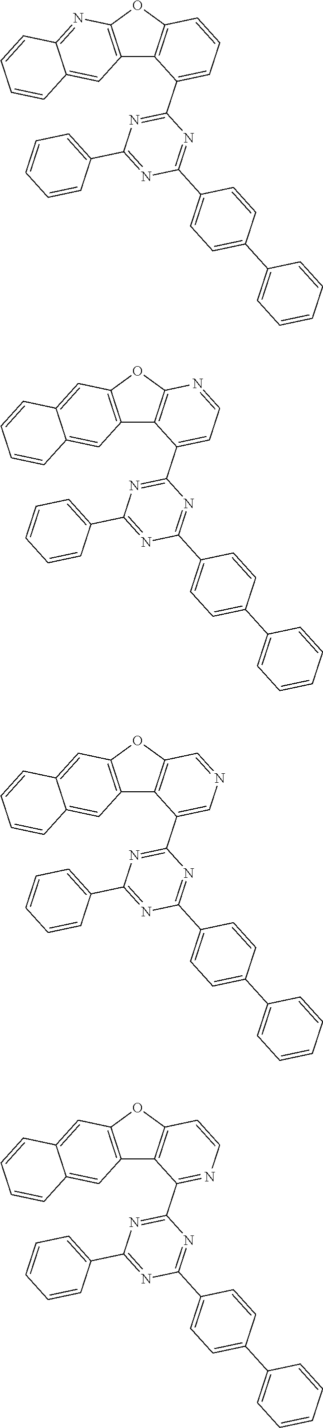 [SMILES: c1ccc(-c2ccc(-c3nc(-c4ccccc4)nc(-c4cccc5oc6nc7ccccc7cc6c45)n3)cc2)cc1 ; c1ccc(-c2ccc(-c3nc(-c4ccccc4)nc(-c4ccnc5oc6cc7ccccc7cc6c45)n3)cc2)cc1 ; c1ccc(-c2ccc(-c3nc(-c4ccccc4)nc(-c4cncc5oc6cc7ccccc7cc6c45)n3)cc2)cc1 ; c1ccc(-c2ccc(-c3nc(-c4ccccc4)nc(-c4nccc5oc6cc7ccccc7cc6c45)n3)cc2)cc1]

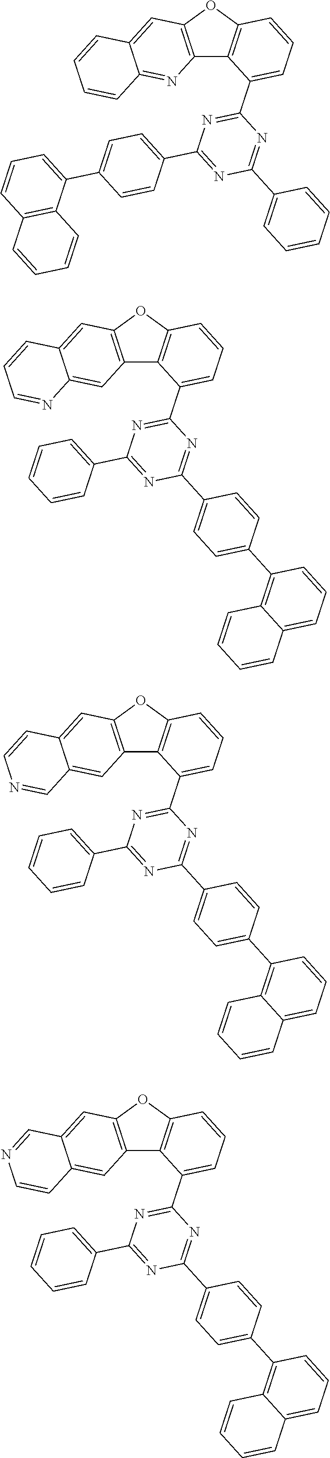 [SMILES: c1ccc(-c2nc(-c3ccc(-c4cccc5ccccc45)cc3)nc(-c3cccc4oc5cc6ccccc6nc5c34)n2)cc1 ; c1ccc(-c2nc(-c3ccc(-c4cccc5ccccc45)cc3)nc(-c3cccc4oc5cc6cccnc6cc5c34)n2)cc1 ; c1ccc(-c2nc(-c3ccc(-c4cccc5ccccc45)cc3)nc(-c3cccc4oc5cc6ccncc6cc5c34)n2)cc1 ; c1ccc(-c2nc(-c3ccc(-c4cccc5ccccc45)cc3)nc(-c3cccc4oc5cc6cnccc6cc5c34)n2)cc1]

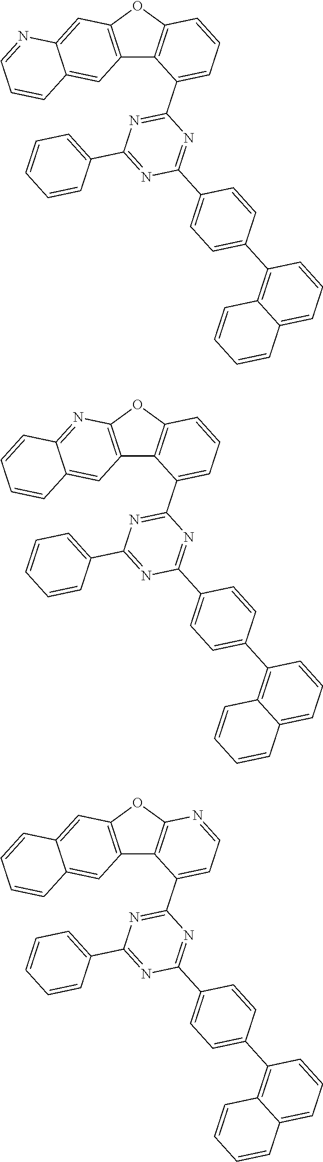 [SMILES: c1ccc(-c2nc(-c3ccc(-c4cccc5ccccc45)cc3)nc(-c3cccc4oc5cc6ncccc6cc5c34)n2)cc1 ; c1ccc(-c2nc(-c3ccc(-c4cccc5ccccc45)cc3)nc(-c3cccc4oc5nc6ccccc6cc5c34)n2)cc1 ; c1ccc(-c2nc(-c3ccc(-c4cccc5ccccc45)cc3)nc(-c3ccnc4oc5cc6ccccc6cc5c34)n2)cc1]

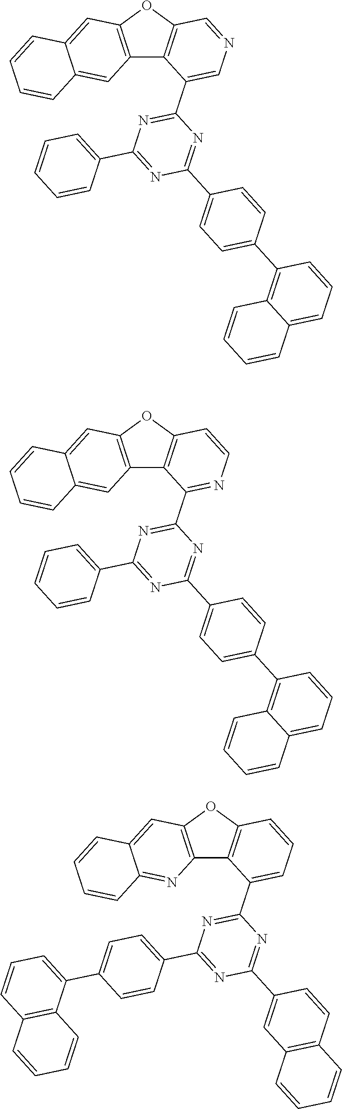 [SMILES: c1ccc(-c2nc(-c3ccc(-c4cccc5ccccc45)cc3)nc(-c3cncc4oc5cc6ccccc6cc5c34)n2)cc1 ; c1ccc(-c2nc(-c3ccc(-c4cccc5ccccc45)cc3)nc(-c3nccc4oc5cc6ccccc6cc5c34)n2)cc1 ; c1ccc2cc(-c3nc(-c4ccc(-c5cccc6ccccc56)cc4)nc(-c4cccc5oc6cc7ccccc7nc6c45)n3)ccc2c1]

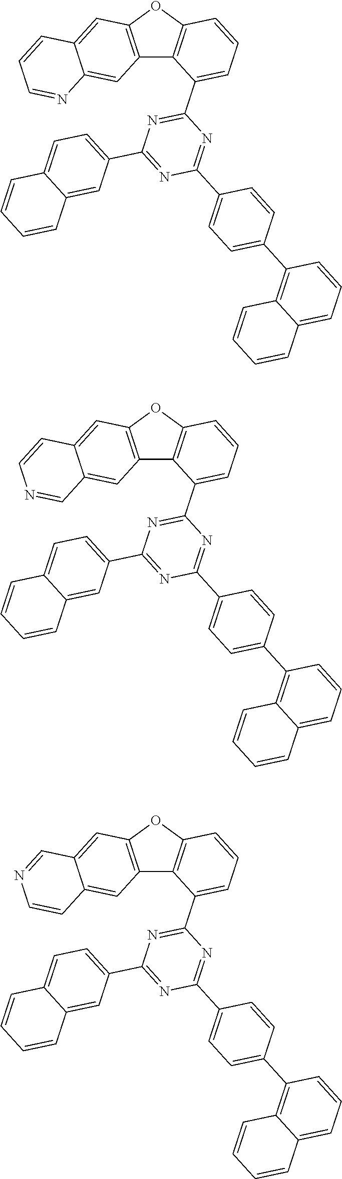 [SMILES: c1ccc2cc(-c3nc(-c4ccc(-c5cccc6ccccc56)cc4)nc(-c4cccc5oc6cc7cccnc7cc6c45)n3)ccc2c1 ; c1ccc2cc(-c3nc(-c4ccc(-c5cccc6ccccc56)cc4)nc(-c4cccc5oc6cc7ccncc7cc6c45)n3)ccc2c1 ; c1ccc2cc(-c3nc(-c4ccc(-c5cccc6ccccc56)cc4)nc(-c4cccc5oc6cc7cnccc7cc6c45)n3)ccc2c1]

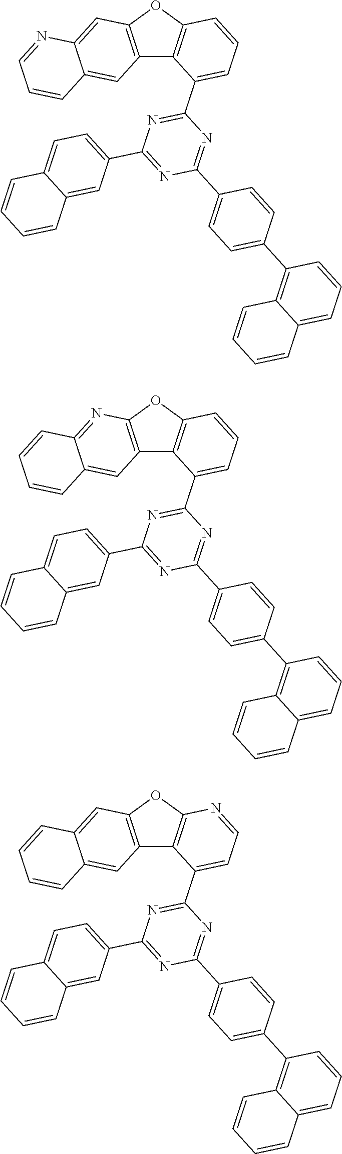 [SMILES: c1ccc2cc(-c3nc(-c4ccc(-c5cccc6ccccc56)cc4)nc(-c4cccc5oc6cc7ncccc7cc6c45)n3)ccc2c1 ; c1ccc2cc(-c3nc(-c4ccc(-c5cccc6ccccc56)cc4)nc(-c4cccc5oc6nc7ccccc7cc6c45)n3)ccc2c1 ; c1ccc2cc(-c3nc(-c4ccc(-c5cccc6ccccc56)cc4)nc(-c4ccnc5oc6cc7ccccc7cc6c45)n3)ccc2c1]

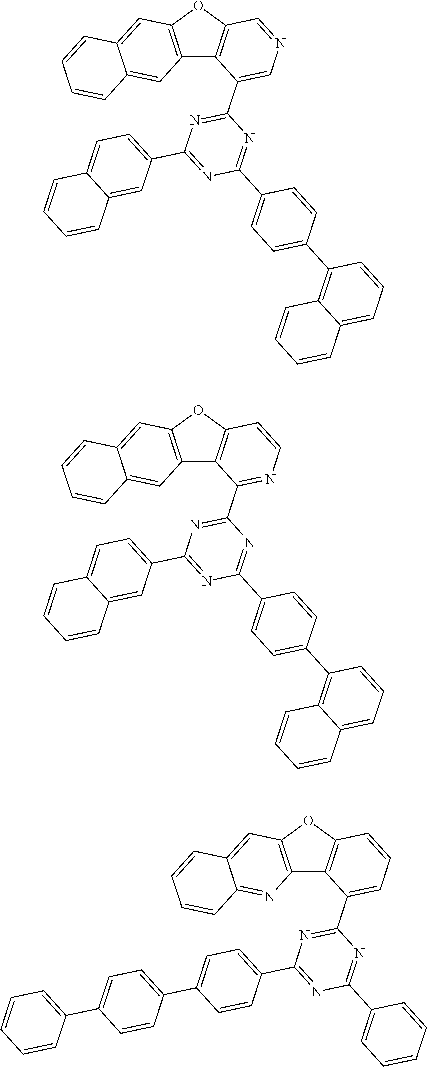 [SMILES: c1ccc2cc(-c3nc(-c4ccc(-c5cccc6ccccc56)cc4)nc(-c4cncc5oc6cc7ccccc7cc6c45)n3)ccc2c1 ; c1ccc2cc(-c3nc(-c4ccc(-c5cccc6ccccc56)cc4)nc(-c4nccc5oc6cc7ccccc7cc6c45)n3)ccc2c1 ; c1ccc(-c2ccc(-c3ccc(-c4nc(-c5ccccc5)nc(-c5cccc6oc7cc8ccccc8nc7c56)n4)cc3)cc2)cc1]

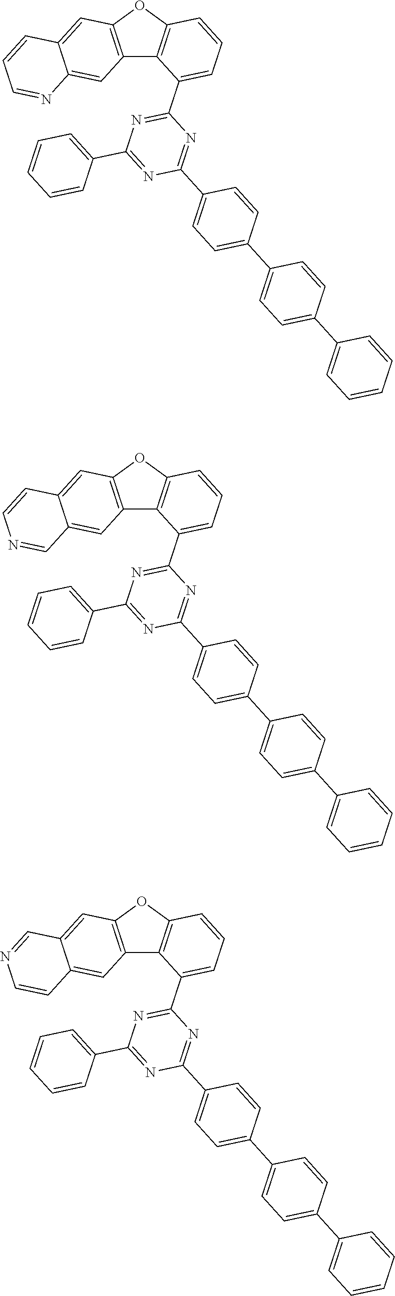 [SMILES: c1ccc(-c2ccc(-c3ccc(-c4nc(-c5ccccc5)nc(-c5cccc6oc7cc8cccnc8cc7c56)n4)cc3)cc2)cc1 ; c1ccc(-c2ccc(-c3ccc(-c4nc(-c5ccccc5)nc(-c5cccc6oc7cc8ccncc8cc7c56)n4)cc3)cc2)cc1 ; c1ccc(-c2ccc(-c3ccc(-c4nc(-c5ccccc5)nc(-c5cccc6oc7cc8cnccc8cc7c56)n4)cc3)cc2)cc1]

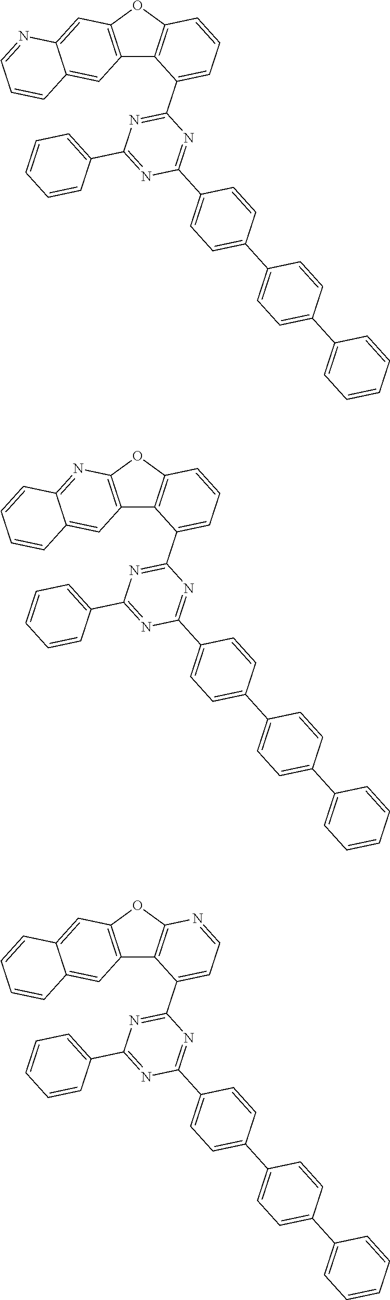 [SMILES: c1ccc(-c2ccc(-c3ccc(-c4nc(-c5ccccc5)nc(-c5cccc6oc7cc8ncccc8cc7c56)n4)cc3)cc2)cc1 ; c1ccc(-c2ccc(-c3ccc(-c4nc(-c5ccccc5)nc(-c5cccc6oc7nc8ccccc8cc7c56)n4)cc3)cc2)cc1 ; c1ccc(-c2ccc(-c3ccc(-c4nc(-c5ccccc5)nc(-c5ccnc6oc7cc8ccccc8cc7c56)n4)cc3)cc2)cc1]

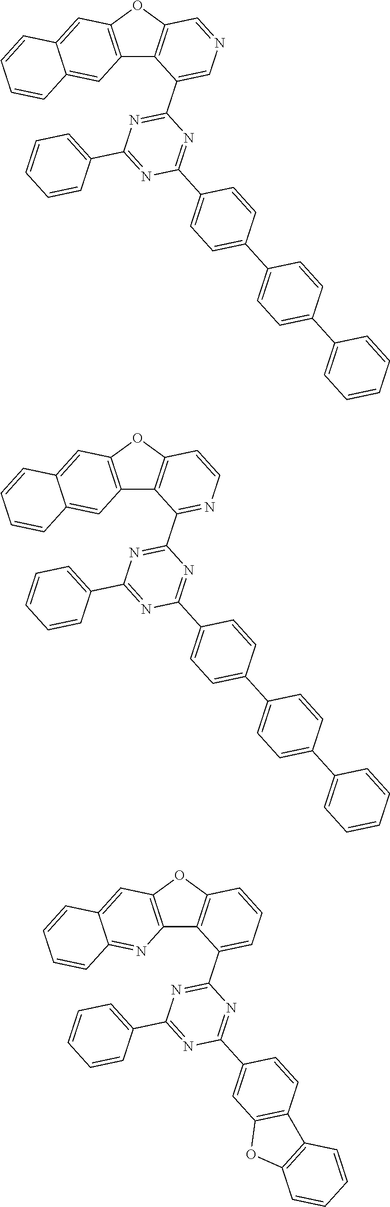 [SMILES: c1ccc(-c2ccc(-c3ccc(-c4nc(-c5ccccc5)nc(-c5cncc6oc7cc8ccccc8cc7c56)n4)cc3)cc2)cc1 ; c1ccc(-c2ccc(-c3ccc(-c4nc(-c5ccccc5)nc(-c5nccc6oc7cc8ccccc8cc7c56)n4)cc3)cc2)cc1 ; c1ccc(-c2nc(-c3ccc4c(c3)oc3ccccc34)nc(-c3cccc4oc5cc6ccccc6nc5c34)n2)cc1]

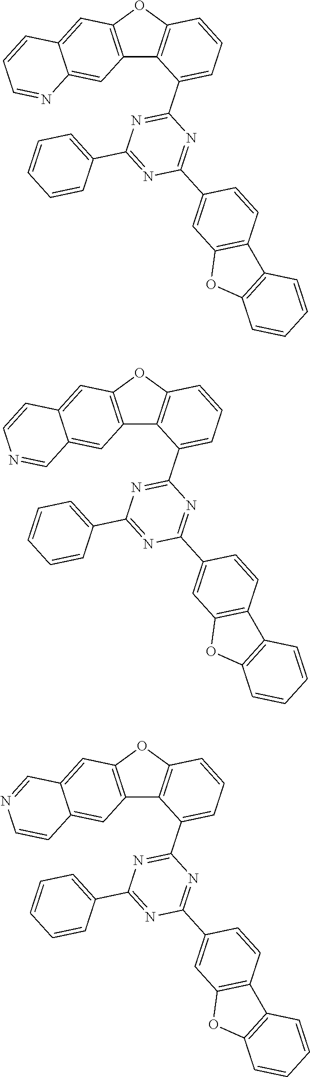 [SMILES: c1ccc(-c2nc(-c3ccc4c(c3)oc3ccccc34)nc(-c3cccc4oc5cc6cccnc6cc5c34)n2)cc1 ; c1ccc(-c2nc(-c3ccc4c(c3)oc3ccccc34)nc(-c3cccc4oc5cc6ccncc6cc5c34)n2)cc1 ; c1ccc(-c2nc(-c3ccc4c(c3)oc3ccccc34)nc(-c3cccc4oc5cc6cnccc6cc5c34)n2)cc1]

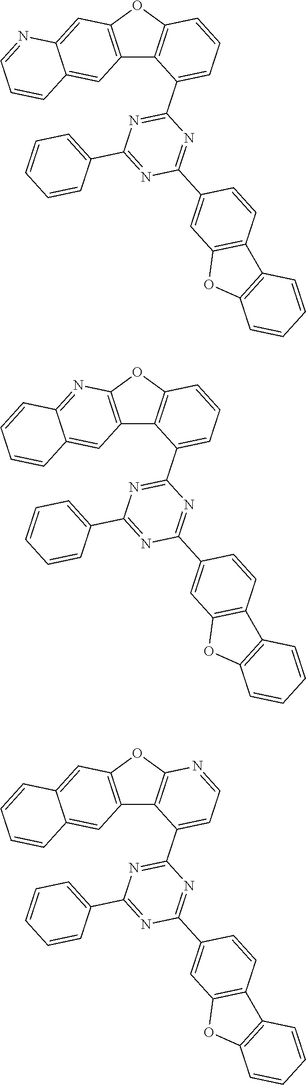 [SMILES: c1ccc(-c2nc(-c3ccc4c(c3)oc3ccccc34)nc(-c3cccc4oc5cc6ncccc6cc5c34)n2)cc1 ; c1ccc(-c2nc(-c3ccc4c(c3)oc3ccccc34)nc(-c3cccc4oc5nc6ccccc6cc5c34)n2)cc1 ; c1ccc(-c2nc(-c3ccc4c(c3)oc3ccccc34)nc(-c3ccnc4oc5cc6ccccc6cc5c34)n2)cc1]

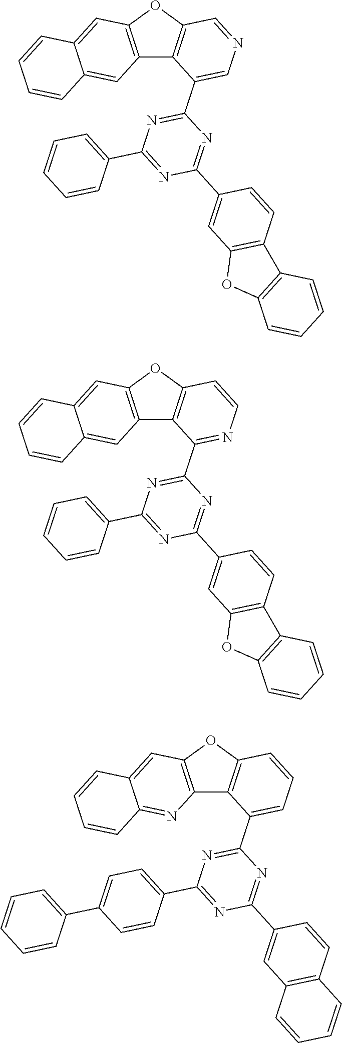 [SMILES: c1ccc(-c2nc(-c3ccc4c(c3)oc3ccccc34)nc(-c3cncc4oc5cc6ccccc6cc5c34)n2)cc1 ; c1ccc(-c2nc(-c3ccc4c(c3)oc3ccccc34)nc(-c3nccc4oc5cc6ccccc6cc5c34)n2)cc1 ; c1ccc(-c2ccc(-c3nc(-c4ccc5ccccc5c4)nc(-c4cccc5oc6cc7ccccc7nc6c45)n3)cc2)cc1]

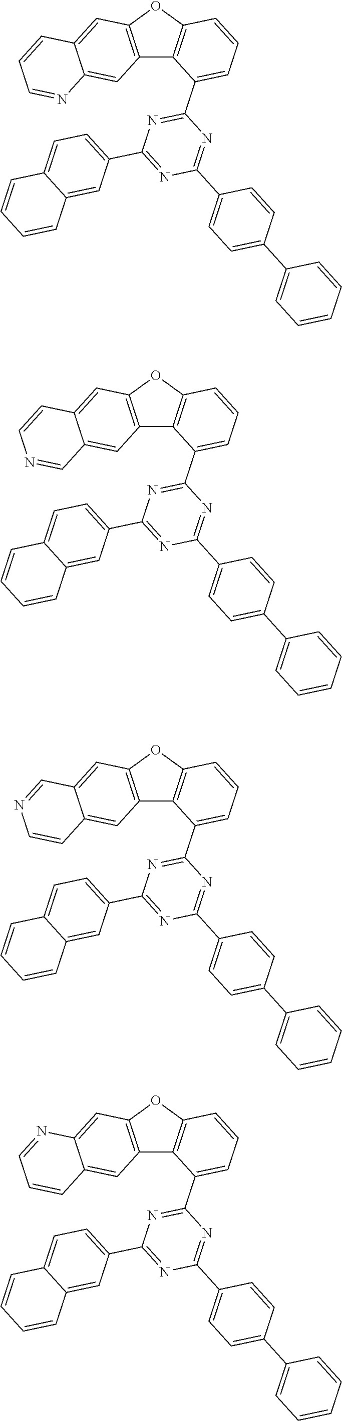 [SMILES: c1ccc(-c2ccc(-c3nc(-c4ccc5ccccc5c4)nc(-c4cccc5oc6cc7cccnc7cc6c45)n3)cc2)cc1 ; c1ccc(-c2ccc(-c3nc(-c4ccc5ccccc5c4)nc(-c4cccc5oc6cc7ccncc7cc6c45)n3)cc2)cc1 ; c1ccc(-c2ccc(-c3nc(-c4ccc5ccccc5c4)nc(-c4cccc5oc6cc7cnccc7cc6c45)n3)cc2)cc1 ; c1ccc(-c2ccc(-c3nc(-c4ccc5ccccc5c4)nc(-c4cccc5oc6cc7ncccc7cc6c45)n3)cc2)cc1]

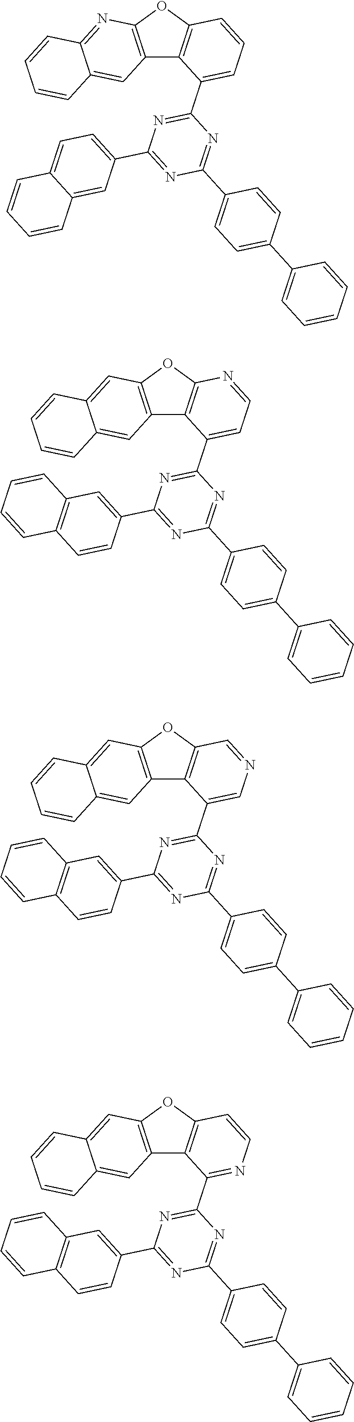 [SMILES: c1ccc(-c2ccc(-c3nc(-c4ccc5ccccc5c4)nc(-c4cccc5oc6nc7ccccc7cc6c45)n3)cc2)cc1 ; c1ccc(-c2ccc(-c3nc(-c4ccc5ccccc5c4)nc(-c4ccnc5oc6cc7ccccc7cc6c45)n3)cc2)cc1 ; c1ccc(-c2ccc(-c3nc(-c4ccc5ccccc5c4)nc(-c4cncc5oc6cc7ccccc7cc6c45)n3)cc2)cc1 ; c1ccc(-c2ccc(-c3nc(-c4ccc5ccccc5c4)nc(-c4nccc5oc6cc7ccccc7cc6c45)n3)cc2)cc1]

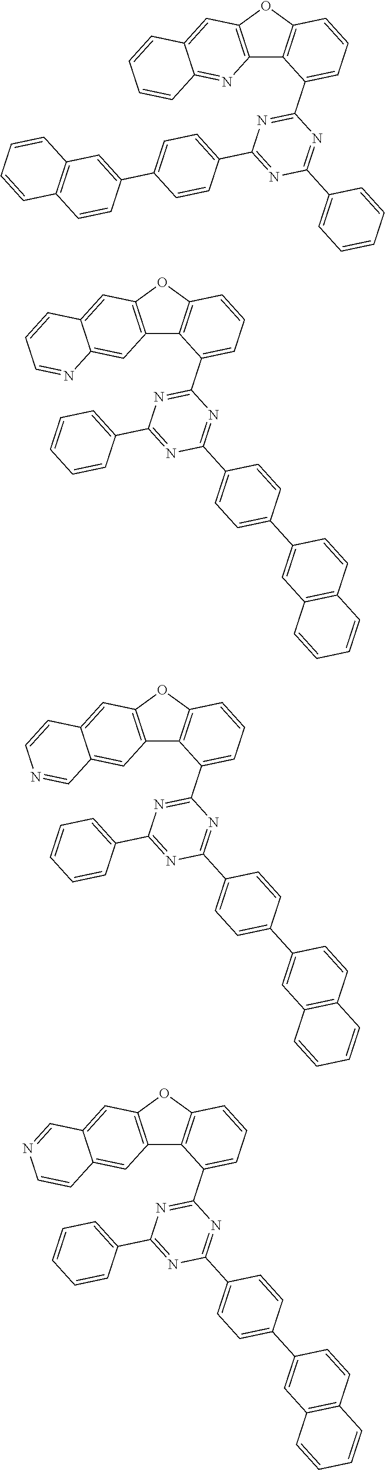 [SMILES: c1ccc(-c2nc(-c3ccc(-c4ccc5ccccc5c4)cc3)nc(-c3cccc4oc5cc6ccccc6nc5c34)n2)cc1 ; c1ccc(-c2nc(-c3ccc(-c4ccc5ccccc5c4)cc3)nc(-c3cccc4oc5cc6cccnc6cc5c34)n2)cc1 ; c1ccc(-c2nc(-c3ccc(-c4ccc5ccccc5c4)cc3)nc(-c3cccc4oc5cc6ccncc6cc5c34)n2)cc1 ; c1ccc(-c2nc(-c3ccc(-c4ccc5ccccc5c4)cc3)nc(-c3cccc4oc5cc6cnccc6cc5c34)n2)cc1]

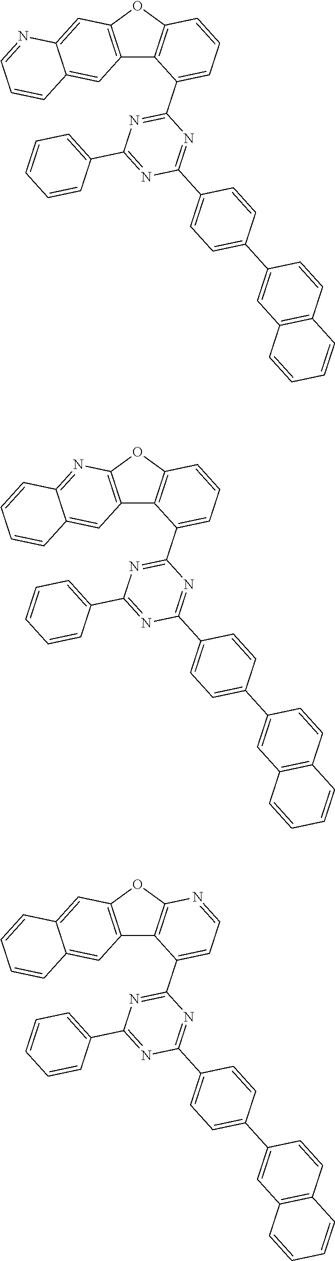 [SMILES: c1ccc(-c2nc(-c3ccc(-c4ccc5ccccc5c4)cc3)nc(-c3cccc4oc5cc6ncccc6cc5c34)n2)cc1 ; c1ccc(-c2nc(-c3ccc(-c4ccc5ccccc5c4)cc3)nc(-c3cccc4oc5nc6ccccc6cc5c34)n2)cc1 ; c1ccc(-c2nc(-c3ccc(-c4ccc5ccccc5c4)cc3)nc(-c3ccnc4oc5cc6ccccc6cc5c34)n2)cc1]

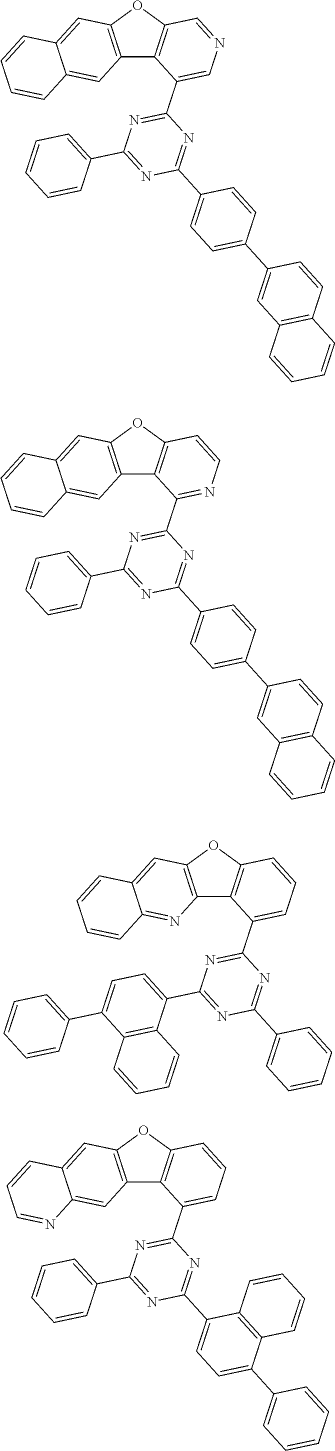

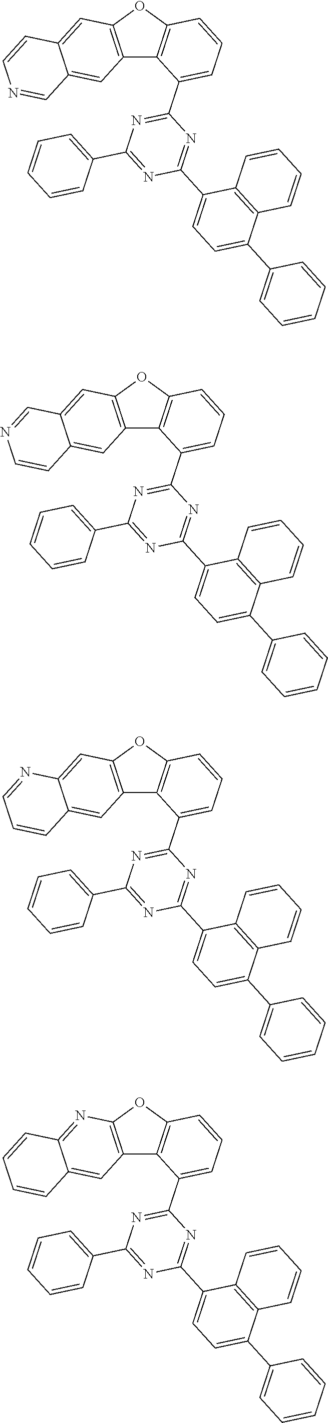 [SMILES: c1ccc(-c2nc(-c3ccc(-c4ccccc4)c4ccccc34)nc(-c3cccc4oc5cc6ccncc6cc5c34)n2)cc1 ; c1ccc(-c2nc(-c3ccc(-c4ccccc4)c4ccccc34)nc(-c3cccc4oc5cc6cnccc6cc5c34)n2)cc1 ; c1ccc(-c2nc(-c3ccc(-c4ccccc4)c4ccccc34)nc(-c3cccc4oc5cc6ncccc6cc5c34)n2)cc1 ; c1ccc(-c2nc(-c3ccc(-c4ccccc4)c4ccccc34)nc(-c3cccc4oc5nc6ccccc6cc5c34)n2)cc1]

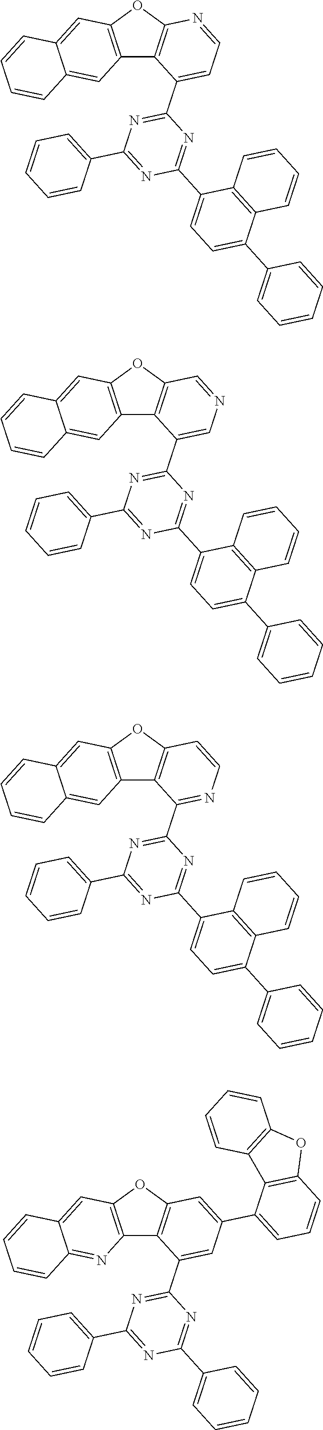 [SMILES: c1ccc(-c2nc(-c3ccc(-c4ccccc4)c4ccccc34)nc(-c3ccnc4oc5cc6ccccc6cc5c34)n2)cc1 ; c1ccc(-c2nc(-c3ccc(-c4ccccc4)c4ccccc34)nc(-c3cncc4oc5cc6ccccc6cc5c34)n2)cc1 ; c1ccc(-c2nc(-c3ccc(-c4ccccc4)c4ccccc34)nc(-c3nccc4oc5cc6ccccc6cc5c34)n2)cc1 ; c1ccc(-c2nc(-c3ccccc3)nc(-c3cc(-c4cccc5oc6ccccc6c45)cc4oc5cc6ccccc6nc5c34)n2)cc1]

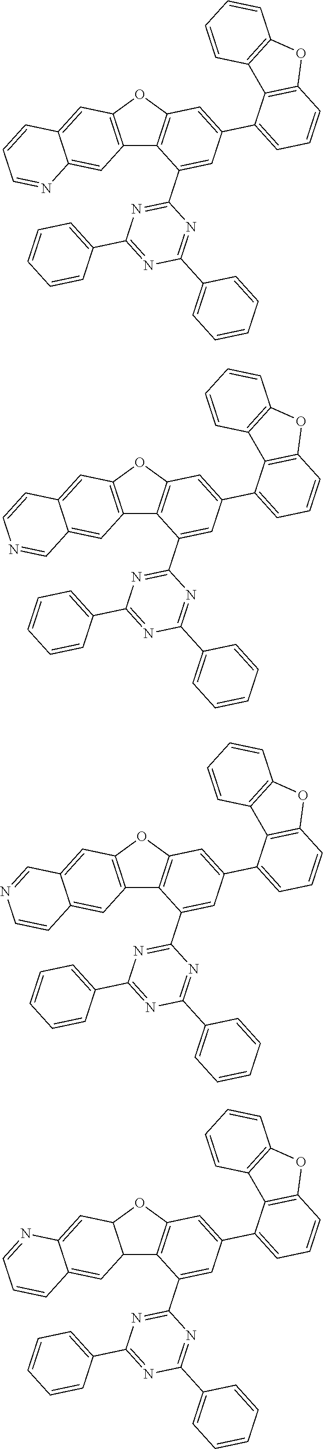

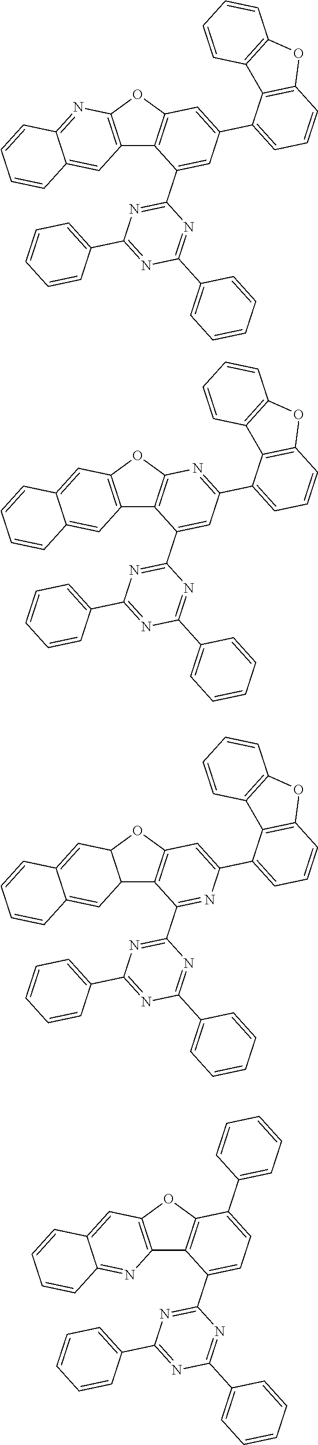

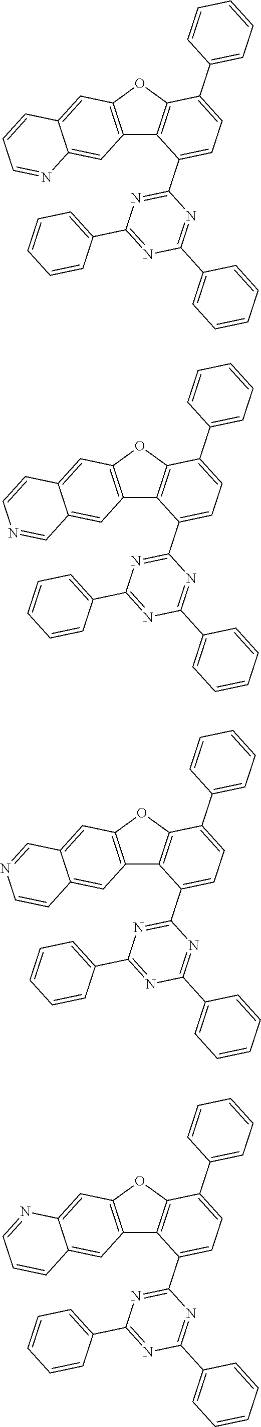

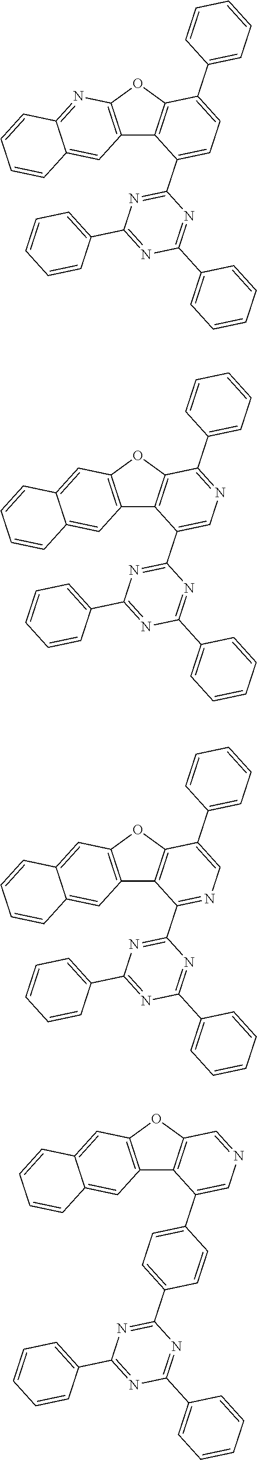

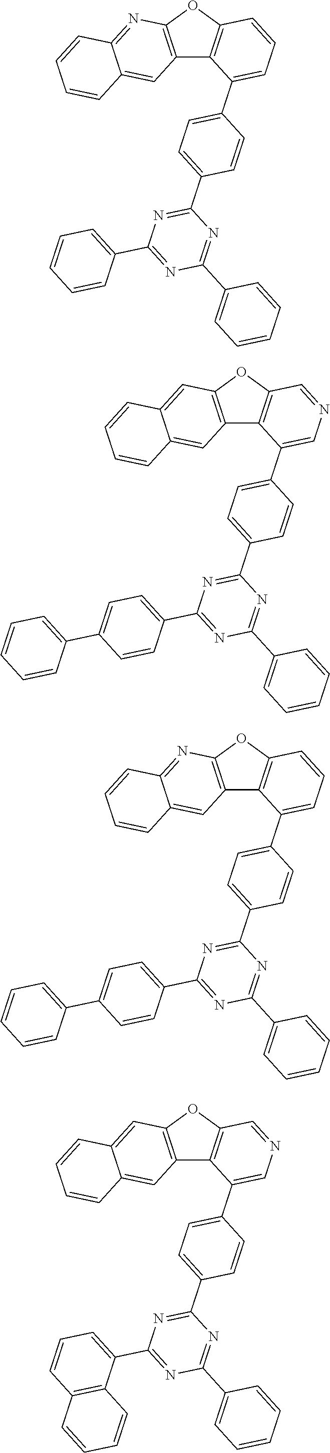

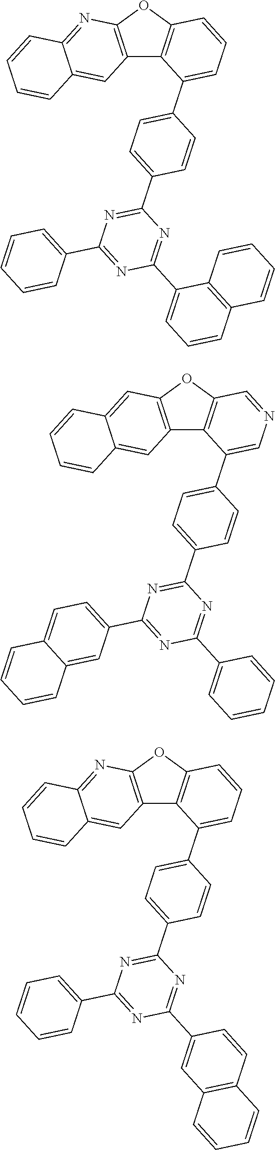

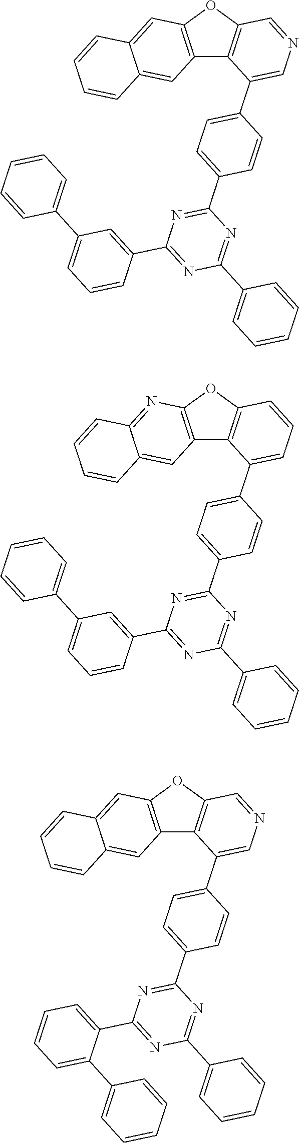 [SMILES: c1ccc(-c2cccc(-c3nc(-c4ccccc4)nc(-c4ccc(-c5cncc6oc7cc8ccccc8cc7c56)cc4)n3)c2)cc1 ; c1ccc(-c2cccc(-c3nc(-c4ccccc4)nc(-c4ccc(-c5cccc6oc7nc8ccccc8cc7c56)cc4)n3)c2)cc1 ; c1ccc(-c2nc(-c3ccc(-c4cncc5oc6cc7ccccc7cc6c45)cc3)nc(-c3ccccc3-c3ccccc3)n2)cc1]

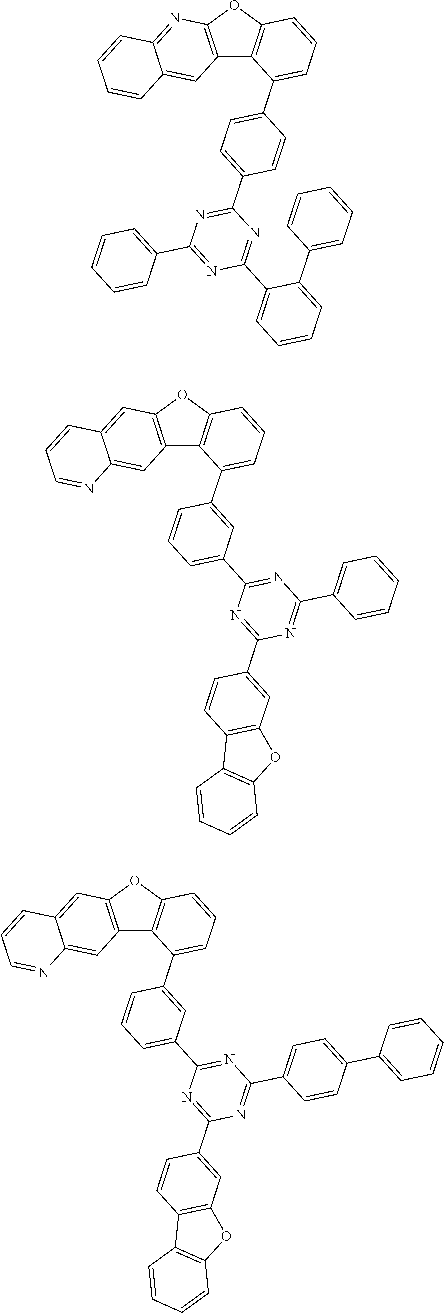 [SMILES: c1ccc(-c2nc(-c3ccc(-c4cccc5oc6nc7ccccc7cc6c45)cc3)nc(-c3ccccc3-c3ccccc3)n2)cc1 ; c1ccc(-c2nc(-c3cccc(-c4cccc5oc6cc7cccnc7cc6c45)c3)nc(-c3ccc4c(c3)oc3ccccc34)n2)cc1 ; c1ccc(-c2ccc(-c3nc(-c4cccc(-c5cccc6oc7cc8cccnc8cc7c56)c4)nc(-c4ccc5c(c4)oc4ccccc45)n3)cc2)cc1]

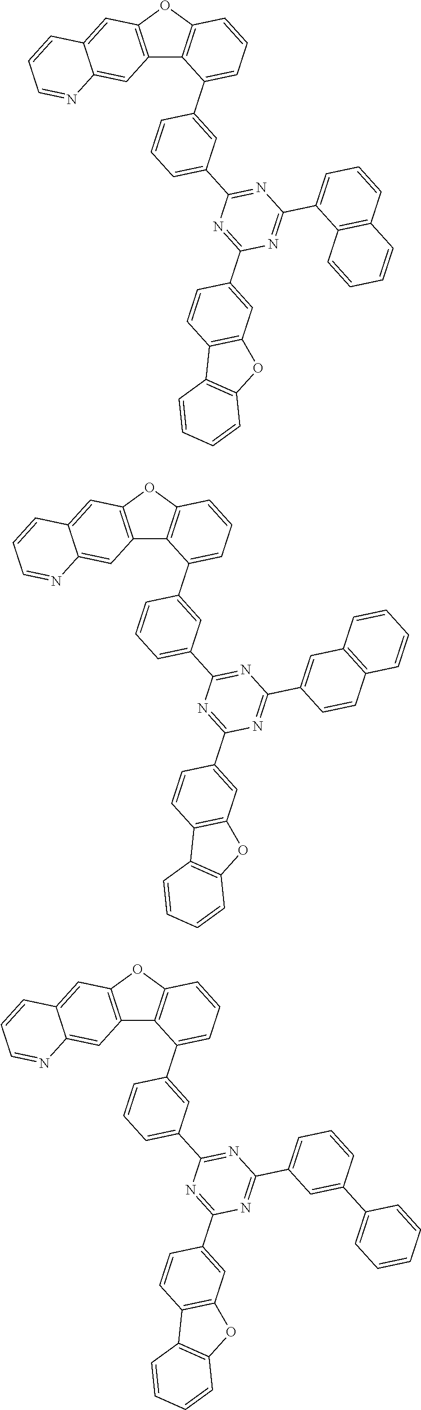 [SMILES: c1cc(-c2nc(-c3ccc4c(c3)oc3ccccc34)nc(-c3cccc4ccccc34)n2)cc(-c2cccc3oc4cc5cccnc5cc4c23)c1 ; c1cc(-c2nc(-c3ccc4ccccc4c3)nc(-c3ccc4c(c3)oc3ccccc34)n2)cc(-c2cccc3oc4cc5cccnc5cc4c23)c1 ; c1ccc(-c2cccc(-c3nc(-c4cccc(-c5cccc6oc7cc8cccnc8cc7c56)c4)nc(-c4ccc5c(c4)oc4ccccc45)n3)c2)cc1]

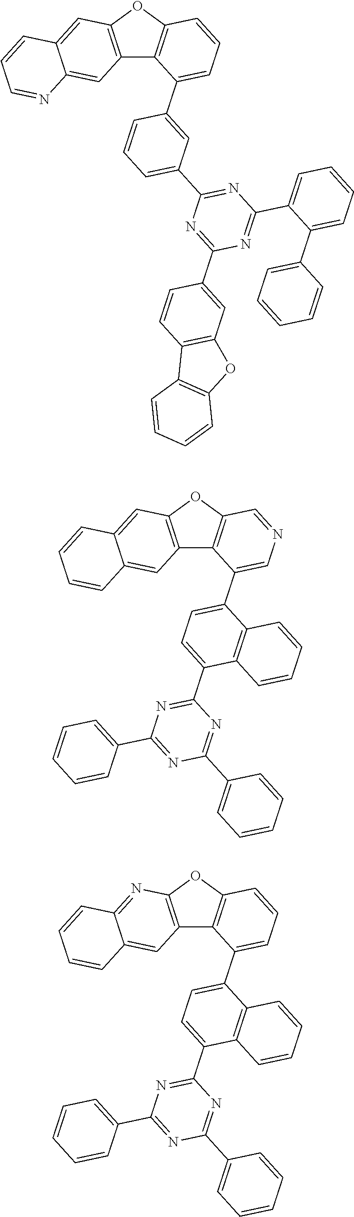 [SMILES: c1ccc(-c2ccccc2-c2nc(-c3cccc(-c4cccc5oc6cc7cccnc7cc6c45)c3)nc(-c3ccc4c(c3)oc3ccccc34)n2)cc1 ; c1ccc(-c2nc(-c3ccccc3)nc(-c3ccc(-c4cncc5oc6cc7ccccc7cc6c45)c4ccccc34)n2)cc1 ; c1ccc(-c2nc(-c3ccccc3)nc(-c3ccc(-c4cccc5oc6nc7ccccc7cc6c45)c4ccccc34)n2)cc1]

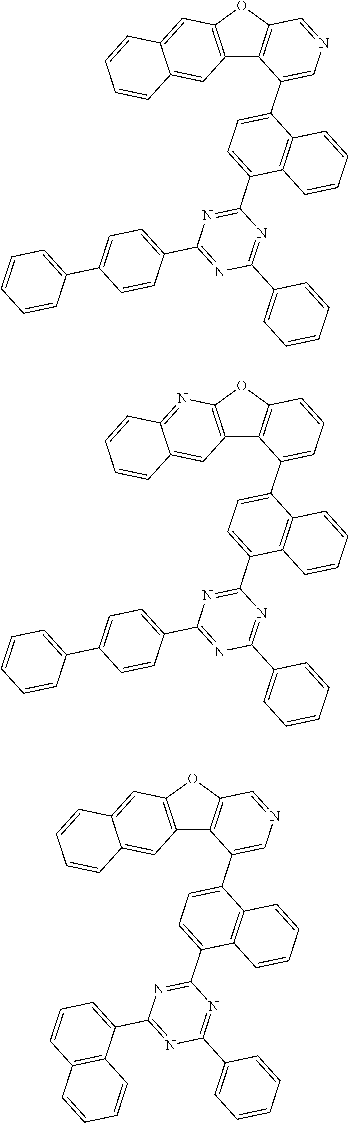 [SMILES: c1ccc(-c2ccc(-c3nc(-c4ccccc4)nc(-c4ccc(-c5cncc6oc7cc8ccccc8cc7c56)c5ccccc45)n3)cc2)cc1 ; c1ccc(-c2ccc(-c3nc(-c4ccccc4)nc(-c4ccc(-c5cccc6oc7nc8ccccc8cc7c56)c5ccccc45)n3)cc2)cc1 ; c1ccc(-c2nc(-c3cccc4ccccc34)nc(-c3ccc(-c4cncc5oc6cc7ccccc7cc6c45)c4ccccc34)n2)cc1]

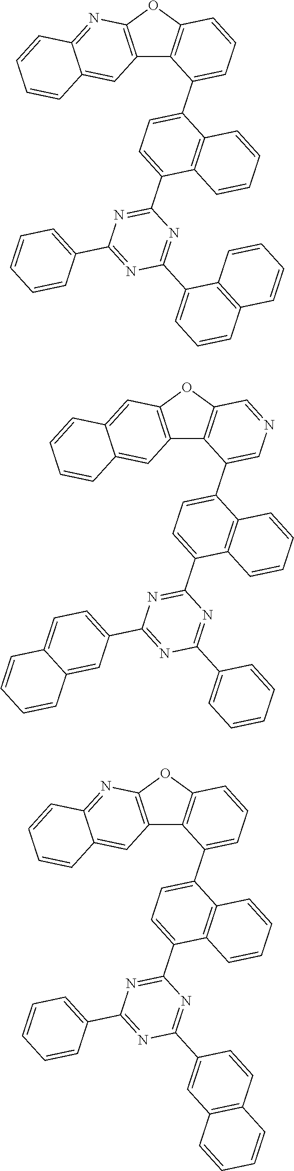

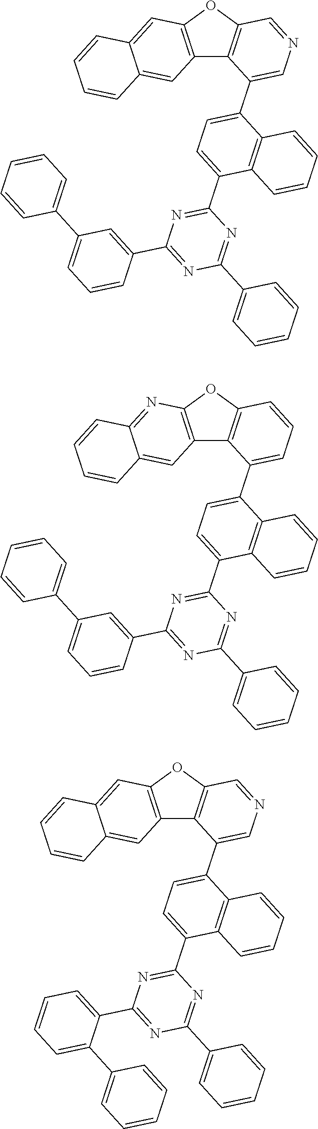 [SMILES: c1ccc(-c2cccc(-c3nc(-c4ccccc4)nc(-c4ccc(-c5cncc6oc7cc8ccccc8cc7c56)c5ccccc45)n3)c2)cc1 ; c1ccc(-c2cccc(-c3nc(-c4ccccc4)nc(-c4ccc(-c5cccc6oc7nc8ccccc8cc7c56)c5ccccc45)n3)c2)cc1 ; c1ccc(-c2nc(-c3ccccc3-c3ccccc3)nc(-c3ccc(-c4cncc5oc6cc7ccccc7cc6c45)c4ccccc34)n2)cc1]

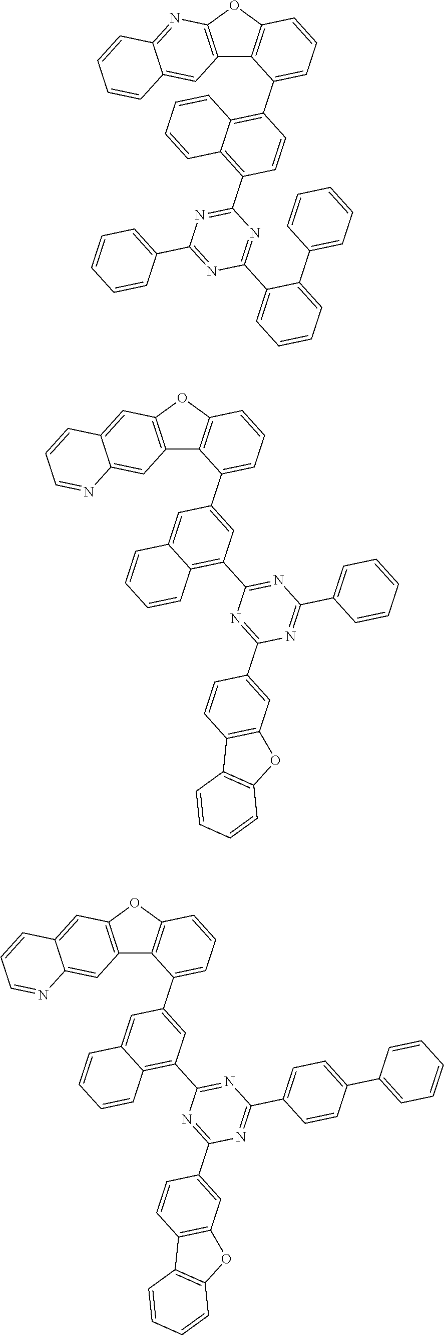 [SMILES: c1ccc(-c2nc(-c3ccccc3-c3ccccc3)nc(-c3ccc(-c4cccc5oc6nc7ccccc7cc6c45)c4ccccc34)n2)cc1 ; c1ccc(-c2nc(-c3ccc4c(c3)oc3ccccc34)nc(-c3cc(-c4cccc5oc6cc7cccnc7cc6c45)cc4ccccc34)n2)cc1 ; c1ccc(-c2ccc(-c3nc(-c4ccc5c(c4)oc4ccccc45)nc(-c4cc(-c5cccc6oc7cc8cccnc8cc7c56)cc5ccccc45)n3)cc2)cc1]

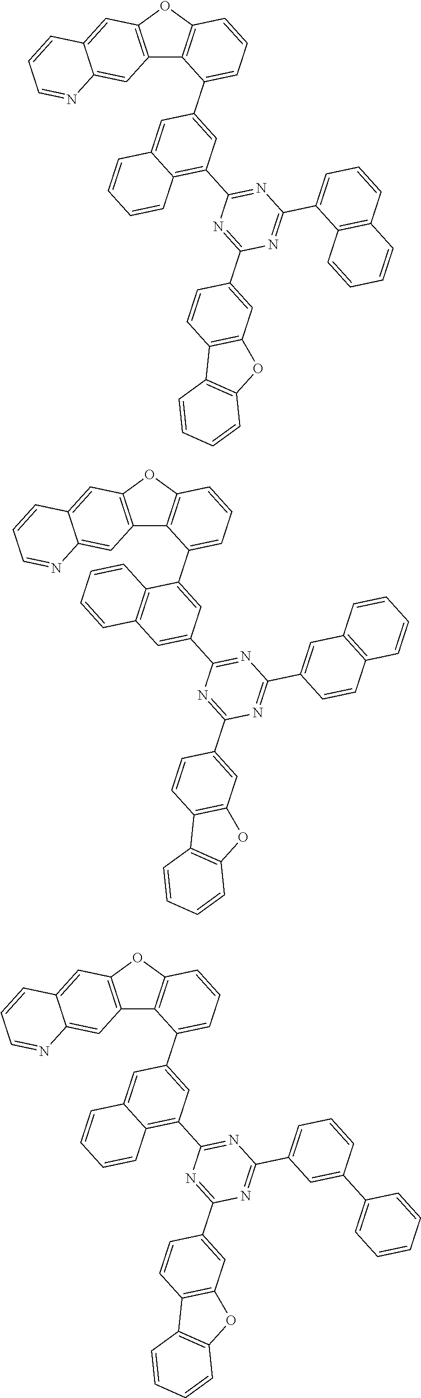

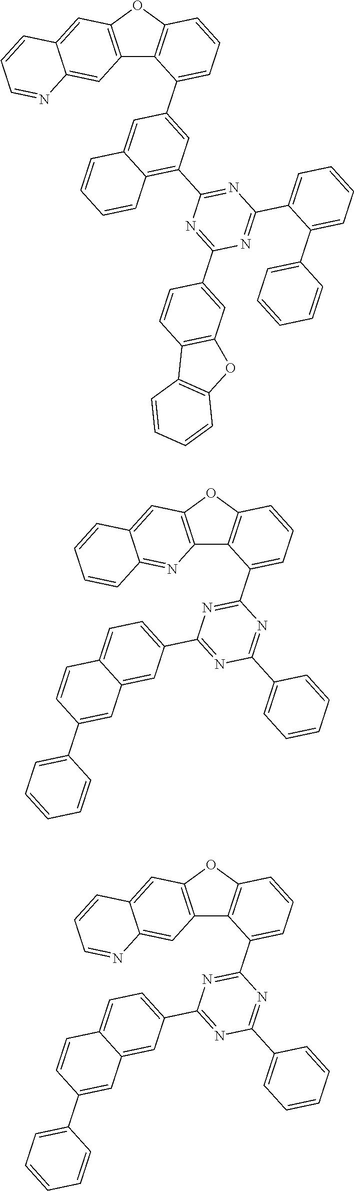 [SMILES: c1ccc(-c2ccccc2-c2nc(-c3ccc4c(c3)oc3ccccc34)nc(-c3cc(-c4cccc5oc6cc7cccnc7cc6c45)cc4ccccc34)n2)cc1 ; c1ccc(-c2ccc3ccc(-c4nc(-c5ccccc5)nc(-c5cccc6oc7cc8ccccc8nc7c56)n4)cc3c2)cc1 ; c1ccc(-c2ccc3ccc(-c4nc(-c5ccccc5)nc(-c5cccc6oc7cc8cccnc8cc7c56)n4)cc3c2)cc1]

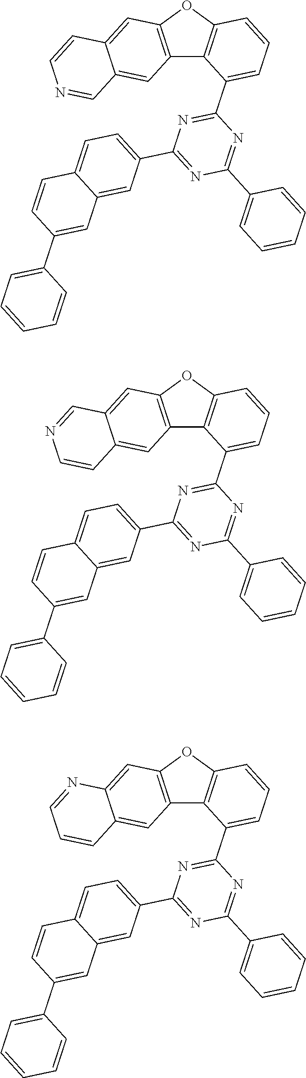 [SMILES: c1ccc(-c2ccc3ccc(-c4nc(-c5ccccc5)nc(-c5cccc6oc7cc8ccncc8cc7c56)n4)cc3c2)cc1 ; c1ccc(-c2ccc3ccc(-c4nc(-c5ccccc5)nc(-c5cccc6oc7cc8cnccc8cc7c56)n4)cc3c2)cc1 ; c1ccc(-c2ccc3ccc(-c4nc(-c5ccccc5)nc(-c5cccc6oc7cc8ncccc8cc7c56)n4)cc3c2)cc1]

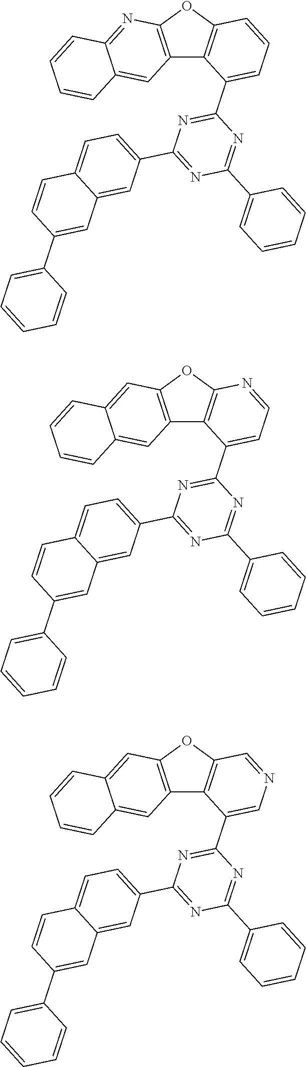 [SMILES: c1ccc(-c2ccc3ccc(-c4nc(-c5ccccc5)nc(-c5cccc6oc7nc8ccccc8cc7c56)n4)cc3c2)cc1 ; c1ccc(-c2ccc3ccc(-c4nc(-c5ccccc5)nc(-c5ccnc6oc7cc8ccccc8cc7c56)n4)cc3c2)cc1 ; c1ccc(-c2ccc3ccc(-c4nc(-c5ccccc5)nc(-c5cncc6oc7cc8ccccc8cc7c56)n4)cc3c2)cc1]

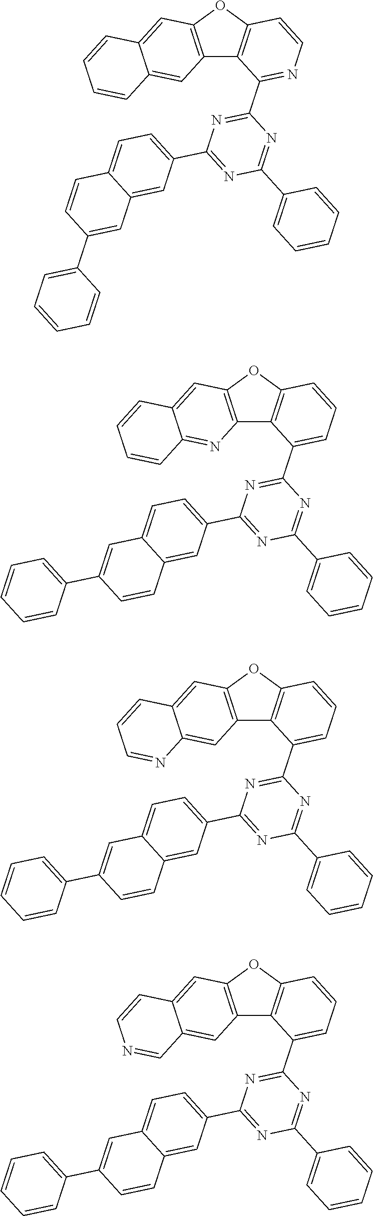 [SMILES: c1ccc(-c2ccc3ccc(-c4nc(-c5ccccc5)nc(-c5nccc6oc7cc8ccccc8cc7c56)n4)cc3c2)cc1 ; c1ccc(-c2ccc3cc(-c4nc(-c5ccccc5)nc(-c5cccc6oc7cc8ccccc8nc7c56)n4)ccc3c2)cc1 ; c1ccc(-c2ccc3cc(-c4nc(-c5ccccc5)nc(-c5cccc6oc7cc8cccnc8cc7c56)n4)ccc3c2)cc1 ; c1ccc(-c2ccc3cc(-c4nc(-c5ccccc5)nc(-c5cccc6oc7cc8ccncc8cc7c56)n4)ccc3c2)cc1]

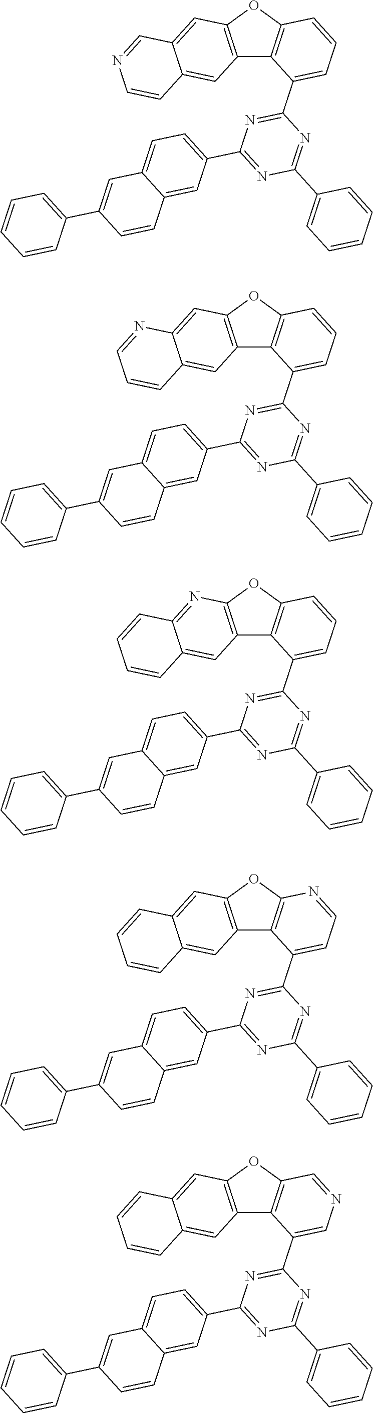 [SMILES: c1ccc(-c2ccc3cc(-c4nc(-c5ccccc5)nc(-c5cccc6oc7cc8cnccc8cc7c56)n4)ccc3c2)cc1 ; c1ccc(-c2ccc3cc(-c4nc(-c5ccccc5)nc(-c5cccc6oc7cc8ncccc8cc7c56)n4)ccc3c2)cc1 ; c1ccc(-c2ccc3cc(-c4nc(-c5ccccc5)nc(-c5cccc6oc7nc8ccccc8cc7c56)n4)ccc3c2)cc1 ; c1ccc(-c2ccc3cc(-c4nc(-c5ccccc5)nc(-c5ccnc6oc7cc8ccccc8cc7c56)n4)ccc3c2)cc1 ; c1ccc(-c2ccc3cc(-c4nc(-c5ccccc5)nc(-c5cncc6oc7cc8ccccc8cc7c56)n4)ccc3c2)cc1]

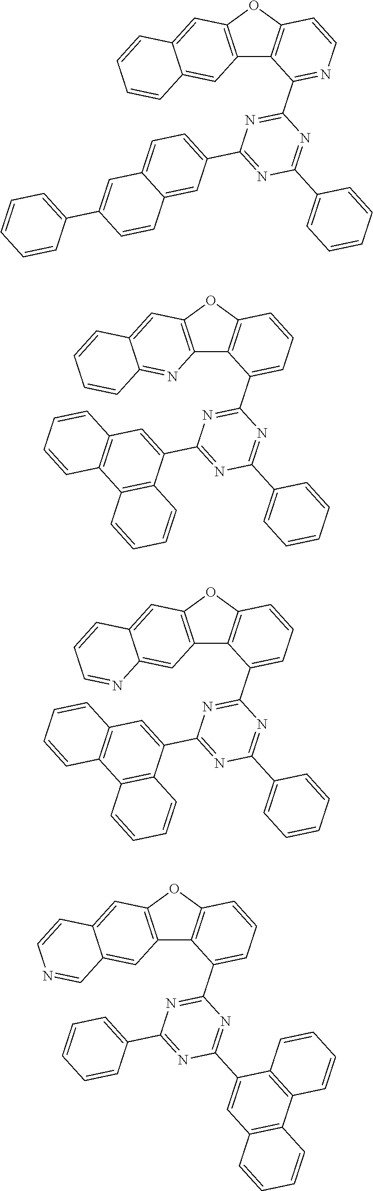 [SMILES: c1ccc(-c2ccc3cc(-c4nc(-c5ccccc5)nc(-c5nccc6oc7cc8ccccc8cc7c56)n4)ccc3c2)cc1 ; c1ccc(-c2nc(-c3cc4ccccc4c4ccccc34)nc(-c3cccc4oc5cc6ccccc6nc5c34)n2)cc1 ; c1ccc(-c2nc(-c3cc4ccccc4c4ccccc34)nc(-c3cccc4oc5cc6cccnc6cc5c34)n2)cc1 ; c1ccc(-c2nc(-c3cc4ccccc4c4ccccc34)nc(-c3cccc4oc5cc6ccncc6cc5c34)n2)cc1]

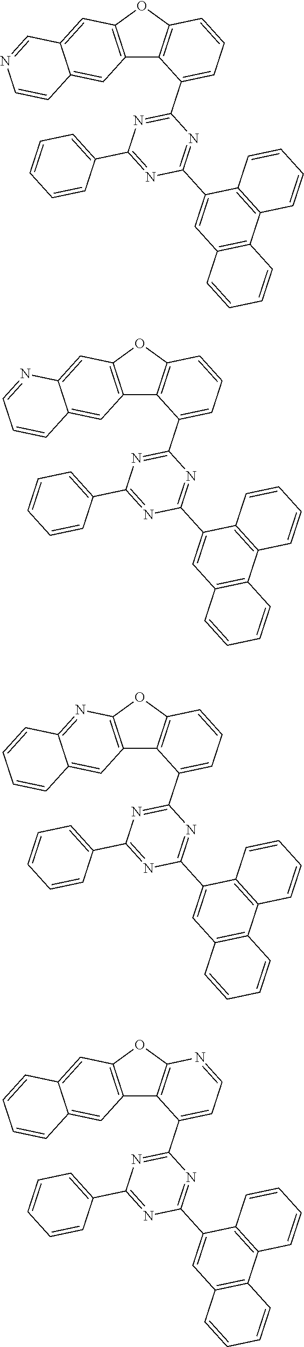 [SMILES: c1ccc(-c2nc(-c3cc4ccccc4c4ccccc34)nc(-c3cccc4oc5cc6cnccc6cc5c34)n2)cc1 ; c1ccc(-c2nc(-c3cc4ccccc4c4ccccc34)nc(-c3cccc4oc5cc6ncccc6cc5c34)n2)cc1 ; c1ccc(-c2nc(-c3cc4ccccc4c4ccccc34)nc(-c3cccc4oc5nc6ccccc6cc5c34)n2)cc1 ; c1ccc(-c2nc(-c3cc4ccccc4c4ccccc34)nc(-c3ccnc4oc5cc6ccccc6cc5c34)n2)cc1]

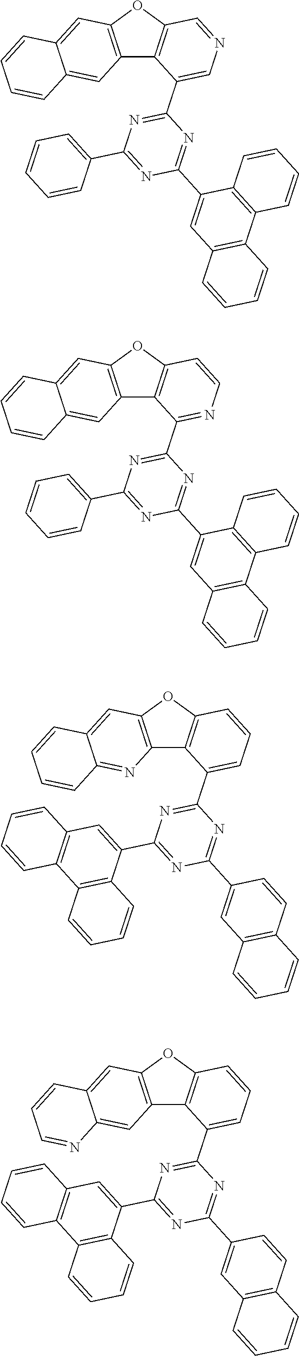 [SMILES: c1ccc(-c2nc(-c3cc4ccccc4c4ccccc34)nc(-c3cncc4oc5cc6ccccc6cc5c34)n2)cc1 ; c1ccc(-c2nc(-c3cc4ccccc4c4ccccc34)nc(-c3nccc4oc5cc6ccccc6cc5c34)n2)cc1 ; c1ccc2cc(-c3nc(-c4cc5ccccc5c5ccccc45)nc(-c4cccc5oc6cc7ccccc7nc6c45)n3)ccc2c1 ; c1ccc2cc(-c3nc(-c4cc5ccccc5c5ccccc45)nc(-c4cccc5oc6cc7cccnc7cc6c45)n3)ccc2c1]

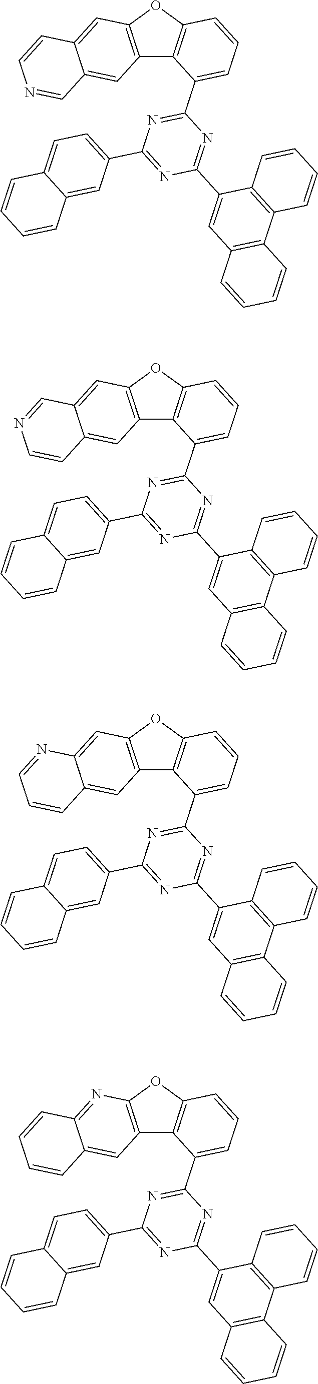 [SMILES: c1ccc2cc(-c3nc(-c4cc5ccccc5c5ccccc45)nc(-c4cccc5oc6cc7ccncc7cc6c45)n3)ccc2c1 ; c1ccc2cc(-c3nc(-c4cc5ccccc5c5ccccc45)nc(-c4cccc5oc6cc7cnccc7cc6c45)n3)ccc2c1 ; c1ccc2cc(-c3nc(-c4cc5ccccc5c5ccccc45)nc(-c4cccc5oc6cc7ncccc7cc6c45)n3)ccc2c1 ; c1ccc2cc(-c3nc(-c4cc5ccccc5c5ccccc45)nc(-c4cccc5oc6nc7ccccc7cc6c45)n3)ccc2c1]

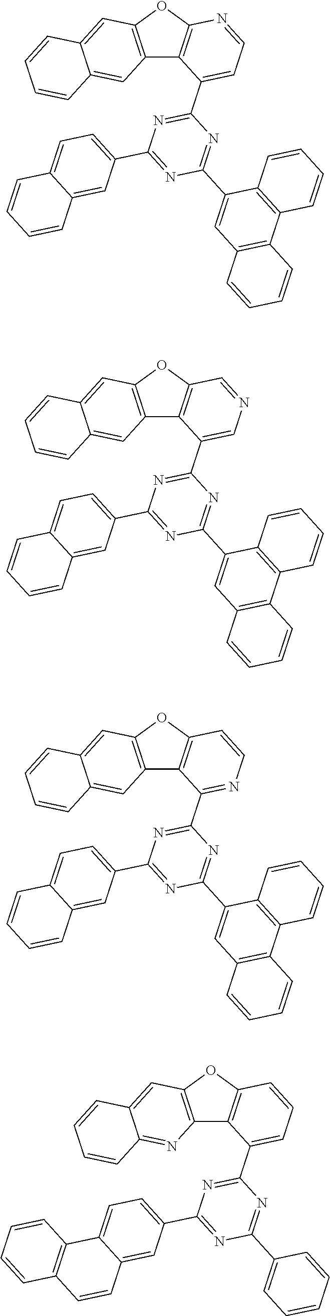 [SMILES: c1ccc2cc(-c3nc(-c4cc5ccccc5c5ccccc45)nc(-c4ccnc5oc6cc7ccccc7cc6c45)n3)ccc2c1 ; c1ccc2cc(-c3nc(-c4cc5ccccc5c5ccccc45)nc(-c4cncc5oc6cc7ccccc7cc6c45)n3)ccc2c1 ; c1ccc2cc(-c3nc(-c4cc5ccccc5c5ccccc45)nc(-c4nccc5oc6cc7ccccc7cc6c45)n3)ccc2c1 ; c1ccc(-c2nc(-c3ccc4c(ccc5ccccc54)c3)nc(-c3cccc4oc5cc6ccccc6nc5c34)n2)cc1]

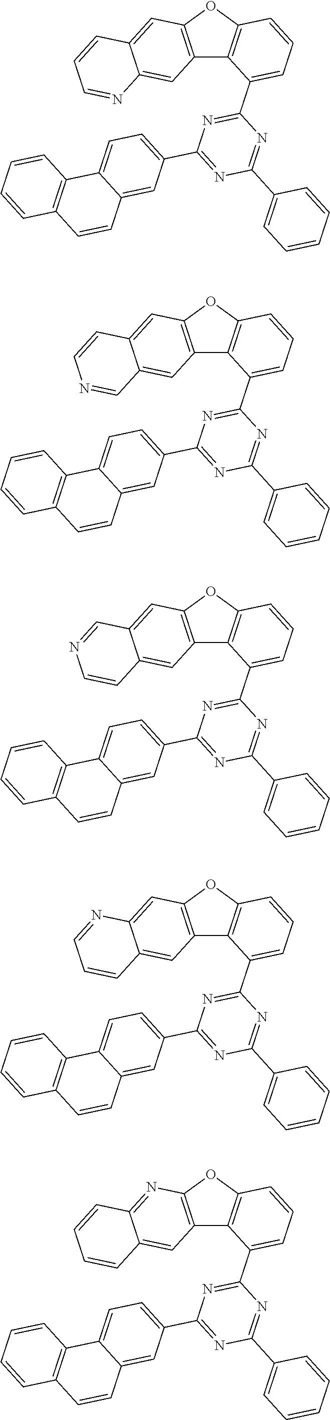 [SMILES: c1ccc(-c2nc(-c3ccc4c(ccc5ccccc54)c3)nc(-c3cccc4oc5cc6cccnc6cc5c34)n2)cc1 ; c1ccc(-c2nc(-c3ccc4c(ccc5ccccc54)c3)nc(-c3cccc4oc5cc6ccncc6cc5c34)n2)cc1 ; c1ccc(-c2nc(-c3ccc4c(ccc5ccccc54)c3)nc(-c3cccc4oc5cc6cnccc6cc5c34)n2)cc1 ; c1ccc(-c2nc(-c3ccc4c(ccc5ccccc54)c3)nc(-c3cccc4oc5cc6ncccc6cc5c34)n2)cc1 ; c1ccc(-c2nc(-c3ccc4c(ccc5ccccc54)c3)nc(-c3cccc4oc5nc6ccccc6cc5c34)n2)cc1]

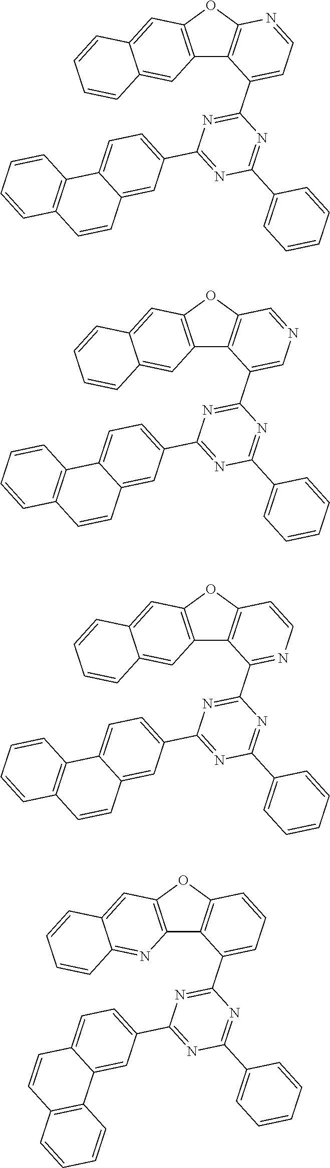 [SMILES: c1ccc(-c2nc(-c3ccc4c(ccc5ccccc54)c3)nc(-c3ccnc4oc5cc6ccccc6cc5c34)n2)cc1 ; c1ccc(-c2nc(-c3ccc4c(ccc5ccccc54)c3)nc(-c3cncc4oc5cc6ccccc6cc5c34)n2)cc1 ; c1ccc(-c2nc(-c3ccc4c(ccc5ccccc54)c3)nc(-c3nccc4oc5cc6ccccc6cc5c34)n2)cc1 ; c1ccc(-c2nc(-c3ccc4ccc5ccccc5c4c3)nc(-c3cccc4oc5cc6ccccc6nc5c34)n2)cc1]

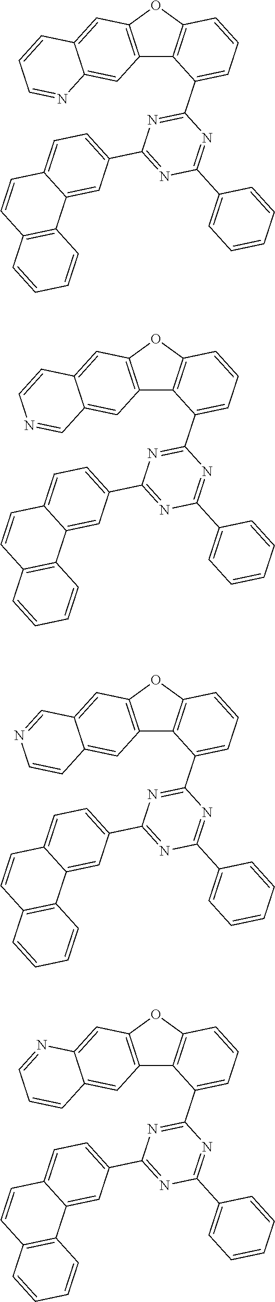 [SMILES: c1ccc(-c2nc(-c3ccc4ccc5ccccc5c4c3)nc(-c3cccc4oc5cc6cccnc6cc5c34)n2)cc1 ; c1ccc(-c2nc(-c3ccc4ccc5ccccc5c4c3)nc(-c3cccc4oc5cc6ccncc6cc5c34)n2)cc1 ; c1ccc(-c2nc(-c3ccc4ccc5ccccc5c4c3)nc(-c3cccc4oc5cc6cnccc6cc5c34)n2)cc1 ; c1ccc(-c2nc(-c3ccc4ccc5ccccc5c4c3)nc(-c3cccc4oc5cc6ncccc6cc5c34)n2)cc1]

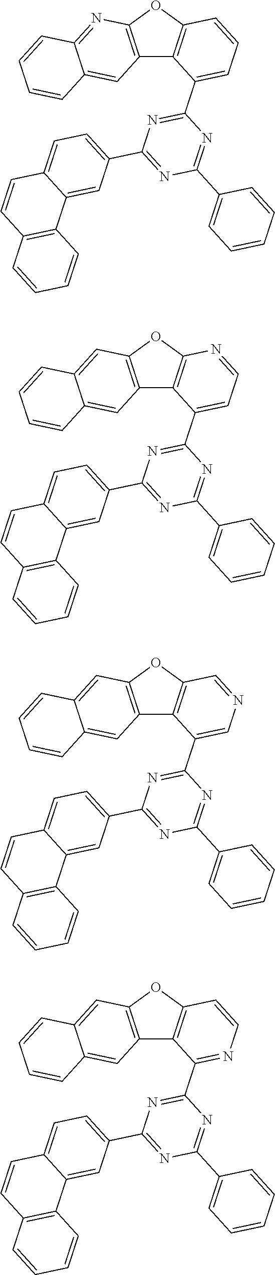 [SMILES: c1ccc(-c2nc(-c3ccc4ccc5ccccc5c4c3)nc(-c3cccc4oc5nc6ccccc6cc5c34)n2)cc1 ; c1ccc(-c2nc(-c3ccc4ccc5ccccc5c4c3)nc(-c3ccnc4oc5cc6ccccc6cc5c34)n2)cc1 ; c1ccc(-c2nc(-c3ccc4ccc5ccccc5c4c3)nc(-c3cncc4oc5cc6ccccc6cc5c34)n2)cc1 ; c1ccc(-c2nc(-c3ccc4ccc5ccccc5c4c3)nc(-c3nccc4oc5cc6ccccc6cc5c34)n2)cc1]

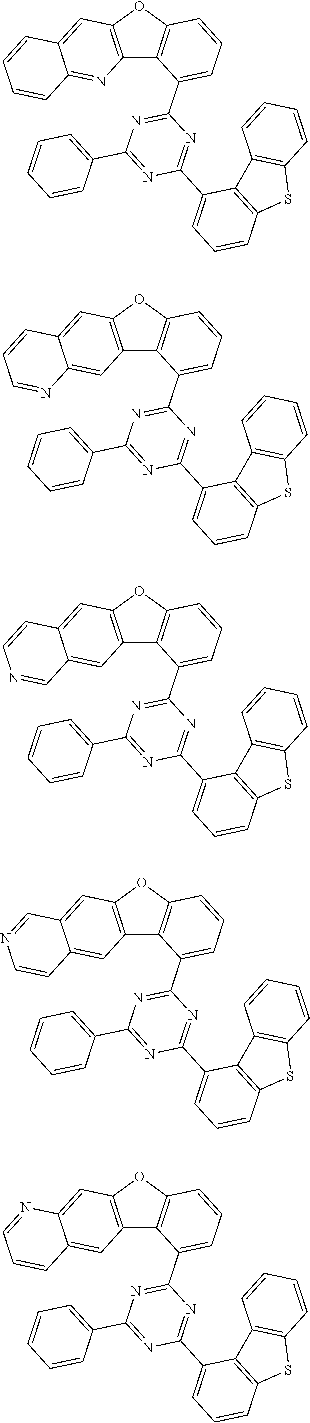 [SMILES: c1ccc(-c2nc(-c3cccc4sc5ccccc5c34)nc(-c3cccc4oc5cc6ccccc6nc5c34)n2)cc1 ; c1ccc(-c2nc(-c3cccc4oc5cc6cccnc6cc5c34)nc(-c3cccc4sc5ccccc5c34)n2)cc1 ; c1ccc(-c2nc(-c3cccc4oc5cc6ccncc6cc5c34)nc(-c3cccc4sc5ccccc5c34)n2)cc1 ; c1ccc(-c2nc(-c3cccc4oc5cc6cnccc6cc5c34)nc(-c3cccc4sc5ccccc5c34)n2)cc1 ; c1ccc(-c2nc(-c3cccc4oc5cc6ncccc6cc5c34)nc(-c3cccc4sc5ccccc5c34)n2)cc1]

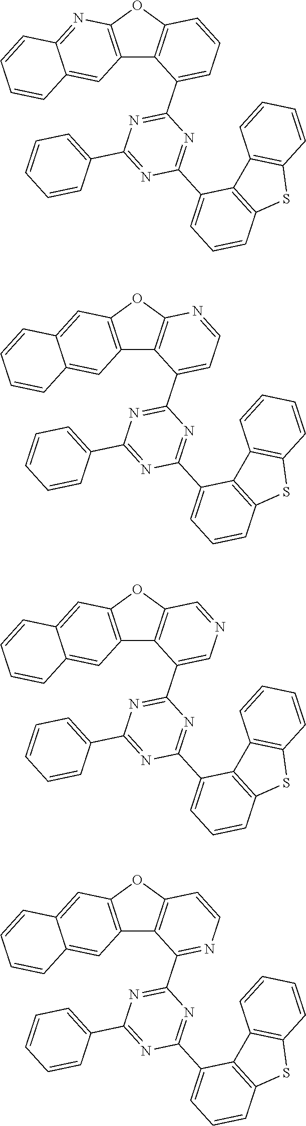 [SMILES: c1ccc(-c2nc(-c3cccc4oc5nc6ccccc6cc5c34)nc(-c3cccc4sc5ccccc5c34)n2)cc1 ; c1ccc(-c2nc(-c3ccnc4oc5cc6ccccc6cc5c34)nc(-c3cccc4sc5ccccc5c34)n2)cc1 ; c1ccc(-c2nc(-c3cncc4oc5cc6ccccc6cc5c34)nc(-c3cccc4sc5ccccc5c34)n2)cc1 ; c1ccc(-c2nc(-c3nccc4oc5cc6ccccc6cc5c34)nc(-c3cccc4sc5ccccc5c34)n2)cc1]

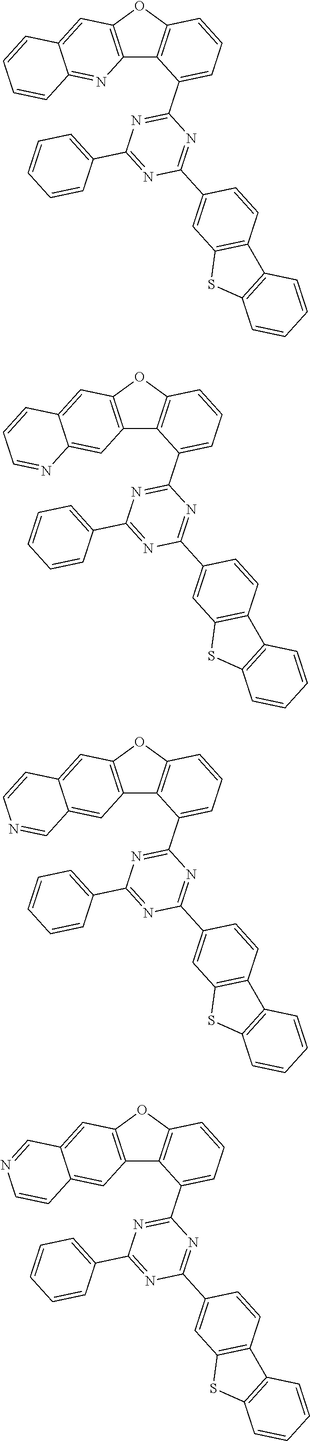 [SMILES: c1ccc(-c2nc(-c3ccc4c(c3)sc3ccccc34)nc(-c3cccc4oc5cc6ccccc6nc5c34)n2)cc1 ; c1ccc(-c2nc(-c3ccc4c(c3)sc3ccccc34)nc(-c3cccc4oc5cc6cccnc6cc5c34)n2)cc1 ; c1ccc(-c2nc(-c3ccc4c(c3)sc3ccccc34)nc(-c3cccc4oc5cc6ccncc6cc5c34)n2)cc1 ; c1ccc(-c2nc(-c3ccc4c(c3)sc3ccccc34)nc(-c3cccc4oc5cc6cnccc6cc5c34)n2)cc1]

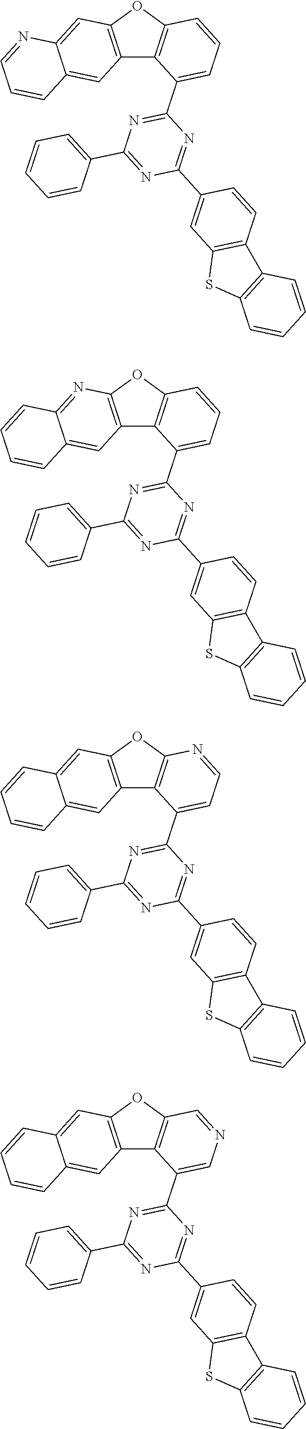 [SMILES: c1ccc(-c2nc(-c3ccc4c(c3)sc3ccccc34)nc(-c3cccc4oc5cc6ncccc6cc5c34)n2)cc1 ; c1ccc(-c2nc(-c3ccc4c(c3)sc3ccccc34)nc(-c3cccc4oc5nc6ccccc6cc5c34)n2)cc1 ; c1ccc(-c2nc(-c3ccc4c(c3)sc3ccccc34)nc(-c3ccnc4oc5cc6ccccc6cc5c34)n2)cc1 ; c1ccc(-c2nc(-c3ccc4c(c3)sc3ccccc34)nc(-c3cncc4oc5cc6ccccc6cc5c34)n2)cc1]

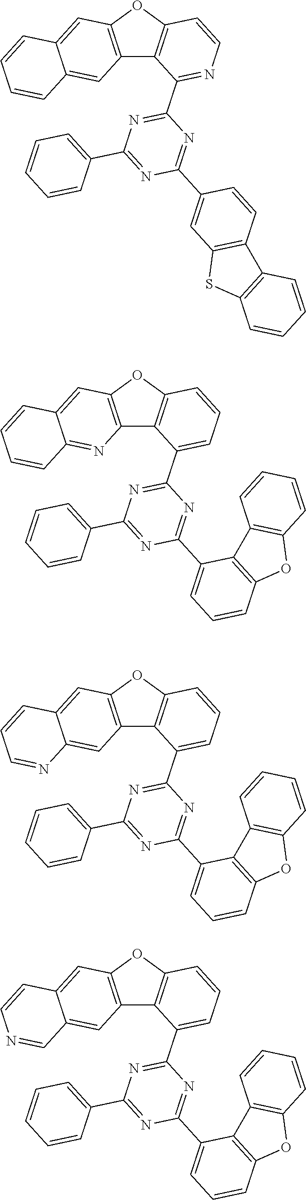 [SMILES: c1ccc(-c2nc(-c3ccc4c(c3)sc3ccccc34)nc(-c3nccc4oc5cc6ccccc6cc5c34)n2)cc1 ; c1ccc(-c2nc(-c3cccc4oc5ccccc5c34)nc(-c3cccc4oc5cc6ccccc6nc5c34)n2)cc1 ; c1ccc(-c2nc(-c3cccc4oc5ccccc5c34)nc(-c3cccc4oc5cc6cccnc6cc5c34)n2)cc1 ; c1ccc(-c2nc(-c3cccc4oc5ccccc5c34)nc(-c3cccc4oc5cc6ccncc6cc5c34)n2)cc1]

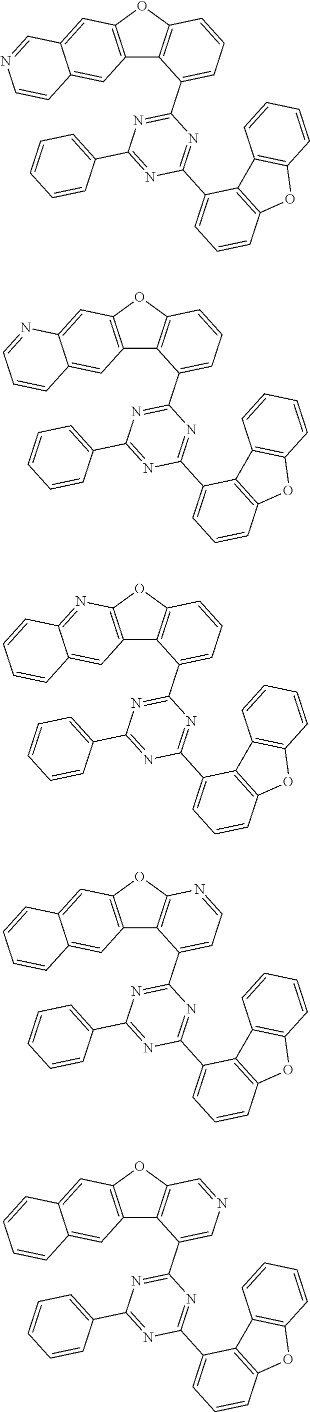 [SMILES: c1ccc(-c2nc(-c3cccc4oc5ccccc5c34)nc(-c3cccc4oc5cc6cnccc6cc5c34)n2)cc1 ; c1ccc(-c2nc(-c3cccc4oc5ccccc5c34)nc(-c3cccc4oc5cc6ncccc6cc5c34)n2)cc1 ; c1ccc(-c2nc(-c3cccc4oc5ccccc5c34)nc(-c3cccc4oc5nc6ccccc6cc5c34)n2)cc1 ; c1ccc(-c2nc(-c3cccc4oc5ccccc5c34)nc(-c3ccnc4oc5cc6ccccc6cc5c34)n2)cc1 ; c1ccc(-c2nc(-c3cccc4oc5ccccc5c34)nc(-c3cncc4oc5cc6ccccc6cc5c34)n2)cc1]

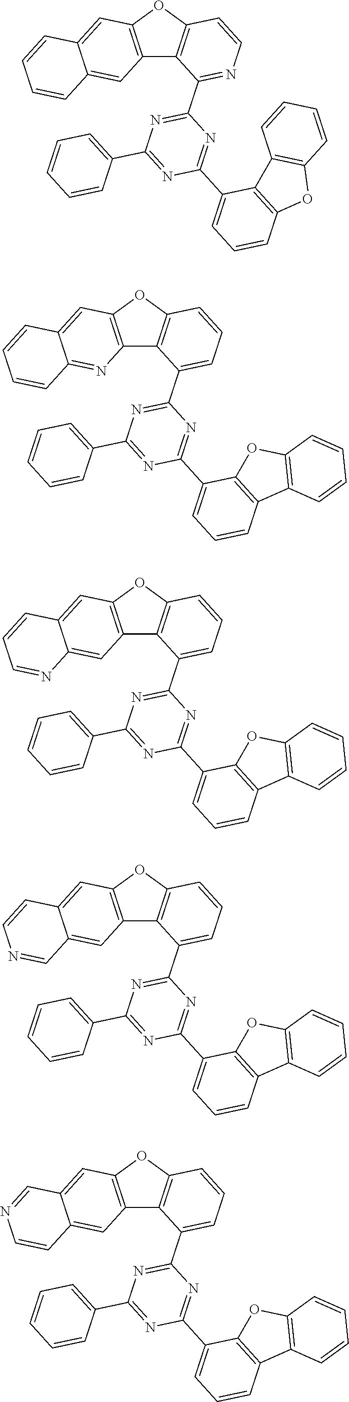 [SMILES: c1ccc(-c2nc(-c3cccc4oc5ccccc5c34)nc(-c3nccc4oc5cc6ccccc6cc5c34)n2)cc1 ; c1ccc(-c2nc(-c3cccc4c3oc3ccccc34)nc(-c3cccc4oc5cc6ccccc6nc5c34)n2)cc1 ; c1ccc(-c2nc(-c3cccc4c3oc3ccccc34)nc(-c3cccc4oc5cc6cccnc6cc5c34)n2)cc1 ; c1ccc(-c2nc(-c3cccc4c3oc3ccccc34)nc(-c3cccc4oc5cc6ccncc6cc5c34)n2)cc1 ; c1ccc(-c2nc(-c3cccc4c3oc3ccccc34)nc(-c3cccc4oc5cc6cnccc6cc5c34)n2)cc1]

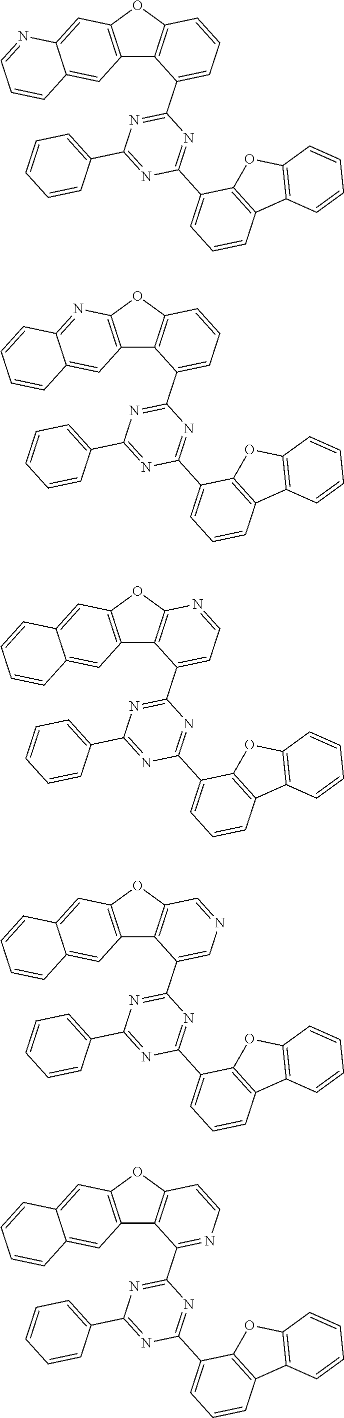 [SMILES: c1ccc(-c2nc(-c3cccc4c3oc3ccccc34)nc(-c3cccc4oc5cc6ncccc6cc5c34)n2)cc1 ; c1ccc(-c2nc(-c3cccc4c3oc3ccccc34)nc(-c3cccc4oc5nc6ccccc6cc5c34)n2)cc1 ; c1ccc(-c2nc(-c3cccc4c3oc3ccccc34)nc(-c3ccnc4oc5cc6ccccc6cc5c34)n2)cc1 ; c1ccc(-c2nc(-c3cccc4c3oc3ccccc34)nc(-c3cncc4oc5cc6ccccc6cc5c34)n2)cc1 ; c1ccc(-c2nc(-c3cccc4c3oc3ccccc34)nc(-c3nccc4oc5cc6ccccc6cc5c34)n2)cc1]

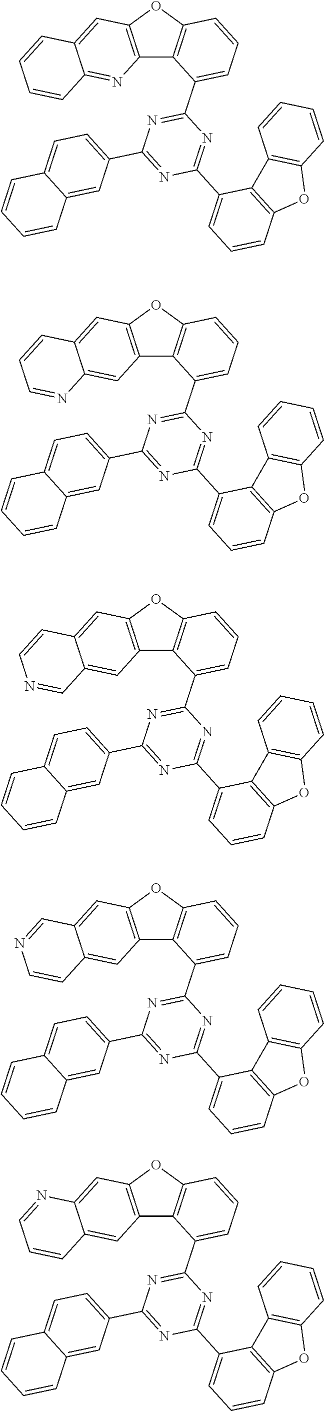

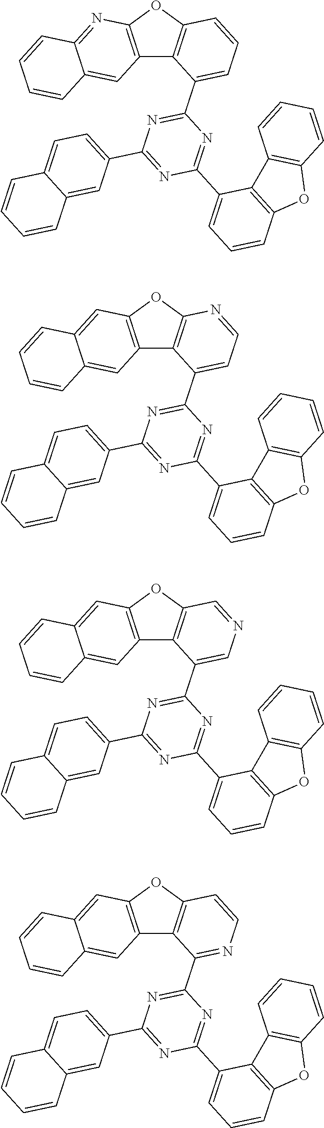 [SMILES: c1ccc2cc(-c3nc(-c4cccc5oc6ccccc6c45)nc(-c4cccc5oc6nc7ccccc7cc6c45)n3)ccc2c1 ; c1ccc2cc(-c3nc(-c4cccc5oc6ccccc6c45)nc(-c4ccnc5oc6cc7ccccc7cc6c45)n3)ccc2c1 ; c1ccc2cc(-c3nc(-c4cccc5oc6ccccc6c45)nc(-c4cncc5oc6cc7ccccc7cc6c45)n3)ccc2c1 ; c1ccc2cc(-c3nc(-c4cccc5oc6ccccc6c45)nc(-c4nccc5oc6cc7ccccc7cc6c45)n3)ccc2c1]

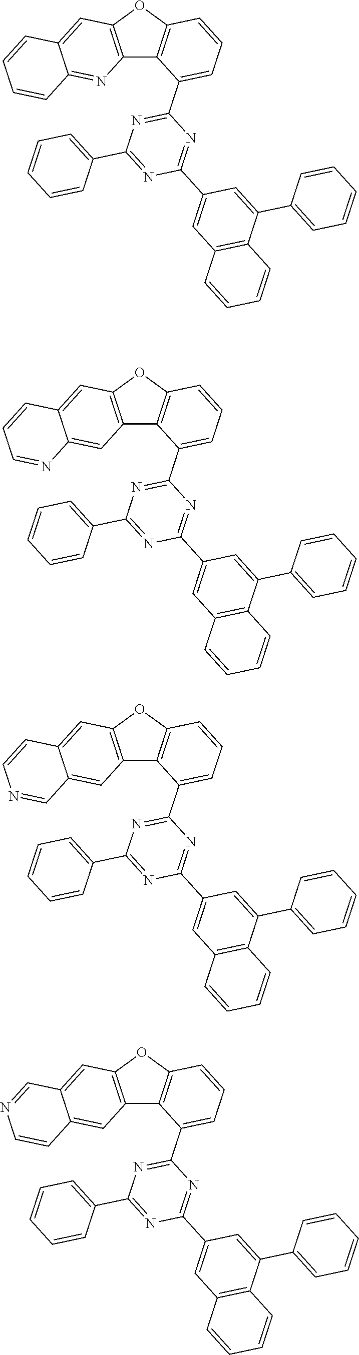 [SMILES: c1ccc(-c2nc(-c3cc(-c4ccccc4)c4ccccc4c3)nc(-c3cccc4oc5cc6ccccc6nc5c34)n2)cc1 ; c1ccc(-c2nc(-c3cc(-c4ccccc4)c4ccccc4c3)nc(-c3cccc4oc5cc6cccnc6cc5c34)n2)cc1 ; c1ccc(-c2nc(-c3cc(-c4ccccc4)c4ccccc4c3)nc(-c3cccc4oc5cc6ccncc6cc5c34)n2)cc1 ; c1ccc(-c2nc(-c3cc(-c4ccccc4)c4ccccc4c3)nc(-c3cccc4oc5cc6cnccc6cc5c34)n2)cc1]

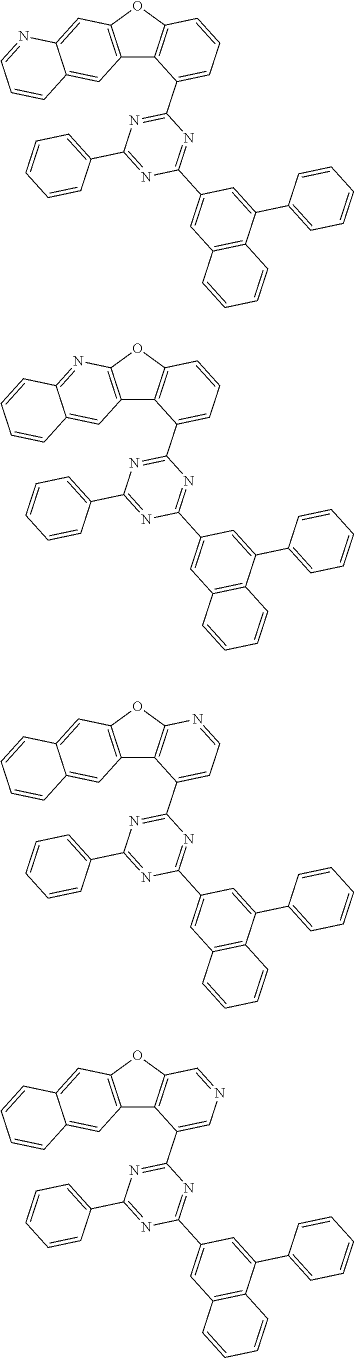 [SMILES: c1ccc(-c2nc(-c3cc(-c4ccccc4)c4ccccc4c3)nc(-c3cccc4oc5cc6ncccc6cc5c34)n2)cc1 ; c1ccc(-c2nc(-c3cc(-c4ccccc4)c4ccccc4c3)nc(-c3cccc4oc5nc6ccccc6cc5c34)n2)cc1 ; c1ccc(-c2nc(-c3cc(-c4ccccc4)c4ccccc4c3)nc(-c3ccnc4oc5cc6ccccc6cc5c34)n2)cc1 ; c1ccc(-c2nc(-c3cc(-c4ccccc4)c4ccccc4c3)nc(-c3cncc4oc5cc6ccccc6cc5c34)n2)cc1]

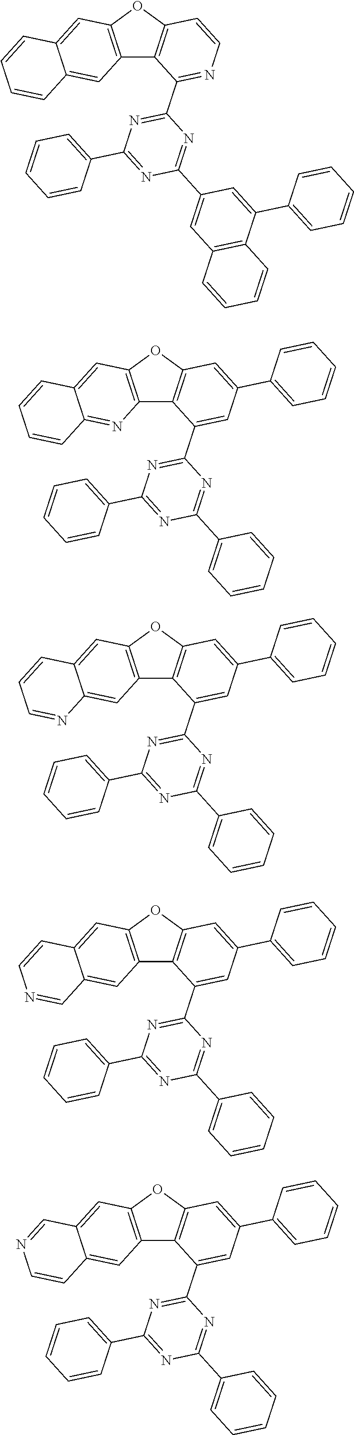

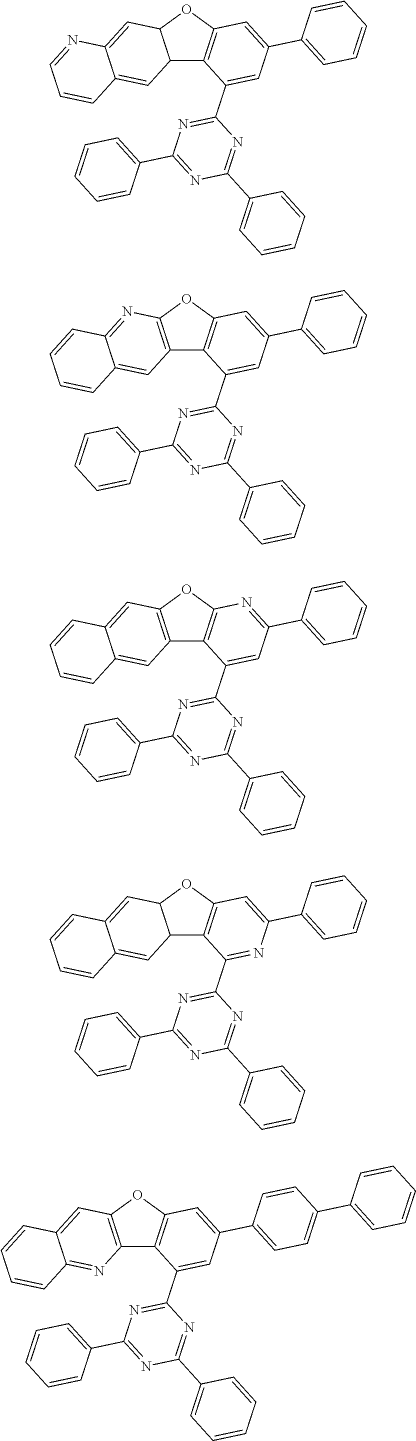 [SMILES: C1=c2cccnc2=CC2Oc3cc(-c4ccccc4)cc(-c4nc(-c5ccccc5)nc(-c5ccccc5)n4)c3C12 ; c1ccc(-c2cc(-c3nc(-c4ccccc4)nc(-c4ccccc4)n3)c3c(c2)oc2nc4ccccc4cc23)cc1 ; c1ccc(-c2cc(-c3nc(-c4ccccc4)nc(-c4ccccc4)n3)c3c(n2)oc2cc4ccccc4cc23)cc1 ; C1=c2ccccc2=CC2c3c(cc(-c4ccccc4)nc3-c3nc(-c4ccccc4)nc(-c4ccccc4)n3)OC12 ; c1ccc(-c2ccc(-c3cc(-c4nc(-c5ccccc5)nc(-c5ccccc5)n4)c4c(c3)oc3cc5ccccc5nc34)cc2)cc1]

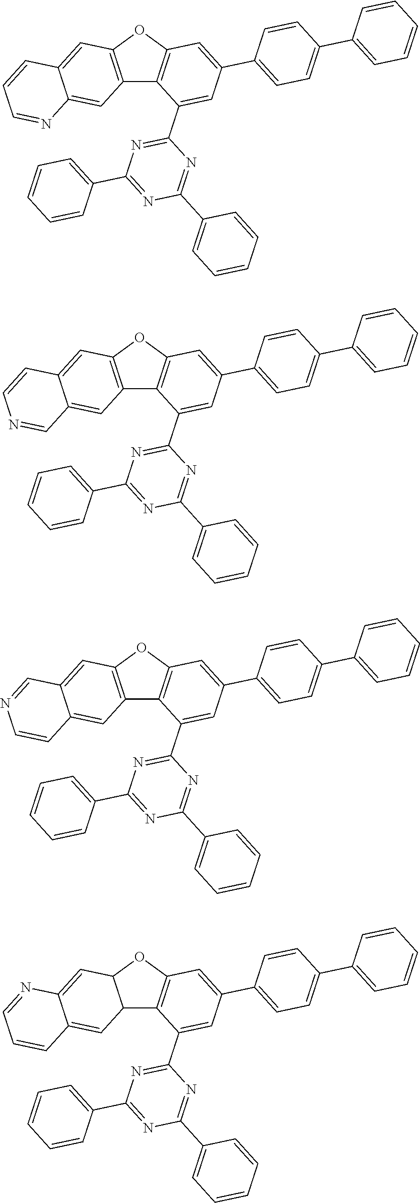 [SMILES: c1ccc(-c2ccc(-c3cc(-c4nc(-c5ccccc5)nc(-c5ccccc5)n4)c4c(c3)oc3cc5cccnc5cc34)cc2)cc1 ; c1ccc(-c2ccc(-c3cc(-c4nc(-c5ccccc5)nc(-c5ccccc5)n4)c4c(c3)oc3cc5ccncc5cc34)cc2)cc1 ; c1ccc(-c2ccc(-c3cc(-c4nc(-c5ccccc5)nc(-c5ccccc5)n4)c4c(c3)oc3cc5cnccc5cc34)cc2)cc1 ; C1=c2cccnc2=CC2Oc3cc(-c4ccc(-c5ccccc5)cc4)cc(-c4nc(-c5ccccc5)nc(-c5ccccc5)n4)c3C12]

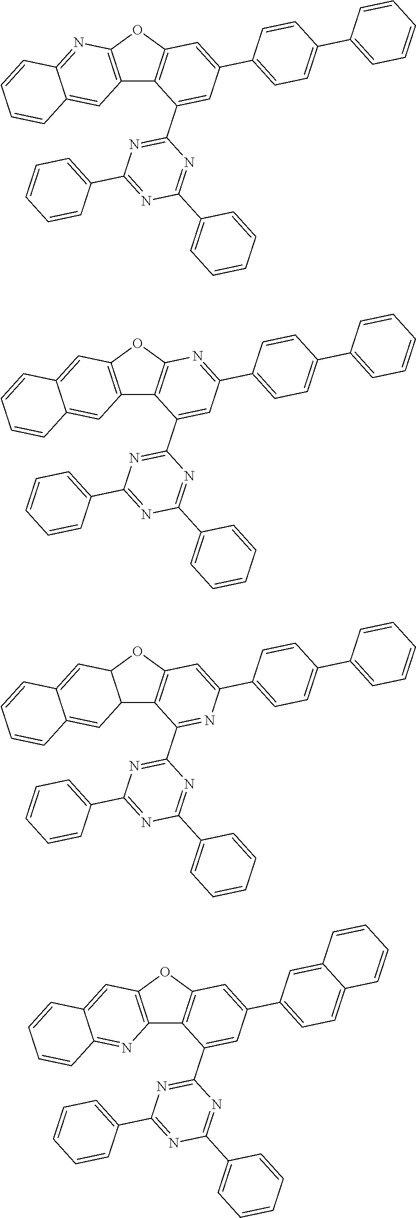 [SMILES: c1ccc(-c2ccc(-c3cc(-c4nc(-c5ccccc5)nc(-c5ccccc5)n4)c4c(c3)oc3nc5ccccc5cc34)cc2)cc1 ; c1ccc(-c2ccc(-c3cc(-c4nc(-c5ccccc5)nc(-c5ccccc5)n4)c4c(n3)oc3cc5ccccc5cc34)cc2)cc1 ; C1=c2ccccc2=CC2c3c(cc(-c4ccc(-c5ccccc5)cc4)nc3-c3nc(-c4ccccc4)nc(-c4ccccc4)n3)OC12 ; c1ccc(-c2nc(-c3ccccc3)nc(-c3cc(-c4ccc5ccccc5c4)cc4oc5cc6ccccc6nc5c34)n2)cc1]

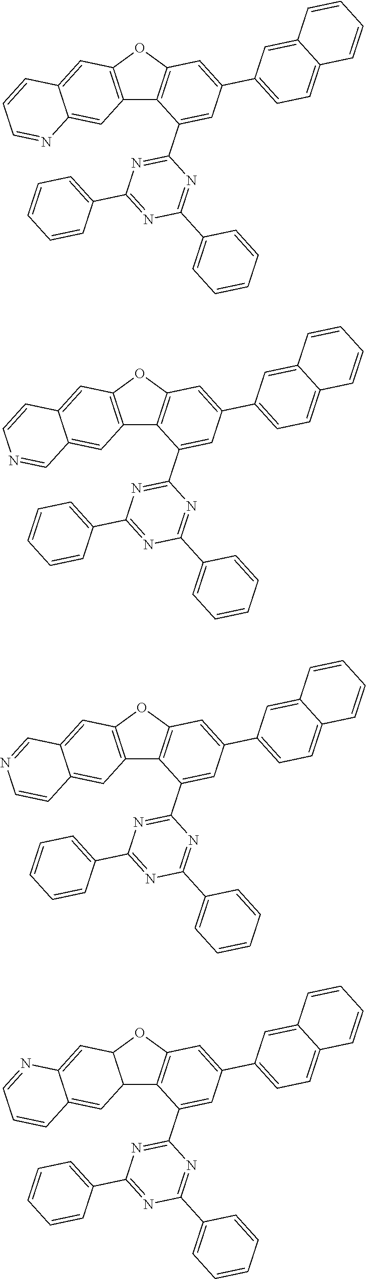 [SMILES: c1ccc(-c2nc(-c3ccccc3)nc(-c3cc(-c4ccc5ccccc5c4)cc4oc5cc6cccnc6cc5c34)n2)cc1 ; c1ccc(-c2nc(-c3ccccc3)nc(-c3cc(-c4ccc5ccccc5c4)cc4oc5cc6ccncc6cc5c34)n2)cc1 ; c1ccc(-c2nc(-c3ccccc3)nc(-c3cc(-c4ccc5ccccc5c4)cc4oc5cc6cnccc6cc5c34)n2)cc1 ; C1=c2cccnc2=CC2Oc3cc(-c4ccc5ccccc5c4)cc(-c4nc(-c5ccccc5)nc(-c5ccccc5)n4)c3C12]

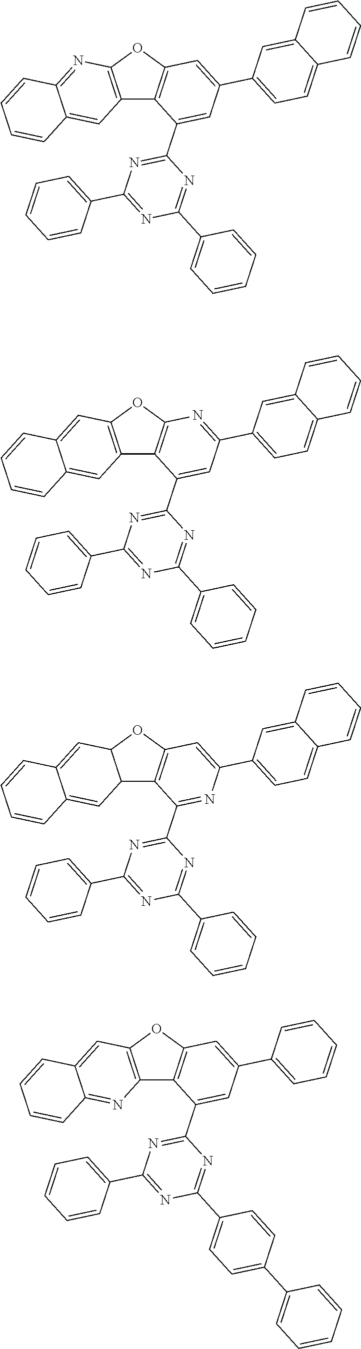 [SMILES: c1ccc(-c2nc(-c3ccccc3)nc(-c3cc(-c4ccc5ccccc5c4)cc4oc5nc6ccccc6cc5c34)n2)cc1 ; c1ccc(-c2nc(-c3ccccc3)nc(-c3cc(-c4ccc5ccccc5c4)nc4oc5cc6ccccc6cc5c34)n2)cc1 ; C1=c2ccccc2=CC2c3c(cc(-c4ccc5ccccc5c4)nc3-c3nc(-c4ccccc4)nc(-c4ccccc4)n3)OC12 ; c1ccc(-c2ccc(-c3nc(-c4ccccc4)nc(-c4cc(-c5ccccc5)cc5oc6cc7ccccc7nc6c45)n3)cc2)cc1]

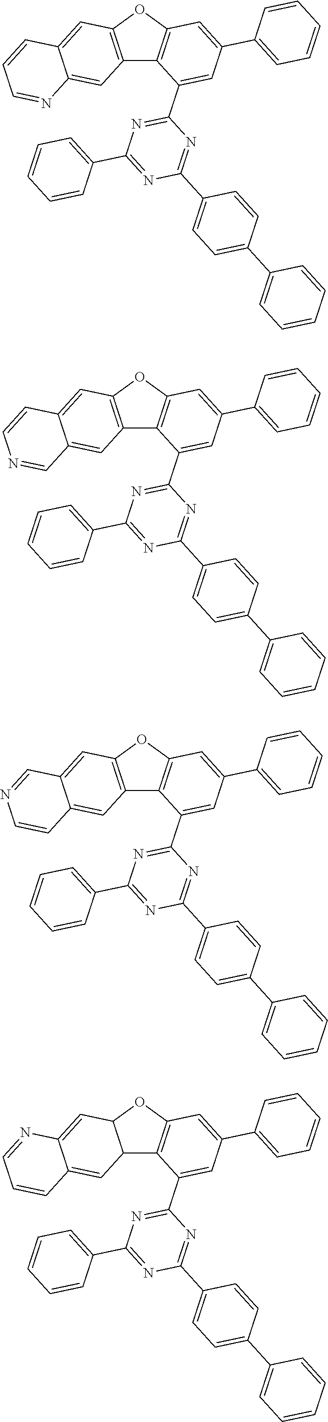

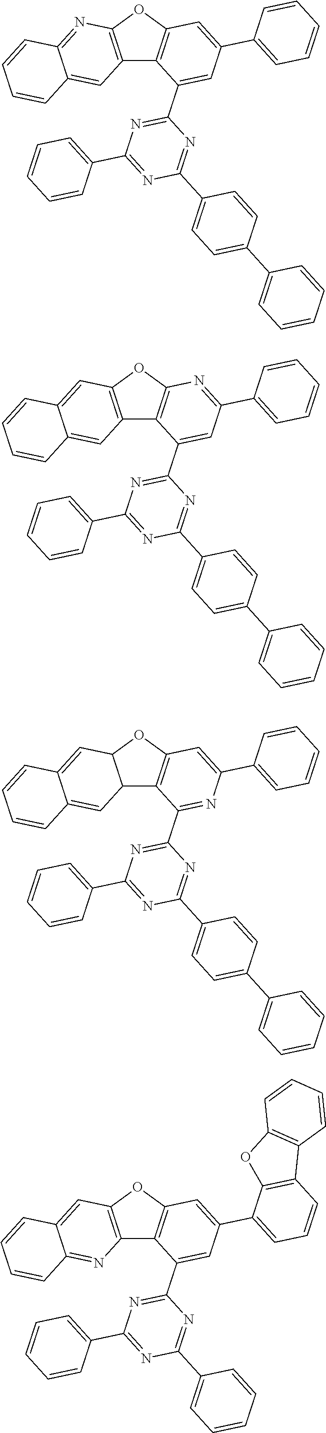

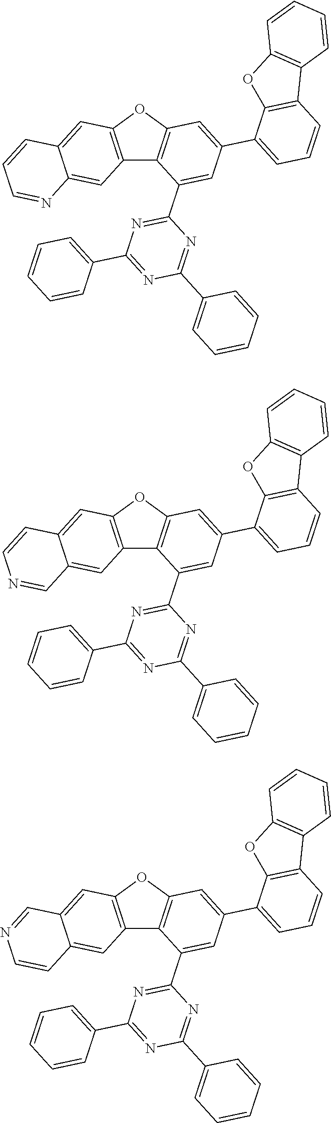 [SMILES: c1ccc(-c2nc(-c3ccccc3)nc(-c3cc(-c4cccc5c4oc4ccccc45)cc4oc5cc6cccnc6cc5c34)n2)cc1 ; c1ccc(-c2nc(-c3ccccc3)nc(-c3cc(-c4cccc5c4oc4ccccc45)cc4oc5cc6ccncc6cc5c34)n2)cc1 ; c1ccc(-c2nc(-c3ccccc3)nc(-c3cc(-c4cccc5c4oc4ccccc45)cc4oc5cc6cnccc6cc5c34)n2)cc1]

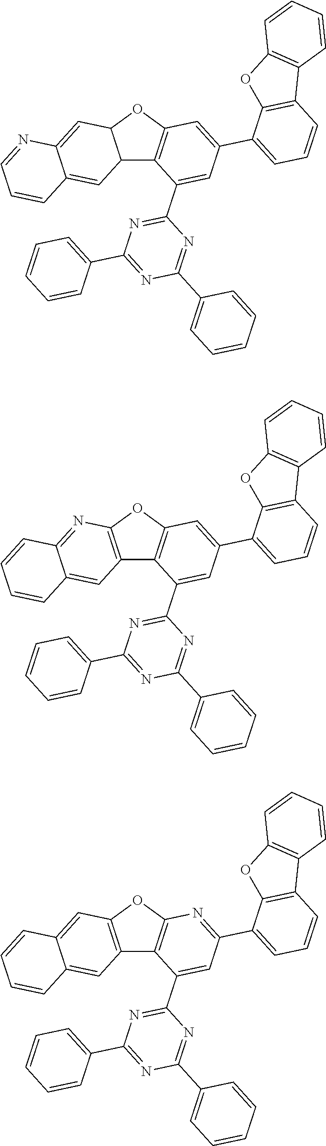 [SMILES: C1=c2cccnc2=CC2Oc3cc(-c4cccc5c4oc4ccccc45)cc(-c4nc(-c5ccccc5)nc(-c5ccccc5)n4)c3C12 ; c1ccc(-c2nc(-c3ccccc3)nc(-c3cc(-c4cccc5c4oc4ccccc45)cc4oc5nc6ccccc6cc5c34)n2)cc1 ; c1ccc(-c2nc(-c3ccccc3)nc(-c3cc(-c4cccc5c4oc4ccccc45)nc4oc5cc6ccccc6cc5c34)n2)cc1]

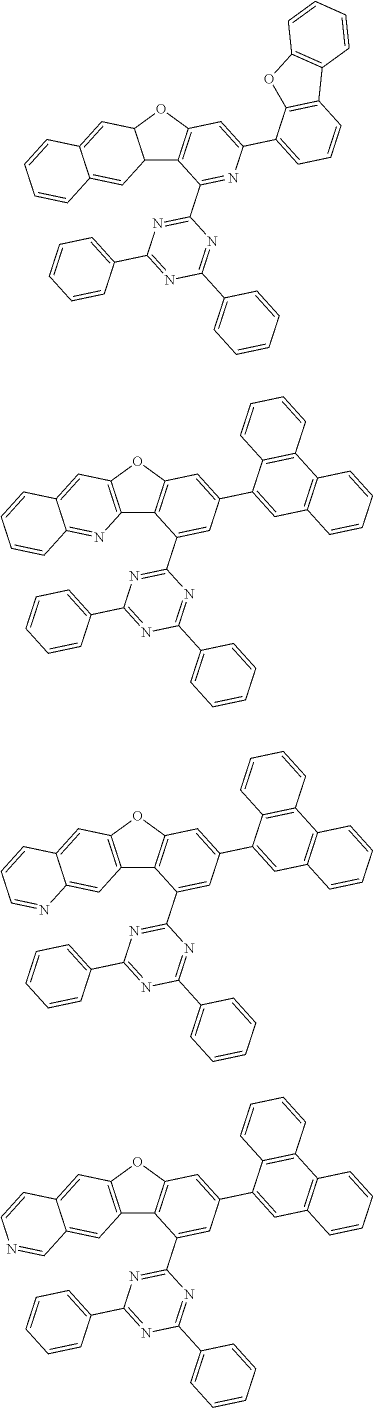 [SMILES: C1=c2ccccc2=CC2c3c(cc(-c4cccc5c4oc4ccccc45)nc3-c3nc(-c4ccccc4)nc(-c4ccccc4)n3)OC12 ; c1ccc(-c2nc(-c3ccccc3)nc(-c3cc(-c4cc5ccccc5c5ccccc45)cc4oc5cc6ccccc6nc5c34)n2)cc1 ; c1ccc(-c2nc(-c3ccccc3)nc(-c3cc(-c4cc5ccccc5c5ccccc45)cc4oc5cc6cccnc6cc5c34)n2)cc1 ; c1ccc(-c2nc(-c3ccccc3)nc(-c3cc(-c4cc5ccccc5c5ccccc45)cc4oc5cc6ccncc6cc5c34)n2)cc1]

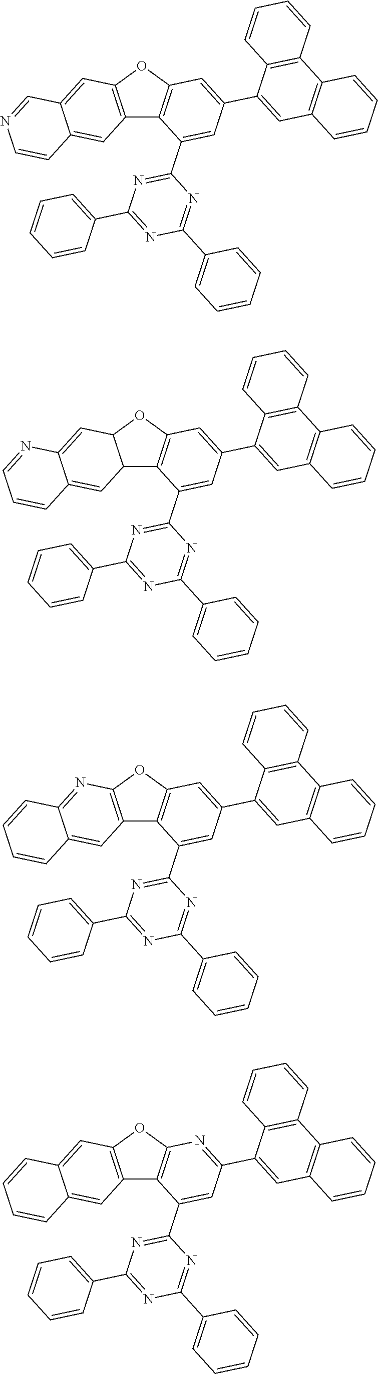 [SMILES: c1ccc(-c2nc(-c3ccccc3)nc(-c3cc(-c4cc5ccccc5c5ccccc45)cc4oc5cc6cnccc6cc5c34)n2)cc1 ; C1=c2cccnc2=CC2Oc3cc(-c4cc5ccccc5c5ccccc45)cc(-c4nc(-c5ccccc5)nc(-c5ccccc5)n4)c3C12 ; c1ccc(-c2nc(-c3ccccc3)nc(-c3cc(-c4cc5ccccc5c5ccccc45)cc4oc5nc6ccccc6cc5c34)n2)cc1 ; c1ccc(-c2nc(-c3ccccc3)nc(-c3cc(-c4cc5ccccc5c5ccccc45)nc4oc5cc6ccccc6cc5c34)n2)cc1]

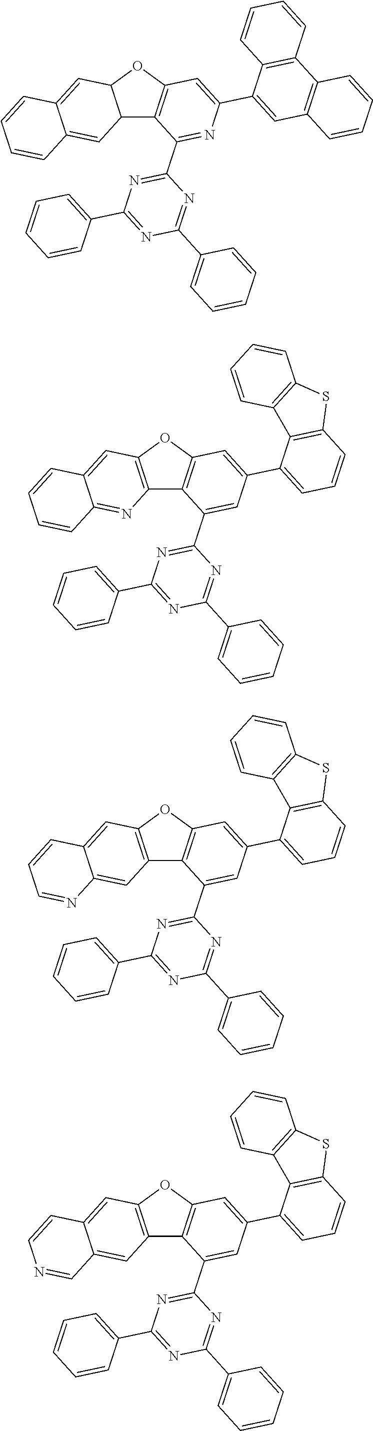

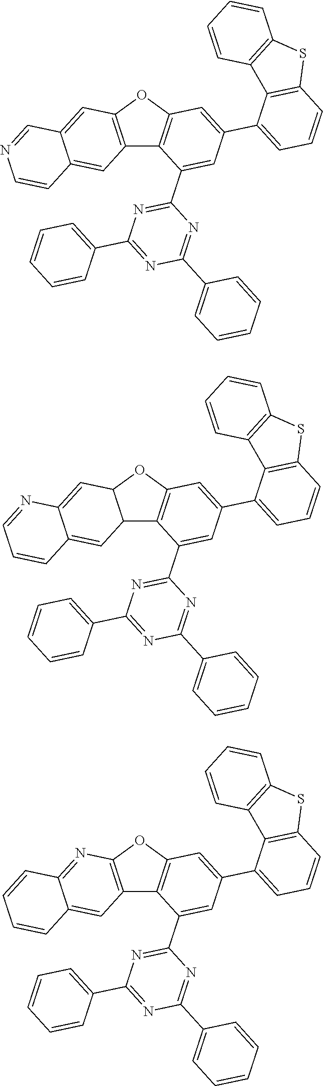

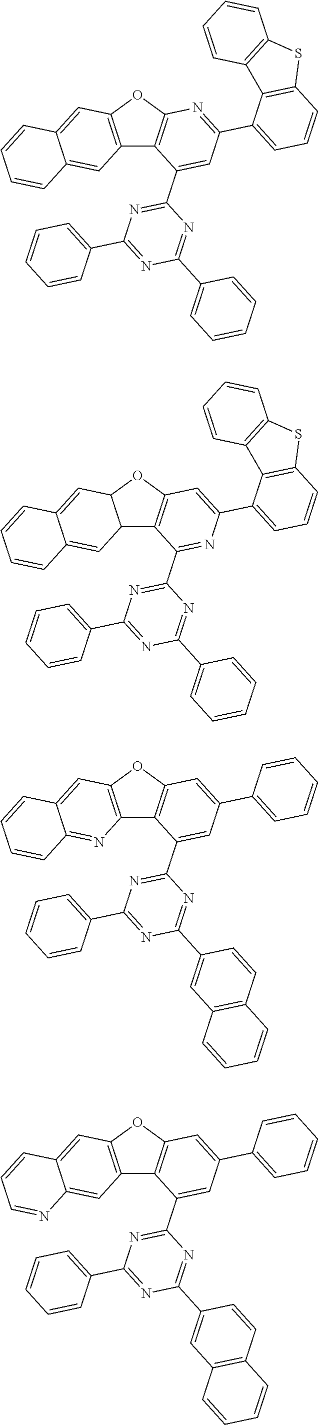

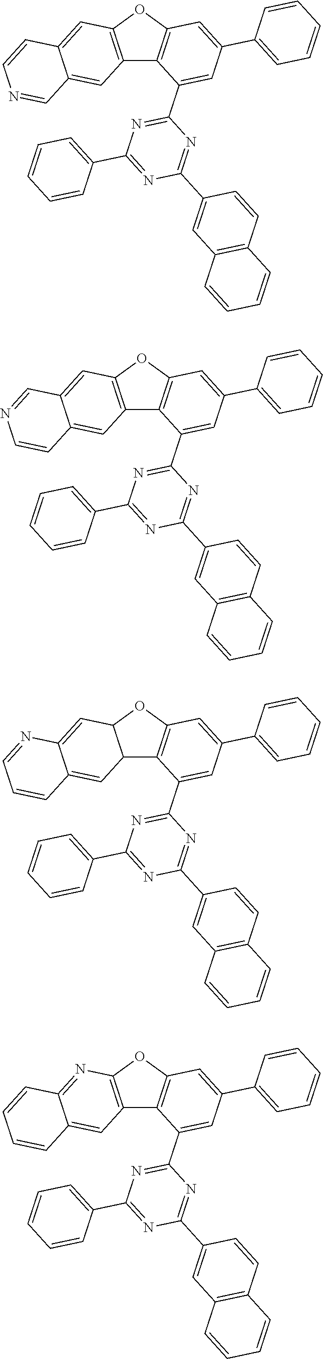 [SMILES: c1ccc(-c2cc(-c3nc(-c4ccccc4)nc(-c4ccc5ccccc5c4)n3)c3c(c2)oc2cc4ccncc4cc23)cc1 ; c1ccc(-c2cc(-c3nc(-c4ccccc4)nc(-c4ccc5ccccc5c4)n3)c3c(c2)oc2cc4cnccc4cc23)cc1 ; C1=c2cccnc2=CC2Oc3cc(-c4ccccc4)cc(-c4nc(-c5ccccc5)nc(-c5ccc6ccccc6c5)n4)c3C12 ; c1ccc(-c2cc(-c3nc(-c4ccccc4)nc(-c4ccc5ccccc5c4)n3)c3c(c2)oc2nc4ccccc4cc23)cc1]

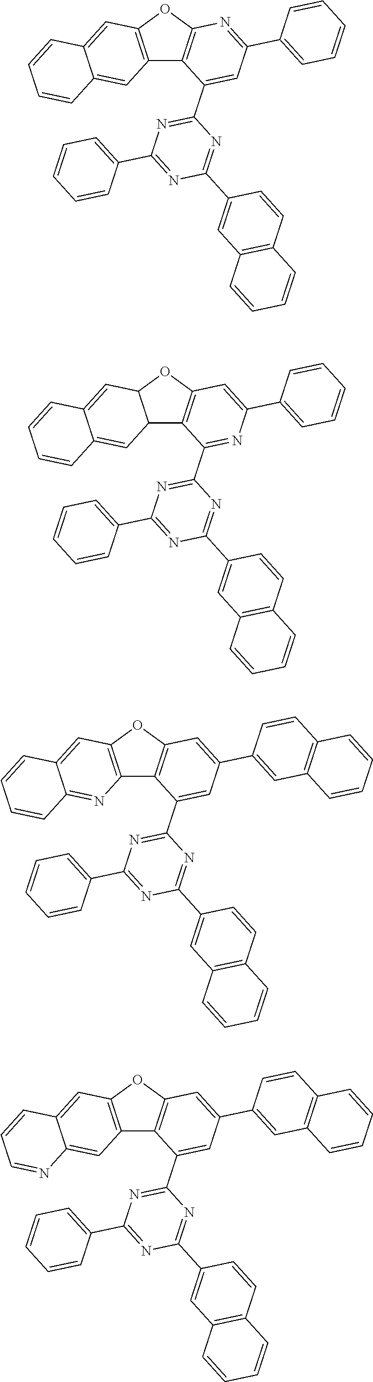 [SMILES: c1ccc(-c2cc(-c3nc(-c4ccccc4)nc(-c4ccc5ccccc5c4)n3)c3c(n2)oc2cc4ccccc4cc23)cc1 ; C1=c2ccccc2=CC2c3c(cc(-c4ccccc4)nc3-c3nc(-c4ccccc4)nc(-c4ccc5ccccc5c4)n3)OC12 ; c1ccc(-c2nc(-c3ccc4ccccc4c3)nc(-c3cc(-c4ccc5ccccc5c4)cc4oc5cc6ccccc6nc5c34)n2)cc1 ; c1ccc(-c2nc(-c3ccc4ccccc4c3)nc(-c3cc(-c4ccc5ccccc5c4)cc4oc5cc6cccnc6cc5c34)n2)cc1]

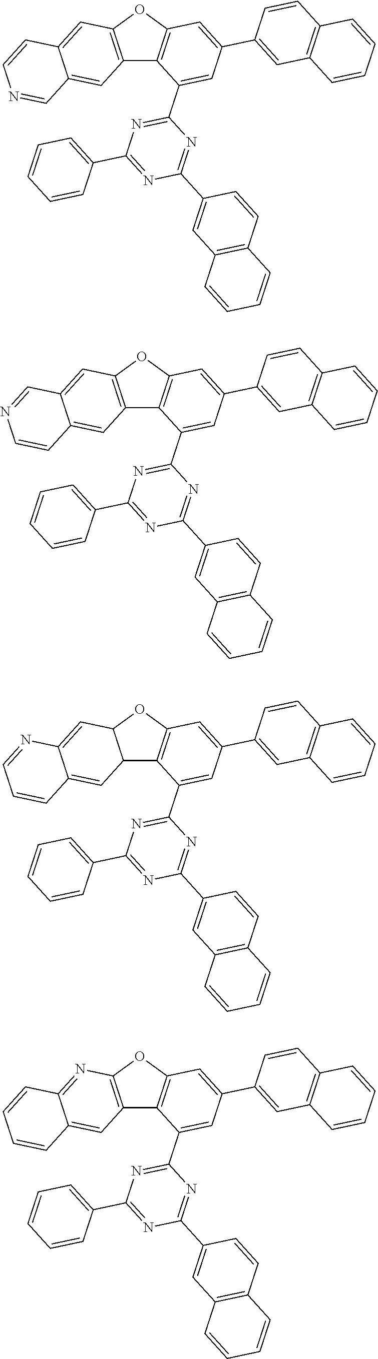 [SMILES: c1ccc(-c2nc(-c3ccc4ccccc4c3)nc(-c3cc(-c4ccc5ccccc5c4)cc4oc5cc6ccncc6cc5c34)n2)cc1 ; c1ccc(-c2nc(-c3ccc4ccccc4c3)nc(-c3cc(-c4ccc5ccccc5c4)cc4oc5cc6cnccc6cc5c34)n2)cc1 ; C1=c2cccnc2=CC2Oc3cc(-c4ccc5ccccc5c4)cc(-c4nc(-c5ccccc5)nc(-c5ccc6ccccc6c5)n4)c3C12 ; c1ccc(-c2nc(-c3ccc4ccccc4c3)nc(-c3cc(-c4ccc5ccccc5c4)cc4oc5nc6ccccc6cc5c34)n2)cc1]

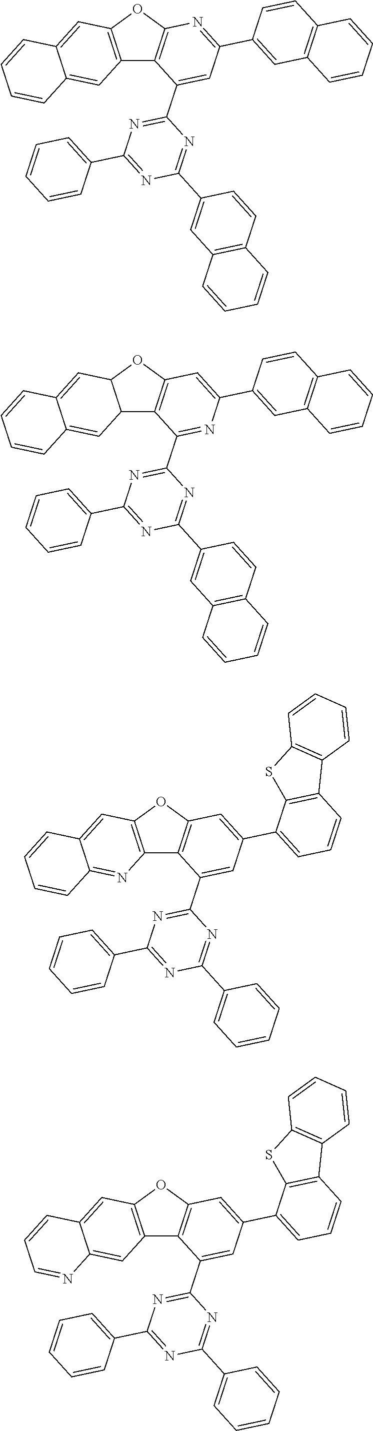 [SMILES: c1ccc(-c2nc(-c3ccc4ccccc4c3)nc(-c3cc(-c4ccc5ccccc5c4)nc4oc5cc6ccccc6cc5c34)n2)cc1 ; C1=c2ccccc2=CC2c3c(cc(-c4ccc5ccccc5c4)nc3-c3nc(-c4ccccc4)nc(-c4ccc5ccccc5c4)n3)OC12 ; c1ccc(-c2nc(-c3ccccc3)nc(-c3cc(-c4cccc5c4sc4ccccc45)cc4oc5cc6ccccc6nc5c34)n2)cc1 ; c1ccc(-c2nc(-c3ccccc3)nc(-c3cc(-c4cccc5c4sc4ccccc45)cc4oc5cc6cccnc6cc5c34)n2)cc1]

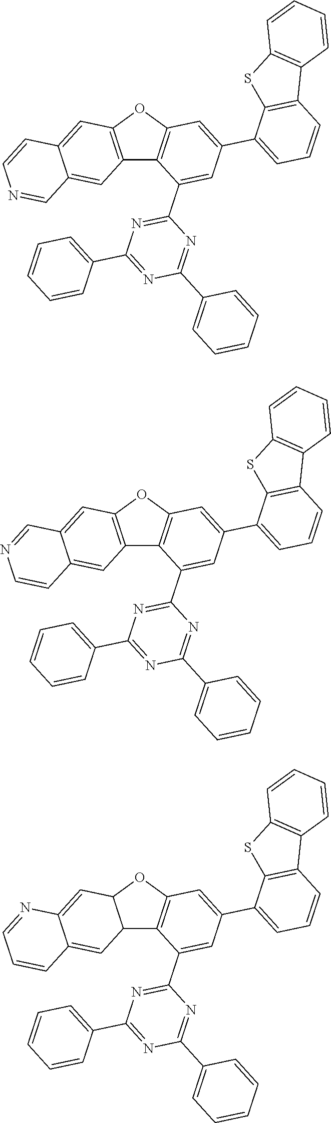 [SMILES: c1ccc(-c2nc(-c3ccccc3)nc(-c3cc(-c4cccc5c4sc4ccccc45)cc4oc5cc6ccncc6cc5c34)n2)cc1 ; c1ccc(-c2nc(-c3ccccc3)nc(-c3cc(-c4cccc5c4sc4ccccc45)cc4oc5cc6cnccc6cc5c34)n2)cc1 ; C1=c2cccnc2=CC2Oc3cc(-c4cccc5c4sc4ccccc45)cc(-c4nc(-c5ccccc5)nc(-c5ccccc5)n4)c3C12]

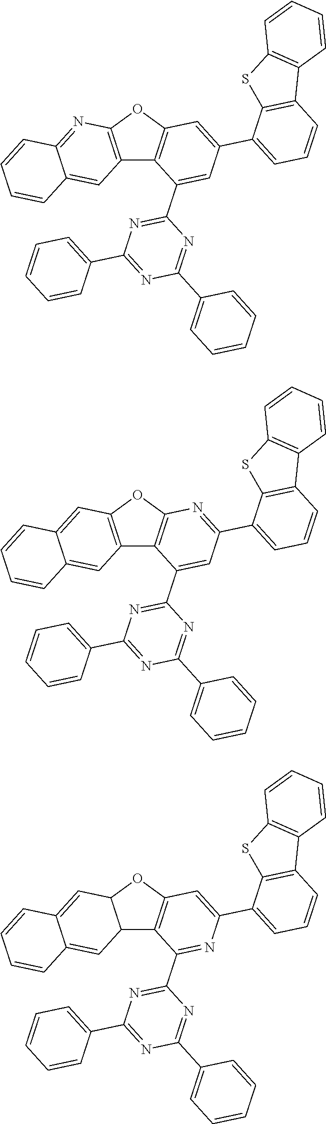 [SMILES: c1ccc(-c2nc(-c3ccccc3)nc(-c3cc(-c4cccc5c4sc4ccccc45)cc4oc5nc6ccccc6cc5c34)n2)cc1 ; c1ccc(-c2nc(-c3ccccc3)nc(-c3cc(-c4cccc5c4sc4ccccc45)nc4oc5cc6ccccc6cc5c34)n2)cc1 ; C1=c2ccccc2=CC2c3c(cc(-c4cccc5c4sc4ccccc45)nc3-c3nc(-c4ccccc4)nc(-c4ccccc4)n3)OC12]

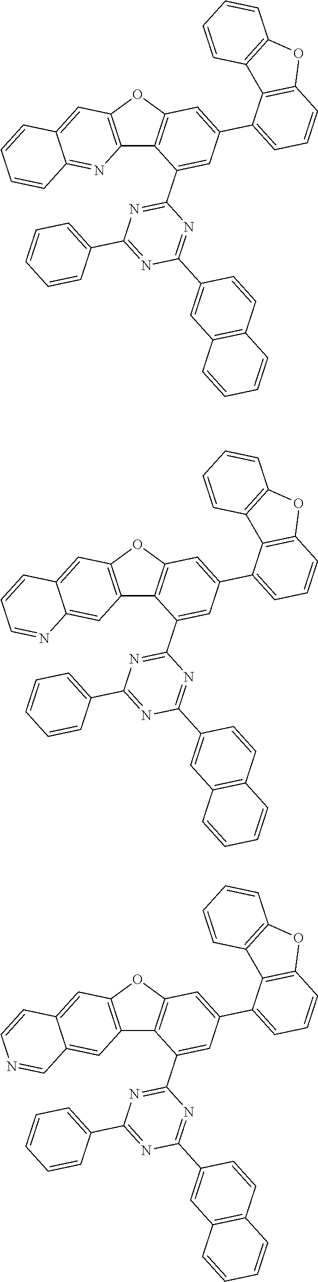

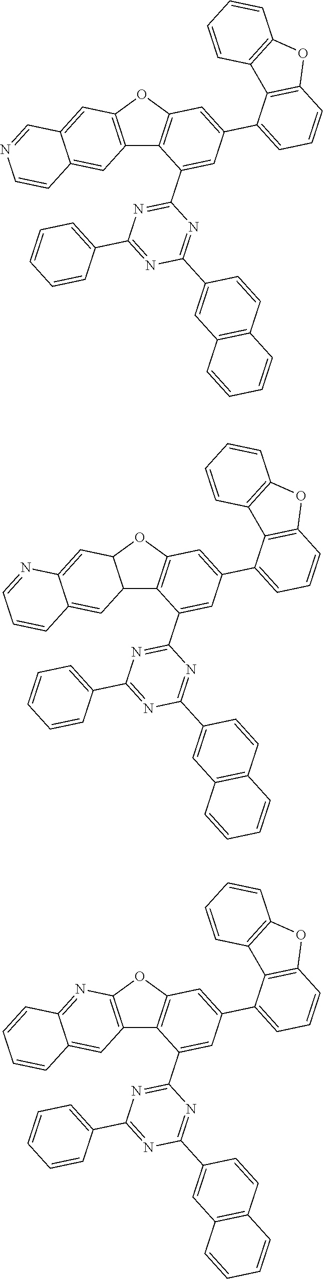

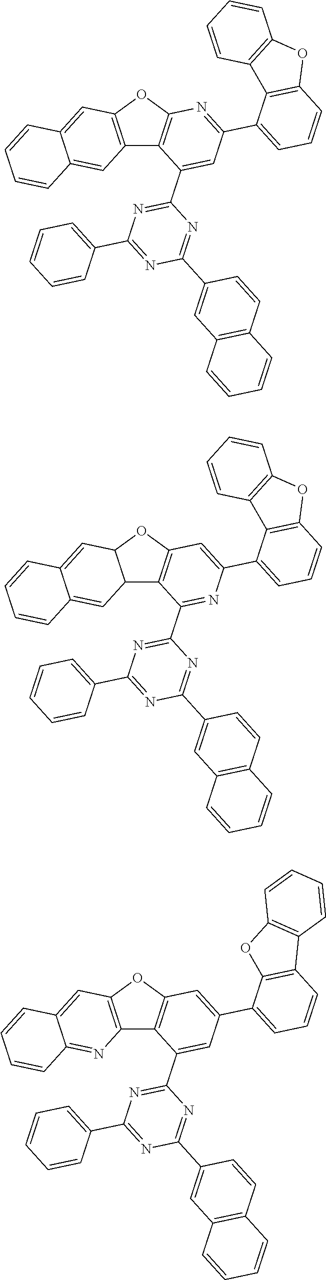

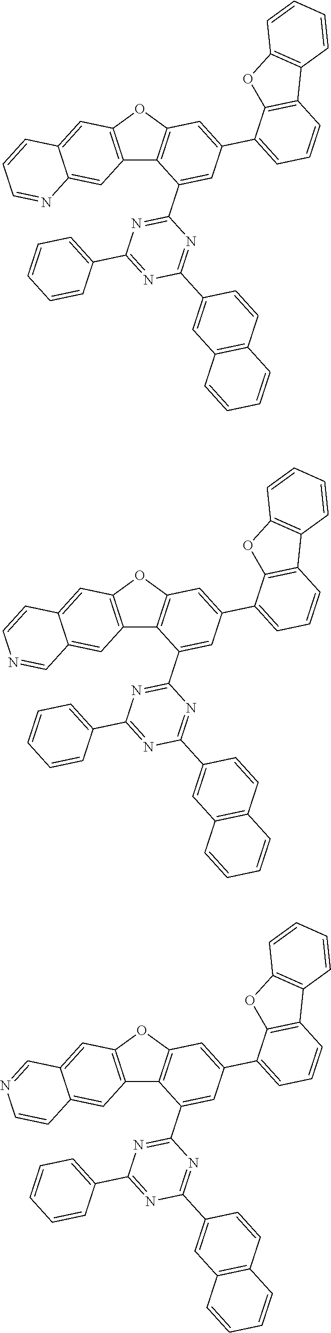 [SMILES: c1ccc(-c2nc(-c3ccc4ccccc4c3)nc(-c3cc(-c4cccc5c4oc4ccccc45)cc4oc5cc6cccnc6cc5c34)n2)cc1 ; c1ccc(-c2nc(-c3ccc4ccccc4c3)nc(-c3cc(-c4cccc5c4oc4ccccc45)cc4oc5cc6ccncc6cc5c34)n2)cc1 ; c1ccc(-c2nc(-c3ccc4ccccc4c3)nc(-c3cc(-c4cccc5c4oc4ccccc45)cc4oc5cc6cnccc6cc5c34)n2)cc1]

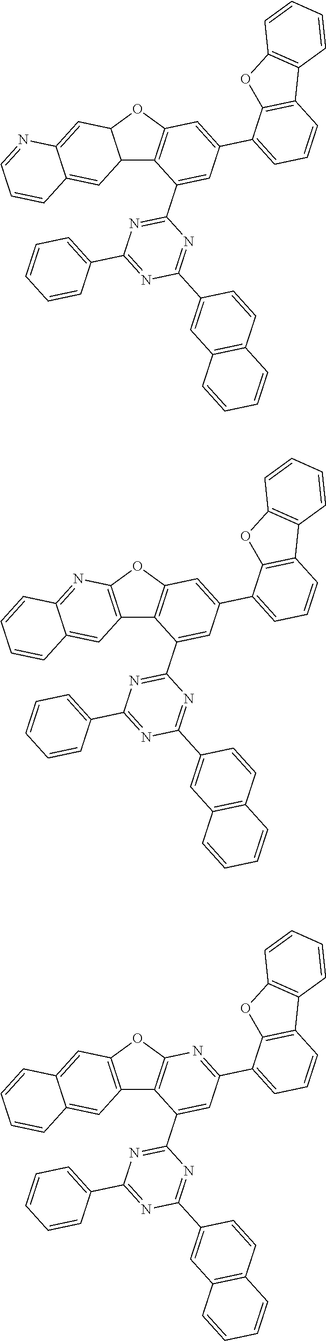 [SMILES: C1=c2cccnc2=CC2Oc3cc(-c4cccc5c4oc4ccccc45)cc(-c4nc(-c5ccccc5)nc(-c5ccc6ccccc6c5)n4)c3C12 ; c1ccc(-c2nc(-c3ccc4ccccc4c3)nc(-c3cc(-c4cccc5c4oc4ccccc45)cc4oc5nc6ccccc6cc5c34)n2)cc1 ; c1ccc(-c2nc(-c3ccc4ccccc4c3)nc(-c3cc(-c4cccc5c4oc4ccccc45)nc4oc5cc6ccccc6cc5c34)n2)cc1]

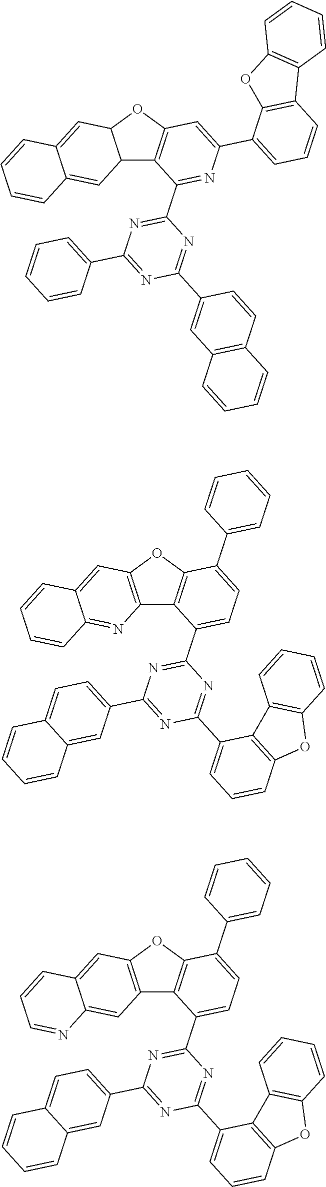 [SMILES: C1=c2ccccc2=CC2c3c(cc(-c4cccc5c4oc4ccccc45)nc3-c3nc(-c4ccccc4)nc(-c4ccc5ccccc5c4)n3)OC12 ; c1ccc(-c2ccc(-c3nc(-c4ccc5ccccc5c4)nc(-c4cccc5oc6ccccc6c45)n3)c3c2oc2cc4ccccc4nc23)cc1 ; c1ccc(-c2ccc(-c3nc(-c4ccc5ccccc5c4)nc(-c4cccc5oc6ccccc6c45)n3)c3c2oc2cc4cccnc4cc23)cc1]

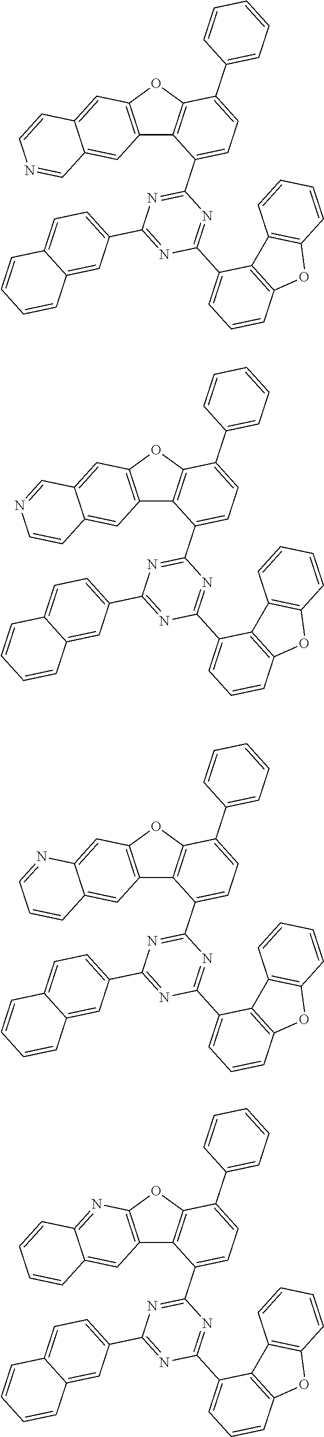

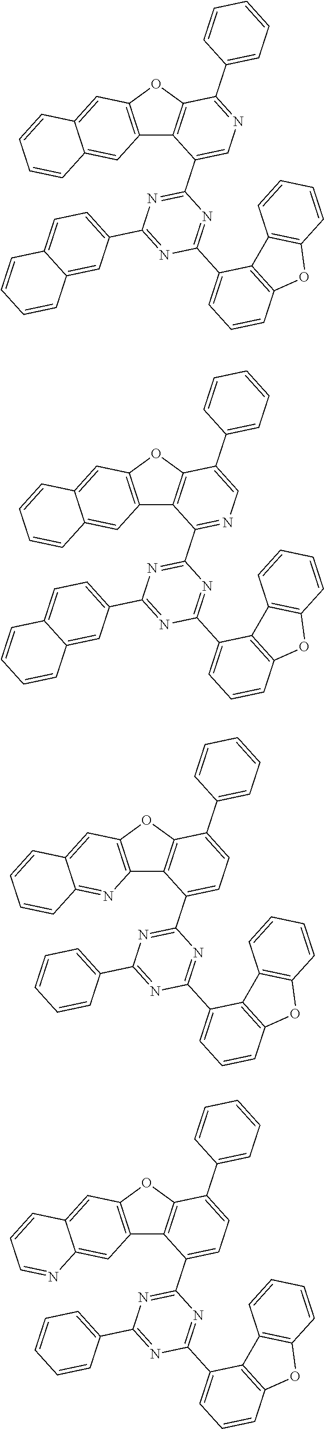

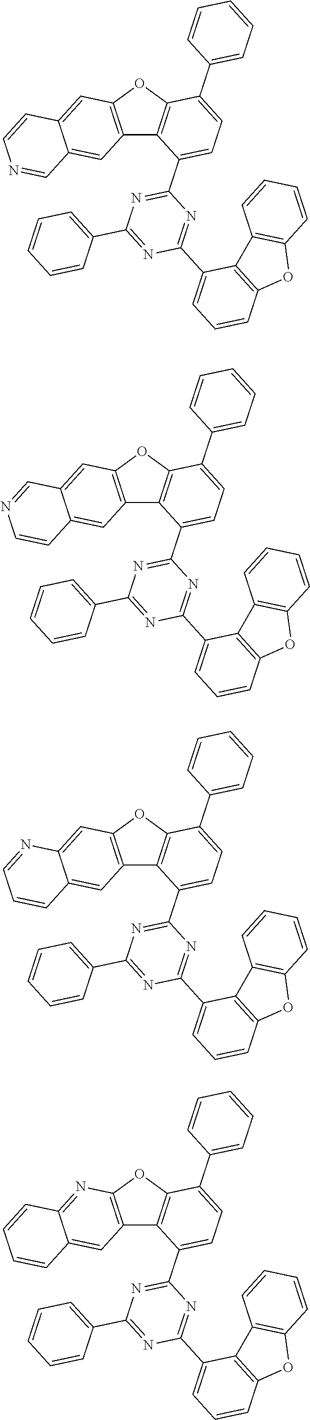

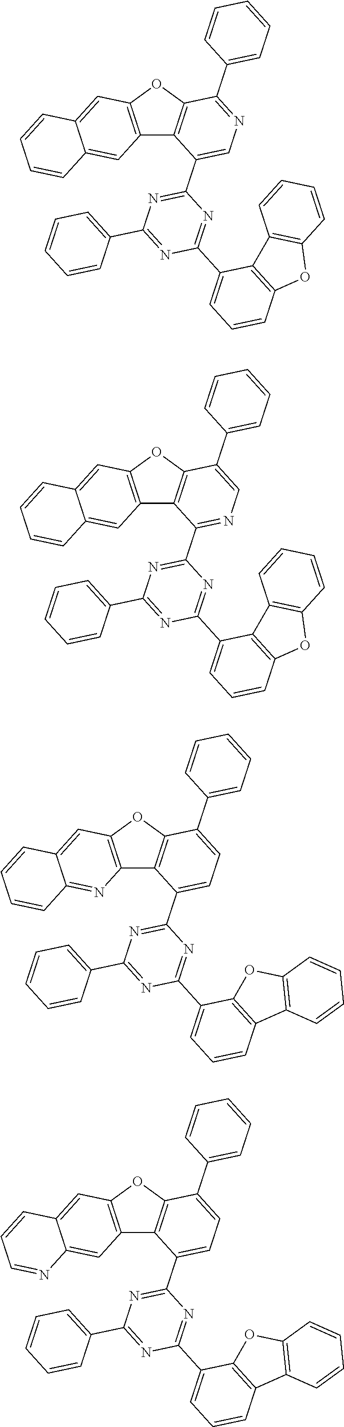

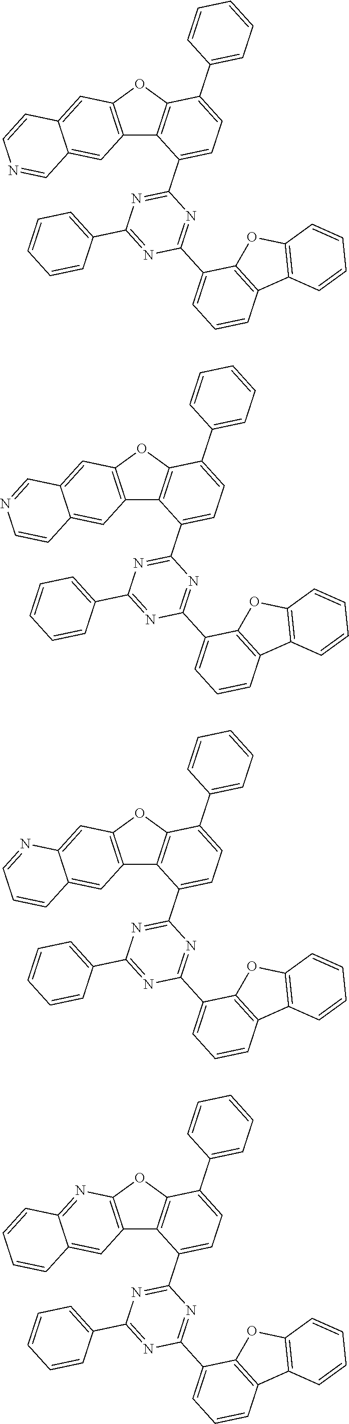

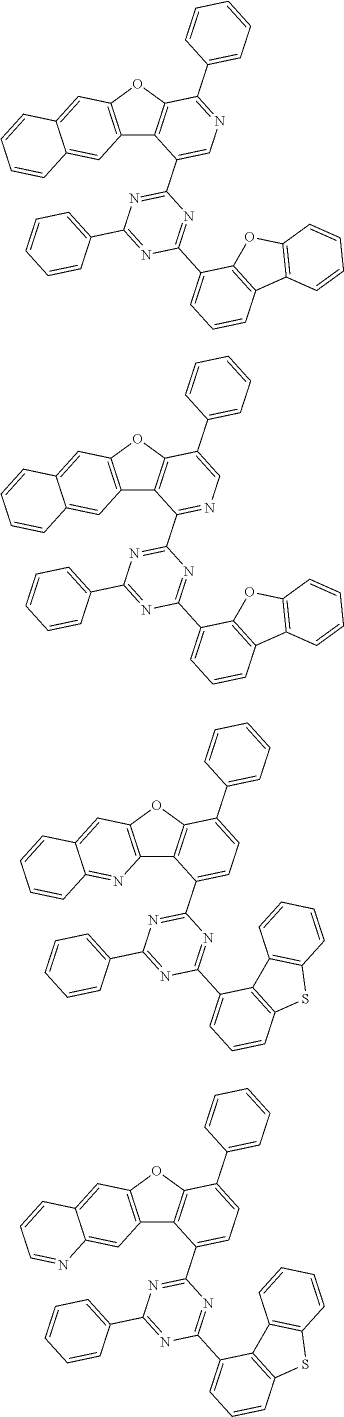

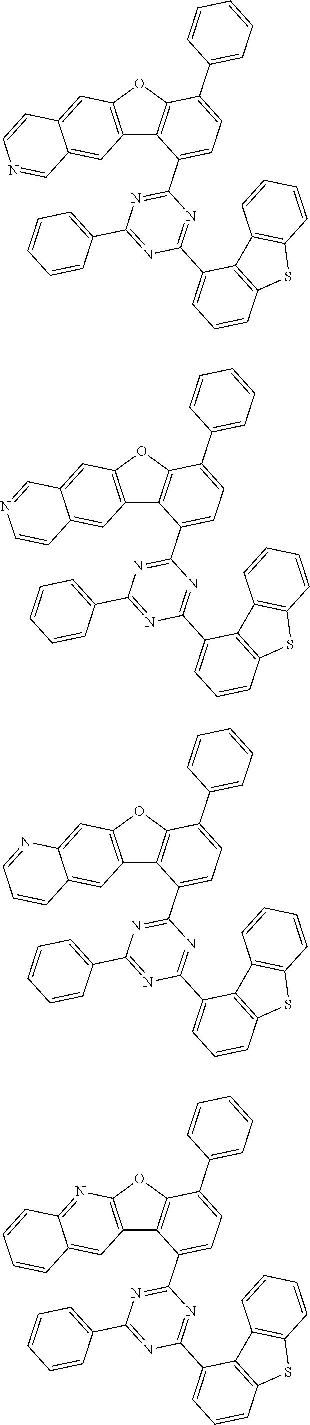

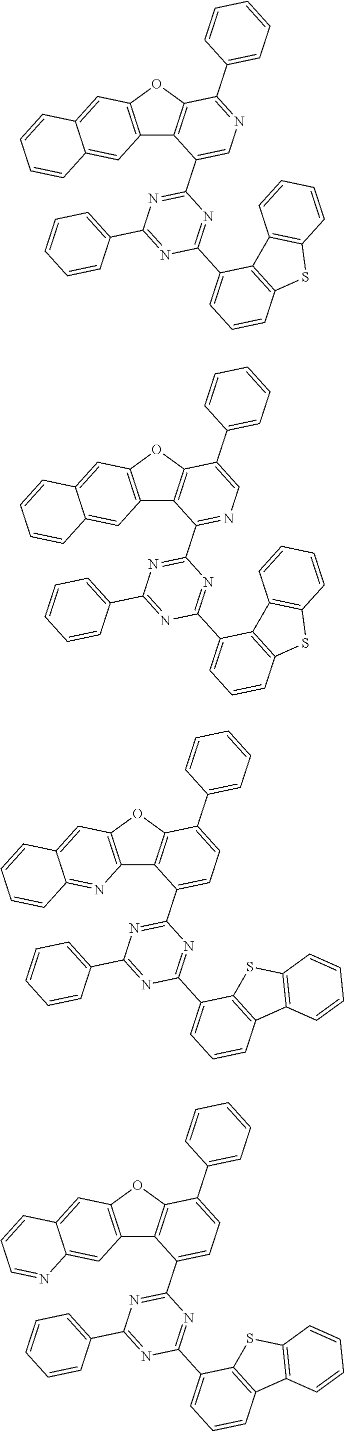

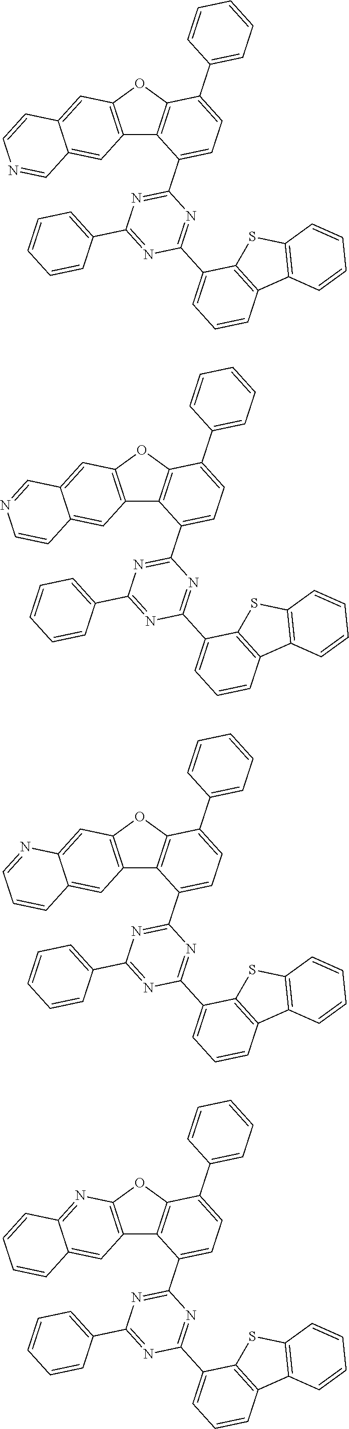

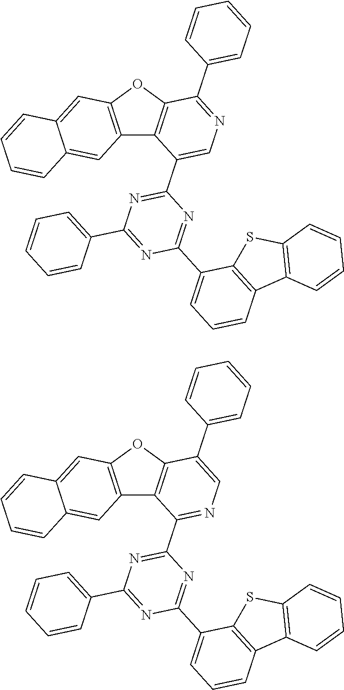

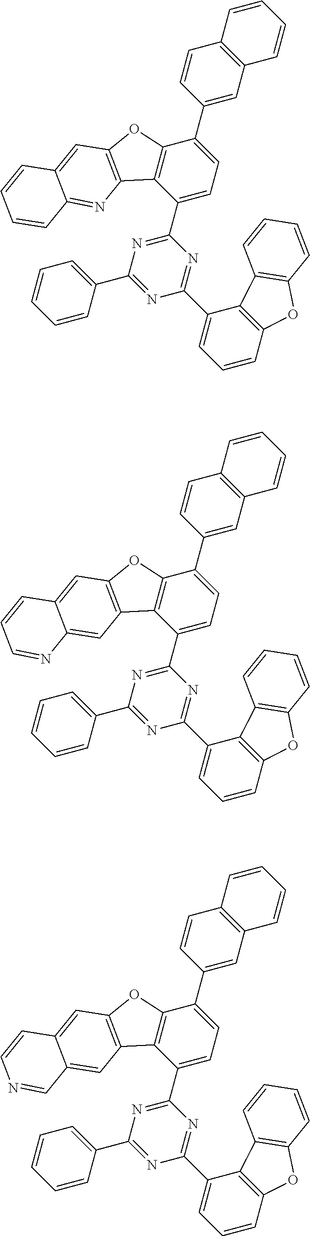 [SMILES: c1ccc(-c2nc(-c3cccc4oc5ccccc5c34)nc(-c3ccc(-c4ccc5ccccc5c4)c4oc5cc6ccccc6nc5c34)n2)cc1 ; c1ccc(-c2nc(-c3cccc4oc5ccccc5c34)nc(-c3ccc(-c4ccc5ccccc5c4)c4oc5cc6cccnc6cc5c34)n2)cc1 ; c1ccc(-c2nc(-c3cccc4oc5ccccc5c34)nc(-c3ccc(-c4ccc5ccccc5c4)c4oc5cc6ccncc6cc5c34)n2)cc1]

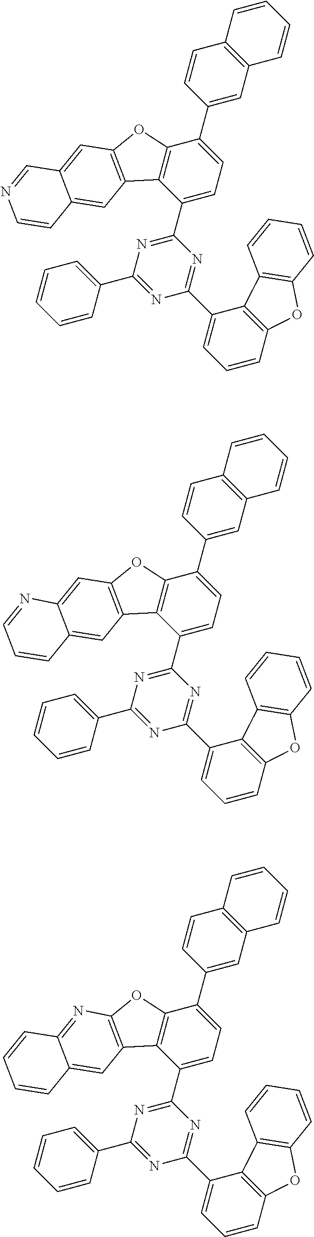 [SMILES: c1ccc(-c2nc(-c3cccc4oc5ccccc5c34)nc(-c3ccc(-c4ccc5ccccc5c4)c4oc5cc6cnccc6cc5c34)n2)cc1 ; c1ccc(-c2nc(-c3cccc4oc5ccccc5c34)nc(-c3ccc(-c4ccc5ccccc5c4)c4oc5cc6ncccc6cc5c34)n2)cc1 ; c1ccc(-c2nc(-c3cccc4oc5ccccc5c34)nc(-c3ccc(-c4ccc5ccccc5c4)c4oc5nc6ccccc6cc5c34)n2)cc1]

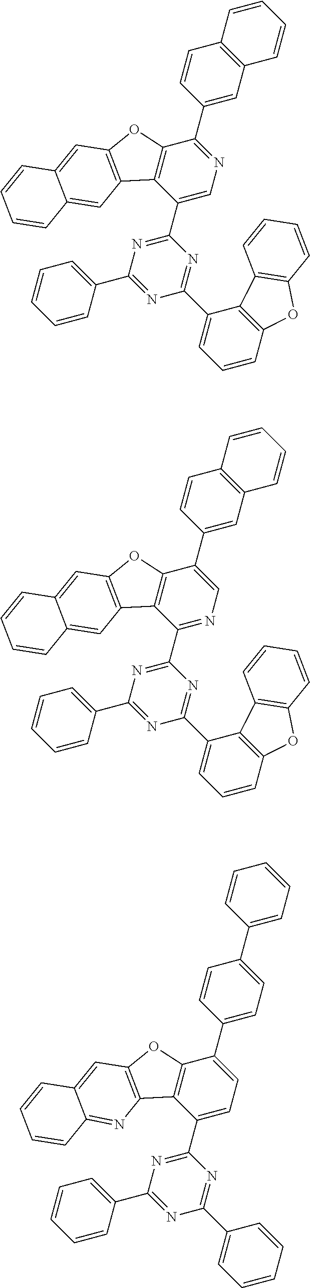 [SMILES: c1ccc(-c2nc(-c3cccc4oc5ccccc5c34)nc(-c3cnc(-c4ccc5ccccc5c4)c4oc5cc6ccccc6cc5c34)n2)cc1 ; c1ccc(-c2nc(-c3cccc4oc5ccccc5c34)nc(-c3ncc(-c4ccc5ccccc5c4)c4oc5cc6ccccc6cc5c34)n2)cc1 ; c1ccc(-c2ccc(-c3ccc(-c4nc(-c5ccccc5)nc(-c5ccccc5)n4)c4c3oc3cc5ccccc5nc34)cc2)cc1]

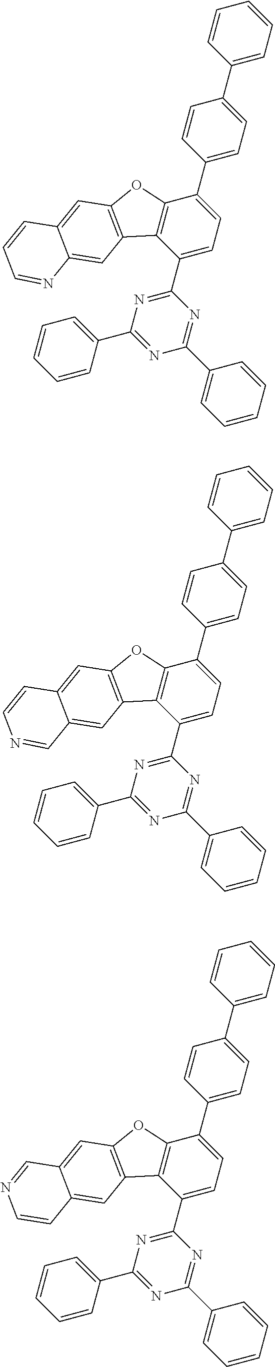 [SMILES: c1ccc(-c2ccc(-c3ccc(-c4nc(-c5ccccc5)nc(-c5ccccc5)n4)c4c3oc3cc5cccnc5cc34)cc2)cc1 ; c1ccc(-c2ccc(-c3ccc(-c4nc(-c5ccccc5)nc(-c5ccccc5)n4)c4c3oc3cc5ccncc5cc34)cc2)cc1 ; c1ccc(-c2ccc(-c3ccc(-c4nc(-c5ccccc5)nc(-c5ccccc5)n4)c4c3oc3cc5cnccc5cc34)cc2)cc1]

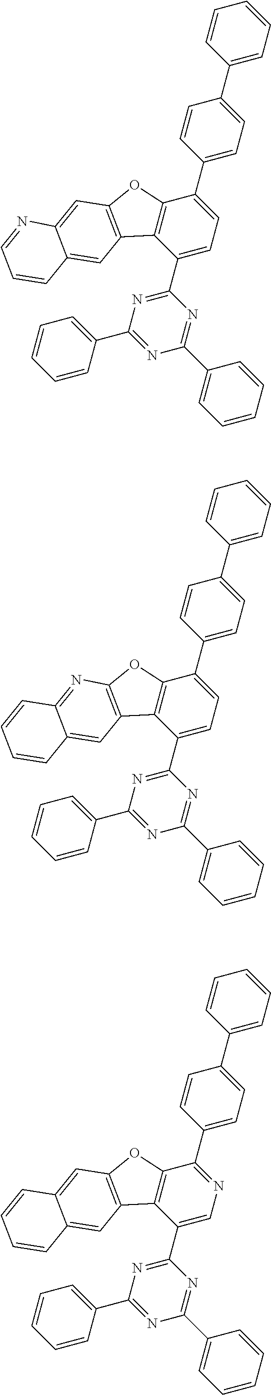 [SMILES: c1ccc(-c2ccc(-c3ccc(-c4nc(-c5ccccc5)nc(-c5ccccc5)n4)c4c3oc3cc5ncccc5cc34)cc2)cc1 ; c1ccc(-c2ccc(-c3ccc(-c4nc(-c5ccccc5)nc(-c5ccccc5)n4)c4c3oc3nc5ccccc5cc34)cc2)cc1 ; c1ccc(-c2ccc(-c3ncc(-c4nc(-c5ccccc5)nc(-c5ccccc5)n4)c4c3oc3cc5ccccc5cc34)cc2)cc1]

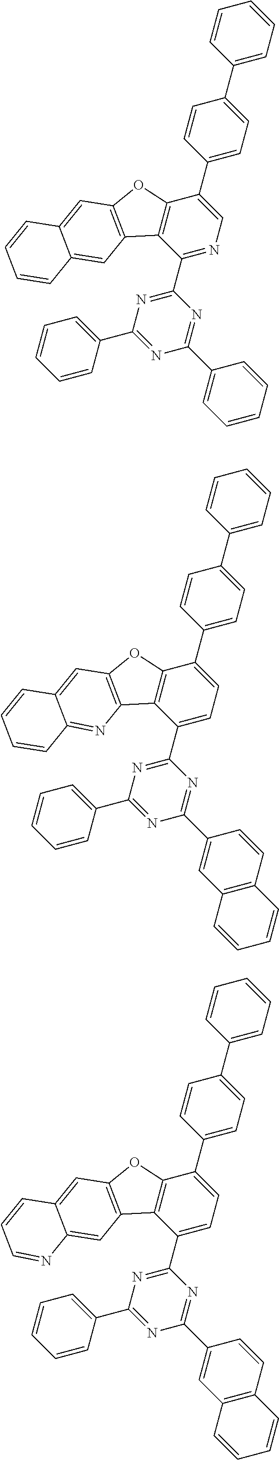

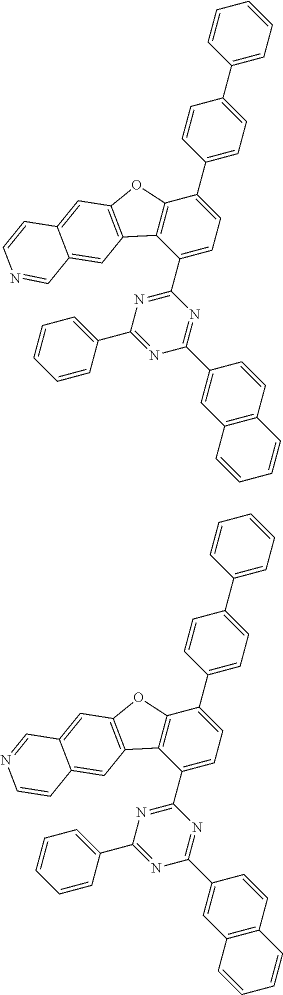

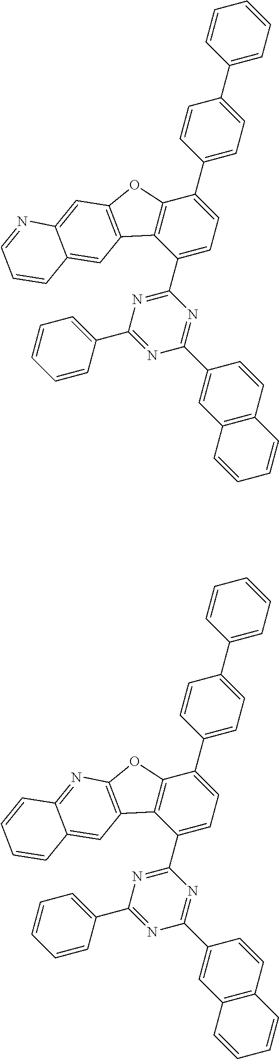 [SMILES: c1ccc(-c2ccc(-c3ccc(-c4nc(-c5ccccc5)nc(-c5ccc6ccccc6c5)n4)c4c3oc3cc5ncccc5cc34)cc2)cc1 ; c1ccc(-c2ccc(-c3ccc(-c4nc(-c5ccccc5)nc(-c5ccc6ccccc6c5)n4)c4c3oc3nc5ccccc5cc34)cc2)cc1]

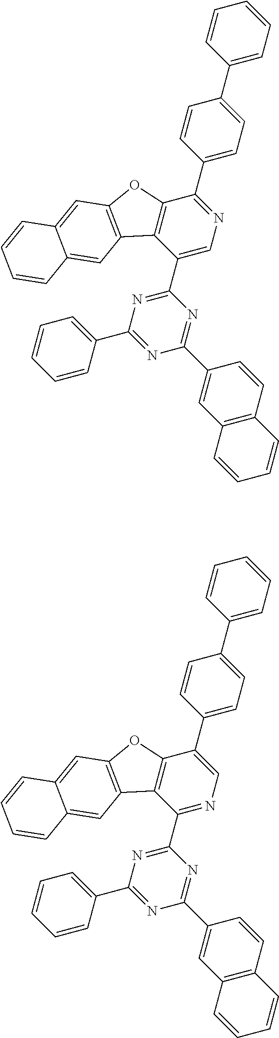 [SMILES: c1ccc(-c2ccc(-c3ncc(-c4nc(-c5ccccc5)nc(-c5ccc6ccccc6c5)n4)c4c3oc3cc5ccccc5cc34)cc2)cc1 ; c1ccc(-c2ccc(-c3cnc(-c4nc(-c5ccccc5)nc(-c5ccc6ccccc6c5)n4)c4c3oc3cc5ccccc5cc34)cc2)cc1]

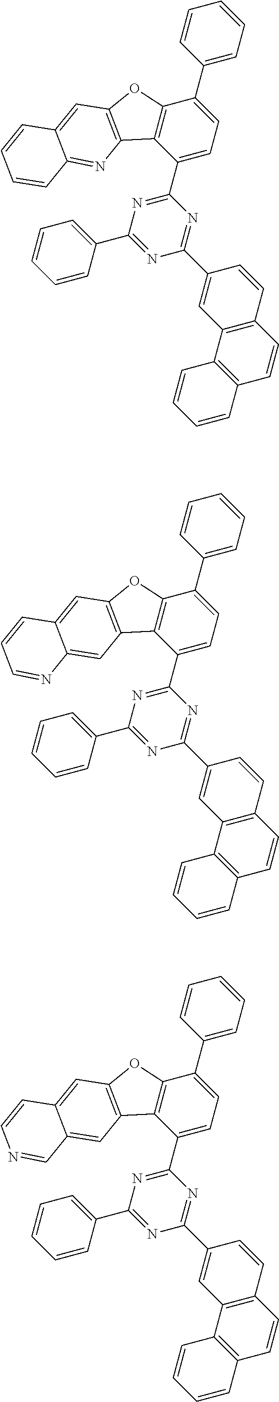 [SMILES: c1ccc(-c2nc(-c3ccc4ccc5ccccc5c4c3)nc(-c3ccc(-c4ccccc4)c4oc5cc6ccccc6nc5c34)n2)cc1 ; c1ccc(-c2nc(-c3ccc4ccc5ccccc5c4c3)nc(-c3ccc(-c4ccccc4)c4oc5cc6cccnc6cc5c34)n2)cc1 ; c1ccc(-c2nc(-c3ccc4ccc5ccccc5c4c3)nc(-c3ccc(-c4ccccc4)c4oc5cc6ccncc6cc5c34)n2)cc1]

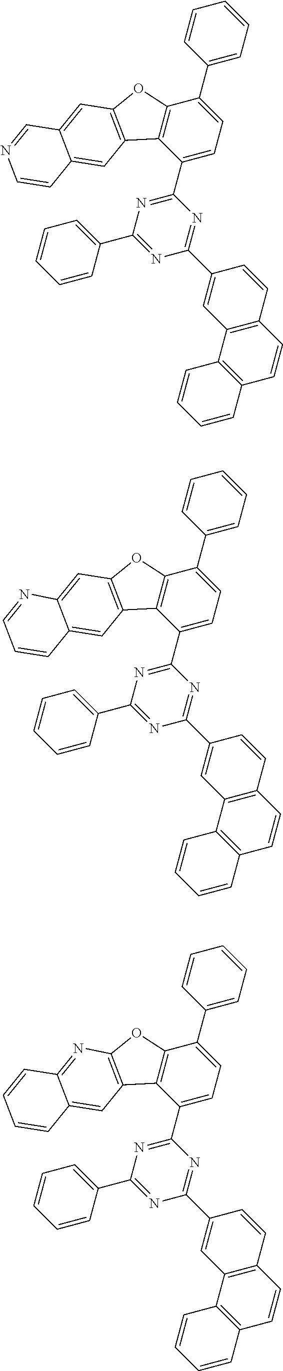 [SMILES: c1ccc(-c2nc(-c3ccc4ccc5ccccc5c4c3)nc(-c3ccc(-c4ccccc4)c4oc5cc6cnccc6cc5c34)n2)cc1 ; c1ccc(-c2nc(-c3ccc4ccc5ccccc5c4c3)nc(-c3ccc(-c4ccccc4)c4oc5cc6ncccc6cc5c34)n2)cc1 ; c1ccc(-c2nc(-c3ccc4ccc5ccccc5c4c3)nc(-c3ccc(-c4ccccc4)c4oc5nc6ccccc6cc5c34)n2)cc1]

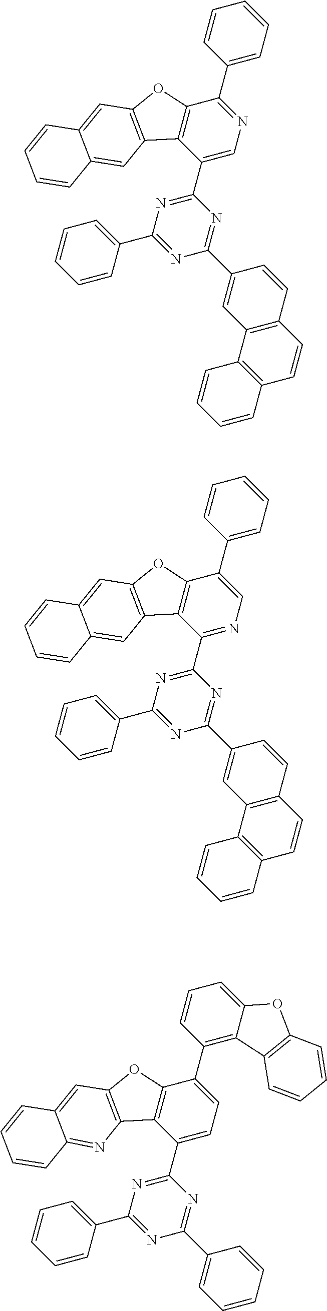 [SMILES: c1ccc(-c2nc(-c3ccc4ccc5ccccc5c4c3)nc(-c3cnc(-c4ccccc4)c4oc5cc6ccccc6cc5c34)n2)cc1 ; c1ccc(-c2nc(-c3ccc4ccc5ccccc5c4c3)nc(-c3ncc(-c4ccccc4)c4oc5cc6ccccc6cc5c34)n2)cc1 ; c1ccc(-c2nc(-c3ccccc3)nc(-c3ccc(-c4cccc5oc6ccccc6c45)c4oc5cc6ccccc6nc5c34)n2)cc1]

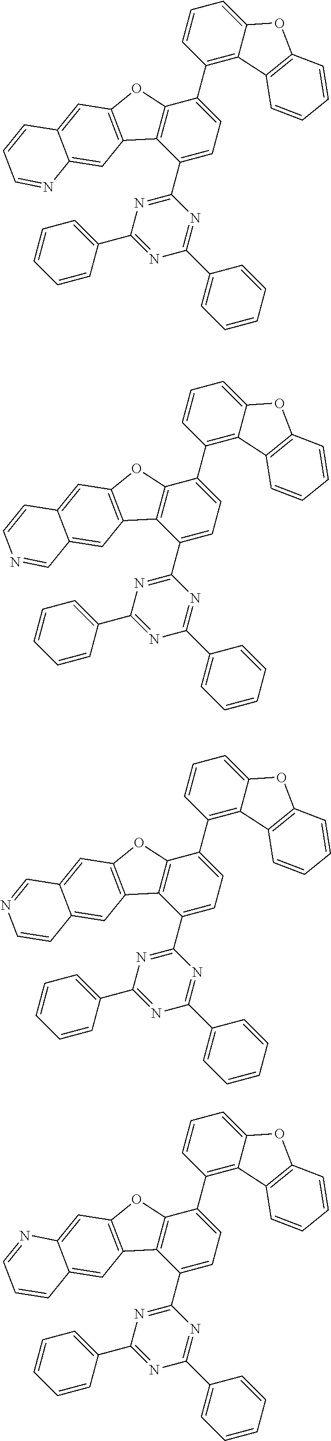

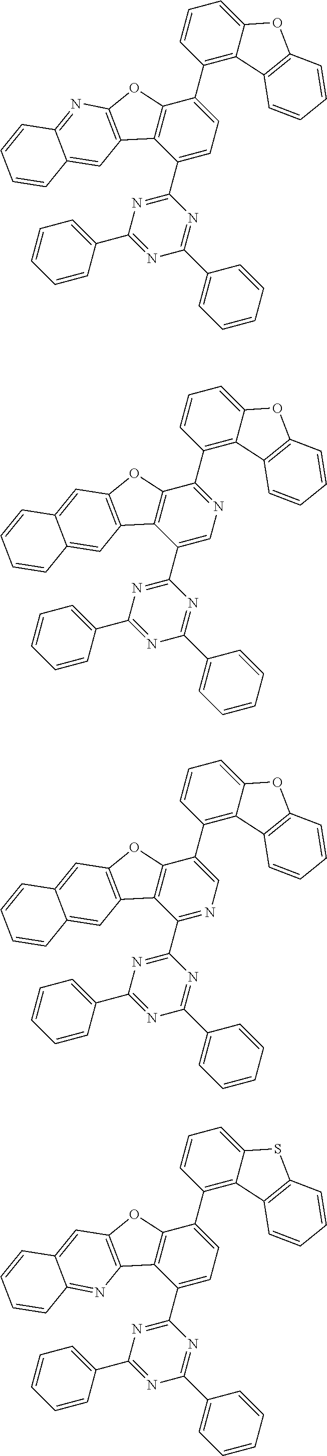

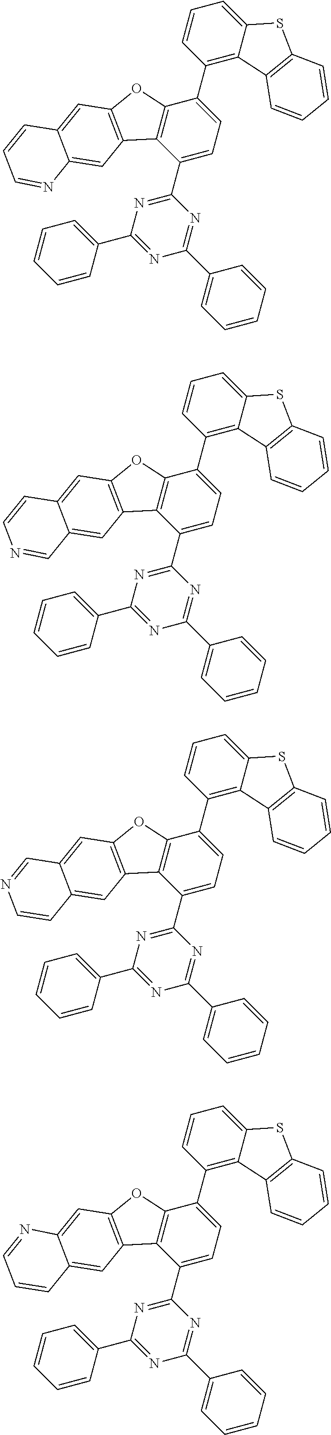

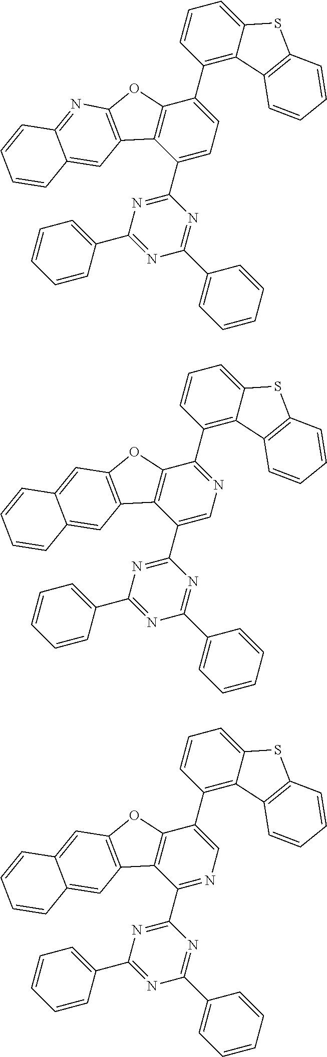

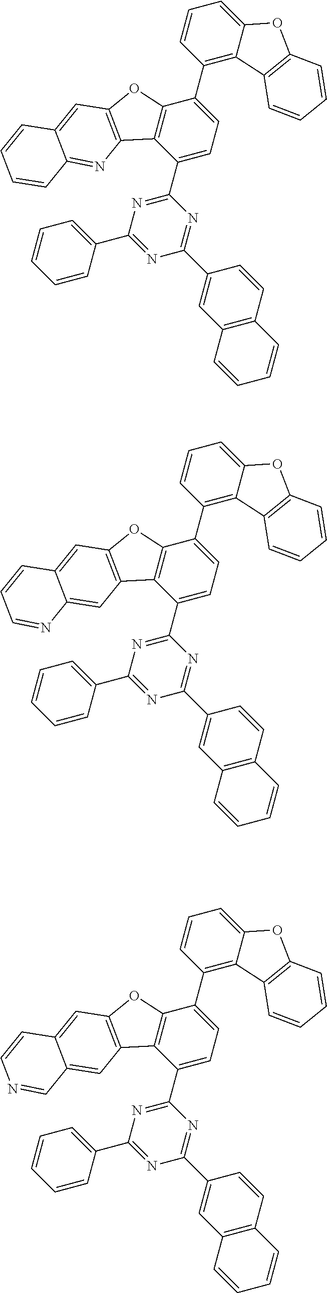 [SMILES: c1ccc(-c2nc(-c3ccc4ccccc4c3)nc(-c3ccc(-c4cccc5oc6ccccc6c45)c4oc5cc6ccccc6nc5c34)n2)cc1 ; c1ccc(-c2nc(-c3ccc4ccccc4c3)nc(-c3ccc(-c4cccc5oc6ccccc6c45)c4oc5cc6cccnc6cc5c34)n2)cc1 ; c1ccc(-c2nc(-c3ccc4ccccc4c3)nc(-c3ccc(-c4cccc5oc6ccccc6c45)c4oc5cc6ccncc6cc5c34)n2)cc1]

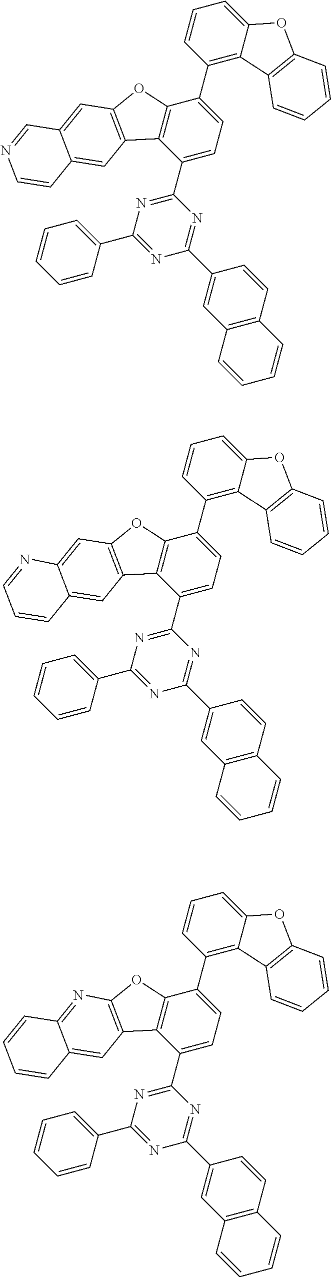 [SMILES: c1ccc(-c2nc(-c3ccc4ccccc4c3)nc(-c3ccc(-c4cccc5oc6ccccc6c45)c4oc5cc6cnccc6cc5c34)n2)cc1 ; c1ccc(-c2nc(-c3ccc4ccccc4c3)nc(-c3ccc(-c4cccc5oc6ccccc6c45)c4oc5cc6ncccc6cc5c34)n2)cc1 ; c1ccc(-c2nc(-c3ccc4ccccc4c3)nc(-c3ccc(-c4cccc5oc6ccccc6c45)c4oc5nc6ccccc6cc5c34)n2)cc1]

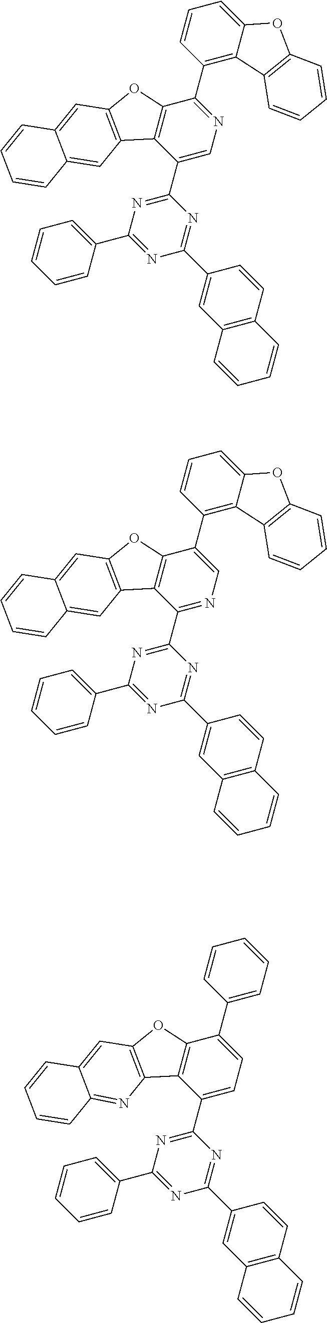 [SMILES: c1ccc(-c2nc(-c3ccc4ccccc4c3)nc(-c3cnc(-c4cccc5oc6ccccc6c45)c4oc5cc6ccccc6cc5c34)n2)cc1 ; c1ccc(-c2nc(-c3ccc4ccccc4c3)nc(-c3ncc(-c4cccc5oc6ccccc6c45)c4oc5cc6ccccc6cc5c34)n2)cc1 ; c1ccc(-c2nc(-c3ccc4ccccc4c3)nc(-c3ccc(-c4ccccc4)c4oc5cc6ccccc6nc5c34)n2)cc1]

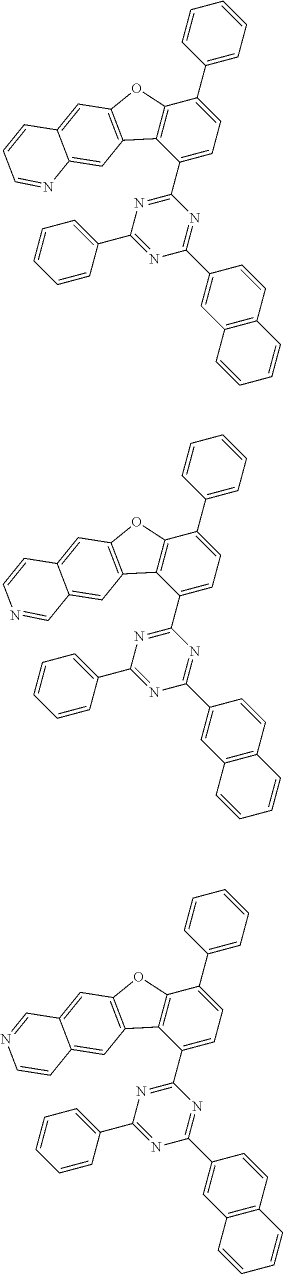 [SMILES: c1ccc(-c2nc(-c3ccc4ccccc4c3)nc(-c3ccc(-c4ccccc4)c4oc5cc6cccnc6cc5c34)n2)cc1 ; c1ccc(-c2nc(-c3ccc4ccccc4c3)nc(-c3ccc(-c4ccccc4)c4oc5cc6ccncc6cc5c34)n2)cc1 ; c1ccc(-c2nc(-c3ccc4ccccc4c3)nc(-c3ccc(-c4ccccc4)c4oc5cc6cnccc6cc5c34)n2)cc1]

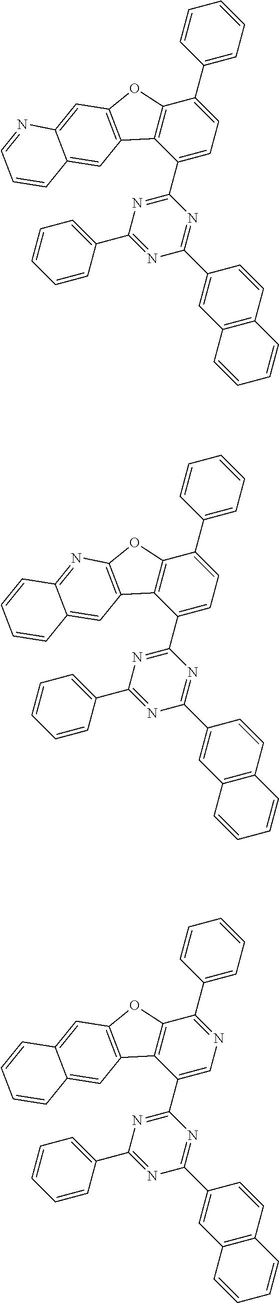 [SMILES: c1ccc(-c2nc(-c3ccc4ccccc4c3)nc(-c3ccc(-c4ccccc4)c4oc5cc6ncccc6cc5c34)n2)cc1 ; c1ccc(-c2nc(-c3ccc4ccccc4c3)nc(-c3ccc(-c4ccccc4)c4oc5nc6ccccc6cc5c34)n2)cc1 ; c1ccc(-c2nc(-c3ccc4ccccc4c3)nc(-c3cnc(-c4ccccc4)c4oc5cc6ccccc6cc5c34)n2)cc1]

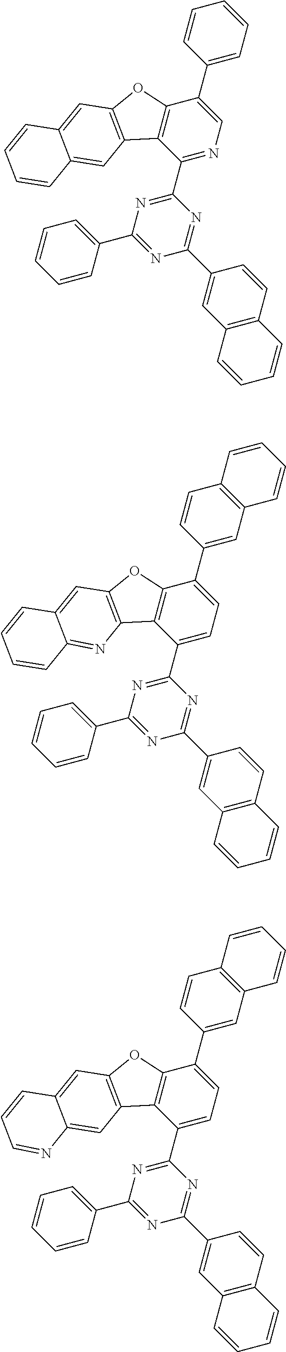 [SMILES: c1ccc(-c2nc(-c3ccc4ccccc4c3)nc(-c3ncc(-c4ccccc4)c4oc5cc6ccccc6cc5c34)n2)cc1 ; c1ccc(-c2nc(-c3ccc4ccccc4c3)nc(-c3ccc(-c4ccc5ccccc5c4)c4oc5cc6ccccc6nc5c34)n2)cc1 ; c1ccc(-c2nc(-c3ccc4ccccc4c3)nc(-c3ccc(-c4ccc5ccccc5c4)c4oc5cc6cccnc6cc5c34)n2)cc1]

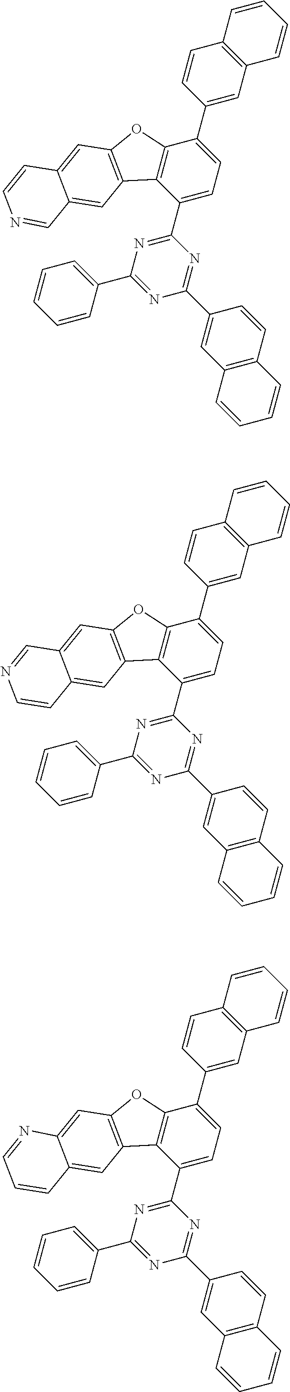 [SMILES: c1ccc(-c2nc(-c3ccc4ccccc4c3)nc(-c3ccc(-c4ccc5ccccc5c4)c4oc5cc6ccncc6cc5c34)n2)cc1 ; c1ccc(-c2nc(-c3ccc4ccccc4c3)nc(-c3ccc(-c4ccc5ccccc5c4)c4oc5cc6cnccc6cc5c34)n2)cc1 ; c1ccc(-c2nc(-c3ccc4ccccc4c3)nc(-c3ccc(-c4ccc5ccccc5c4)c4oc5cc6ncccc6cc5c34)n2)cc1]

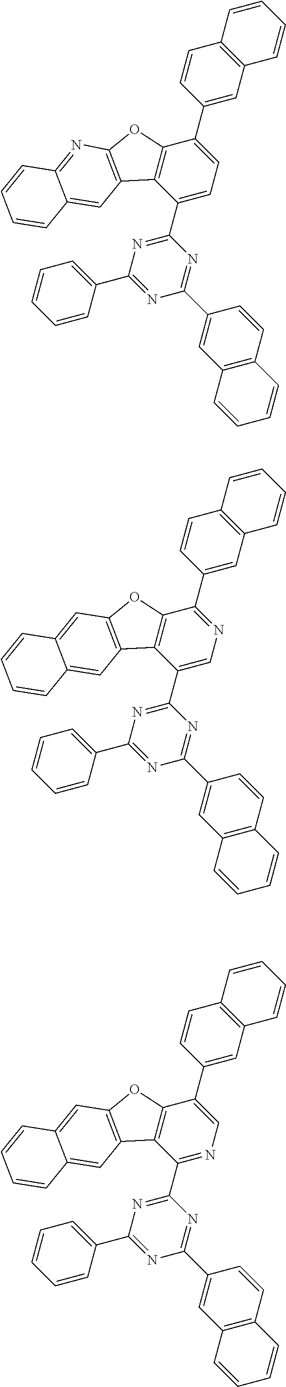 [SMILES: c1ccc(-c2nc(-c3ccc4ccccc4c3)nc(-c3ccc(-c4ccc5ccccc5c4)c4oc5nc6ccccc6cc5c34)n2)cc1 ; c1ccc(-c2nc(-c3ccc4ccccc4c3)nc(-c3cnc(-c4ccc5ccccc5c4)c4oc5cc6ccccc6cc5c34)n2)cc1 ; c1ccc(-c2nc(-c3ccc4ccccc4c3)nc(-c3ncc(-c4ccc5ccccc5c4)c4oc5cc6ccccc6cc5c34)n2)cc1]

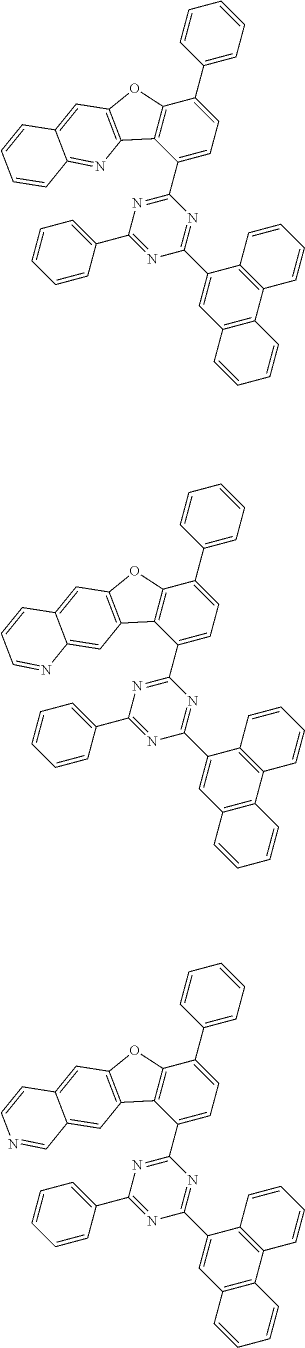 [SMILES: c1ccc(-c2nc(-c3cc4ccccc4c4ccccc34)nc(-c3ccc(-c4ccccc4)c4oc5cc6ccccc6nc5c34)n2)cc1 ; c1ccc(-c2nc(-c3cc4ccccc4c4ccccc34)nc(-c3ccc(-c4ccccc4)c4oc5cc6cccnc6cc5c34)n2)cc1 ; c1ccc(-c2nc(-c3cc4ccccc4c4ccccc34)nc(-c3ccc(-c4ccccc4)c4oc5cc6ccncc6cc5c34)n2)cc1]

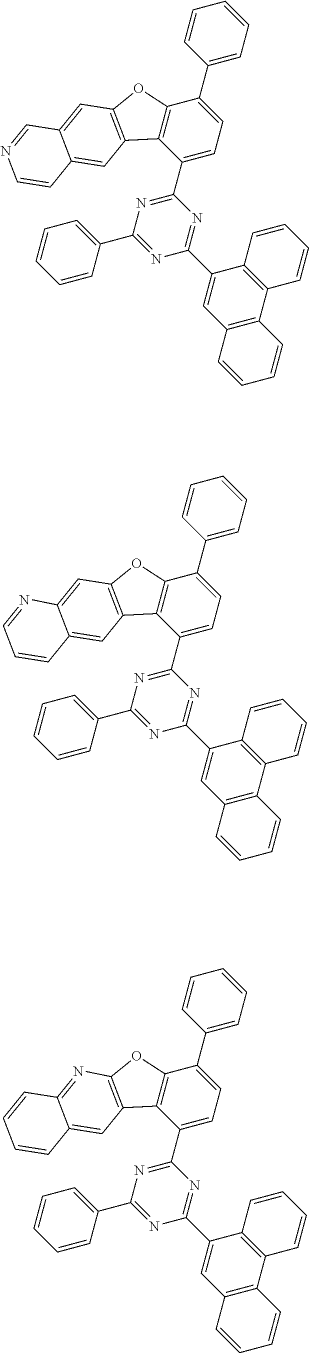 [SMILES: c1ccc(-c2nc(-c3cc4ccccc4c4ccccc34)nc(-c3ccc(-c4ccccc4)c4oc5cc6cnccc6cc5c34)n2)cc1 ; c1ccc(-c2nc(-c3cc4ccccc4c4ccccc34)nc(-c3ccc(-c4ccccc4)c4oc5cc6ncccc6cc5c34)n2)cc1 ; c1ccc(-c2nc(-c3cc4ccccc4c4ccccc34)nc(-c3ccc(-c4ccccc4)c4oc5nc6ccccc6cc5c34)n2)cc1]

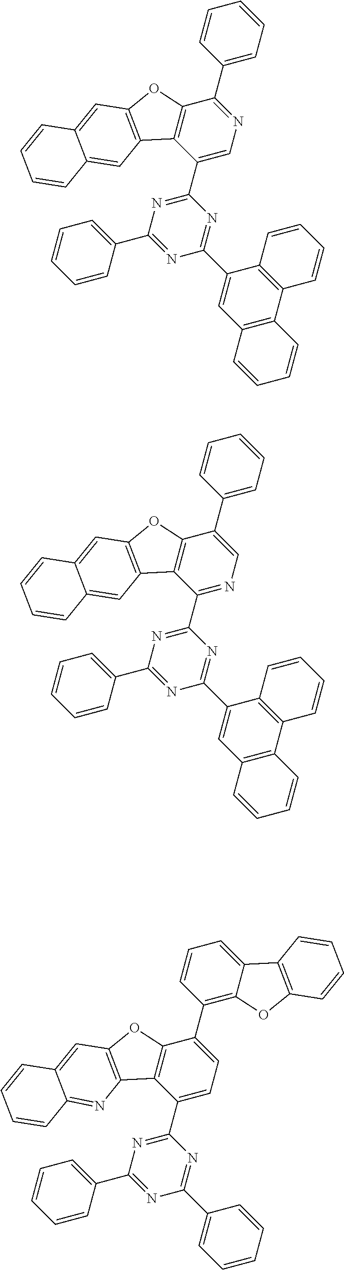 [SMILES: c1ccc(-c2nc(-c3cc4ccccc4c4ccccc34)nc(-c3cnc(-c4ccccc4)c4oc5cc6ccccc6cc5c34)n2)cc1 ; c1ccc(-c2nc(-c3cc4ccccc4c4ccccc34)nc(-c3ncc(-c4ccccc4)c4oc5cc6ccccc6cc5c34)n2)cc1 ; c1ccc(-c2nc(-c3ccccc3)nc(-c3ccc(-c4cccc5c4oc4ccccc45)c4oc5cc6ccccc6nc5c34)n2)cc1]

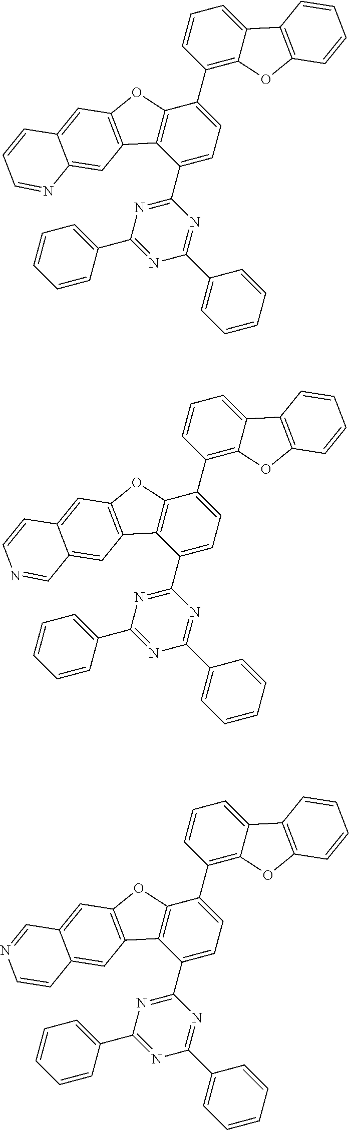 [SMILES: c1ccc(-c2nc(-c3ccccc3)nc(-c3ccc(-c4cccc5c4oc4ccccc45)c4oc5cc6cccnc6cc5c34)n2)cc1 ; c1ccc(-c2nc(-c3ccccc3)nc(-c3ccc(-c4cccc5c4oc4ccccc45)c4oc5cc6ccncc6cc5c34)n2)cc1 ; c1ccc(-c2nc(-c3ccccc3)nc(-c3ccc(-c4cccc5c4oc4ccccc45)c4oc5cc6cnccc6cc5c34)n2)cc1]

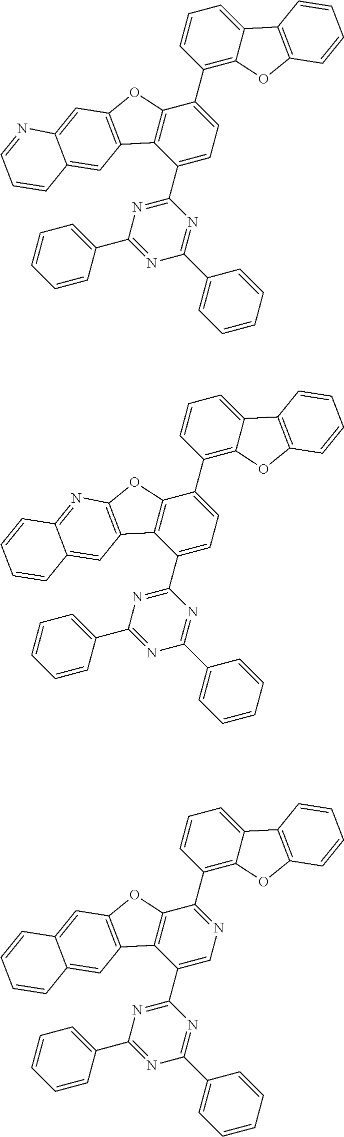 [SMILES: c1ccc(-c2nc(-c3ccccc3)nc(-c3ccc(-c4cccc5c4oc4ccccc45)c4oc5cc6ncccc6cc5c34)n2)cc1 ; c1ccc(-c2nc(-c3ccccc3)nc(-c3ccc(-c4cccc5c4oc4ccccc45)c4oc5nc6ccccc6cc5c34)n2)cc1 ; c1ccc(-c2nc(-c3ccccc3)nc(-c3cnc(-c4cccc5c4oc4ccccc45)c4oc5cc6ccccc6cc5c34)n2)cc1]

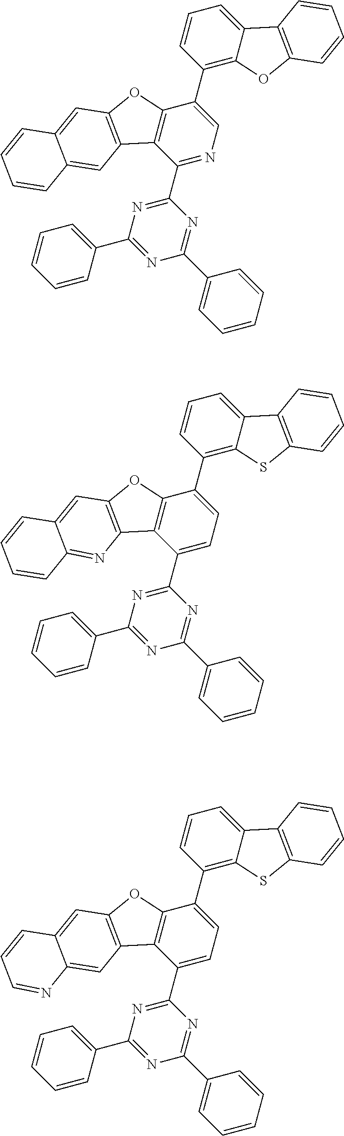 [SMILES: c1ccc(-c2nc(-c3ccccc3)nc(-c3ncc(-c4cccc5c4oc4ccccc45)c4oc5cc6ccccc6cc5c34)n2)cc1 ; c1ccc(-c2nc(-c3ccccc3)nc(-c3ccc(-c4cccc5c4sc4ccccc45)c4oc5cc6ccccc6nc5c34)n2)cc1 ; c1ccc(-c2nc(-c3ccccc3)nc(-c3ccc(-c4cccc5c4sc4ccccc45)c4oc5cc6cccnc6cc5c34)n2)cc1]

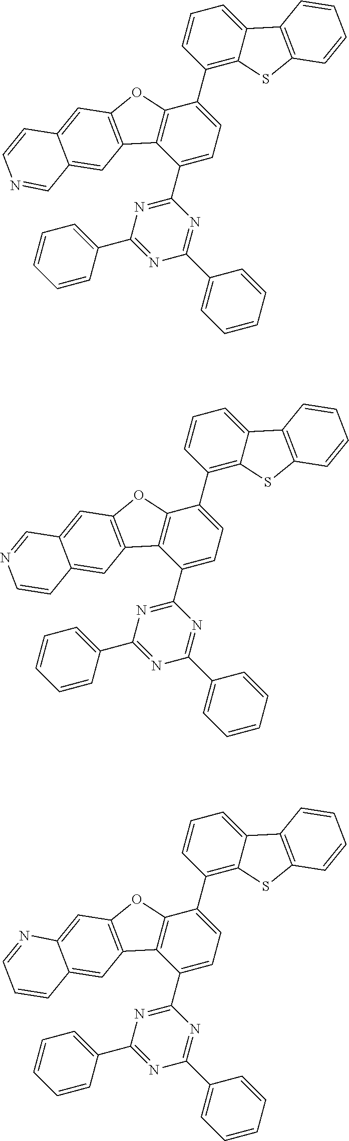 [SMILES: c1ccc(-c2nc(-c3ccccc3)nc(-c3ccc(-c4cccc5c4sc4ccccc45)c4oc5cc6ccncc6cc5c34)n2)cc1 ; c1ccc(-c2nc(-c3ccccc3)nc(-c3ccc(-c4cccc5c4sc4ccccc45)c4oc5cc6cnccc6cc5c34)n2)cc1 ; c1ccc(-c2nc(-c3ccccc3)nc(-c3ccc(-c4cccc5c4sc4ccccc45)c4oc5cc6ncccc6cc5c34)n2)cc1]

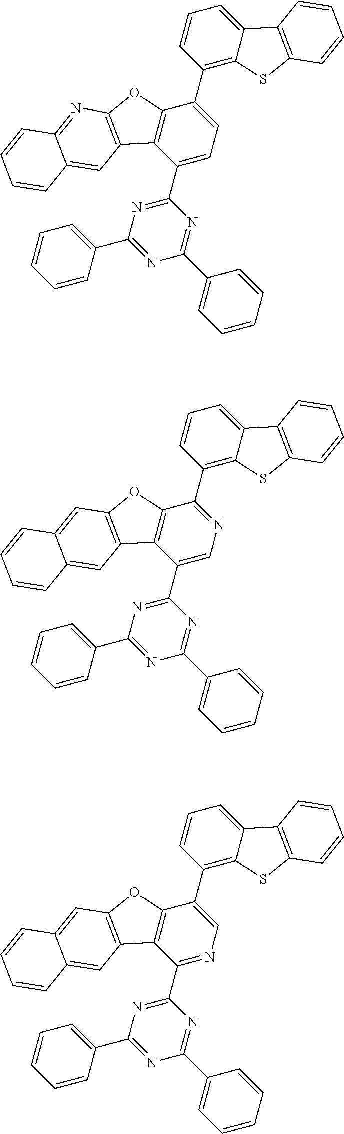 [SMILES: c1ccc(-c2nc(-c3ccccc3)nc(-c3ccc(-c4cccc5c4sc4ccccc45)c4oc5nc6ccccc6cc5c34)n2)cc1 ; c1ccc(-c2nc(-c3ccccc3)nc(-c3cnc(-c4cccc5c4sc4ccccc45)c4oc5cc6ccccc6cc5c34)n2)cc1 ; c1ccc(-c2nc(-c3ccccc3)nc(-c3ncc(-c4cccc5c4sc4ccccc45)c4oc5cc6ccccc6cc5c34)n2)cc1]

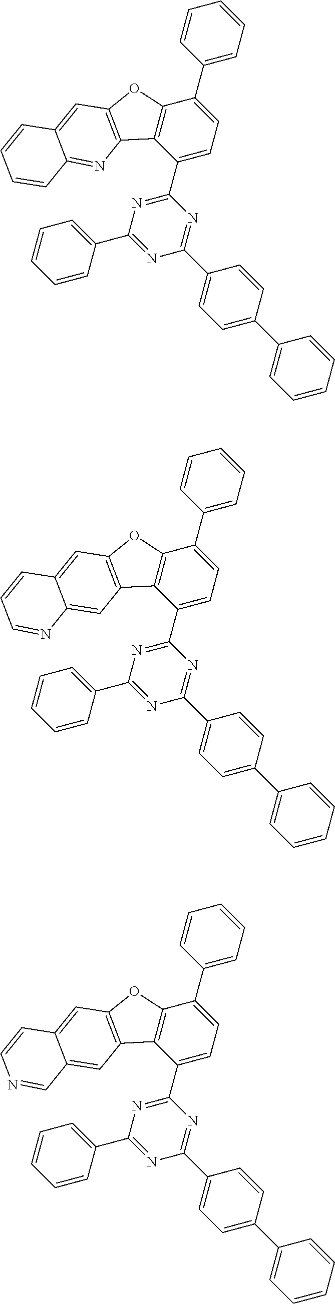 [SMILES: c1ccc(-c2ccc(-c3nc(-c4ccccc4)nc(-c4ccc(-c5ccccc5)c5oc6cc7ccccc7nc6c45)n3)cc2)cc1 ; c1ccc(-c2ccc(-c3nc(-c4ccccc4)nc(-c4ccc(-c5ccccc5)c5oc6cc7cccnc7cc6c45)n3)cc2)cc1 ; c1ccc(-c2ccc(-c3nc(-c4ccccc4)nc(-c4ccc(-c5ccccc5)c5oc6cc7ccncc7cc6c45)n3)cc2)cc1]

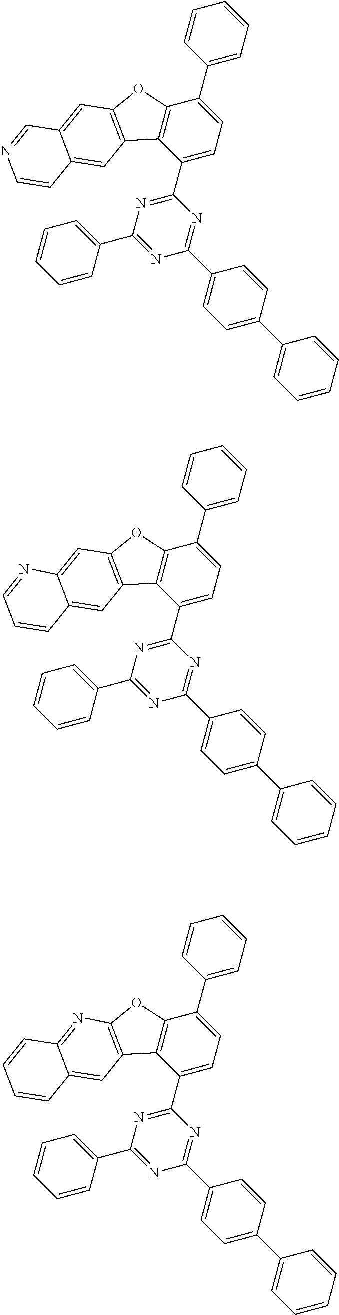 [SMILES: c1ccc(-c2ccc(-c3nc(-c4ccccc4)nc(-c4ccc(-c5ccccc5)c5oc6cc7cnccc7cc6c45)n3)cc2)cc1 ; c1ccc(-c2ccc(-c3nc(-c4ccccc4)nc(-c4ccc(-c5ccccc5)c5oc6cc7ncccc7cc6c45)n3)cc2)cc1 ; c1ccc(-c2ccc(-c3nc(-c4ccccc4)nc(-c4ccc(-c5ccccc5)c5oc6nc7ccccc7cc6c45)n3)cc2)cc1]

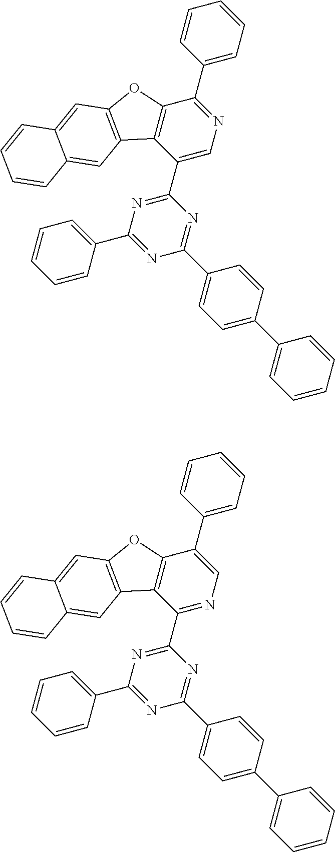 [SMILES: c1ccc(-c2ccc(-c3nc(-c4ccccc4)nc(-c4cnc(-c5ccccc5)c5oc6cc7ccccc7cc6c45)n3)cc2)cc1 ; c1ccc(-c2ccc(-c3nc(-c4ccccc4)nc(-c4ncc(-c5ccccc5)c5oc6cc7ccccc7cc6c45)n3)cc2)cc1]

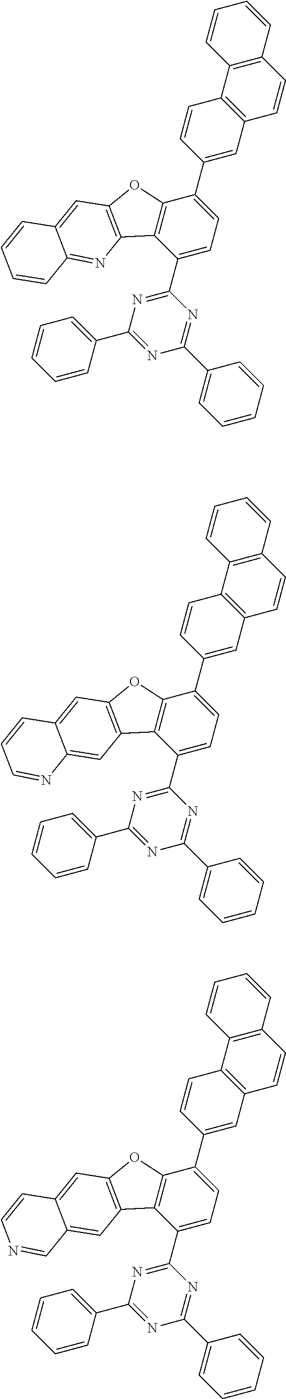 [SMILES: c1ccc(-c2nc(-c3ccccc3)nc(-c3ccc(-c4ccc5c(ccc6ccccc65)c4)c4oc5cc6ccccc6nc5c34)n2)cc1 ; c1ccc(-c2nc(-c3ccccc3)nc(-c3ccc(-c4ccc5c(ccc6ccccc65)c4)c4oc5cc6cccnc6cc5c34)n2)cc1 ; c1ccc(-c2nc(-c3ccccc3)nc(-c3ccc(-c4ccc5c(ccc6ccccc65)c4)c4oc5cc6ccncc6cc5c34)n2)cc1]

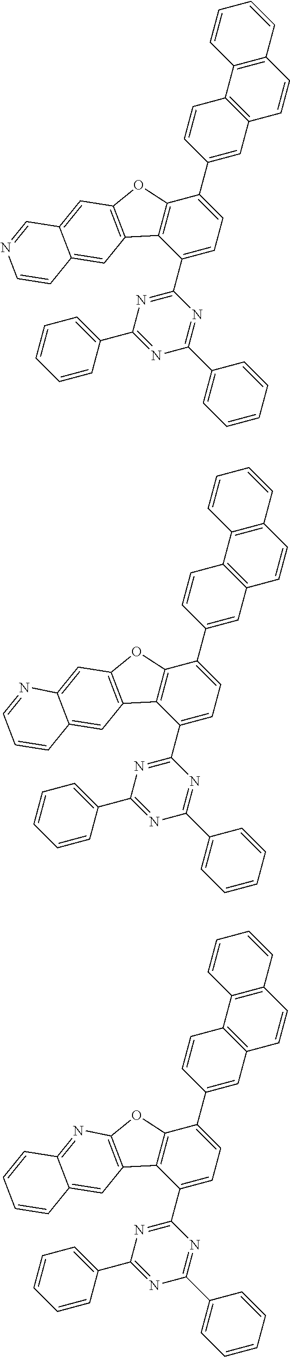

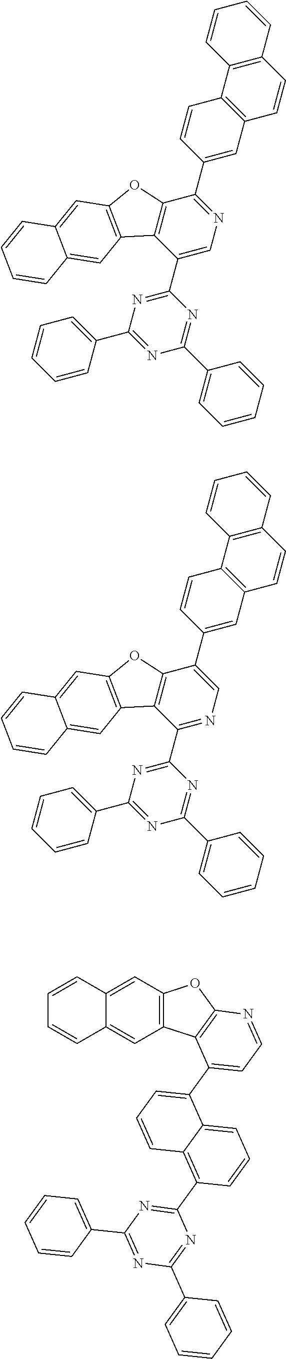 [SMILES: c1ccc(-c2nc(-c3ccccc3)nc(-c3cnc(-c4ccc5c(ccc6ccccc65)c4)c4oc5cc6ccccc6cc5c34)n2)cc1 ; c1ccc(-c2nc(-c3ccccc3)nc(-c3ncc(-c4ccc5c(ccc6ccccc65)c4)c4oc5cc6ccccc6cc5c34)n2)cc1 ; c1ccc(-c2nc(-c3ccccc3)nc(-c3cccc4c(-c5ccnc6oc7cc8ccccc8cc7c56)cccc34)n2)cc1]

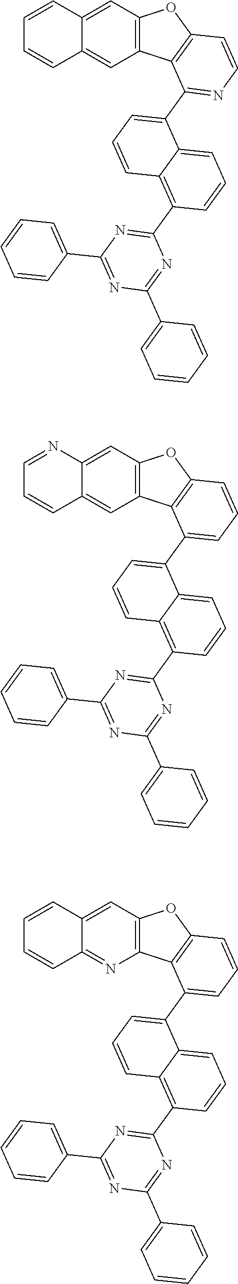 [SMILES: c1ccc(-c2nc(-c3ccccc3)nc(-c3cccc4c(-c5nccc6oc7cc8ccccc8cc7c56)cccc34)n2)cc1 ; c1ccc(-c2nc(-c3ccccc3)nc(-c3cccc4c(-c5cccc6oc7cc8ncccc8cc7c56)cccc34)n2)cc1 ; c1ccc(-c2nc(-c3ccccc3)nc(-c3cccc4c(-c5cccc6oc7cc8ccccc8nc7c56)cccc34)n2)cc1]

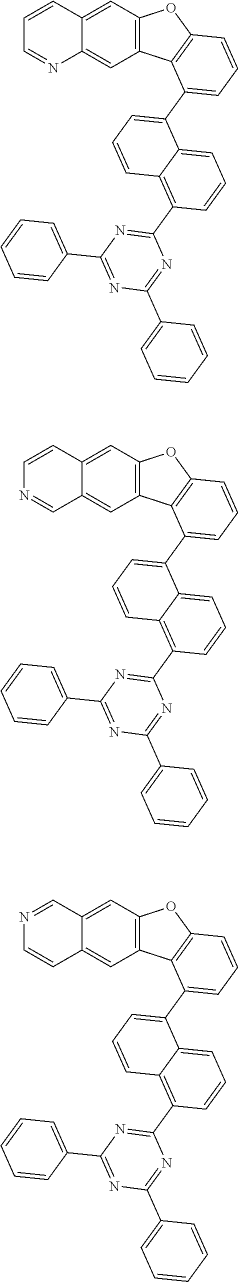 [SMILES: c1ccc(-c2nc(-c3ccccc3)nc(-c3cccc4c(-c5cccc6oc7cc8cccnc8cc7c56)cccc34)n2)cc1 ; c1ccc(-c2nc(-c3ccccc3)nc(-c3cccc4c(-c5cccc6oc7cc8ccncc8cc7c56)cccc34)n2)cc1 ; c1ccc(-c2nc(-c3ccccc3)nc(-c3cccc4c(-c5cccc6oc7cc8cnccc8cc7c56)cccc34)n2)cc1]

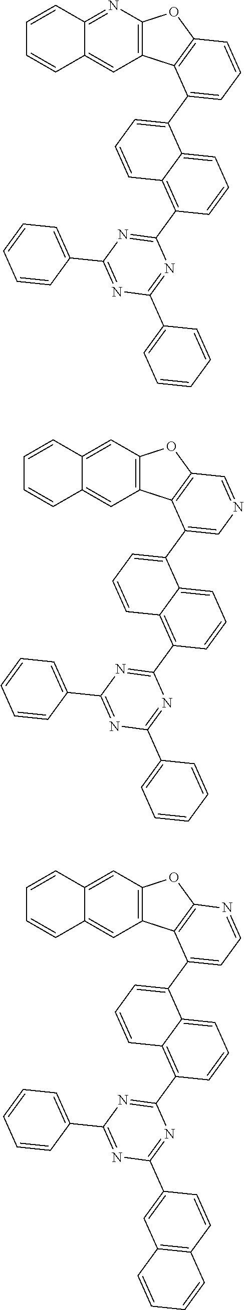 [SMILES: c1ccc(-c2nc(-c3ccccc3)nc(-c3cccc4c(-c5cccc6oc7nc8ccccc8cc7c56)cccc34)n2)cc1 ; c1ccc(-c2nc(-c3ccccc3)nc(-c3cccc4c(-c5cncc6oc7cc8ccccc8cc7c56)cccc34)n2)cc1 ; c1ccc(-c2nc(-c3ccc4ccccc4c3)nc(-c3cccc4c(-c5ccnc6oc7cc8ccccc8cc7c56)cccc34)n2)cc1]

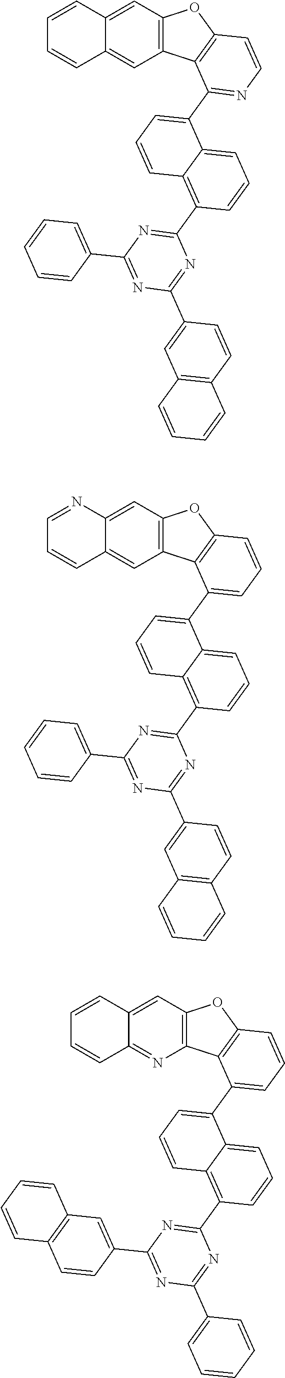 [SMILES: c1ccc(-c2nc(-c3ccc4ccccc4c3)nc(-c3cccc4c(-c5nccc6oc7cc8ccccc8cc7c56)cccc34)n2)cc1 ; c1ccc(-c2nc(-c3ccc4ccccc4c3)nc(-c3cccc4c(-c5cccc6oc7cc8ncccc8cc7c56)cccc34)n2)cc1 ; c1ccc(-c2nc(-c3ccc4ccccc4c3)nc(-c3cccc4c(-c5cccc6oc7cc8ccccc8nc7c56)cccc34)n2)cc1]

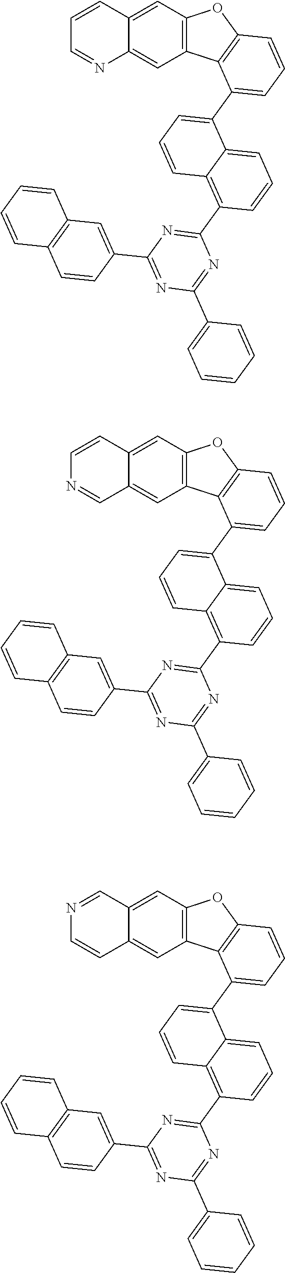 [SMILES: c1ccc(-c2nc(-c3ccc4ccccc4c3)nc(-c3cccc4c(-c5cccc6oc7cc8cccnc8cc7c56)cccc34)n2)cc1 ; c1ccc(-c2nc(-c3ccc4ccccc4c3)nc(-c3cccc4c(-c5cccc6oc7cc8ccncc8cc7c56)cccc34)n2)cc1 ; c1ccc(-c2nc(-c3ccc4ccccc4c3)nc(-c3cccc4c(-c5cccc6oc7cc8cnccc8cc7c56)cccc34)n2)cc1]

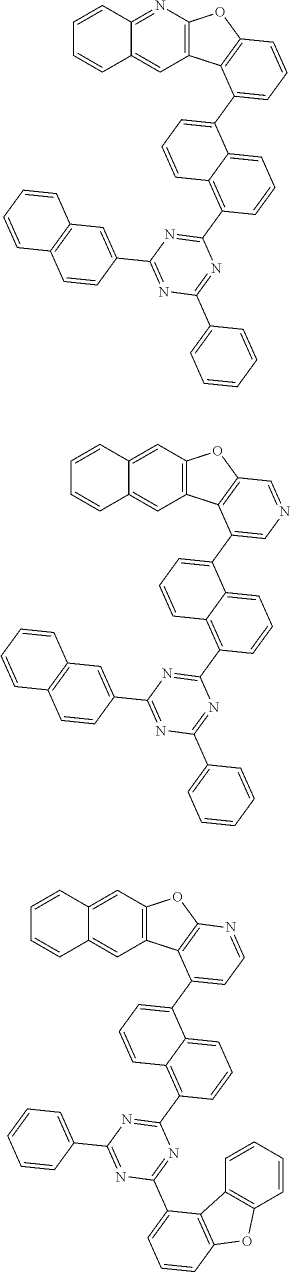 [SMILES: c1ccc(-c2nc(-c3ccc4ccccc4c3)nc(-c3cccc4c(-c5cccc6oc7nc8ccccc8cc7c56)cccc34)n2)cc1 ; c1ccc(-c2nc(-c3ccc4ccccc4c3)nc(-c3cccc4c(-c5cncc6oc7cc8ccccc8cc7c56)cccc34)n2)cc1 ; c1ccc(-c2nc(-c3cccc4c(-c5ccnc6oc7cc8ccccc8cc7c56)cccc34)nc(-c3cccc4oc5ccccc5c34)n2)cc1]

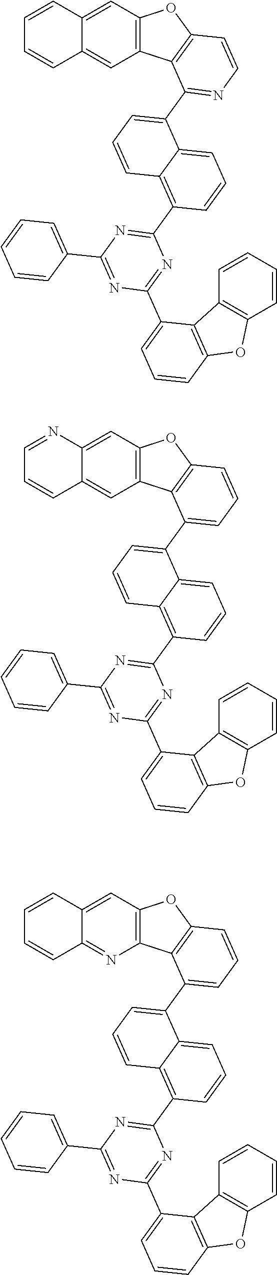 [SMILES: c1ccc(-c2nc(-c3cccc4c(-c5nccc6oc7cc8ccccc8cc7c56)cccc34)nc(-c3cccc4oc5ccccc5c34)n2)cc1 ; c1ccc(-c2nc(-c3cccc4c(-c5cccc6oc7cc8ncccc8cc7c56)cccc34)nc(-c3cccc4oc5ccccc5c34)n2)cc1 ; c1ccc(-c2nc(-c3cccc4c(-c5cccc6oc7cc8ccccc8nc7c56)cccc34)nc(-c3cccc4oc5ccccc5c34)n2)cc1]

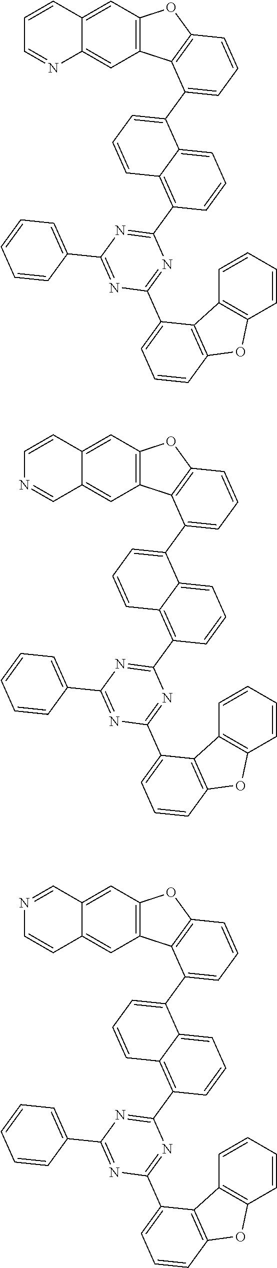 [SMILES: c1ccc(-c2nc(-c3cccc4c(-c5cccc6oc7cc8cccnc8cc7c56)cccc34)nc(-c3cccc4oc5ccccc5c34)n2)cc1 ; c1ccc(-c2nc(-c3cccc4c(-c5cccc6oc7cc8ccncc8cc7c56)cccc34)nc(-c3cccc4oc5ccccc5c34)n2)cc1 ; c1ccc(-c2nc(-c3cccc4c(-c5cccc6oc7cc8cnccc8cc7c56)cccc34)nc(-c3cccc4oc5ccccc5c34)n2)cc1]

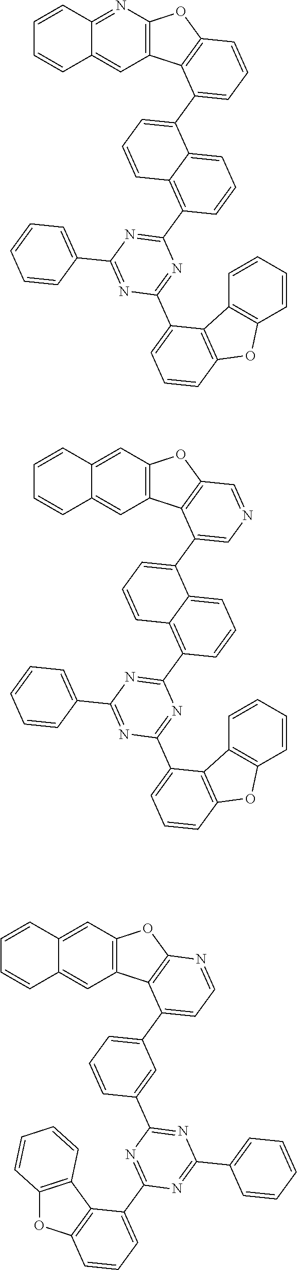 [SMILES: c1ccc(-c2nc(-c3cccc4c(-c5cccc6oc7nc8ccccc8cc7c56)cccc34)nc(-c3cccc4oc5ccccc5c34)n2)cc1 ; c1ccc(-c2nc(-c3cccc4c(-c5cncc6oc7cc8ccccc8cc7c56)cccc34)nc(-c3cccc4oc5ccccc5c34)n2)cc1 ; c1ccc(-c2nc(-c3cccc(-c4ccnc5oc6cc7ccccc7cc6c45)c3)nc(-c3cccc4oc5ccccc5c34)n2)cc1]

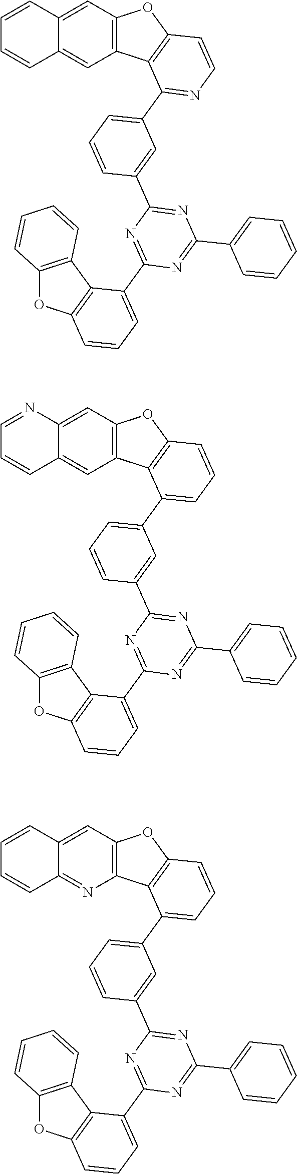 [SMILES: c1ccc(-c2nc(-c3cccc(-c4nccc5oc6cc7ccccc7cc6c45)c3)nc(-c3cccc4oc5ccccc5c34)n2)cc1 ; c1ccc(-c2nc(-c3cccc(-c4cccc5oc6cc7ncccc7cc6c45)c3)nc(-c3cccc4oc5ccccc5c34)n2)cc1 ; c1ccc(-c2nc(-c3cccc(-c4cccc5oc6cc7ccccc7nc6c45)c3)nc(-c3cccc4oc5ccccc5c34)n2)cc1]

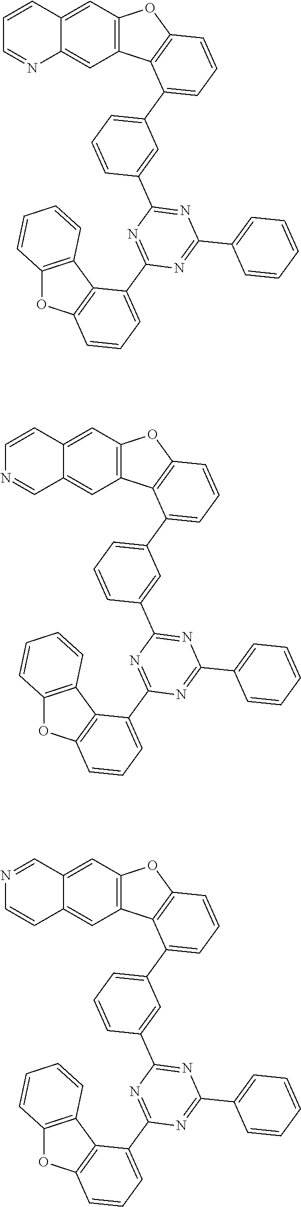 [SMILES: c1ccc(-c2nc(-c3cccc(-c4cccc5oc6cc7cccnc7cc6c45)c3)nc(-c3cccc4oc5ccccc5c34)n2)cc1 ; c1ccc(-c2nc(-c3cccc(-c4cccc5oc6cc7ccncc7cc6c45)c3)nc(-c3cccc4oc5ccccc5c34)n2)cc1 ; c1ccc(-c2nc(-c3cccc(-c4cccc5oc6cc7cnccc7cc6c45)c3)nc(-c3cccc4oc5ccccc5c34)n2)cc1]

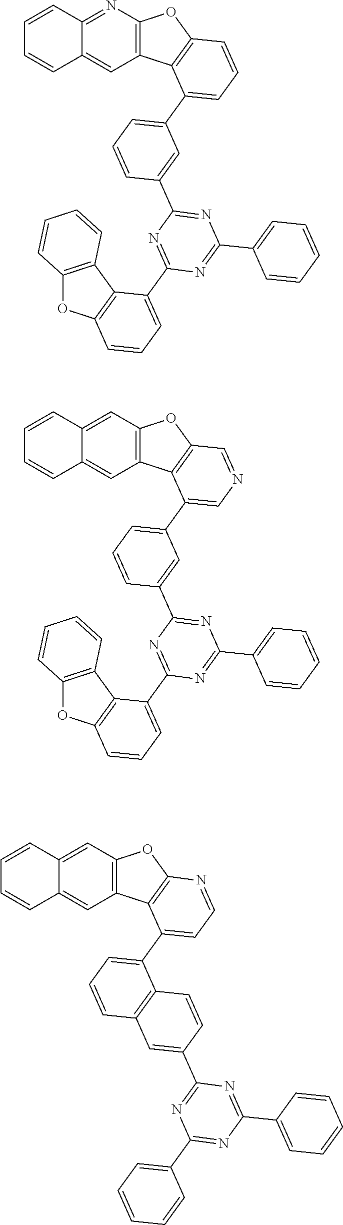 [SMILES: c1ccc(-c2nc(-c3cccc(-c4cccc5oc6nc7ccccc7cc6c45)c3)nc(-c3cccc4oc5ccccc5c34)n2)cc1 ; c1ccc(-c2nc(-c3cccc(-c4cncc5oc6cc7ccccc7cc6c45)c3)nc(-c3cccc4oc5ccccc5c34)n2)cc1 ; c1ccc(-c2nc(-c3ccccc3)nc(-c3ccc4c(-c5ccnc6oc7cc8ccccc8cc7c56)cccc4c3)n2)cc1]

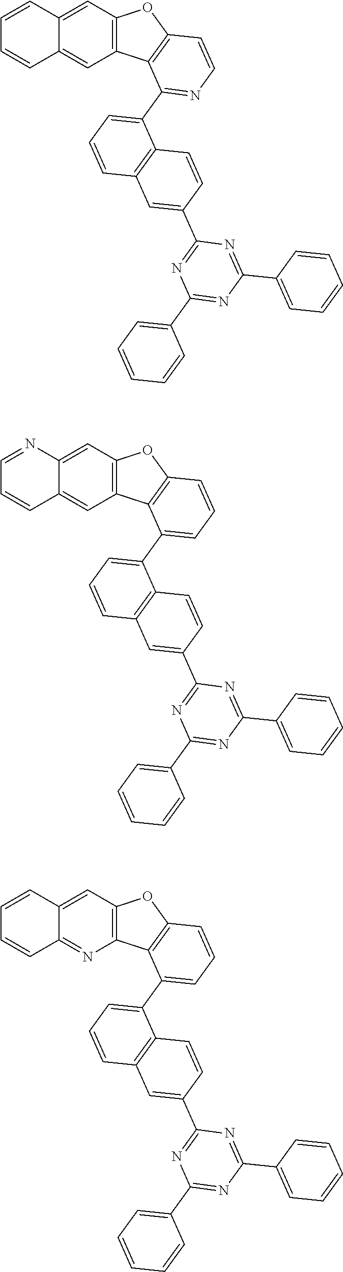 [SMILES: c1ccc(-c2nc(-c3ccccc3)nc(-c3ccc4c(-c5nccc6oc7cc8ccccc8cc7c56)cccc4c3)n2)cc1 ; c1ccc(-c2nc(-c3ccccc3)nc(-c3ccc4c(-c5cccc6oc7cc8ncccc8cc7c56)cccc4c3)n2)cc1 ; c1ccc(-c2nc(-c3ccccc3)nc(-c3ccc4c(-c5cccc6oc7cc8ccccc8nc7c56)cccc4c3)n2)cc1]

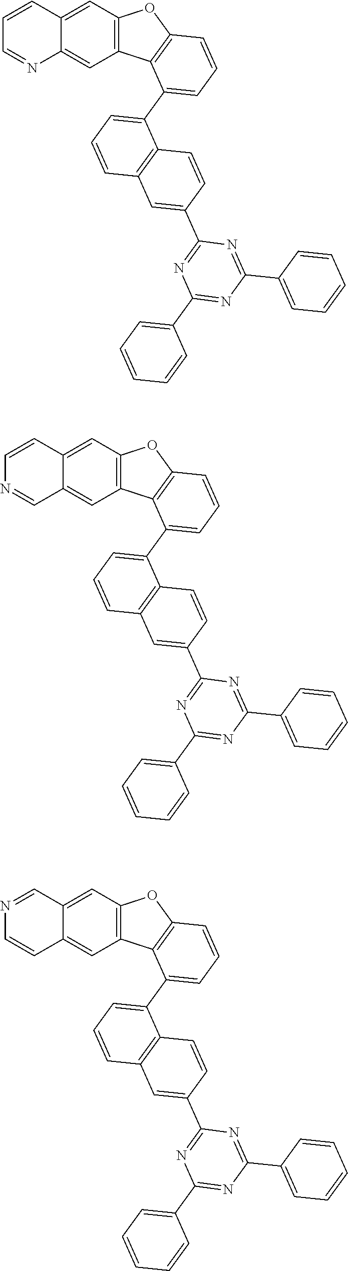 [SMILES: c1ccc(-c2nc(-c3ccccc3)nc(-c3ccc4c(-c5cccc6oc7cc8cccnc8cc7c56)cccc4c3)n2)cc1 ; c1ccc(-c2nc(-c3ccccc3)nc(-c3ccc4c(-c5cccc6oc7cc8ccncc8cc7c56)cccc4c3)n2)cc1 ; c1ccc(-c2nc(-c3ccccc3)nc(-c3ccc4c(-c5cccc6oc7cc8cnccc8cc7c56)cccc4c3)n2)cc1]

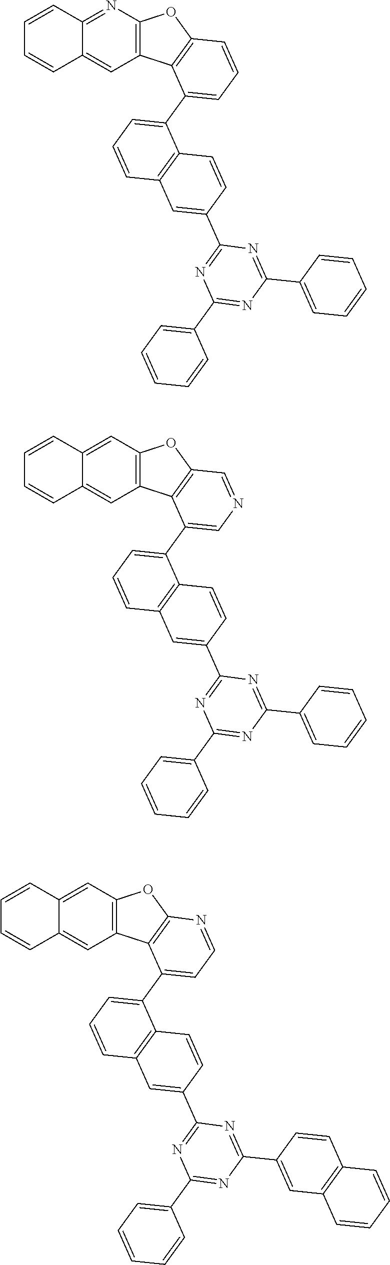 [SMILES: c1ccc(-c2nc(-c3ccccc3)nc(-c3ccc4c(-c5cccc6oc7nc8ccccc8cc7c56)cccc4c3)n2)cc1 ; c1ccc(-c2nc(-c3ccccc3)nc(-c3ccc4c(-c5cncc6oc7cc8ccccc8cc7c56)cccc4c3)n2)cc1 ; c1ccc(-c2nc(-c3ccc4ccccc4c3)nc(-c3ccc4c(-c5ccnc6oc7cc8ccccc8cc7c56)cccc4c3)n2)cc1]

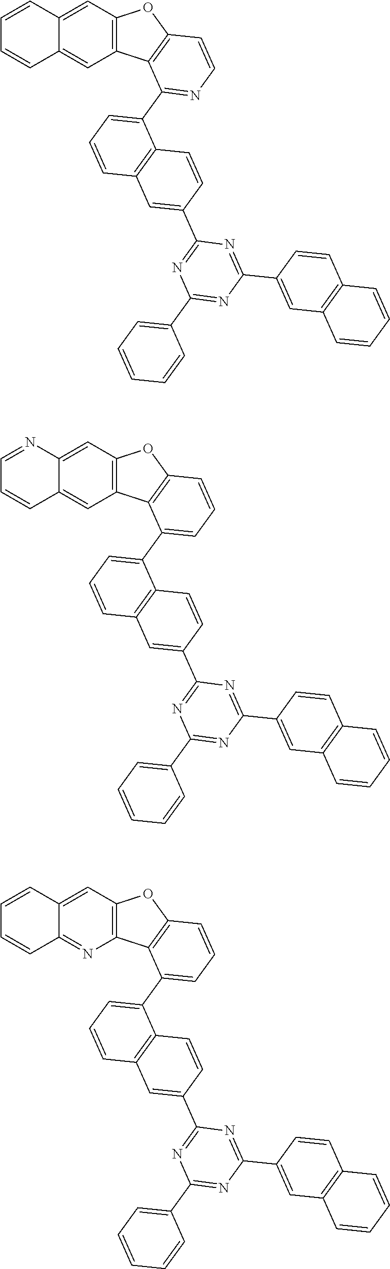 [SMILES: c1ccc(-c2nc(-c3ccc4ccccc4c3)nc(-c3ccc4c(-c5nccc6oc7cc8ccccc8cc7c56)cccc4c3)n2)cc1 ; c1ccc(-c2nc(-c3ccc4ccccc4c3)nc(-c3ccc4c(-c5cccc6oc7cc8ncccc8cc7c56)cccc4c3)n2)cc1 ; c1ccc(-c2nc(-c3ccc4ccccc4c3)nc(-c3ccc4c(-c5cccc6oc7cc8ccccc8nc7c56)cccc4c3)n2)cc1]

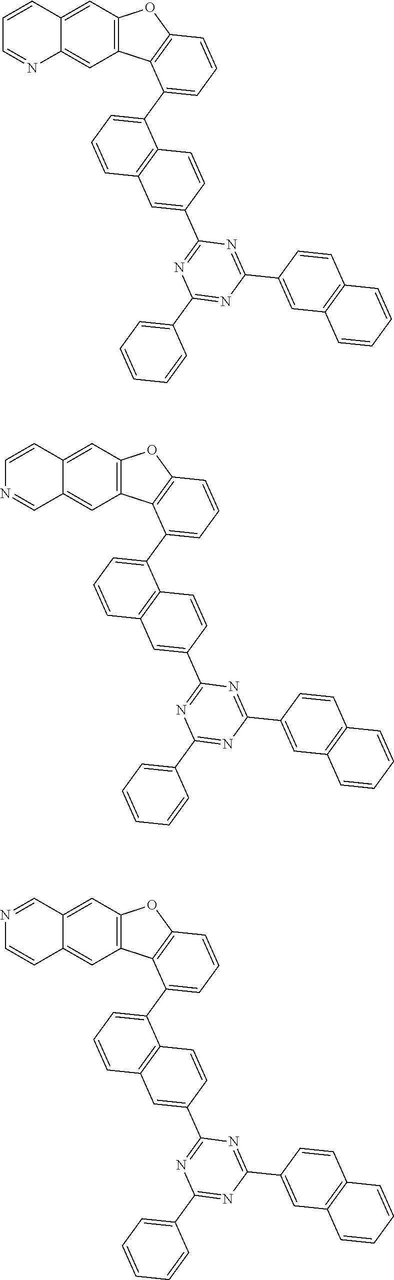 [SMILES: c1ccc(-c2nc(-c3ccc4ccccc4c3)nc(-c3ccc4c(-c5cccc6oc7cc8cccnc8cc7c56)cccc4c3)n2)cc1 ; c1ccc(-c2nc(-c3ccc4ccccc4c3)nc(-c3ccc4c(-c5cccc6oc7cc8ccncc8cc7c56)cccc4c3)n2)cc1 ; c1ccc(-c2nc(-c3ccc4ccccc4c3)nc(-c3ccc4c(-c5cccc6oc7cc8cnccc8cc7c56)cccc4c3)n2)cc1]

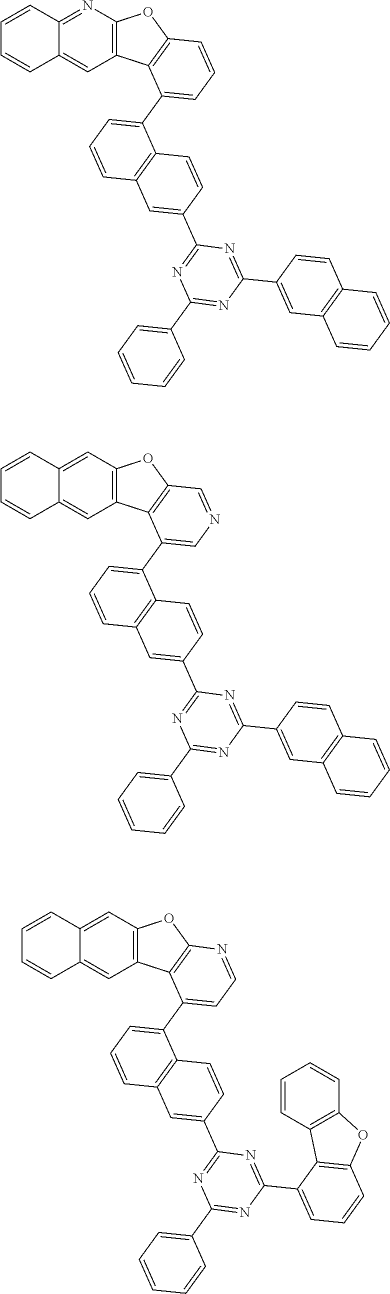 [SMILES: c1ccc(-c2nc(-c3ccc4ccccc4c3)nc(-c3ccc4c(-c5cccc6oc7nc8ccccc8cc7c56)cccc4c3)n2)cc1 ; c1ccc(-c2nc(-c3ccc4ccccc4c3)nc(-c3ccc4c(-c5cncc6oc7cc8ccccc8cc7c56)cccc4c3)n2)cc1 ; c1ccc(-c2nc(-c3ccc4c(-c5ccnc6oc7cc8ccccc8cc7c56)cccc4c3)nc(-c3cccc4oc5ccccc5c34)n2)cc1]

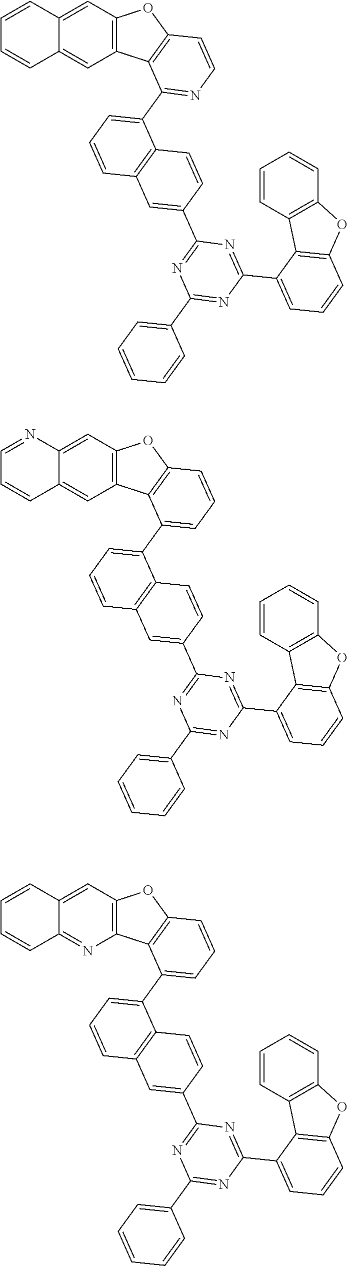 [SMILES: c1ccc(-c2nc(-c3ccc4c(-c5nccc6oc7cc8ccccc8cc7c56)cccc4c3)nc(-c3cccc4oc5ccccc5c34)n2)cc1 ; c1ccc(-c2nc(-c3ccc4c(-c5cccc6oc7cc8ncccc8cc7c56)cccc4c3)nc(-c3cccc4oc5ccccc5c34)n2)cc1 ; c1ccc(-c2nc(-c3ccc4c(-c5cccc6oc7cc8ccccc8nc7c56)cccc4c3)nc(-c3cccc4oc5ccccc5c34)n2)cc1]

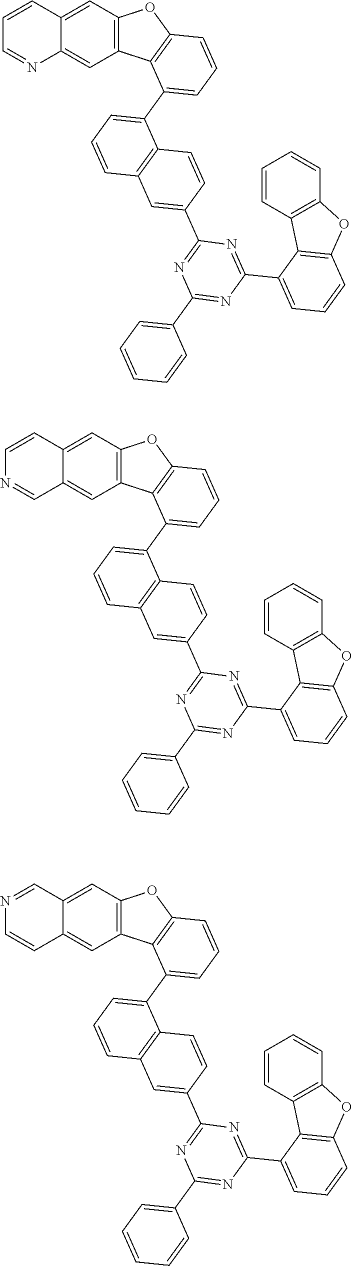 [SMILES: c1ccc(-c2nc(-c3ccc4c(-c5cccc6oc7cc8cccnc8cc7c56)cccc4c3)nc(-c3cccc4oc5ccccc5c34)n2)cc1 ; c1ccc(-c2nc(-c3ccc4c(-c5cccc6oc7cc8ccncc8cc7c56)cccc4c3)nc(-c3cccc4oc5ccccc5c34)n2)cc1 ; c1ccc(-c2nc(-c3ccc4c(-c5cccc6oc7cc8cnccc8cc7c56)cccc4c3)nc(-c3cccc4oc5ccccc5c34)n2)cc1]

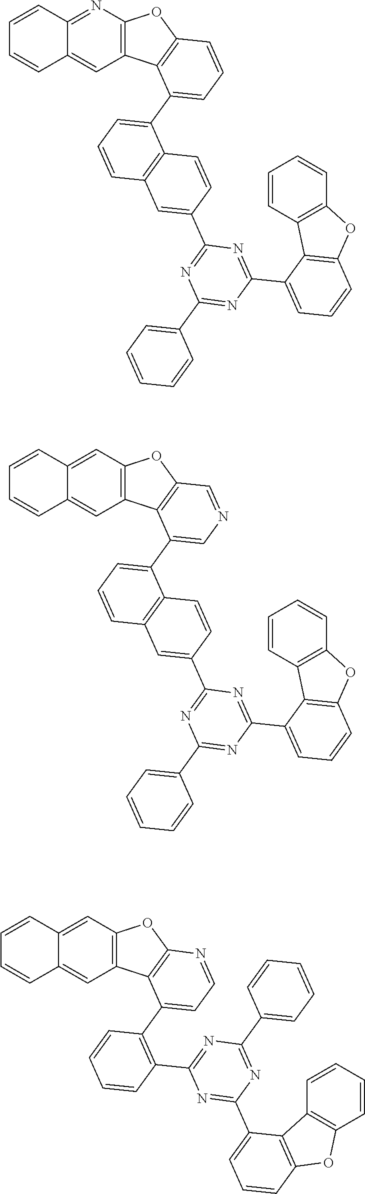 [SMILES: c1ccc(-c2nc(-c3ccc4c(-c5cccc6oc7nc8ccccc8cc7c56)cccc4c3)nc(-c3cccc4oc5ccccc5c34)n2)cc1 ; c1ccc(-c2nc(-c3ccc4c(-c5cncc6oc7cc8ccccc8cc7c56)cccc4c3)nc(-c3cccc4oc5ccccc5c34)n2)cc1 ; c1ccc(-c2nc(-c3ccccc3-c3ccnc4oc5cc6ccccc6cc5c34)nc(-c3cccc4oc5ccccc5c34)n2)cc1]

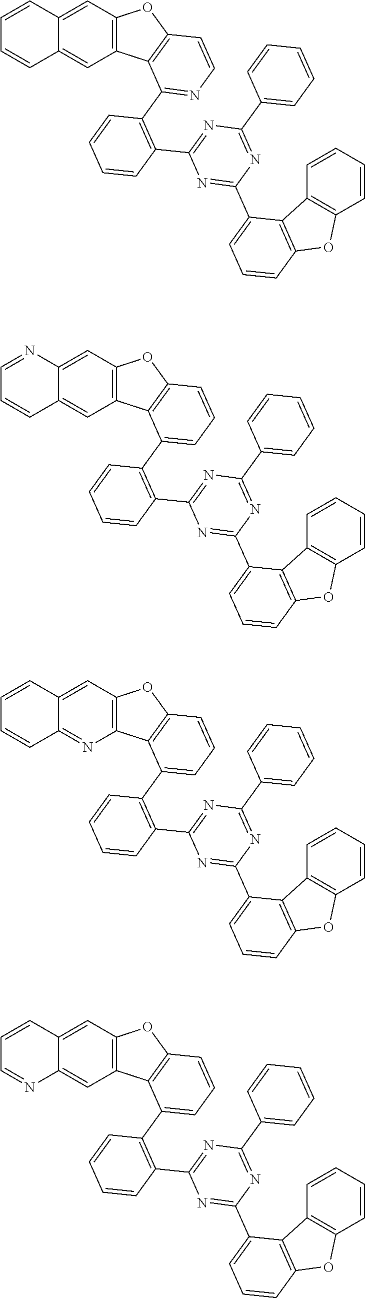 [SMILES: c1ccc(-c2nc(-c3ccccc3-c3nccc4oc5cc6ccccc6cc5c34)nc(-c3cccc4oc5ccccc5c34)n2)cc1 ; c1ccc(-c2nc(-c3ccccc3-c3cccc4oc5cc6ncccc6cc5c34)nc(-c3cccc4oc5ccccc5c34)n2)cc1 ; c1ccc(-c2nc(-c3ccccc3-c3cccc4oc5cc6ccccc6nc5c34)nc(-c3cccc4oc5ccccc5c34)n2)cc1 ; c1ccc(-c2nc(-c3ccccc3-c3cccc4oc5cc6cccnc6cc5c34)nc(-c3cccc4oc5ccccc5c34)n2)cc1]

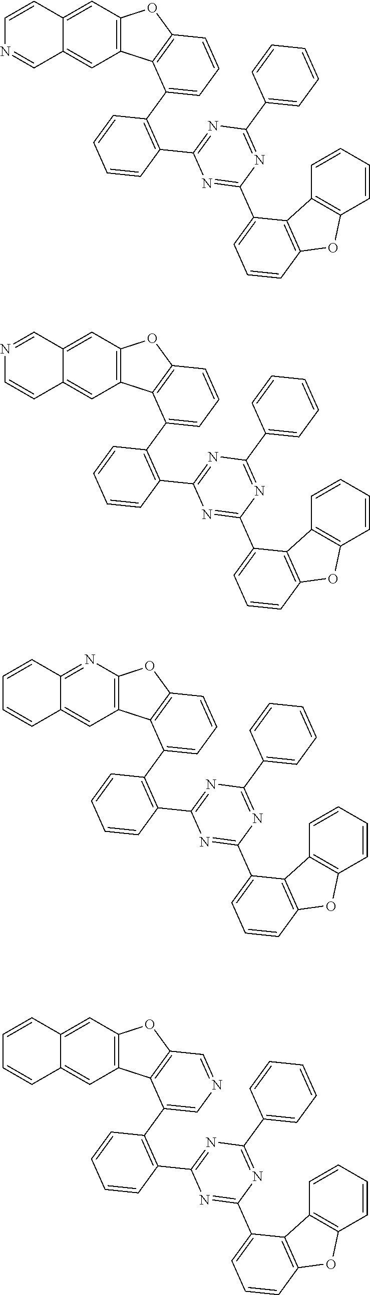 [SMILES: c1ccc(-c2nc(-c3ccccc3-c3cccc4oc5cc6ccncc6cc5c34)nc(-c3cccc4oc5ccccc5c34)n2)cc1 ; c1ccc(-c2nc(-c3ccccc3-c3cccc4oc5cc6cnccc6cc5c34)nc(-c3cccc4oc5ccccc5c34)n2)cc1 ; c1ccc(-c2nc(-c3ccccc3-c3cccc4oc5nc6ccccc6cc5c34)nc(-c3cccc4oc5ccccc5c34)n2)cc1 ; c1ccc(-c2nc(-c3ccccc3-c3cncc4oc5cc6ccccc6cc5c34)nc(-c3cccc4oc5ccccc5c34)n2)cc1]

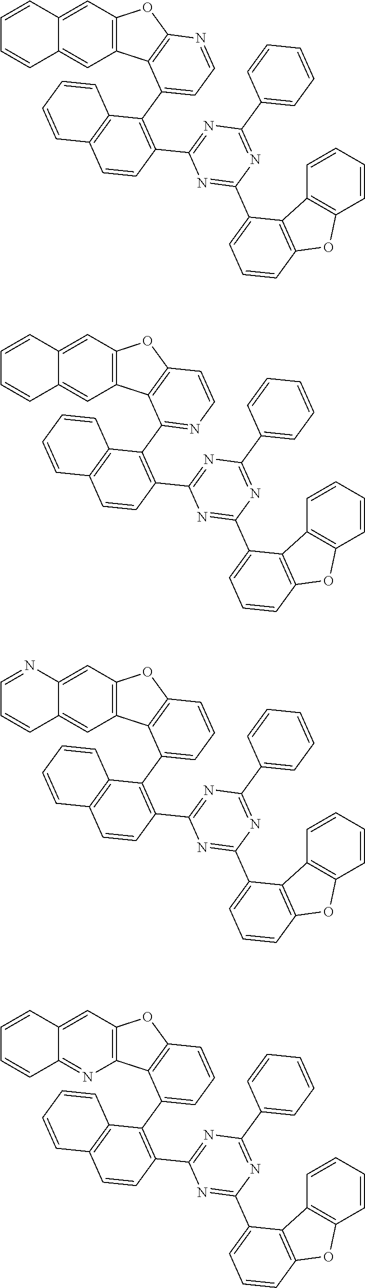 [SMILES: c1ccc(-c2nc(-c3ccc4ccccc4c3-c3ccnc4oc5cc6ccccc6cc5c34)nc(-c3cccc4oc5ccccc5c34)n2)cc1 ; c1ccc(-c2nc(-c3ccc4ccccc4c3-c3nccc4oc5cc6ccccc6cc5c34)nc(-c3cccc4oc5ccccc5c34)n2)cc1 ; c1ccc(-c2nc(-c3ccc4ccccc4c3-c3cccc4oc5cc6ncccc6cc5c34)nc(-c3cccc4oc5ccccc5c34)n2)cc1 ; c1ccc(-c2nc(-c3ccc4ccccc4c3-c3cccc4oc5cc6ccccc6nc5c34)nc(-c3cccc4oc5ccccc5c34)n2)cc1]

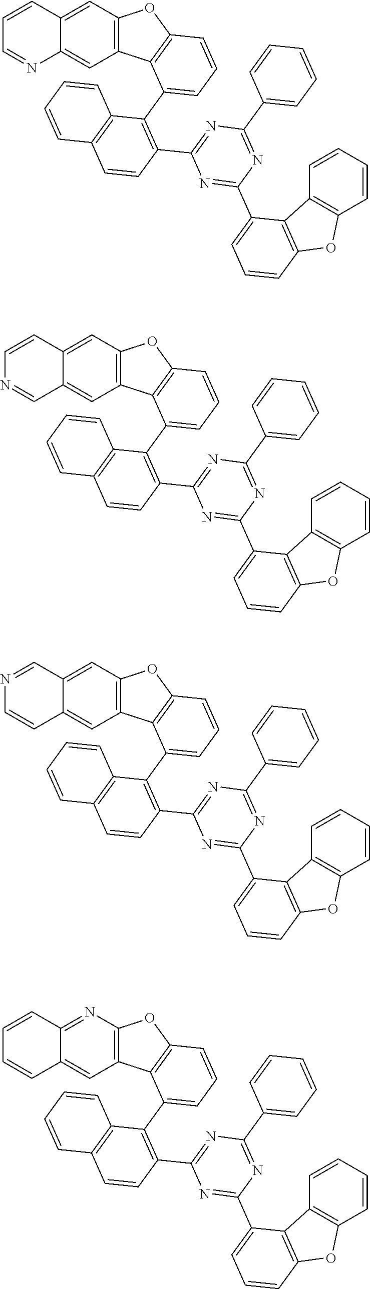 [SMILES: c1ccc(-c2nc(-c3ccc4ccccc4c3-c3cccc4oc5cc6cccnc6cc5c34)nc(-c3cccc4oc5ccccc5c34)n2)cc1 ; c1ccc(-c2nc(-c3ccc4ccccc4c3-c3cccc4oc5cc6ccncc6cc5c34)nc(-c3cccc4oc5ccccc5c34)n2)cc1 ; c1ccc(-c2nc(-c3ccc4ccccc4c3-c3cccc4oc5cc6cnccc6cc5c34)nc(-c3cccc4oc5ccccc5c34)n2)cc1 ; c1ccc(-c2nc(-c3ccc4ccccc4c3-c3cccc4oc5nc6ccccc6cc5c34)nc(-c3cccc4oc5ccccc5c34)n2)cc1]

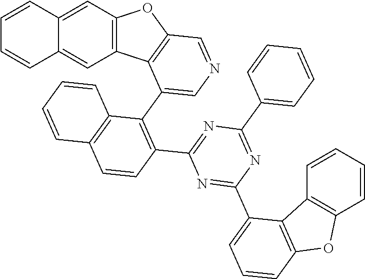 [SMILES: c1ccc(-c2nc(-c3ccc4ccccc4c3-c3cncc4oc5cc6ccccc6cc5c34)nc(-c3cccc4oc5ccccc5c34)n2)cc1]

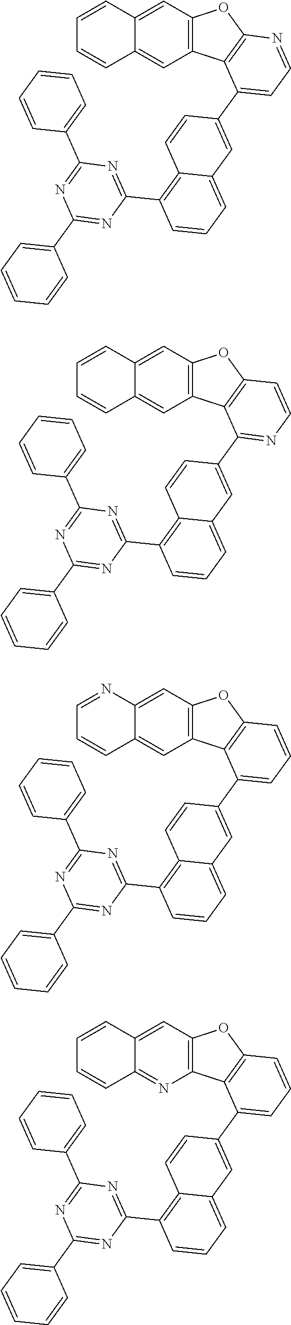 [SMILES: c1ccc(-c2nc(-c3ccccc3)nc(-c3cccc4cc(-c5ccnc6oc7cc8ccccc8cc7c56)ccc34)n2)cc1 ; c1ccc(-c2nc(-c3ccccc3)nc(-c3cccc4cc(-c5nccc6oc7cc8ccccc8cc7c56)ccc34)n2)cc1 ; c1ccc(-c2nc(-c3ccccc3)nc(-c3cccc4cc(-c5cccc6oc7cc8ncccc8cc7c56)ccc34)n2)cc1 ; c1ccc(-c2nc(-c3ccccc3)nc(-c3cccc4cc(-c5cccc6oc7cc8ccccc8nc7c56)ccc34)n2)cc1]

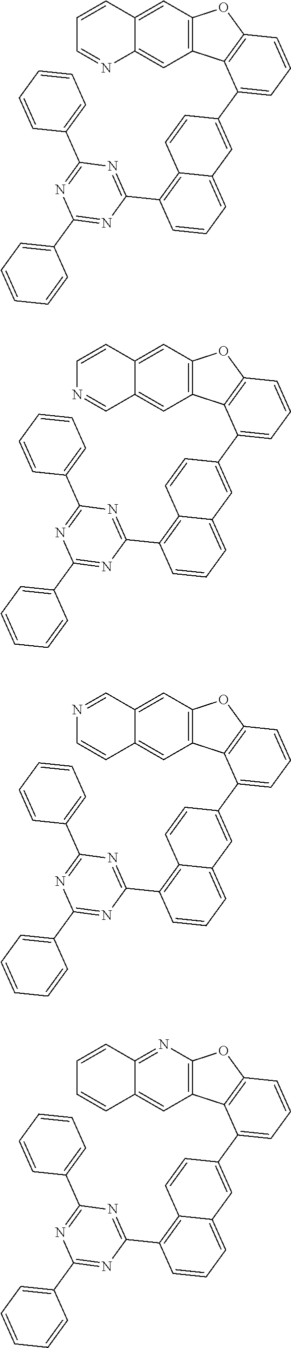 [SMILES: c1ccc(-c2nc(-c3ccccc3)nc(-c3cccc4cc(-c5cccc6oc7cc8cccnc8cc7c56)ccc34)n2)cc1 ; c1ccc(-c2nc(-c3ccccc3)nc(-c3cccc4cc(-c5cccc6oc7cc8ccncc8cc7c56)ccc34)n2)cc1 ; c1ccc(-c2nc(-c3ccccc3)nc(-c3cccc4cc(-c5cccc6oc7cc8cnccc8cc7c56)ccc34)n2)cc1 ; c1ccc(-c2nc(-c3ccccc3)nc(-c3cccc4cc(-c5cccc6oc7nc8ccccc8cc7c56)ccc34)n2)cc1]

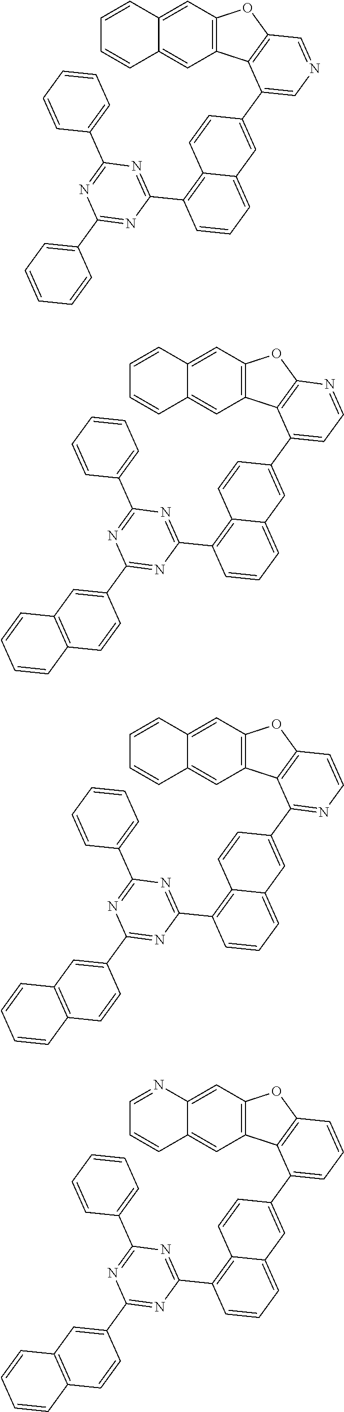 [SMILES: c1ccc(-c2nc(-c3ccccc3)nc(-c3cccc4cc(-c5cncc6oc7cc8ccccc8cc7c56)ccc34)n2)cc1 ; c1ccc(-c2nc(-c3ccc4ccccc4c3)nc(-c3cccc4cc(-c5ccnc6oc7cc8ccccc8cc7c56)ccc34)n2)cc1 ; c1ccc(-c2nc(-c3ccc4ccccc4c3)nc(-c3cccc4cc(-c5nccc6oc7cc8ccccc8cc7c56)ccc34)n2)cc1 ; c1ccc(-c2nc(-c3ccc4ccccc4c3)nc(-c3cccc4cc(-c5cccc6oc7cc8ncccc8cc7c56)ccc34)n2)cc1]

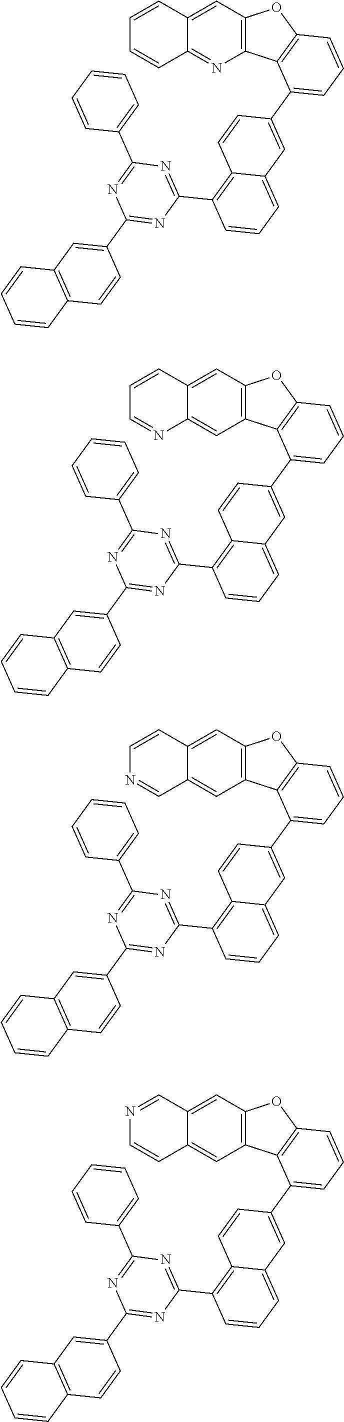 [SMILES: c1ccc(-c2nc(-c3ccc4ccccc4c3)nc(-c3cccc4cc(-c5cccc6oc7cc8ccccc8nc7c56)ccc34)n2)cc1 ; c1ccc(-c2nc(-c3ccc4ccccc4c3)nc(-c3cccc4cc(-c5cccc6oc7cc8cccnc8cc7c56)ccc34)n2)cc1 ; c1ccc(-c2nc(-c3ccc4ccccc4c3)nc(-c3cccc4cc(-c5cccc6oc7cc8ccncc8cc7c56)ccc34)n2)cc1 ; c1ccc(-c2nc(-c3ccc4ccccc4c3)nc(-c3cccc4cc(-c5cccc6oc7cc8cnccc8cc7c56)ccc34)n2)cc1]

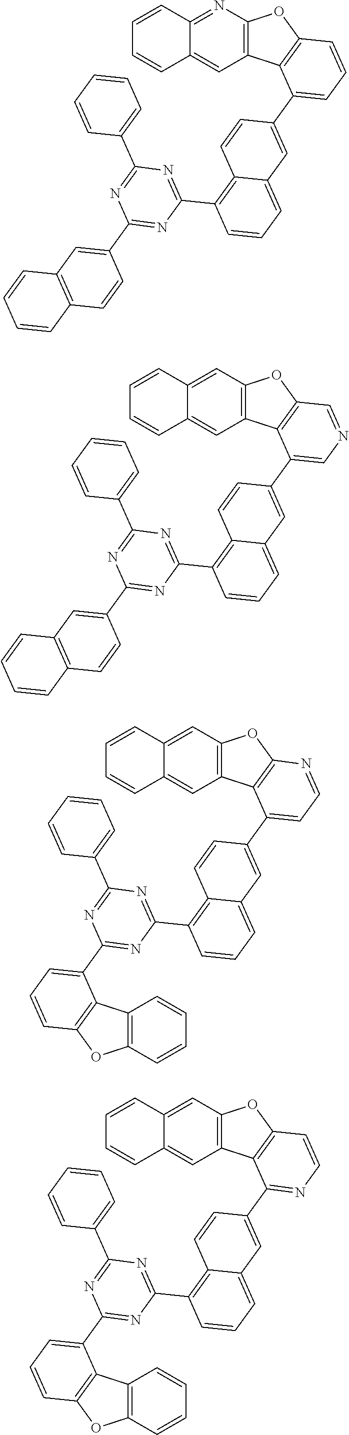

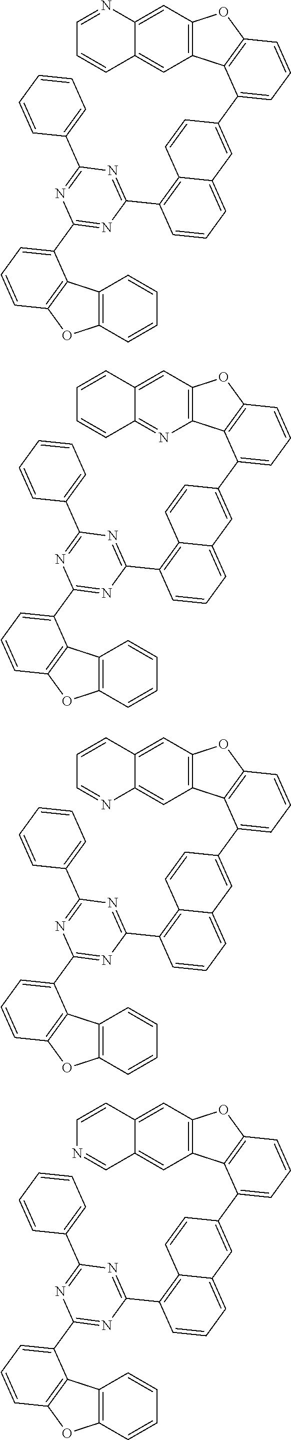

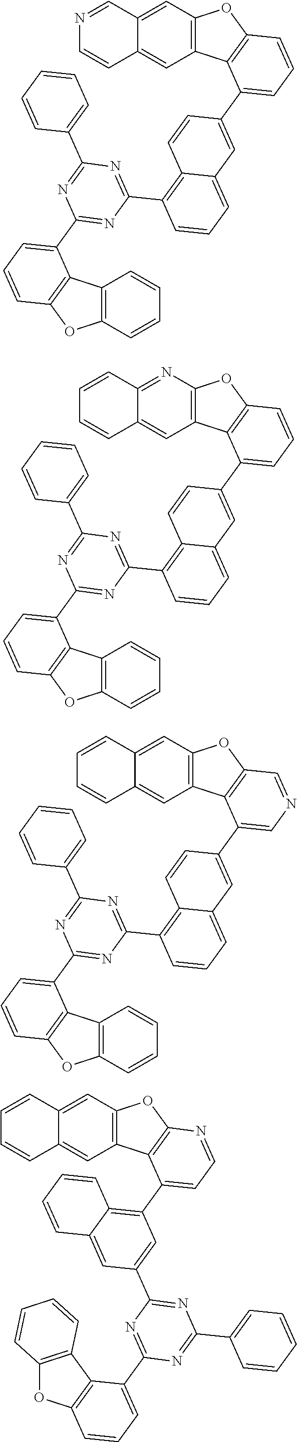

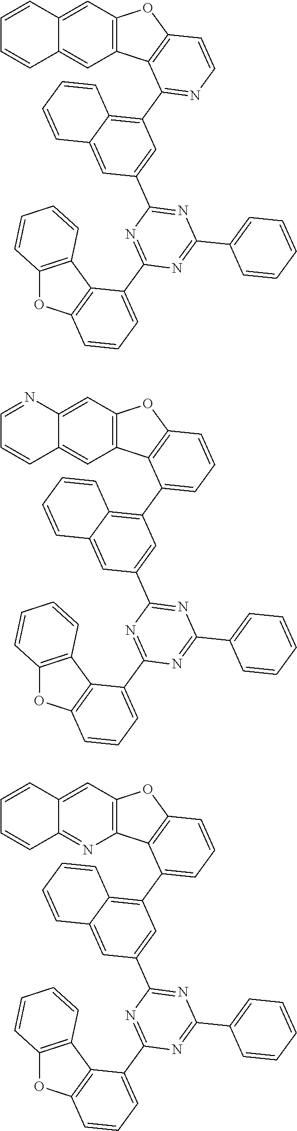

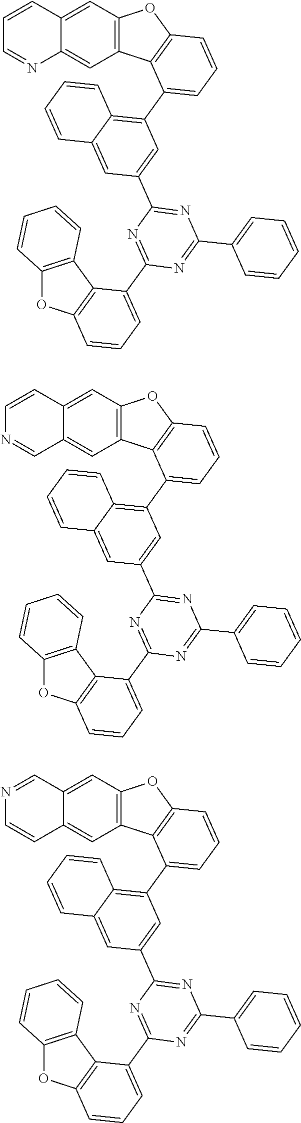

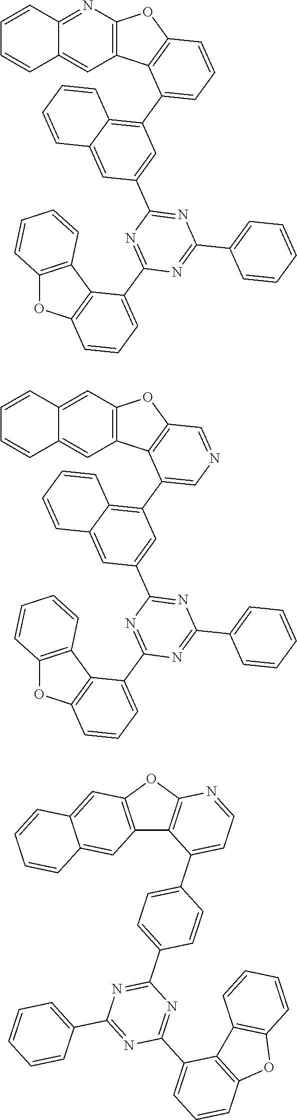

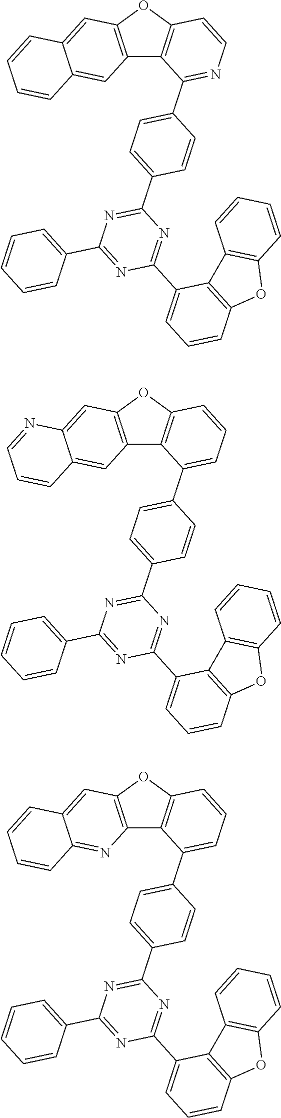 [SMILES: c1ccc(-c2nc(-c3ccc(-c4nccc5oc6cc7ccccc7cc6c45)cc3)nc(-c3cccc4oc5ccccc5c34)n2)cc1 ; c1ccc(-c2nc(-c3ccc(-c4cccc5oc6cc7ncccc7cc6c45)cc3)nc(-c3cccc4oc5ccccc5c34)n2)cc1 ; c1ccc(-c2nc(-c3ccc(-c4cccc5oc6cc7ccccc7nc6c45)cc3)nc(-c3cccc4oc5ccccc5c34)n2)cc1]

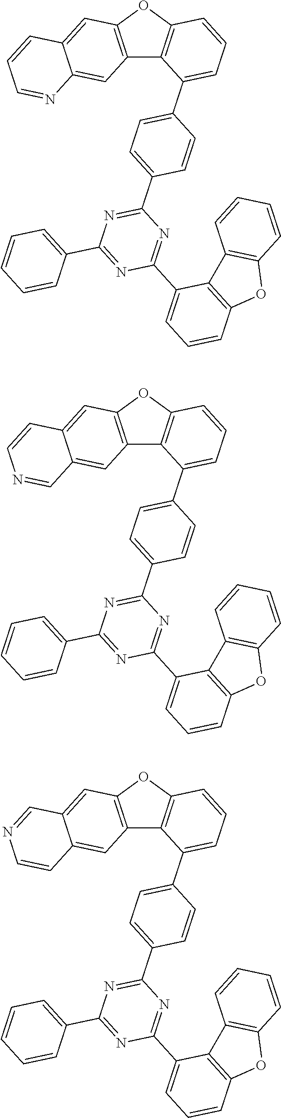 [SMILES: c1ccc(-c2nc(-c3ccc(-c4cccc5oc6cc7cccnc7cc6c45)cc3)nc(-c3cccc4oc5ccccc5c34)n2)cc1 ; c1ccc(-c2nc(-c3ccc(-c4cccc5oc6cc7ccncc7cc6c45)cc3)nc(-c3cccc4oc5ccccc5c34)n2)cc1 ; c1ccc(-c2nc(-c3ccc(-c4cccc5oc6cc7cnccc7cc6c45)cc3)nc(-c3cccc4oc5ccccc5c34)n2)cc1]

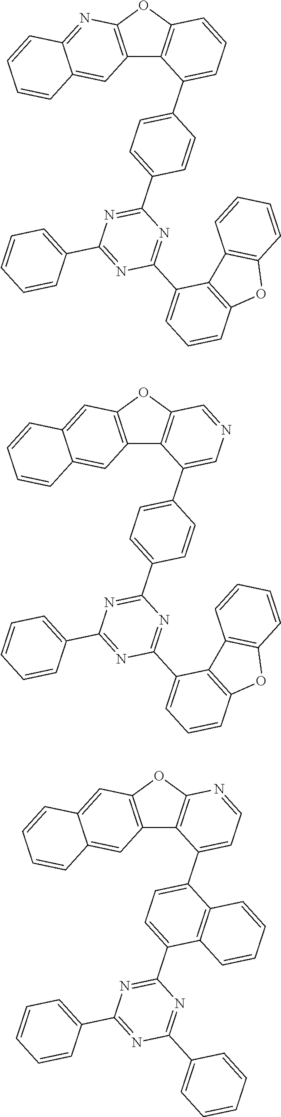 [SMILES: c1ccc(-c2nc(-c3ccc(-c4cccc5oc6nc7ccccc7cc6c45)cc3)nc(-c3cccc4oc5ccccc5c34)n2)cc1 ; c1ccc(-c2nc(-c3ccc(-c4cncc5oc6cc7ccccc7cc6c45)cc3)nc(-c3cccc4oc5ccccc5c34)n2)cc1 ; c1ccc(-c2nc(-c3ccccc3)nc(-c3ccc(-c4ccnc5oc6cc7ccccc7cc6c45)c4ccccc34)n2)cc1]

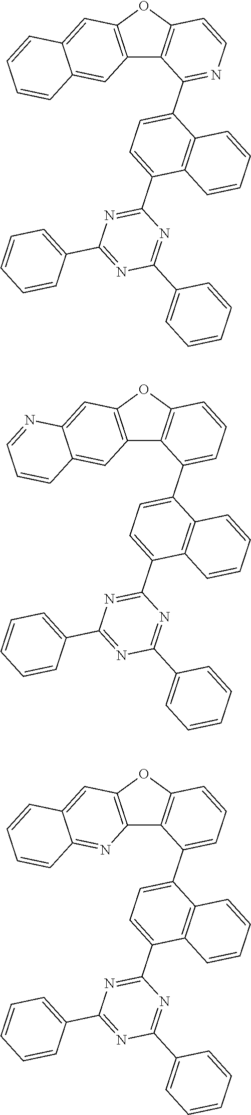 [SMILES: c1ccc(-c2nc(-c3ccccc3)nc(-c3ccc(-c4nccc5oc6cc7ccccc7cc6c45)c4ccccc34)n2)cc1 ; c1ccc(-c2nc(-c3ccccc3)nc(-c3ccc(-c4cccc5oc6cc7ncccc7cc6c45)c4ccccc34)n2)cc1 ; c1ccc(-c2nc(-c3ccccc3)nc(-c3ccc(-c4cccc5oc6cc7ccccc7nc6c45)c4ccccc34)n2)cc1]

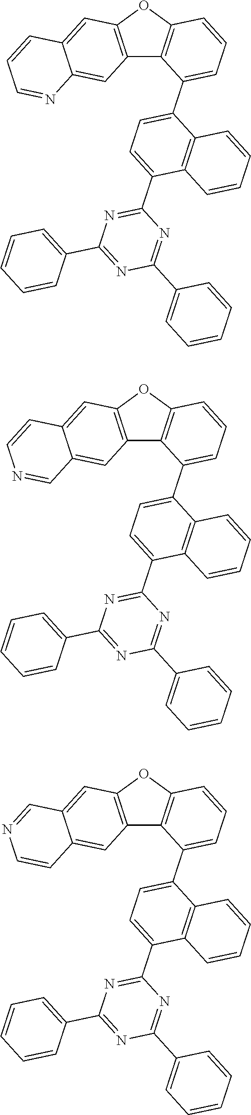 [SMILES: c1ccc(-c2nc(-c3ccccc3)nc(-c3ccc(-c4cccc5oc6cc7cccnc7cc6c45)c4ccccc34)n2)cc1 ; c1ccc(-c2nc(-c3ccccc3)nc(-c3ccc(-c4cccc5oc6cc7ccncc7cc6c45)c4ccccc34)n2)cc1 ; c1ccc(-c2nc(-c3ccccc3)nc(-c3ccc(-c4cccc5oc6cc7cnccc7cc6c45)c4ccccc34)n2)cc1]

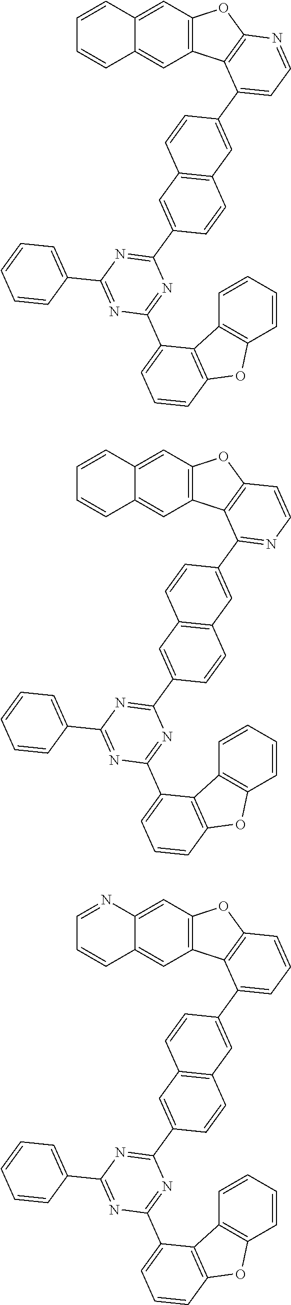 [SMILES: c1ccc(-c2nc(-c3ccc4cc(-c5ccnc6oc7cc8ccccc8cc7c56)ccc4c3)nc(-c3cccc4oc5ccccc5c34)n2)cc1 ; c1ccc(-c2nc(-c3ccc4cc(-c5nccc6oc7cc8ccccc8cc7c56)ccc4c3)nc(-c3cccc4oc5ccccc5c34)n2)cc1 ; c1ccc(-c2nc(-c3ccc4cc(-c5cccc6oc7cc8ncccc8cc7c56)ccc4c3)nc(-c3cccc4oc5ccccc5c34)n2)cc1]

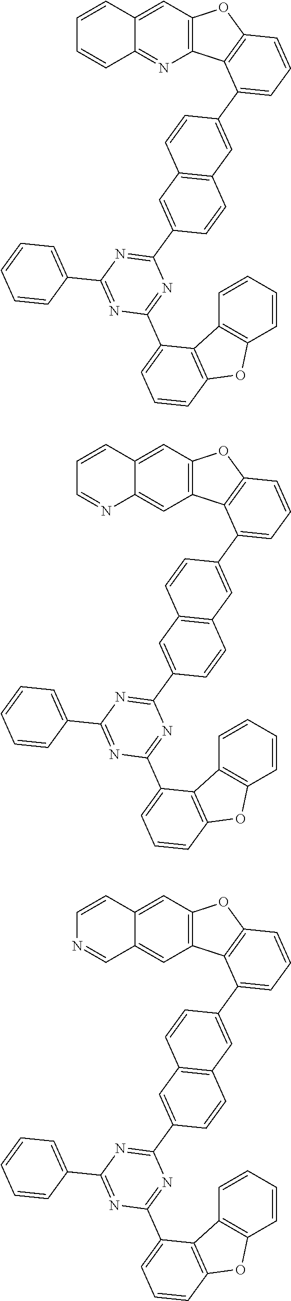

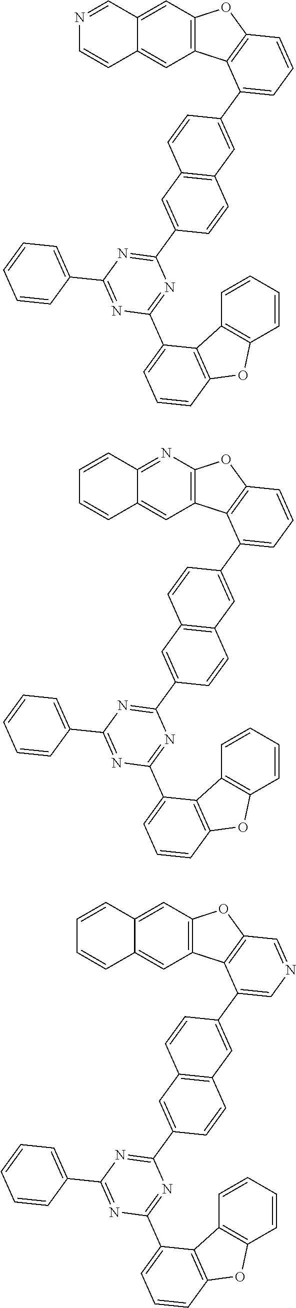 [SMILES: c1ccc(-c2nc(-c3ccc4cc(-c5cccc6oc7cc8cnccc8cc7c56)ccc4c3)nc(-c3cccc4oc5ccccc5c34)n2)cc1 ; c1ccc(-c2nc(-c3ccc4cc(-c5cccc6oc7nc8ccccc8cc7c56)ccc4c3)nc(-c3cccc4oc5ccccc5c34)n2)cc1 ; c1ccc(-c2nc(-c3ccc4cc(-c5cncc6oc7cc8ccccc8cc7c56)ccc4c3)nc(-c3cccc4oc5ccccc5c34)n2)cc1]

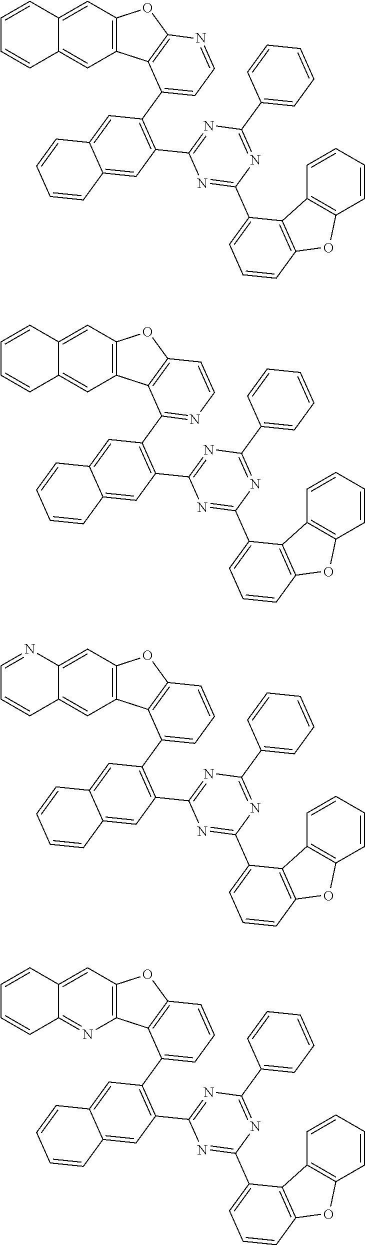 [SMILES: c1ccc(-c2nc(-c3cc4ccccc4cc3-c3ccnc4oc5cc6ccccc6cc5c34)nc(-c3cccc4oc5ccccc5c34)n2)cc1 ; c1ccc(-c2nc(-c3cc4ccccc4cc3-c3nccc4oc5cc6ccccc6cc5c34)nc(-c3cccc4oc5ccccc5c34)n2)cc1 ; c1ccc(-c2nc(-c3cc4ccccc4cc3-c3cccc4oc5cc6ncccc6cc5c34)nc(-c3cccc4oc5ccccc5c34)n2)cc1 ; c1ccc(-c2nc(-c3cc4ccccc4cc3-c3cccc4oc5cc6ccccc6nc5c34)nc(-c3cccc4oc5ccccc5c34)n2)cc1]

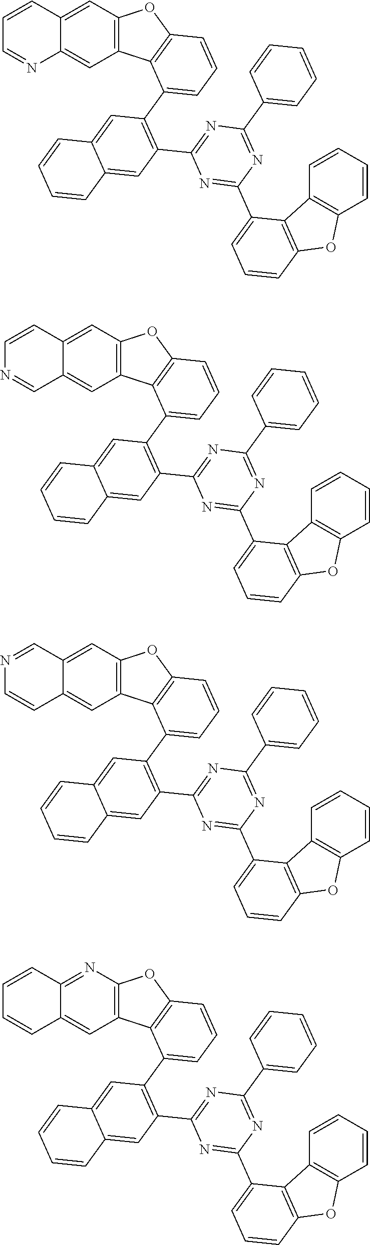 [SMILES: c1ccc(-c2nc(-c3cc4ccccc4cc3-c3cccc4oc5cc6cccnc6cc5c34)nc(-c3cccc4oc5ccccc5c34)n2)cc1 ; c1ccc(-c2nc(-c3cc4ccccc4cc3-c3cccc4oc5cc6ccncc6cc5c34)nc(-c3cccc4oc5ccccc5c34)n2)cc1 ; c1ccc(-c2nc(-c3cc4ccccc4cc3-c3cccc4oc5cc6cnccc6cc5c34)nc(-c3cccc4oc5ccccc5c34)n2)cc1 ; c1ccc(-c2nc(-c3cc4ccccc4cc3-c3cccc4oc5nc6ccccc6cc5c34)nc(-c3cccc4oc5ccccc5c34)n2)cc1]

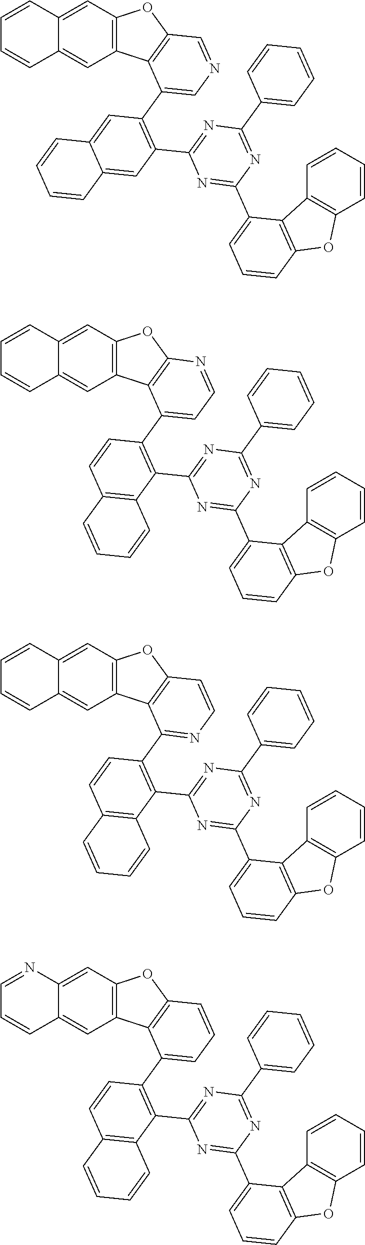 [SMILES: c1ccc(-c2nc(-c3cc4ccccc4cc3-c3cncc4oc5cc6ccccc6cc5c34)nc(-c3cccc4oc5ccccc5c34)n2)cc1 ; c1ccc(-c2nc(-c3c(-c4ccnc5oc6cc7ccccc7cc6c45)ccc4ccccc34)nc(-c3cccc4oc5ccccc5c34)n2)cc1 ; c1ccc(-c2nc(-c3c(-c4nccc5oc6cc7ccccc7cc6c45)ccc4ccccc34)nc(-c3cccc4oc5ccccc5c34)n2)cc1 ; c1ccc(-c2nc(-c3c(-c4cccc5oc6cc7ncccc7cc6c45)ccc4ccccc34)nc(-c3cccc4oc5ccccc5c34)n2)cc1]

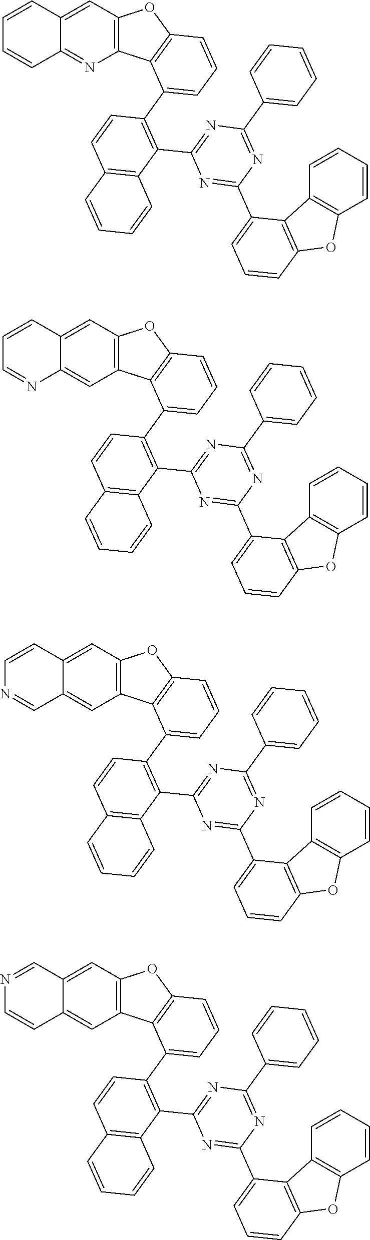 [SMILES: c1ccc(-c2nc(-c3c(-c4cccc5oc6cc7ccccc7nc6c45)ccc4ccccc34)nc(-c3cccc4oc5ccccc5c34)n2)cc1 ; c1ccc(-c2nc(-c3c(-c4cccc5oc6cc7cccnc7cc6c45)ccc4ccccc34)nc(-c3cccc4oc5ccccc5c34)n2)cc1 ; c1ccc(-c2nc(-c3c(-c4cccc5oc6cc7ccncc7cc6c45)ccc4ccccc34)nc(-c3cccc4oc5ccccc5c34)n2)cc1 ; c1ccc(-c2nc(-c3c(-c4cccc5oc6cc7cnccc7cc6c45)ccc4ccccc34)nc(-c3cccc4oc5ccccc5c34)n2)cc1]

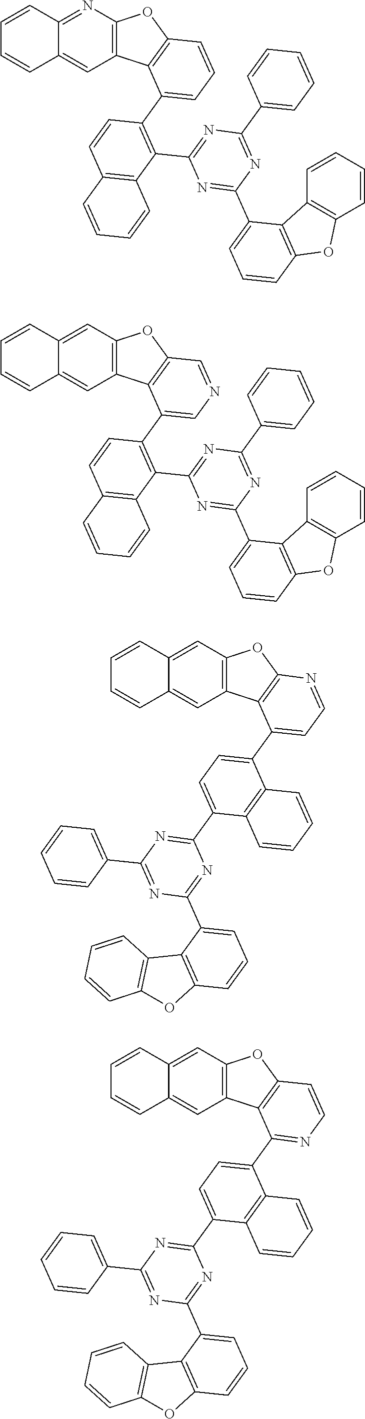 [SMILES: c1ccc(-c2nc(-c3c(-c4cccc5oc6nc7ccccc7cc6c45)ccc4ccccc34)nc(-c3cccc4oc5ccccc5c34)n2)cc1 ; c1ccc(-c2nc(-c3c(-c4cncc5oc6cc7ccccc7cc6c45)ccc4ccccc34)nc(-c3cccc4oc5ccccc5c34)n2)cc1 ; c1ccc(-c2nc(-c3ccc(-c4ccnc5oc6cc7ccccc7cc6c45)c4ccccc34)nc(-c3cccc4oc5ccccc5c34)n2)cc1 ; c1ccc(-c2nc(-c3ccc(-c4nccc5oc6cc7ccccc7cc6c45)c4ccccc34)nc(-c3cccc4oc5ccccc5c34)n2)cc1]

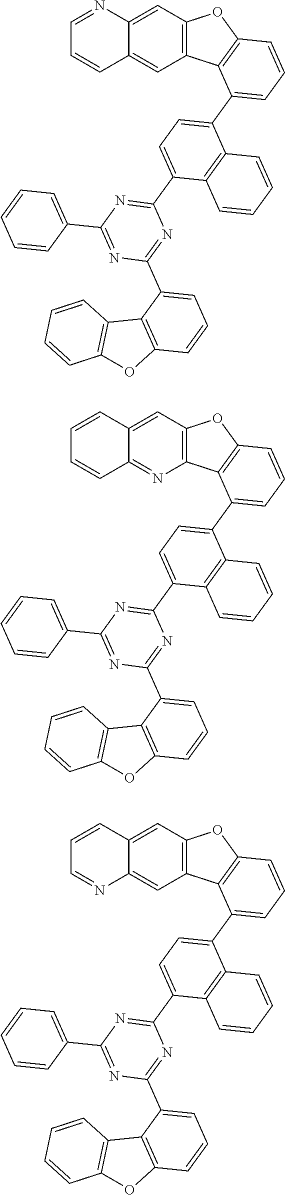 [SMILES: c1ccc(-c2nc(-c3ccc(-c4cccc5oc6cc7ncccc7cc6c45)c4ccccc34)nc(-c3cccc4oc5ccccc5c34)n2)cc1 ; c1ccc(-c2nc(-c3ccc(-c4cccc5oc6cc7ccccc7nc6c45)c4ccccc34)nc(-c3cccc4oc5ccccc5c34)n2)cc1 ; c1ccc(-c2nc(-c3ccc(-c4cccc5oc6cc7cccnc7cc6c45)c4ccccc34)nc(-c3cccc4oc5ccccc5c34)n2)cc1]

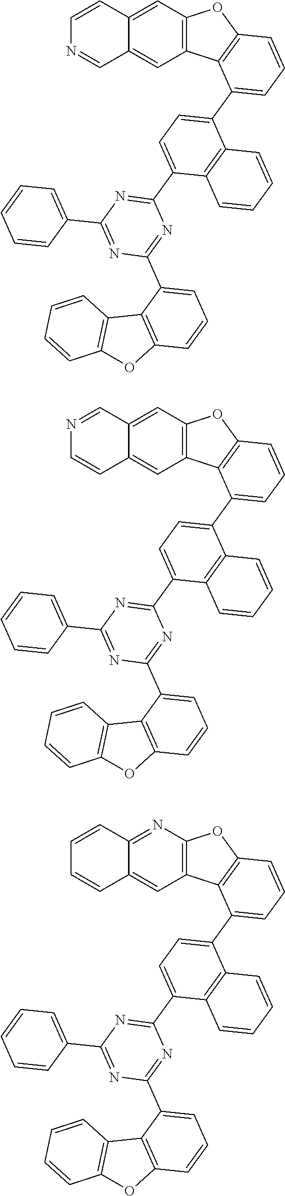 [SMILES: c1ccc(-c2nc(-c3ccc(-c4cccc5oc6cc7ccncc7cc6c45)c4ccccc34)nc(-c3cccc4oc5ccccc5c34)n2)cc1 ; c1ccc(-c2nc(-c3ccc(-c4cccc5oc6cc7cnccc7cc6c45)c4ccccc34)nc(-c3cccc4oc5ccccc5c34)n2)cc1 ; c1ccc(-c2nc(-c3ccc(-c4cccc5oc6nc7ccccc7cc6c45)c4ccccc34)nc(-c3cccc4oc5ccccc5c34)n2)cc1]

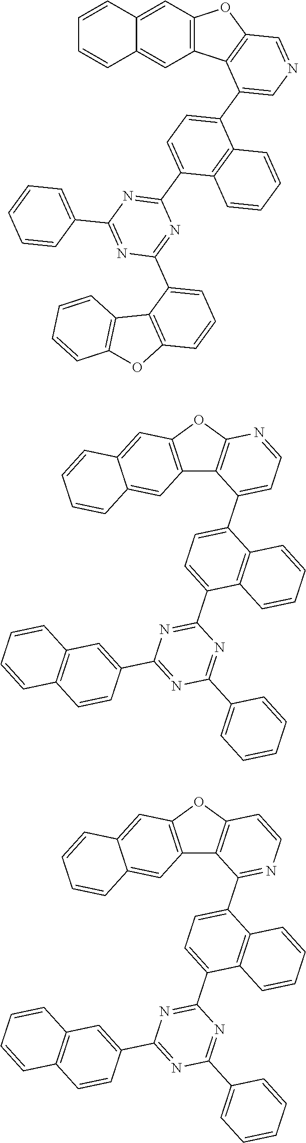 [SMILES: c1ccc(-c2nc(-c3ccc(-c4cncc5oc6cc7ccccc7cc6c45)c4ccccc34)nc(-c3cccc4oc5ccccc5c34)n2)cc1 ; c1ccc(-c2nc(-c3ccc4ccccc4c3)nc(-c3ccc(-c4ccnc5oc6cc7ccccc7cc6c45)c4ccccc34)n2)cc1 ; c1ccc(-c2nc(-c3ccc4ccccc4c3)nc(-c3ccc(-c4nccc5oc6cc7ccccc7cc6c45)c4ccccc34)n2)cc1]

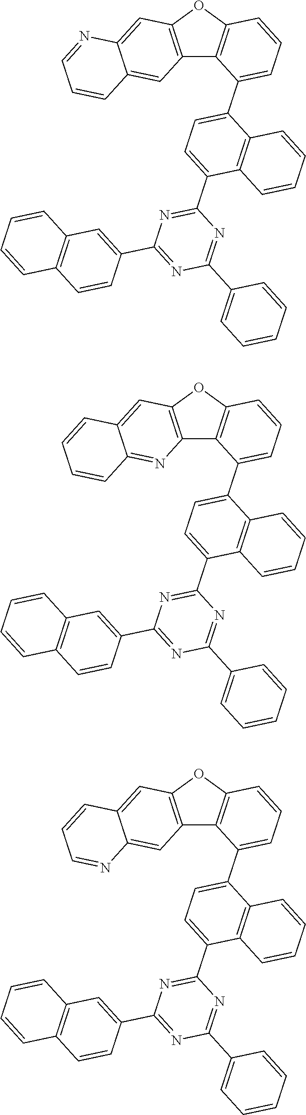 [SMILES: c1ccc(-c2nc(-c3ccc4ccccc4c3)nc(-c3ccc(-c4cccc5oc6cc7ncccc7cc6c45)c4ccccc34)n2)cc1 ; c1ccc(-c2nc(-c3ccc4ccccc4c3)nc(-c3ccc(-c4cccc5oc6cc7ccccc7nc6c45)c4ccccc34)n2)cc1 ; c1ccc(-c2nc(-c3ccc4ccccc4c3)nc(-c3ccc(-c4cccc5oc6cc7cccnc7cc6c45)c4ccccc34)n2)cc1]

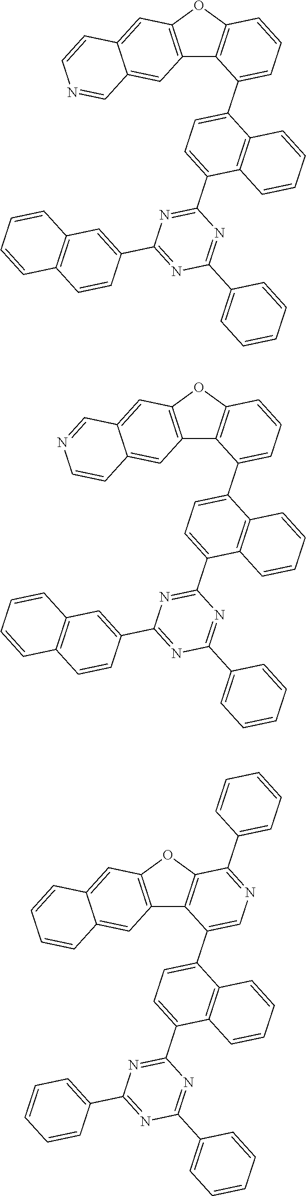 [SMILES: c1ccc(-c2nc(-c3ccc4ccccc4c3)nc(-c3ccc(-c4cccc5oc6cc7ccncc7cc6c45)c4ccccc34)n2)cc1 ; c1ccc(-c2nc(-c3ccc4ccccc4c3)nc(-c3ccc(-c4cccc5oc6cc7cnccc7cc6c45)c4ccccc34)n2)cc1 ; c1ccc(-c2nc(-c3ccccc3)nc(-c3ccc(-c4cnc(-c5ccccc5)c5oc6cc7ccccc7cc6c45)c4ccccc34)n2)cc1]

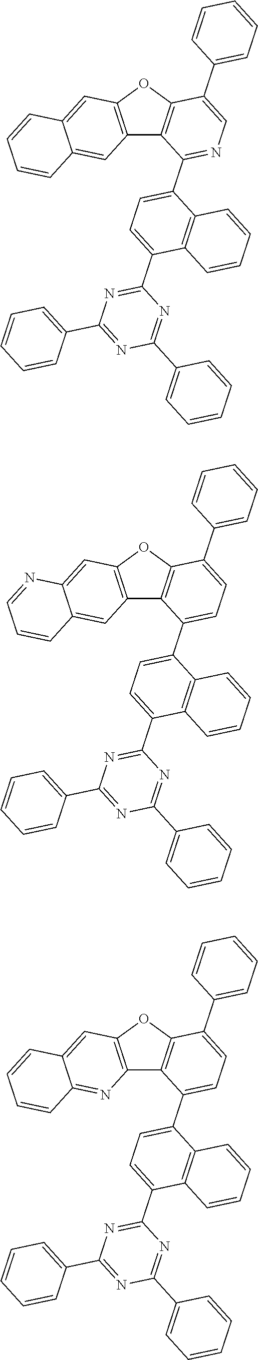 [SMILES: c1ccc(-c2nc(-c3ccccc3)nc(-c3ccc(-c4ncc(-c5ccccc5)c5oc6cc7ccccc7cc6c45)c4ccccc34)n2)cc1 ; c1ccc(-c2nc(-c3ccccc3)nc(-c3ccc(-c4ccc(-c5ccccc5)c5oc6cc7ncccc7cc6c45)c4ccccc34)n2)cc1 ; c1ccc(-c2nc(-c3ccccc3)nc(-c3ccc(-c4ccc(-c5ccccc5)c5oc6cc7ccccc7nc6c45)c4ccccc34)n2)cc1]

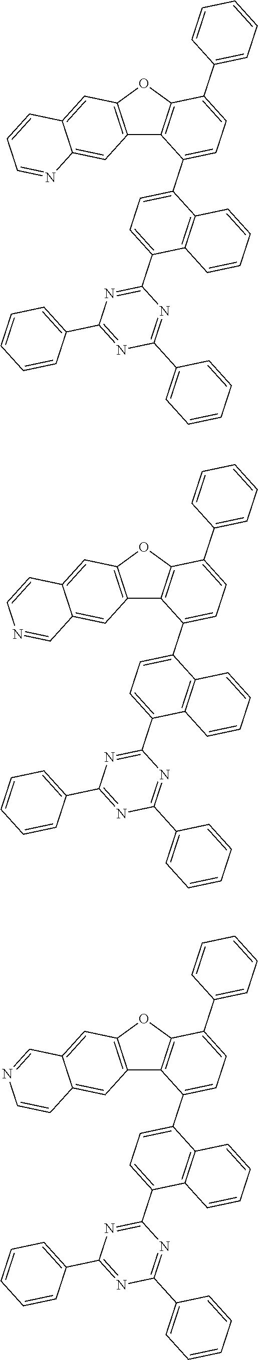 [SMILES: c1ccc(-c2nc(-c3ccccc3)nc(-c3ccc(-c4ccc(-c5ccccc5)c5oc6cc7cccnc7cc6c45)c4ccccc34)n2)cc1 ; c1ccc(-c2nc(-c3ccccc3)nc(-c3ccc(-c4ccc(-c5ccccc5)c5oc6cc7ccncc7cc6c45)c4ccccc34)n2)cc1 ; c1ccc(-c2nc(-c3ccccc3)nc(-c3ccc(-c4ccc(-c5ccccc5)c5oc6cc7cnccc7cc6c45)c4ccccc34)n2)cc1]

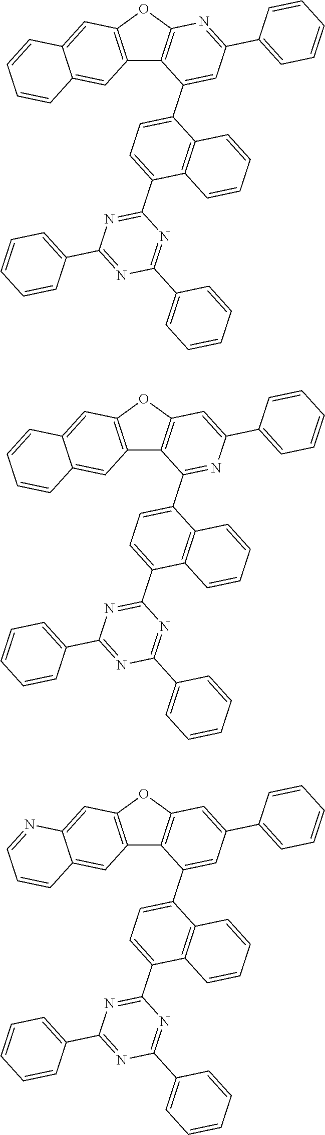 [SMILES: c1ccc(-c2cc(-c3ccc(-c4nc(-c5ccccc5)nc(-c5ccccc5)n4)c4ccccc34)c3c(n2)oc2cc4ccccc4cc23)cc1 ; c1ccc(-c2cc3oc4cc5ccccc5cc4c3c(-c3ccc(-c4nc(-c5ccccc5)nc(-c5ccccc5)n4)c4ccccc34)n2)cc1 ; c1ccc(-c2cc(-c3ccc(-c4nc(-c5ccccc5)nc(-c5ccccc5)n4)c4ccccc34)c3c(c2)oc2cc4ncccc4cc23)cc1]

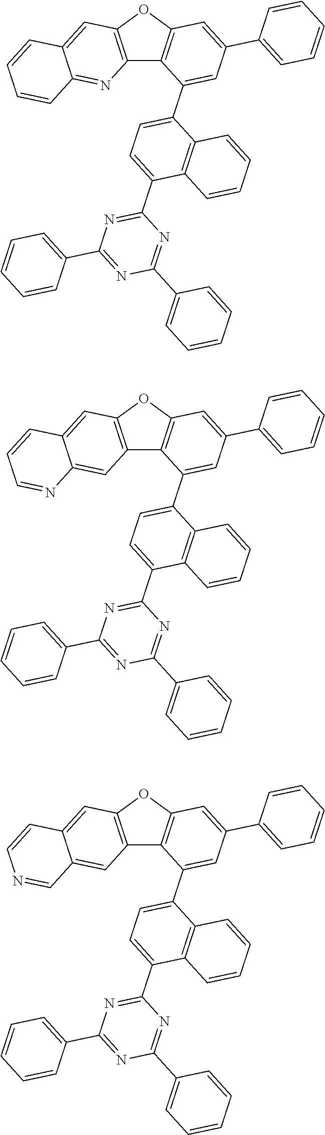 [SMILES: c1ccc(-c2cc(-c3ccc(-c4nc(-c5ccccc5)nc(-c5ccccc5)n4)c4ccccc34)c3c(c2)oc2cc4ccccc4nc23)cc1 ; c1ccc(-c2cc(-c3ccc(-c4nc(-c5ccccc5)nc(-c5ccccc5)n4)c4ccccc34)c3c(c2)oc2cc4cccnc4cc23)cc1 ; c1ccc(-c2cc(-c3ccc(-c4nc(-c5ccccc5)nc(-c5ccccc5)n4)c4ccccc34)c3c(c2)oc2cc4ccncc4cc23)cc1]

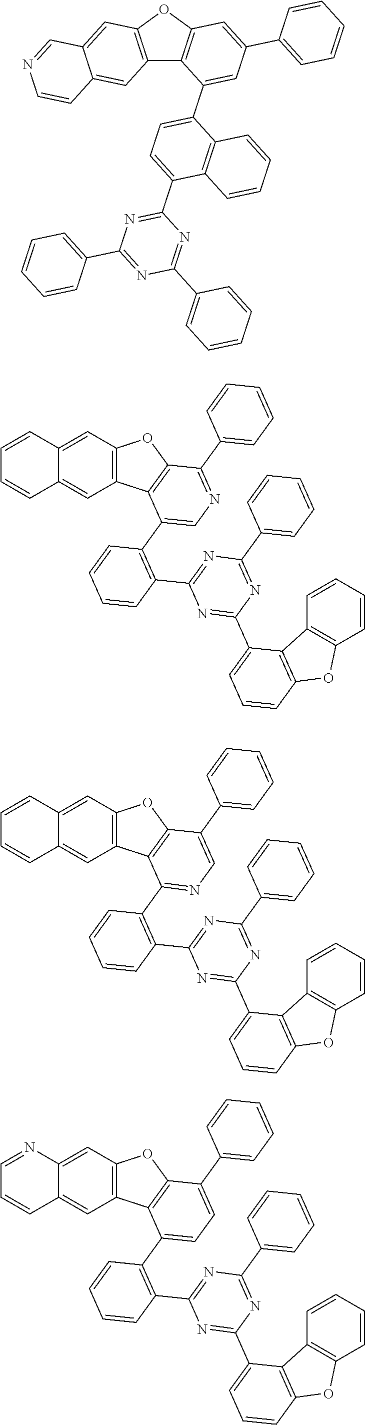

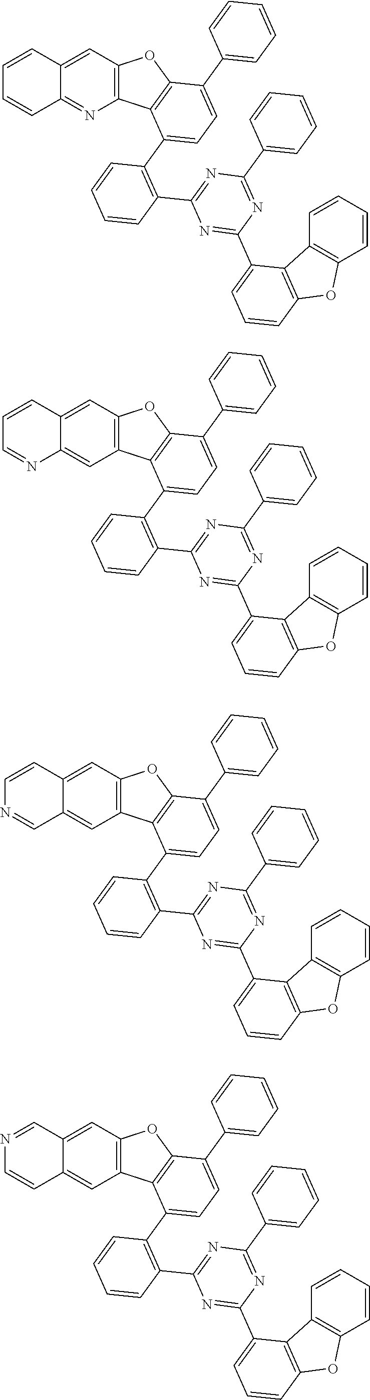

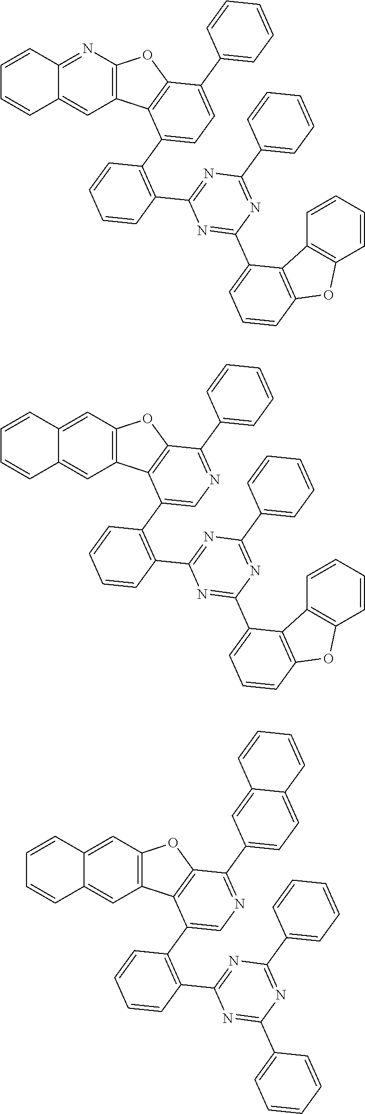

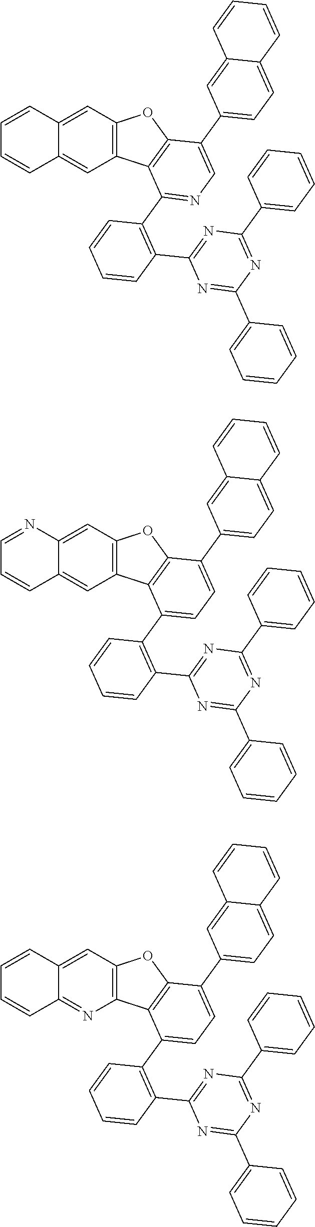

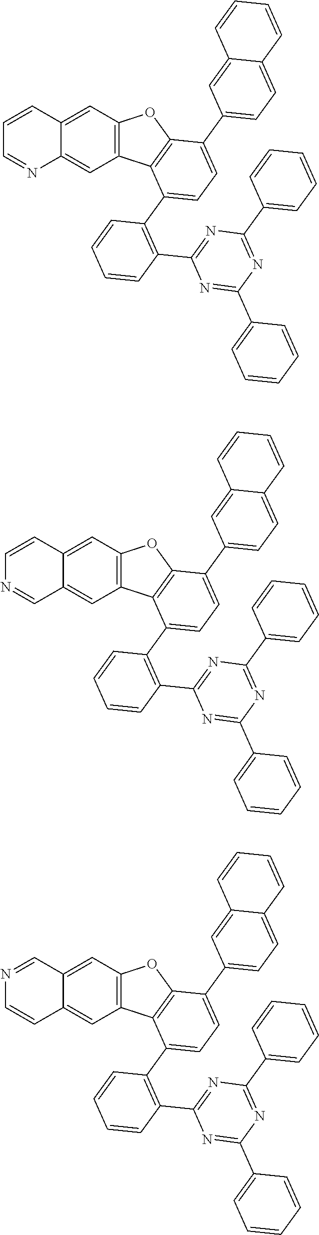

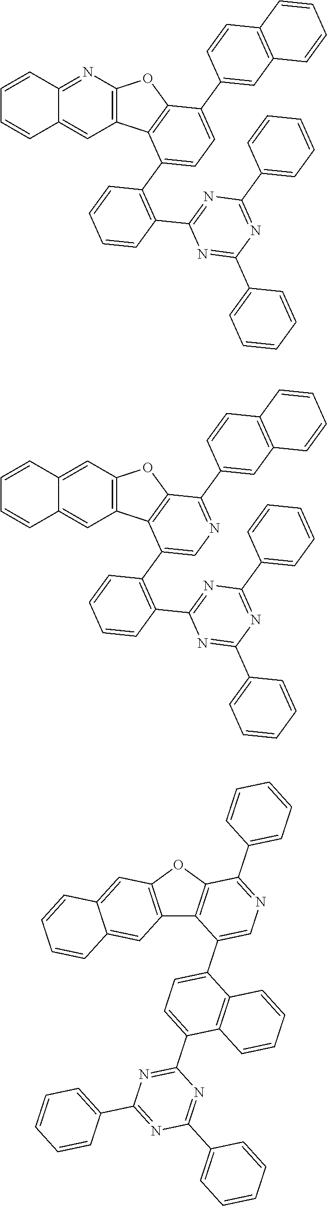

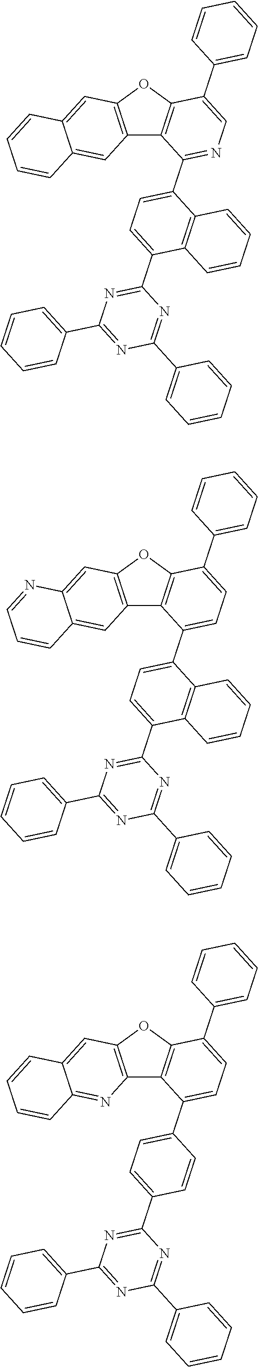 [SMILES: c1ccc(-c2nc(-c3ccccc3)nc(-c3ccc(-c4ncc(-c5ccccc5)c5oc6cc7ccccc7cc6c45)c4ccccc34)n2)cc1 ; c1ccc(-c2nc(-c3ccccc3)nc(-c3ccc(-c4ccc(-c5ccccc5)c5oc6cc7ncccc7cc6c45)c4ccccc34)n2)cc1 ; c1ccc(-c2nc(-c3ccccc3)nc(-c3ccc(-c4ccc(-c5ccccc5)c5oc6cc7ccccc7nc6c45)cc3)n2)cc1]

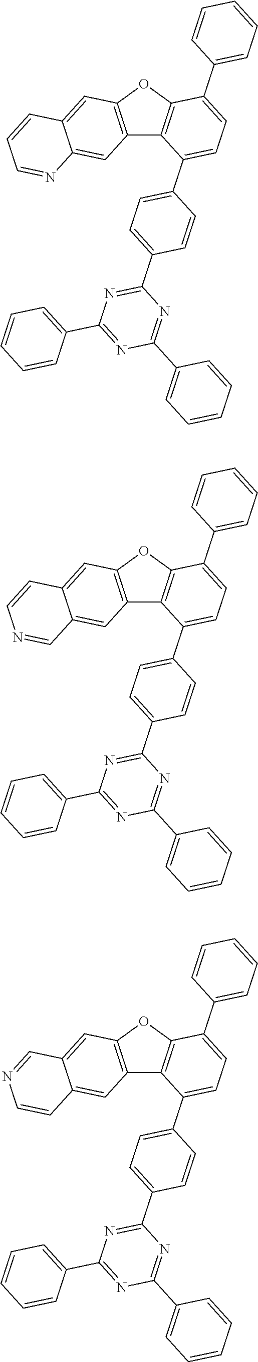 [SMILES: c1ccc(-c2nc(-c3ccccc3)nc(-c3ccc(-c4ccc(-c5ccccc5)c5oc6cc7cccnc7cc6c45)cc3)n2)cc1 ; c1ccc(-c2nc(-c3ccccc3)nc(-c3ccc(-c4ccc(-c5ccccc5)c5oc6cc7ccncc7cc6c45)cc3)n2)cc1 ; c1ccc(-c2nc(-c3ccccc3)nc(-c3ccc(-c4ccc(-c5ccccc5)c5oc6cc7cnccc7cc6c45)cc3)n2)cc1]

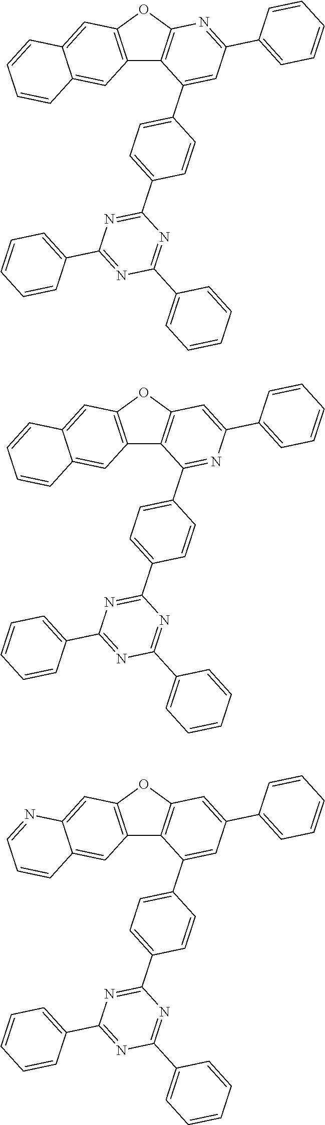 [SMILES: c1ccc(-c2cc(-c3ccc(-c4nc(-c5ccccc5)nc(-c5ccccc5)n4)cc3)c3c(n2)oc2cc4ccccc4cc23)cc1 ; c1ccc(-c2cc3oc4cc5ccccc5cc4c3c(-c3ccc(-c4nc(-c5ccccc5)nc(-c5ccccc5)n4)cc3)n2)cc1 ; c1ccc(-c2cc(-c3ccc(-c4nc(-c5ccccc5)nc(-c5ccccc5)n4)cc3)c3c(c2)oc2cc4ncccc4cc23)cc1]

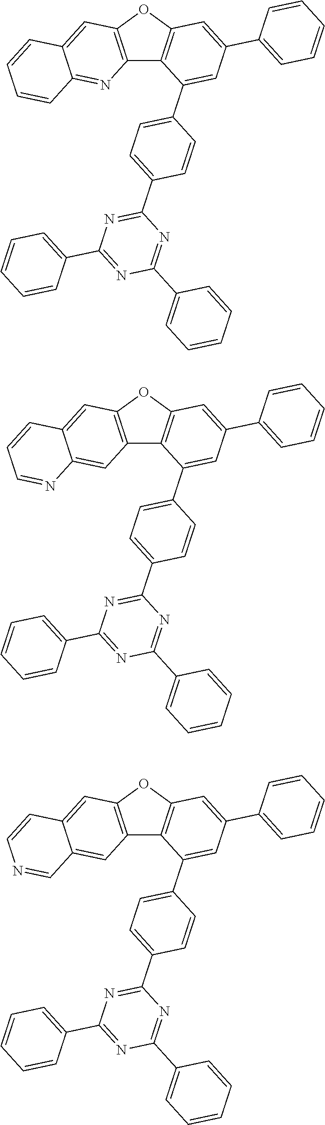 [SMILES: c1ccc(-c2cc(-c3ccc(-c4nc(-c5ccccc5)nc(-c5ccccc5)n4)cc3)c3c(c2)oc2cc4ccccc4nc23)cc1 ; c1ccc(-c2cc(-c3ccc(-c4nc(-c5ccccc5)nc(-c5ccccc5)n4)cc3)c3c(c2)oc2cc4cccnc4cc23)cc1 ; c1ccc(-c2cc(-c3ccc(-c4nc(-c5ccccc5)nc(-c5ccccc5)n4)cc3)c3c(c2)oc2cc4ccncc4cc23)cc1]

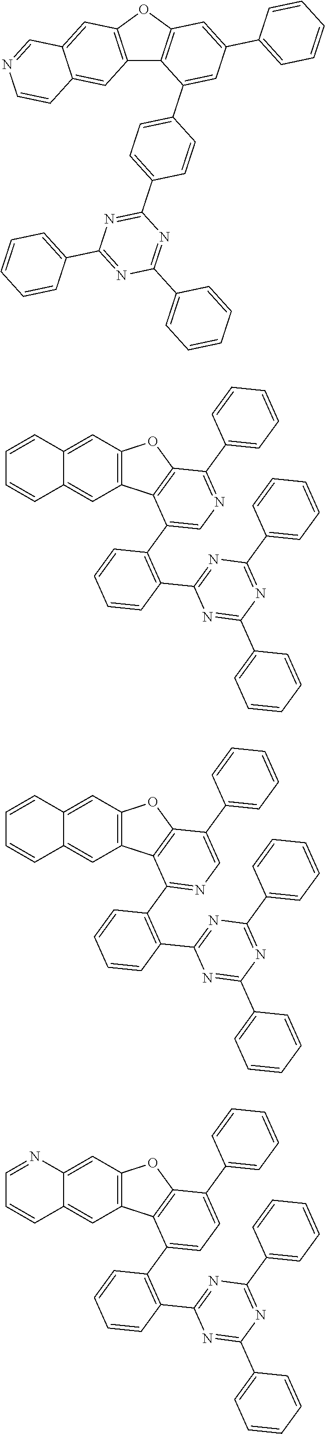

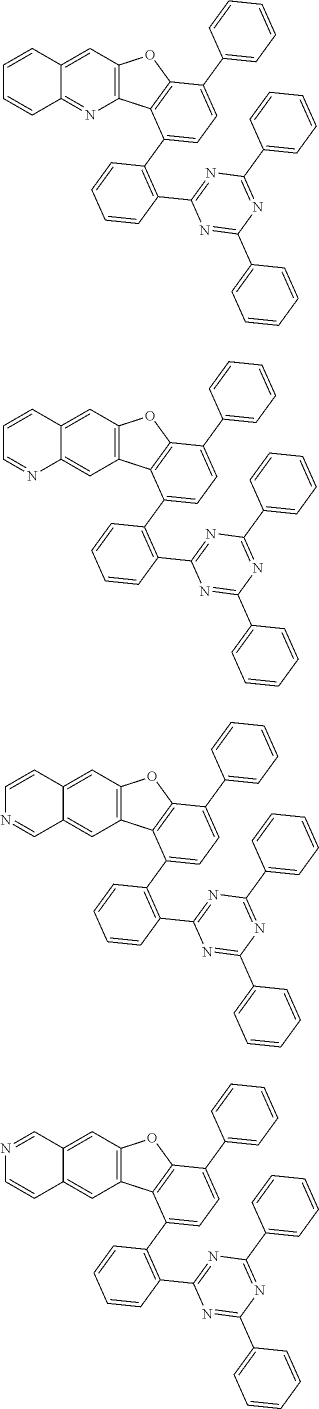

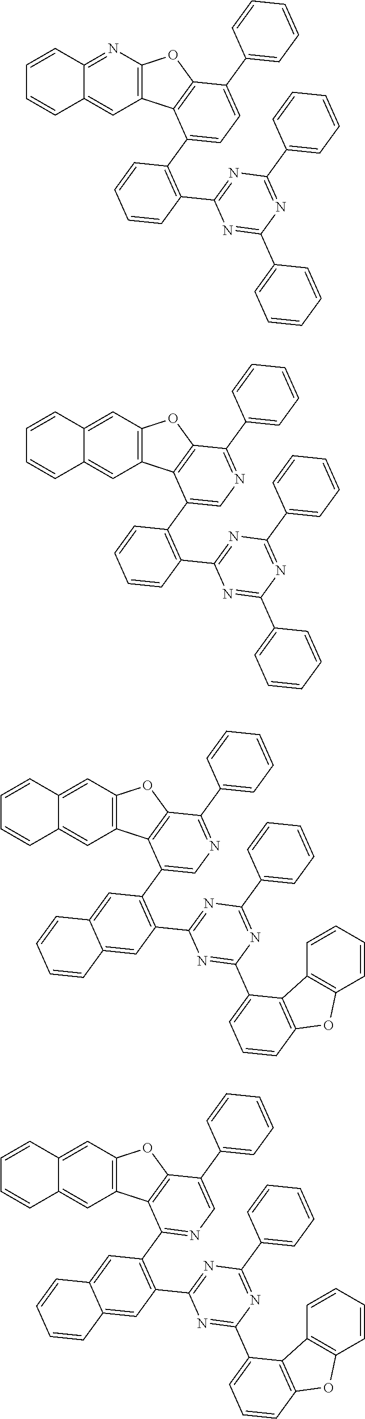

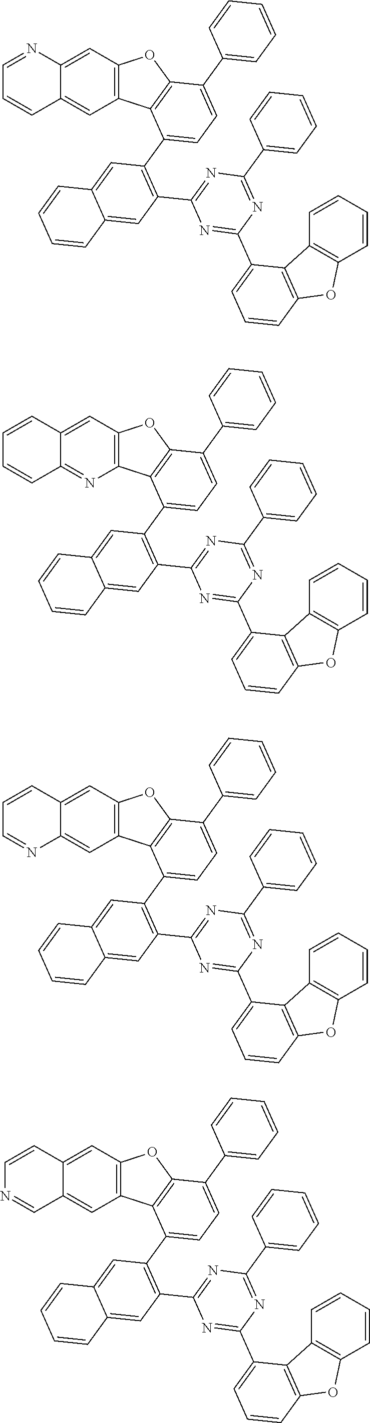

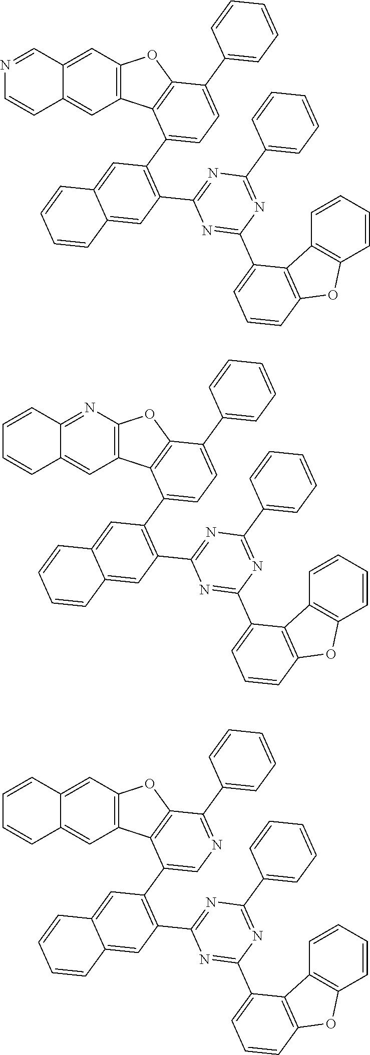

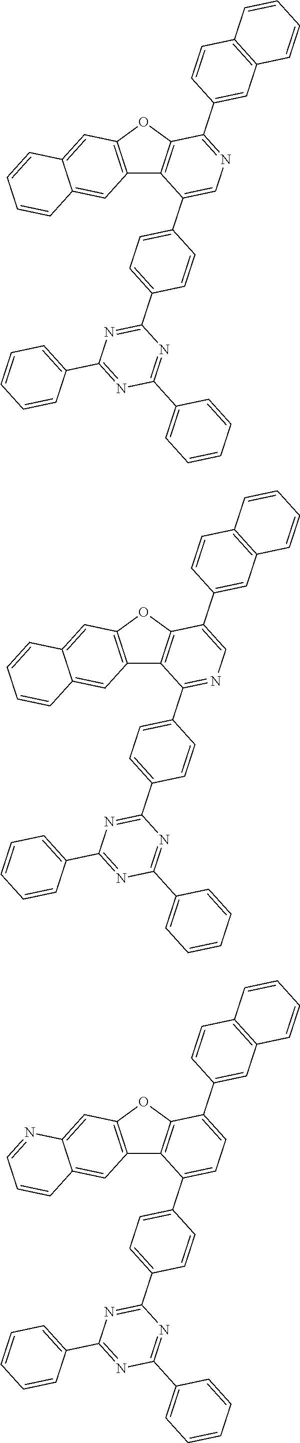 [SMILES: c1ccc(-c2nc(-c3ccccc3)nc(-c3ccc(-c4cnc(-c5ccc6ccccc6c5)c5oc6cc7ccccc7cc6c45)cc3)n2)cc1 ; c1ccc(-c2nc(-c3ccccc3)nc(-c3ccc(-c4ncc(-c5ccc6ccccc6c5)c5oc6cc7ccccc7cc6c45)cc3)n2)cc1 ; c1ccc(-c2nc(-c3ccccc3)nc(-c3ccc(-c4ccc(-c5ccc6ccccc6c5)c5oc6cc7ncccc7cc6c45)cc3)n2)cc1]

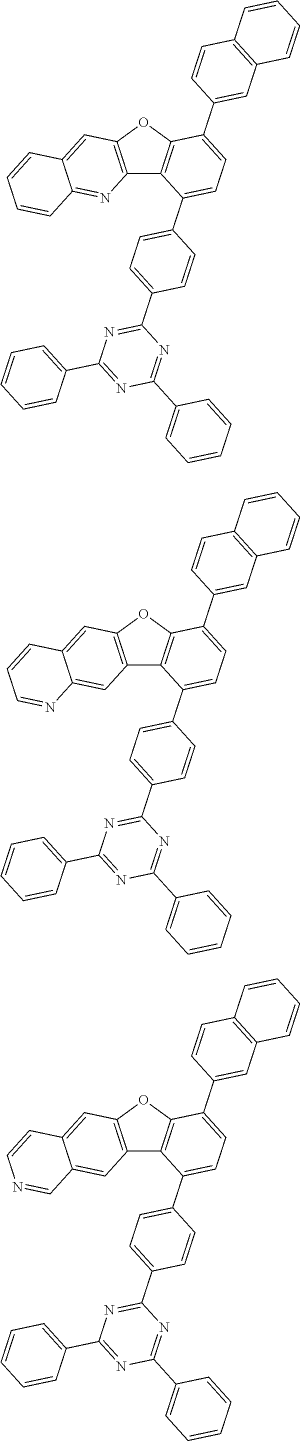

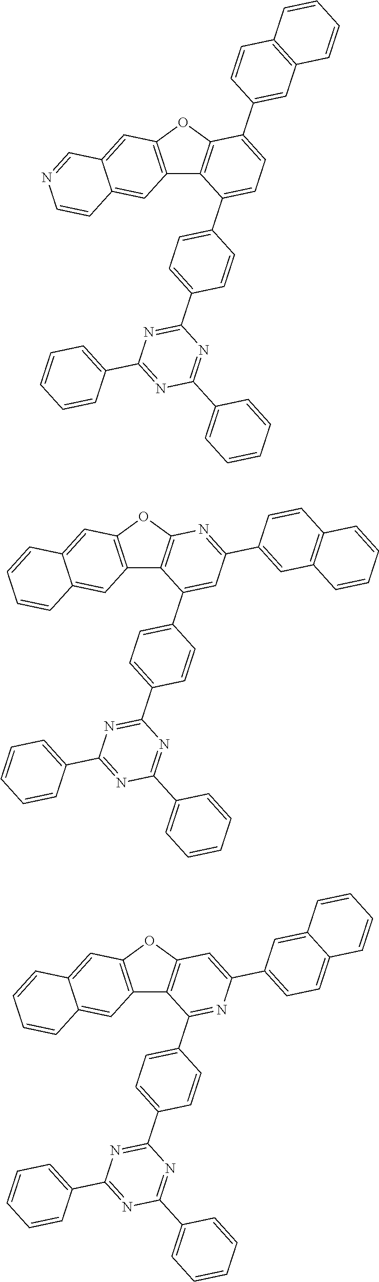 [SMILES: c1ccc(-c2nc(-c3ccccc3)nc(-c3ccc(-c4ccc(-c5ccc6ccccc6c5)c5oc6cc7cnccc7cc6c45)cc3)n2)cc1 ; c1ccc(-c2nc(-c3ccccc3)nc(-c3ccc(-c4cc(-c5ccc6ccccc6c5)nc5oc6cc7ccccc7cc6c45)cc3)n2)cc1 ; c1ccc(-c2nc(-c3ccccc3)nc(-c3ccc(-c4nc(-c5ccc6ccccc6c5)cc5oc6cc7ccccc7cc6c45)cc3)n2)cc1]

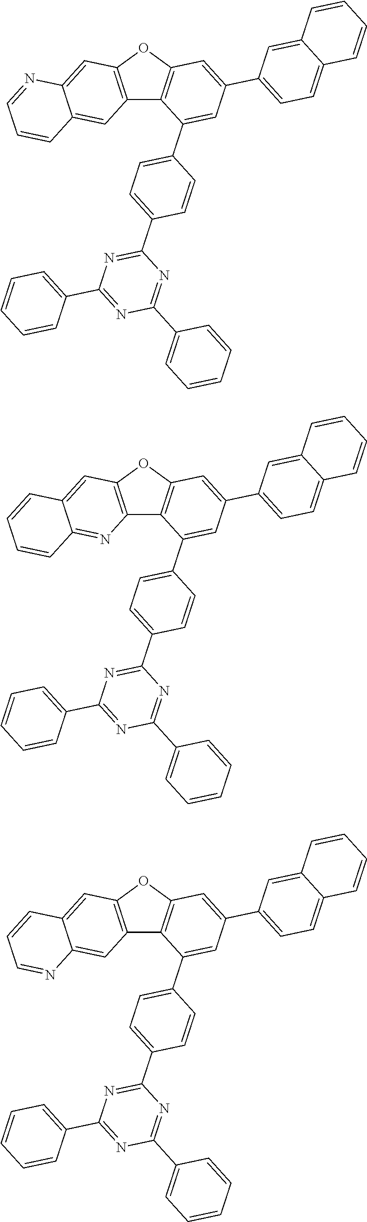 [SMILES: c1ccc(-c2nc(-c3ccccc3)nc(-c3ccc(-c4cc(-c5ccc6ccccc6c5)cc5oc6cc7ncccc7cc6c45)cc3)n2)cc1 ; c1ccc(-c2nc(-c3ccccc3)nc(-c3ccc(-c4cc(-c5ccc6ccccc6c5)cc5oc6cc7ccccc7nc6c45)cc3)n2)cc1 ; c1ccc(-c2nc(-c3ccccc3)nc(-c3ccc(-c4cc(-c5ccc6ccccc6c5)cc5oc6cc7cccnc7cc6c45)cc3)n2)cc1]

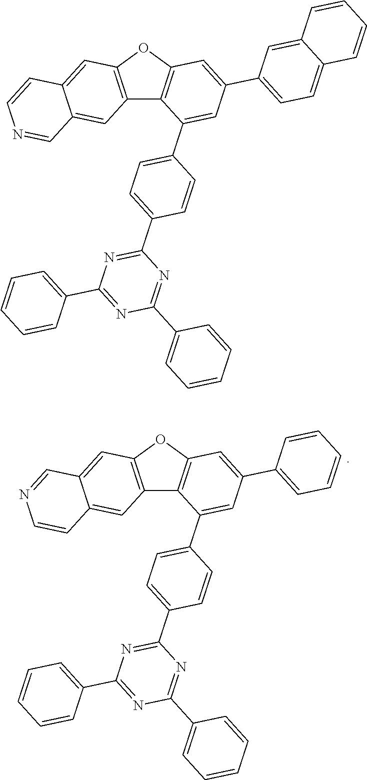 [SMILES: c1ccc(-c2nc(-c3ccccc3)nc(-c3ccc(-c4cc(-c5ccc6ccccc6c5)cc5oc6cc7ccncc7cc6c45)cc3)n2)cc1 ; c1ccc(-c2cc(-c3ccc(-c4nc(-c5ccccc5)nc(-c5ccccc5)n4)cc3)c3c(c2)oc2cc4cnccc4cc23)cc1]

Meanwhile, the compound of Chemical Formula 1 can be prepared, for example, by the preparation method as shown in the following Reaction Scheme 1.

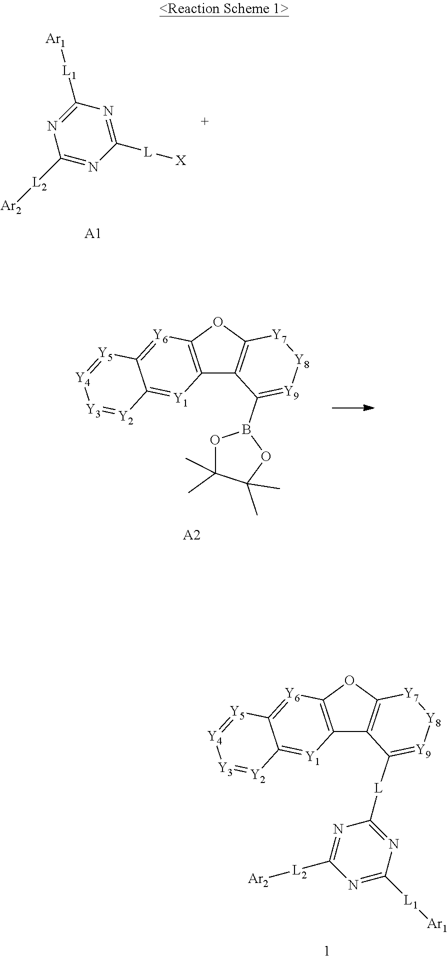

In Reaction Scheme 1, X is halogen, preferably bromo, or chloro, and the definitions of other substituents are the same as described above.

Specifically, the compound of Chemical Formula 1 can be prepared by subjecting the starting materials A1 and A2 to a Suzuki coupling reaction. The Suzuki coupling reaction is preferably carried out in the presence of a palladium catalyst and a base, and a reactive group for the Suzuki coupling reaction can be appropriately modified, and the method for preparing the compound of Chemical Formula 1 can be further embodied in Preparation Examples described hereinafter.

(Organic Light Emitting Device)

Further, the present disclosure provides an organic light emitting device comprising the compound of Chemical Formula 1. In one example, the present disclosure provides an organic light emitting device comprising: a first electrode; a second electrode that is provided opposite to the first electrode; and one or more organic material layers that are provided between the first electrode and the second electrode, wherein one or more layers of the organic material layers includes the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, an electron transport layer, an electron injection layer and the like as the organic material layer. However, the structure of the organic light emitting device is not limited thereto, and it can include a smaller number of organic layers.

In one embodiment, the organic material layer can include a light emitting layer, wherein the organic material layer including the above compound can be a light emitting layer.

In another embodiment, the organic material layer can include a hole injection layer, a hole transport layer, a light emitting layer and an electron injection and transport layer, wherein the organic material layer including the above compound can be a light emitting layer.

In another embodiment, the organic material layer can include a hole injection layer, a hole transport layer, an electron blocking layer, a light emitting layer and an electron injection and transport layer, wherein the organic material layer including the above compound can be a light emitting layer.

In yet another embodiment, the organic material layer can include a hole injection layer, a hole transport layer, an electron blocking layer, a light emitting layer, a hole blocking layer, and an electron injection and transport layer, wherein the organic material layer including the above compound can be a light emitting layer.

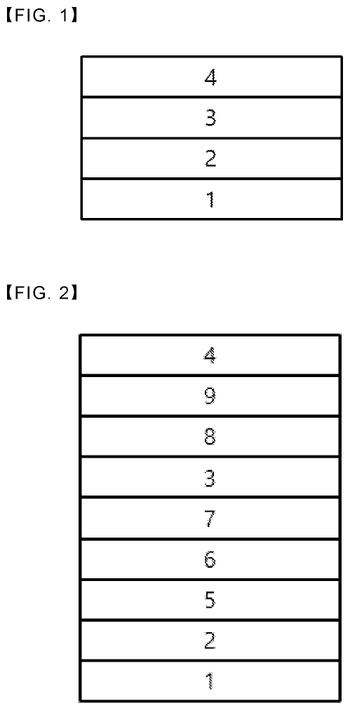

Further, the organic light emitting device according to the present disclosure can be a normal type organic light emitting device in which an anode, one or more organic material layers and a cathode are sequentially stacked on a substrate wherein the first electrode is an anode, and the second electrode is a cathode. Further, the organic light emitting device according to the present disclosure can be an inverted type organic light emitting device in which a cathode, one or more organic material layers and an anode are sequentially stacked on a substrate wherein the first electrode is a cathode and the second electrode is an anode. For example, the structure of an organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4. In such a structure, the compound of Chemical Formula 1 can be included in the light emitting layer.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

The organic light emitting device according to the present disclosure can be manufactured by materials and methods known in the art, except that the light emitting layer includes the compound according to the present disclosure, and is manufactured according to the above-mentioned method.

For example, the organic light emitting device according to the present disclosure can be manufactured by sequentially stacking an anode, an organic material layer and a cathode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon.

In addition to such a method, the organic light emitting device can be manufactured by sequentially depositing a cathode material, an organic material layer and an anode material on a substrate (International Publication WO2003/012890), However, the manufacturing method is not limited thereto.

In one example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methyl-thiophene), poly[3,4-(ethylene-1,2-dioxy)thiophene] (PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/AI or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to an electron injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive compound, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include an arylamine-based organic material, a conductive compound, a block copolymer in which a conjugate portion and a non-conjugate portion are present together, and the like, but are not limited thereto.

The electron blocking layer refers to a layer which is formed on the hole transport layer, preferably provided in contact with the light emitting layer, and serves to adjust the hole mobility, prevent excessive movement of electrons, and increase the probability of hole-electron coupling, thereby improving the efficiency of the organic light emitting device. The electron blocking layer includes an electron blocking material, and examples of such electron blocking material can include an arylamine-based organic material or the like, but is not limited thereto.

The light emitting layer can include a host material and a dopant material. The host material can be the compound of Chemical Formula 1. Further, the host material can be a fused aromatic ring derivative, a heterocycle-containing compound or the like in addition to the compound of Chemical Formula 1. Specific examples of the fused aromatic ring derivatives include anthracenyl derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Further, the dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracenyl, chrycenyl, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

More specifically, the dopant material can include compounds having the following structures, but is not limited thereto:

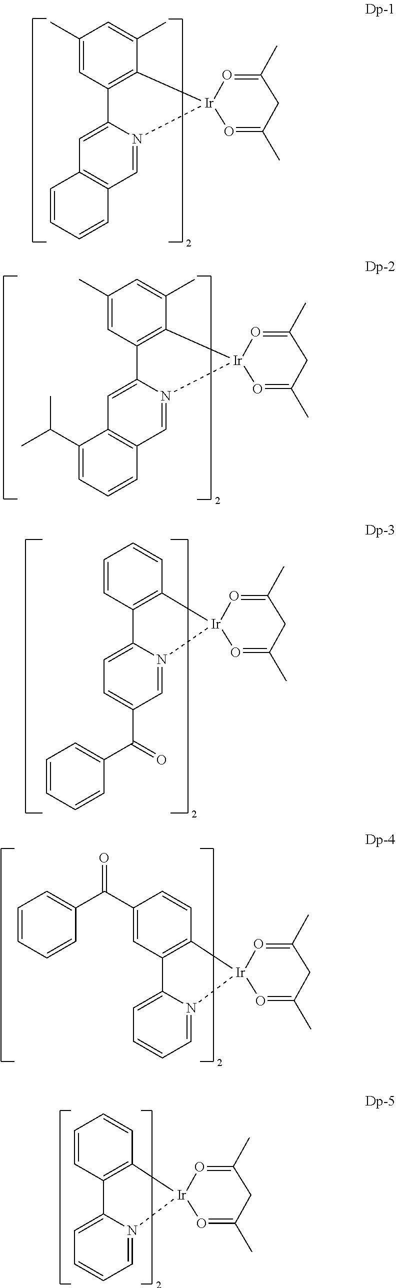

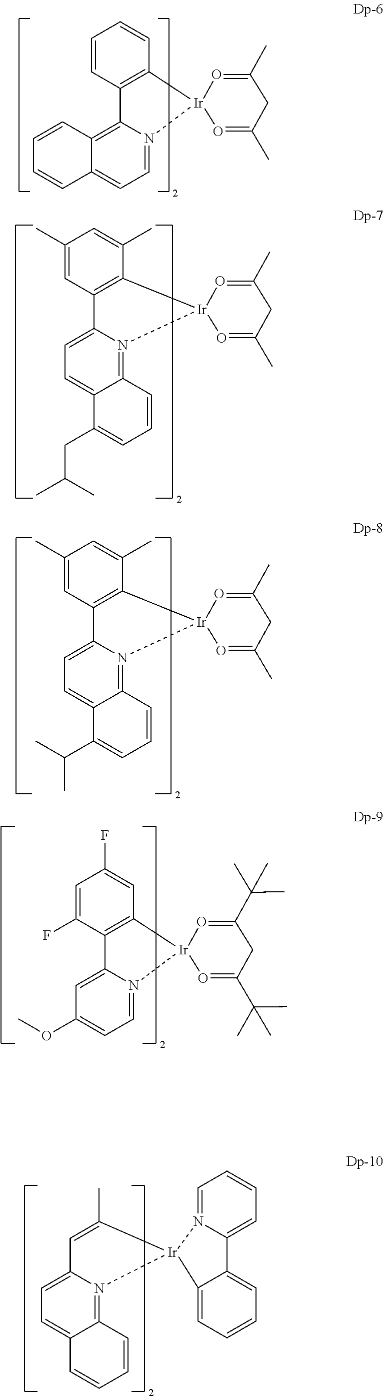

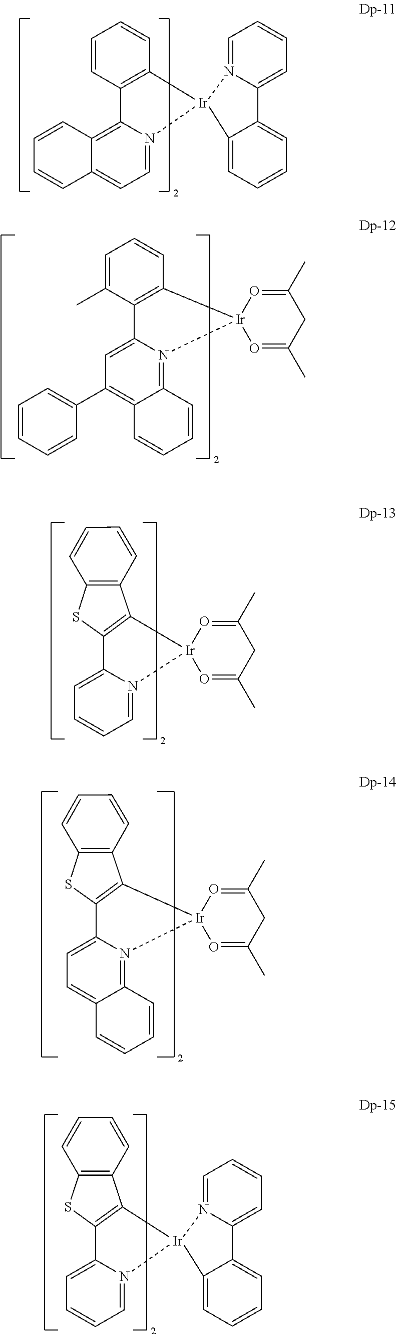

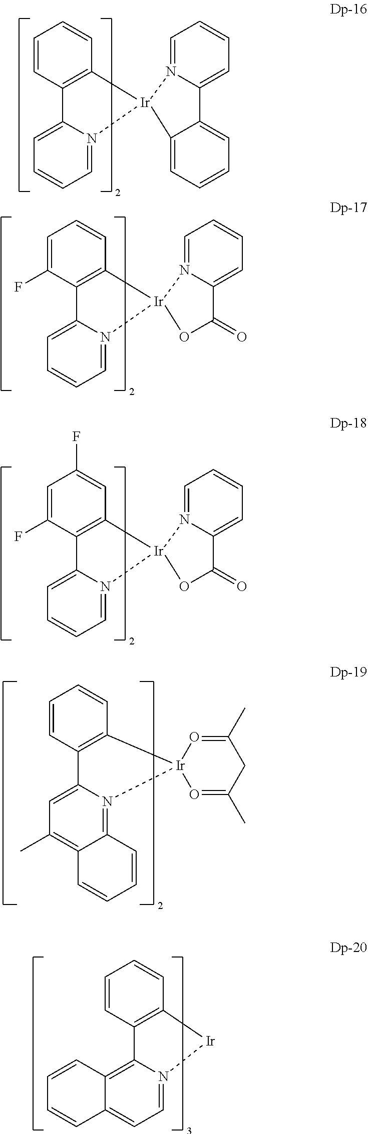

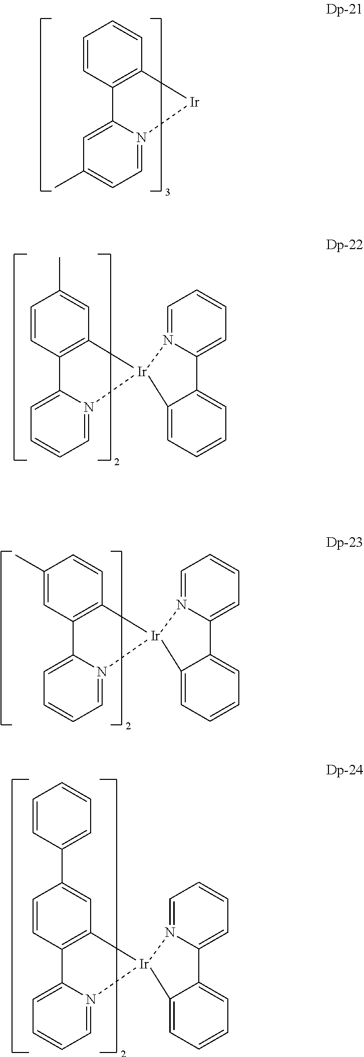

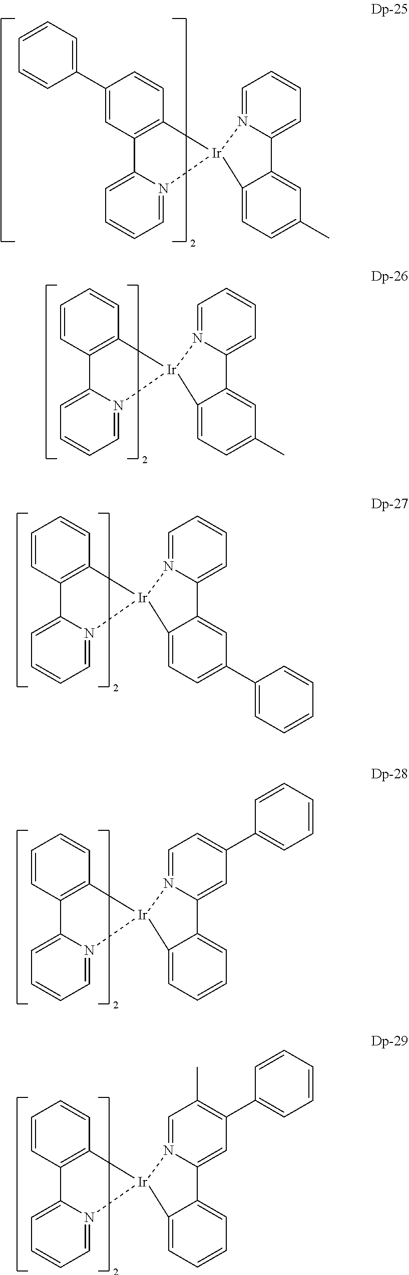

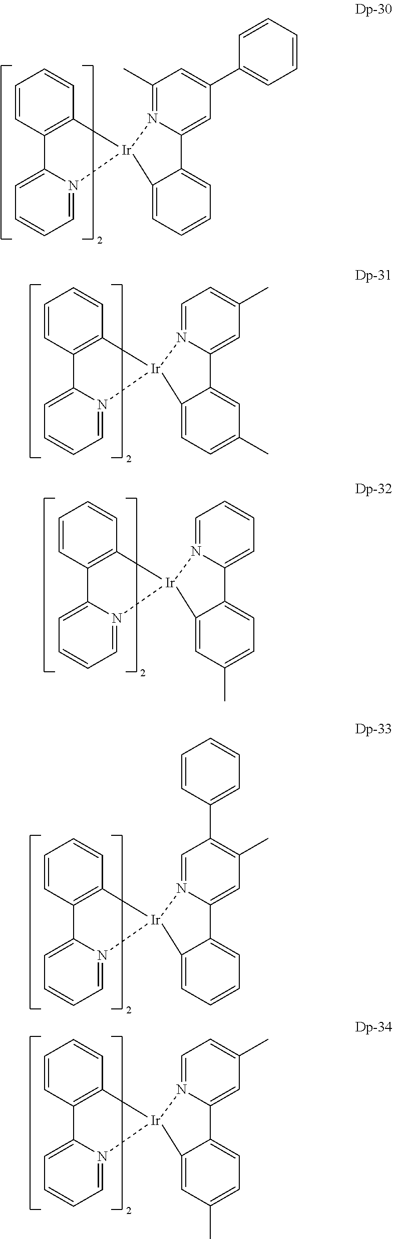

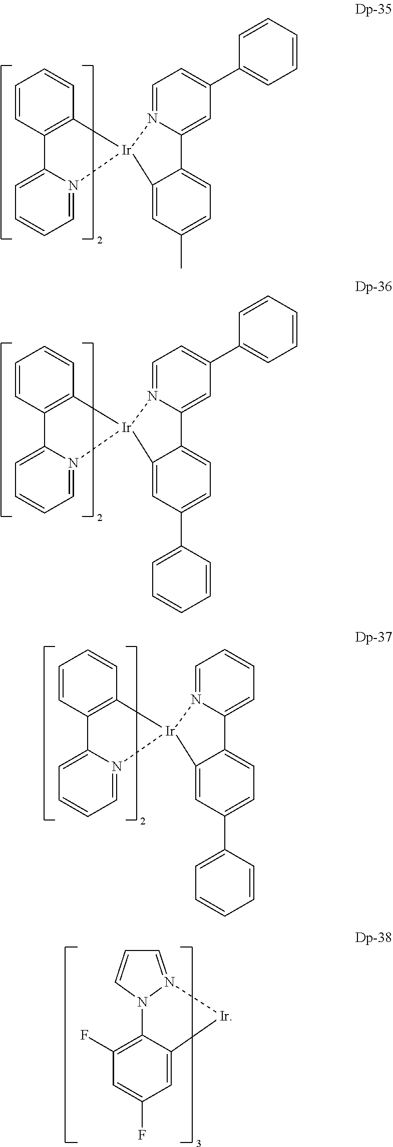

The hole blocking layer refers to a layer which is formed on the light emitting layer, preferably provided in contact with the light emitting layer, and serves to adjust the electron mobility, prevent excessive movement of holes, and increase the probability of hole-electron coupling, thereby improving the efficiency of the organic light emitting device. The hole blocking layer includes a hole blocking material, and examples of such hole blocking material can include a compound having an electron-withdrawing group introduced therein, such as azine derivatives including triazine; triazole derivatives; oxadiazole derivatives; phenanthroline derivatives; and phosphine oxide derivatives, but is not limited thereto.

The electron injection and transport layer is a layer for simultaneously performing the roles of an electron transport layer and an electron injection layer that inject electrons from an electrode and transport the received electrons up to the light emitting layer, and is formed on the light emitting layer or the hole blocking layer. The electron injection and transport material is suitably a material which can receive electrons well from a cathode and transfer the electrons to a light emitting layer, and has a large mobility for electrons. Specific examples of the electron injection and transport material include: an Al complex of 8-hydroxyquinoline, a complex including Alq₃, an organic radical compound, a hydroxyflavone-metal complex, a triazine derivative, and the like, but are not limited thereto. Alternatively, it can be used together with fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The electron injection and transport layer can also be formed as a separate layer such as an electron injection layer and an electron transport layer. In such a case, the electron transport layer is formed on the light emitting layer or the hole blocking layer, and the above-mentioned electron injection and transport material can be used as the electron transport material included in the electron transport layer. In addition, the electron injection layer is formed on the electron transport layer, and examples of the electron injection material included in the electron injection layer include LiF, NaCl, CsF, Li₂O, BaO, fluorenone, anthraquinodimethane, diphenoquinone, thiopyran dioxide, oxazole, oxadiazole, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)alum inum, tris(2-methyl-8-hydroxy-quinolinato)aluminum, tris(8-hydroxyquinolinato)gallium, bis(10-hydroxybenzo[h]quinolinato)beryllium, bis(10-hydroxybenzo[h]-quinolinato)zinc, bis(2-methyl-8-quinolinato)-chlorogallium, bis(2-methyl-8-quinolinato)(o-cresolato)gallium, bis(2-methyl-8-quinolinato)(1-naphtholato)alum inum, bis(2-methyl-8-quinolinato)(2-naphtholato)gallium, and the like, but are not limited thereto.

The organic light emitting device according to the present disclosure can be a bottom emission device, a top emission device, or a double-sided light emitting device, and in particular, can be a bottom emission device that requires relatively high luminous efficiency.

In addition, the compound of Chemical Formula 1 can be included in an organic solar cell or an organic transistor in addition to an organic light emitting device.

The preparation of the compound of Chemical Formula 1 and the organic light emitting device including the same will be described in detail in the following examples. However, these examples are presented for illustrative purposes only, and are not intended to limit the scope of the present disclosure.

### SYNTHESIS EXAMPLE A: SYNTHESIS OF INTERMEDIATE COMPOUND A

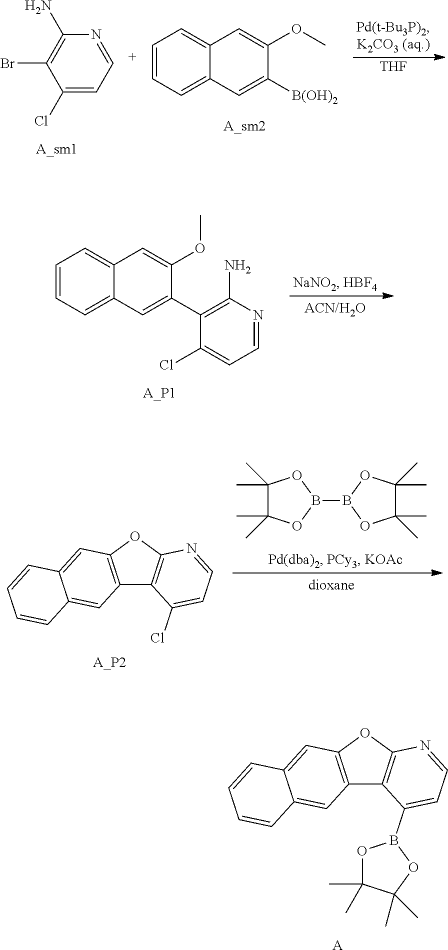

A_sm1 (15 g, 72.3 mmol) and A_sm2 (19 g, 94 mmol) were added to 300 mL of THF under a nitrogen atmosphere, and the mixture was stirred and refluxed. Then, potassium carbonate (30 g, 216.9 mmol) was dissolved in 90 ml of water, added thereto, sufficiently stirred, and then bis(tri-tert-butylphosphine)-paliadium(0) (0.4 g, 0.7 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 14.6 g of A_P1. (Yield: 71%, MS: [M+H]⁺=285).

Next, A_P1 (10 g, 35.1 mmol) and HBF₄ (6.2 g, 70.2 mmol) were added to 100 mL of ACN under a nitrogen atmosphere, and the mixture was stirred. Then, NaNO₂ (4.8 g, 70.2 mmol) was dissolved in 20 mL of H₂O and slowly added at 0° C. After reacting for 10 hours, the mixture was heated up to room temperature, and then diluted by adding 200 mL of water. The solution was completely dissolved in chloroform, washed twice with water, the organic layer was separated, treated with anhydrous magnesium sulfate, filtered, and the filtrate was distilled under reduced pressure. The concentrated compound was purified by silica gel column chromatography to give 6.5 g of A_P2. (Yield: 74%, MS: [M+H]⁺=251).

Next, A_P2 (15 g, 59,1 mmol) and bis(pinacolato)diboron (16.5 g, 65 mmol) were added to 300 mL of 1,4-dioxane under a nitrogen atmosphere, and the mixture was stirred under reflux. Then, potassium acetate (8.7 g, 88.7 mmol) was added thereto, sufficiently stirred, and then bis(dibenzylideneacetone)palladium(0) (1 g, 1.8 mmol) and tricyclohexylphosphine (1 g, 3.5 mmol) were added. After reacting for 10 hours, the reaction mixture was cooled to room temperature, and the organic layer was separated using chloroform and water, and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 14.3 g of Compound A. (Yield: 70%, MS: [M+H]⁺=346).

### SYNTHESIS EXAMPLE B: SYNTHESIS OF INTERMEDIATE COMPOUND B

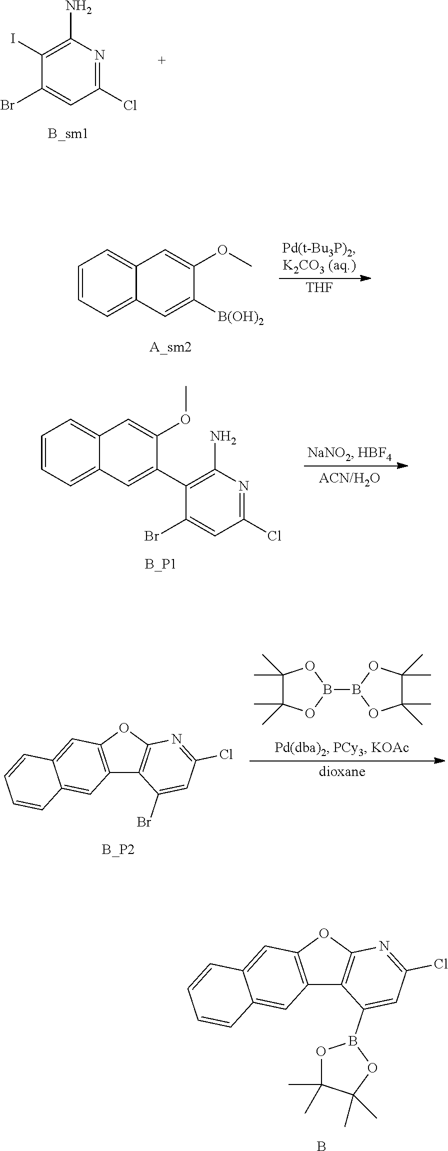

B_sm1 (15 g, 45 mmol) and A_sm2 (9.5 g, 47.2 mmol) were added to 300 mL of THF under a nitrogen atmosphere, and the mixture was stirred and refluxed. Then, potassium carbonate (18.7 g, 135 mmol) was dissolved in 56 ml of water, added thereto, sufficiently stirred, and then bis(tri-tert-butylphosphine)palladium(0) (0.2 g, 0.4 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 10.4 g of B_P1. (Yield: 64%, MS: [M+H]⁺=363).

Next, B_P1 (10 g, 27.5 mmol) and HBF₄ (4.8 g, 55 mmol) were added to 100 mL of ACN under a nitrogen atmosphere, and the mixture was stirred. Then, NaNO₂ (3.8 g, 55 mmol) was dissolved in 20 mL of H₂O and slowly added at 0° C. After reacting for 10 hours, the mixture was heated up to room temperature, and then diluted by adding 200 mL of water. The solution was completely dissolved in chloroform, washed twice with water, the organic layer was separated, treated with anhydrous magnesium sulfate, filtered, and the filtrate was distilled under reduced pressure. The concentrated compound was purified by silica gel column chromatography to give 6.5 g of B_P2. (Yield: 74%, MS: [M+H]⁺=251).

Next, B_P2 (15 g, 45.1 mmol) and bis(pinacolato)diboron (12.6 g, 49.6 mmol) were added to 300 mL of 1,4-dioxane under a nitrogen atmosphere, and the mixture was stirred under reflux. Then, potassium acetate (6.6 g, 67.7 mmol) was added thereto, sufficiently stirred, and then bis(dibenzylideneacetone)palladium(0) (0.8 g, 1.4 mmol) and tricyclohexylphosphine (0.8 g, 2.7 mmol) were added. After reacting for 8 hours, the reaction mixture was cooled to room temperature, and the organic layer was separated using chloroform and water, and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.7 g of Compound B. (Yield: 80%, MS: [M+H]⁺=380).

### SYNTHESIS EXAMPLE C: SYNTHESIS OF INTERMEDIATE COMPOUND C

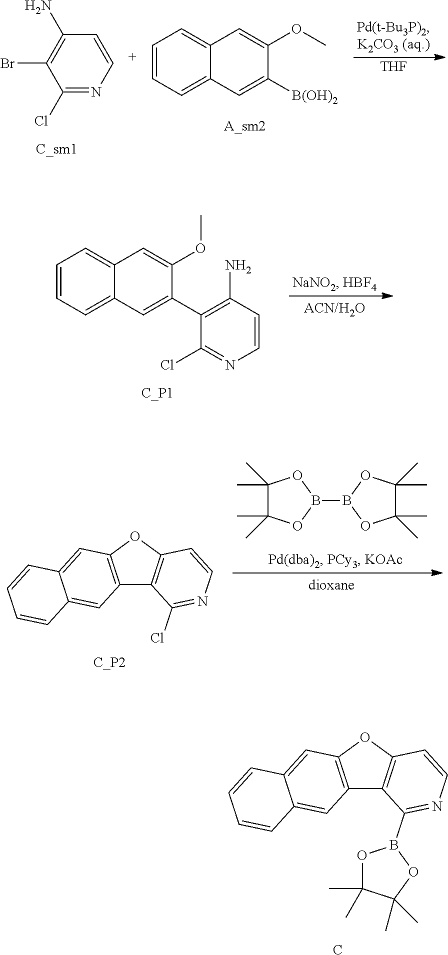

12.2 g of Compound C was prepared in the same manner as in Synthesis Example A, except that C-sm1 was used instead of A-sm1 as a starting material in Synthesis Example A. (Yield: 60%, MS: [M+H]⁺=346).

### SYNTHESIS EXAMPLE D: SYNTHESIS OF INTERMEDIATE COMPOUND D

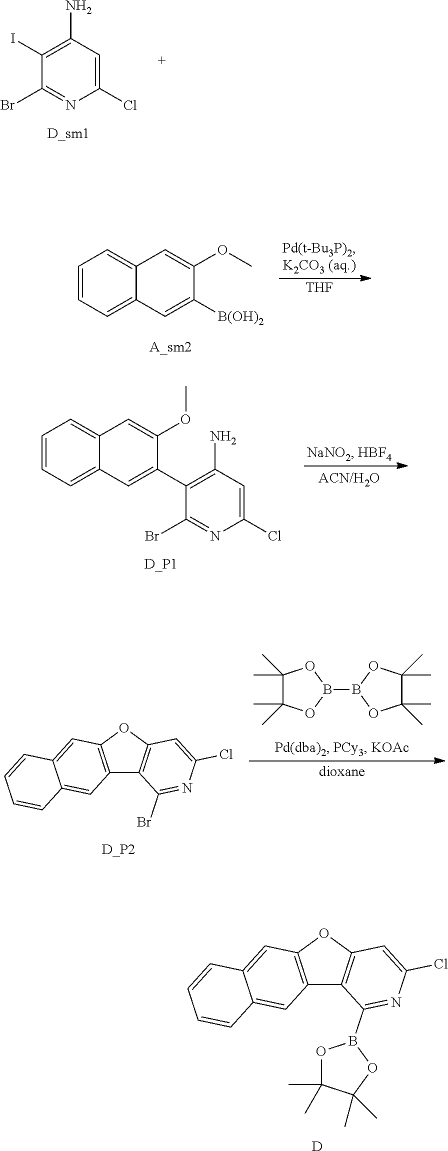

10.9 g of Compound D was prepared in the same manner as in Synthesis Example B, except that D-sm1 was used instead of B-sm1 as a starting material in Synthesis Example B. (Yield: 64%, MS: [M+H]⁺=380).

### SYNTHESIS EXAMPLE E: SYNTHESIS OF INTERMEDIATE COMPOUND E

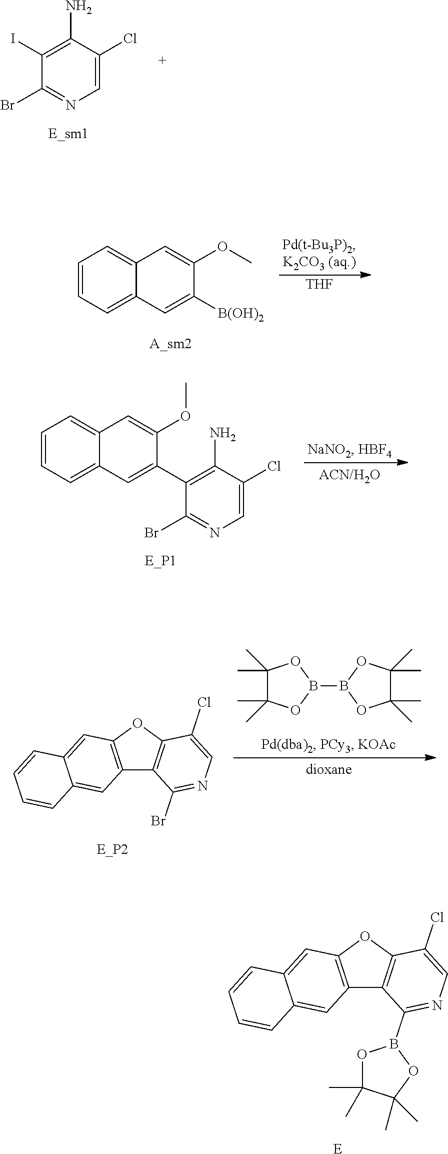

12 g of Compound E was prepared in the same manner as in Synthesis Example B, except that E-sm1 was used instead of B-sm1 as a starting material in Synthesis Example B. (Yield: 70%, MS: [M+H]⁺=380).

### SYNTHESIS EXAMPLE F: SYNTHESIS OF INTERMEDIATE COMPOUND F

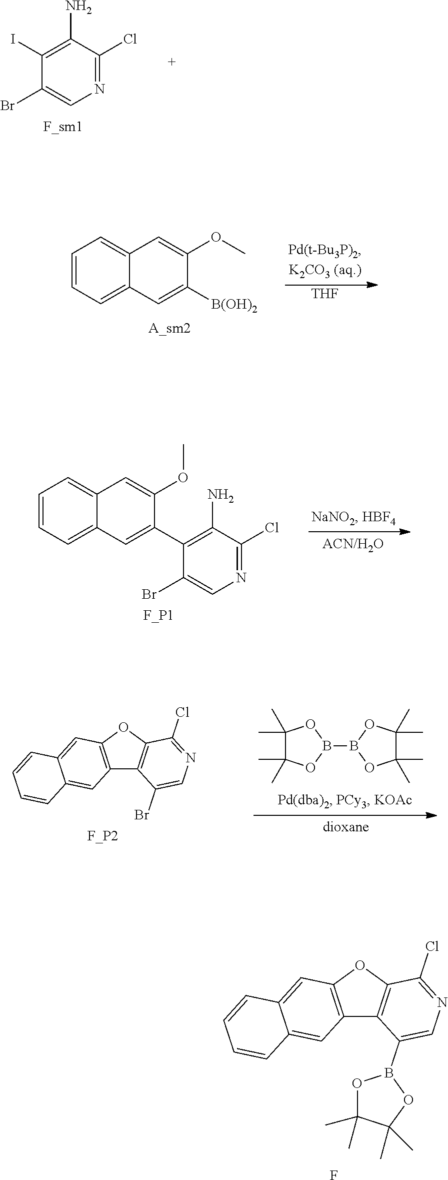

11.5 g of Compound F was prepared in the same manner as in Synthesis Example B, except that F-sm1 was used instead of B-sm1 as a starting material in Synthesis Example B. (Yield: 67%, MS: [M+H]⁺=380).

### SYNTHESIS EXAMPLE G: SYNTHESIS OF INTERMEDIATE COMPOUND G

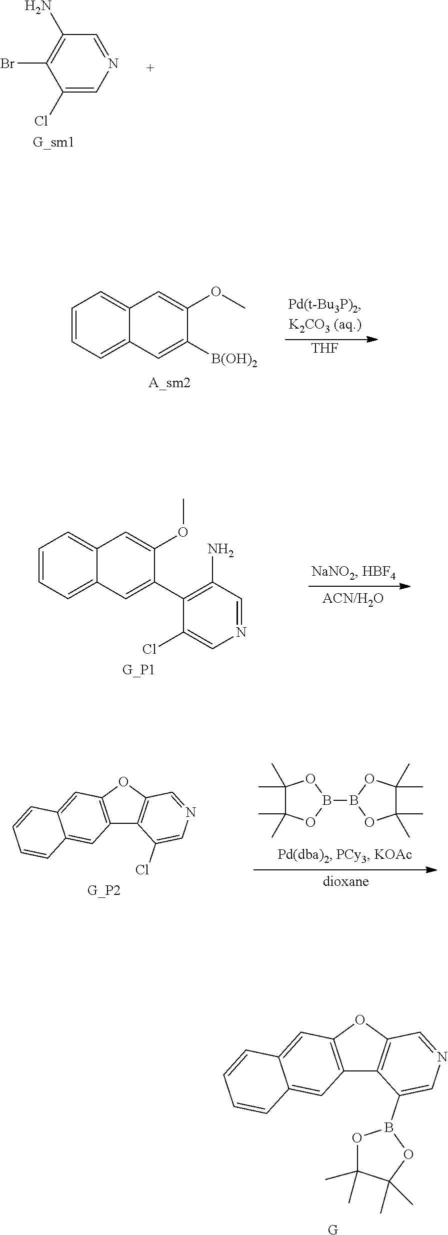

15.5 g of Compound G was prepared in the same manner as in Synthesis Example A, except that G-sm1 was used instead of A-sm1 as a starting material in Synthesis Example A. (Yield: 76%, MS: [M+H]⁺=346).

### SYNTHESIS EXAMPLE H: SYNTHESIS OF INTERMEDIATE COMPOUND H

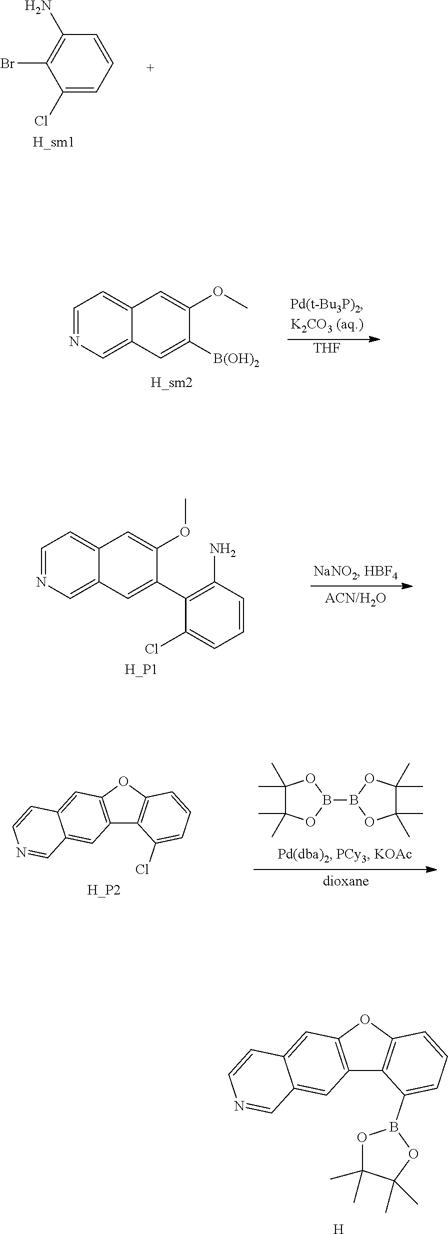

H_sm1(15 g, 72.6 mmol) and H_sm2(19.2 g, 94.4 mmol) were added to 300 mL. of THF under a nitrogen atmosphere, and the mixture was stirred and refluxed. Then, potassium carbonate (30.1 g, 217.9 mmol) was dissolved in 90 ml of water, added thereto, sufficiently stirred, and then bis(tri-tert-butylphosphine)palladium(0) (0.4 g, 0.7 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.4 g of H_P1. (Yield: 73%, MS: [M+H]⁺=254).

Next, H_P1 (10 g, 35.1 mmol) and HBF₄ (6.2 g, 70.2 mmol) were added to 100 mL of ACN under a nitrogen atmosphere, and the mixture was stirred. Then, NaNO₂ (4.8 g, 70.2 mmol) was dissolved in 20 mL of H₂O and slowly added at 0° C. After reacting for 10 hours, the mixture was heated up to room temperature, and then diluted by adding 200 mL of water. The solution was completely dissolved in chloroform, washed twice with water, the organic layer was separated, treated with anhydrous magnesium sulfate, filtered, and the filtrate was distilled under reduced pressure. The concentrated compound was purified by silica gel column chromatography to give 6.3 g of H_P2. (Yield: 72%, MS: [M+H]⁺=251).

Next, H_P2(15 g, 59.1 mmol) and bis(pinacolato)diboron(16.5 g, 65 mmol) were added to 300 mL of 1,4-dioxane under a nitrogen atmosphere, and the mixture was stirred under reflux. Then, potassium acetate (8.7 g, 88.7 mmol) was added thereto, sufficiently stirred, and then bis(dibenzylideneacetone)palladium(0) (1 g, 1.8 mmol) and tricyclohexylphosphine (1 g, 3.5 mmol) were added. After reacting for 10 hours, the reaction mixture was cooled to room temperature, and the organic layer was separated using chloroform and water, and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 15.5 g of Compound H. (Yield: 76%, MS: [M+H]⁺=346).

### SYNTHESIS EXAMPLE I: SYNTHESIS OF INTERMEDIATE COMPOUND I

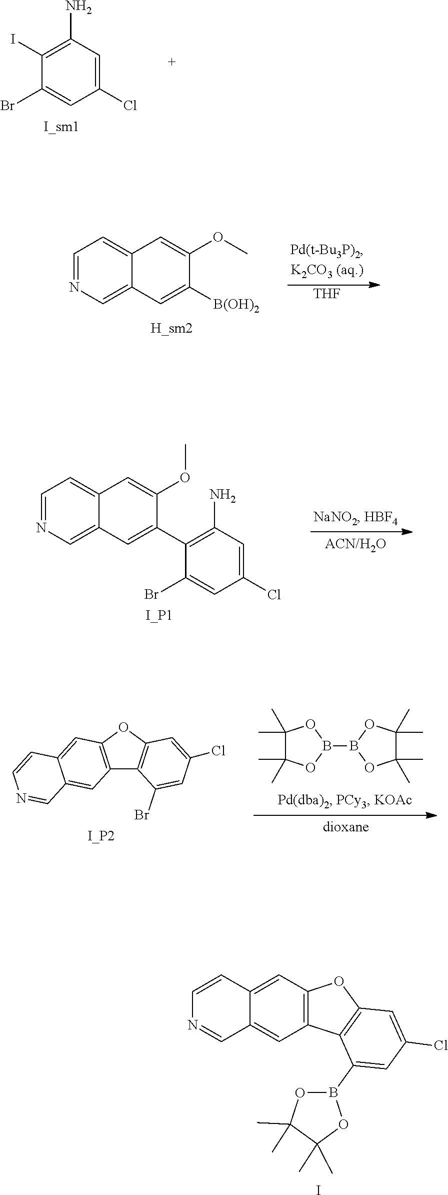

I_sm1 (15 g, 45.1 mmol) and H_sm2 (9.6 g, 47.4 mmol) were added to 300 mL of THF under a nitrogen atmosphere, and the mixture was stirred and refluxed. Then, potassium carbonate (18.7 g, 135.4 mmol) was dissolved in 56 ml of water, added thereto, sufficiently stirred, and then bis(tri-tert-butylphosphine)palladium(0) (0.2 g, 0.5 mmol) was added. After reacting for 11 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 10.9 g of I_P1. (Yield: 67%, MS: [M+H]⁺=363).

Next, I_P1 (10 g, 27.5 mmol) and HBF4 (4.8 g, 55 mmol) were added to 100 mL of ACN under a nitrogen atmosphere, and the mixture was stirred. Then, NaNO₂ (3.8 g, 55 mmol) was dissolved in 20 mL of H₂O and slowly added at 0° C. After reacting for 10 hours, the mixture was heated to room temperature, and then diluted by adding 200 mL of water. The solution was completely dissolved in chloroform, washed twice with water, the organic layer was separated, treated with anhydrous magnesium sulfate, filtered, and the filtrate was distilled under reduced pressure. The concentrated compound was purified by silica gel column chromatography to give 6.5 g of I_P2. (Yield: 74%, MS: [M+H]⁺=251).

Next, I_P2 (15 g, 45.1 mmol) and bis(pinacolato)diboron (12.6 g, 49.6 mmol) were added to 300 mL of 1,4-dioxane under a nitrogen atmosphere, and the mixture was stirred under reflux. Then, potassium acetate (6.6 g, 67.7 mmol) was added thereto, sufficiently stirred, and then bis(dibenzylideneacetone)palladium(0) (0.8 g, 1.4 mmol) and tricyclohexylphosphine (0.8 g, 2.7 mmol) were added. After reacting for 8 hours, the reaction mixture was cooled to room temperature, and the organic layer was separated using chloroform and water, and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.2 g of Compound I. (Yield: 77%, MS: [M+H]⁺=380).

### SYNTHESIS EXAMPLE J: SYNTHESIS OF INTERMEDIATE COMPOUND J

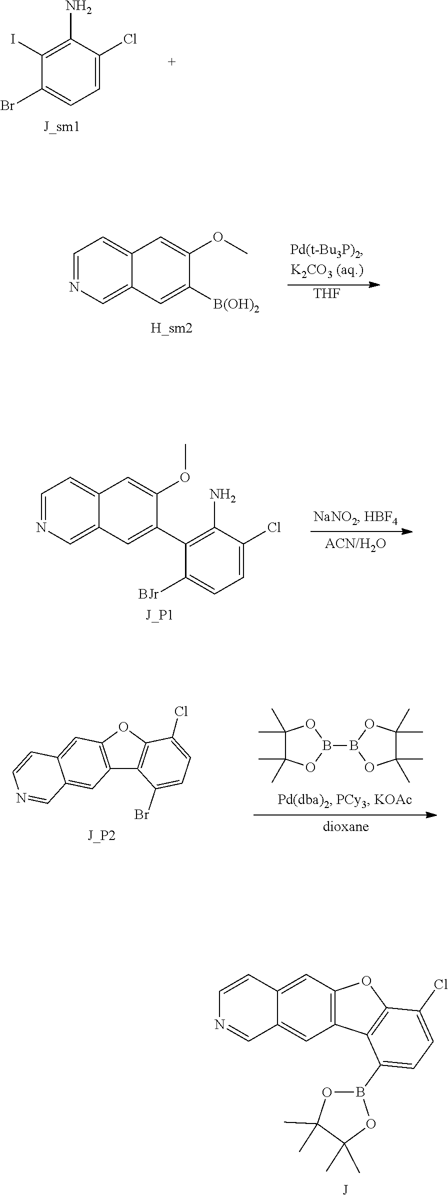

12.5 g of Compound J was prepared in the same manner as in Synthesis Example I, except that J-sm1 was used instead of I-sm1 as a starting material in Synthesis Example I. (Yield: 73%, MS: [M+H]⁺=380).

### SYNTHESIS EXAMPLE K: SYNTHESIS OF INTERMEDIATE COMPOUND K

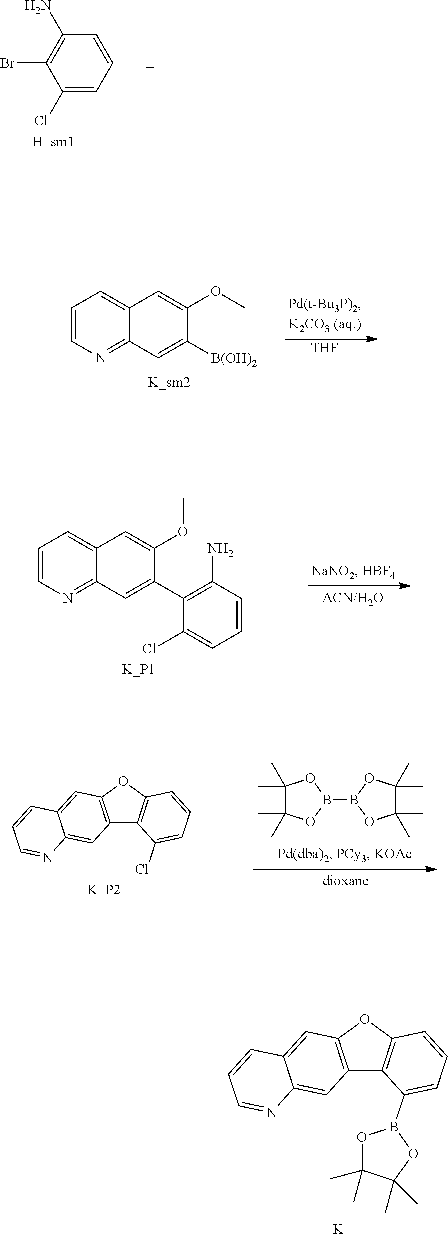

13.7 g of Compound K was prepared in the same manner as in Synthesis Example H, except that K-sm2 was used instead of H-sm2 as a starting material in Synthesis Example H. (Yield: 67%, MS: [M+H]⁺=346).

### SYNTHESIS EXAMPLE L: SYNTHESIS OF INTERMEDIATE COMPOUND L

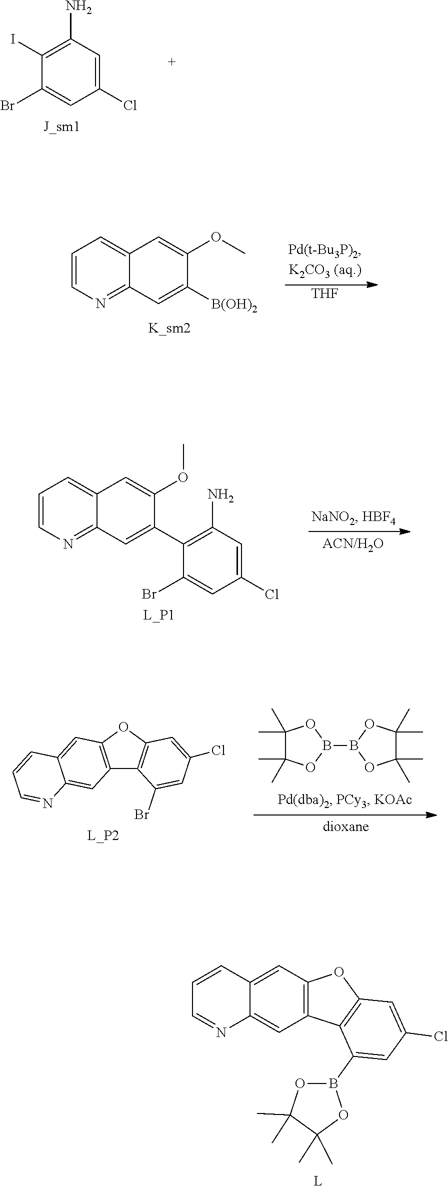

12.7 g of Compound L was prepared in the same manner as in Synthesis Example I, except that J-sm1 was used instead of I-sm1 and K-sm2 was used instead of H-sm2 as a starting material in Synthesis Example I. (Yield: 74%, MS: [M+H]⁺=380).

### SYNTHESIS EXAMPLE M: SYNTHESIS OF INTERMEDIATE COMPOUND M

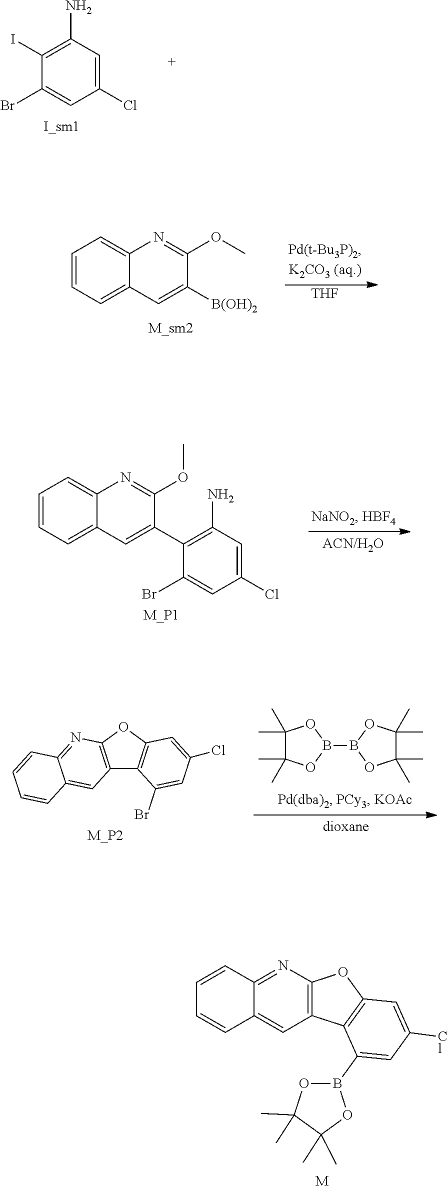

12.5 g of Compound M was prepared in the same manner as in Synthesis Example I, except that M-sm2 was used instead of H-sm2 as a starting material in Synthesis Example I. (Yield: 73%, MS: [M+H]⁺=380).

### SYNTHESIS EXAMPLE N: SYNTHESIS OF INTERMEDIATE COMPOUND N

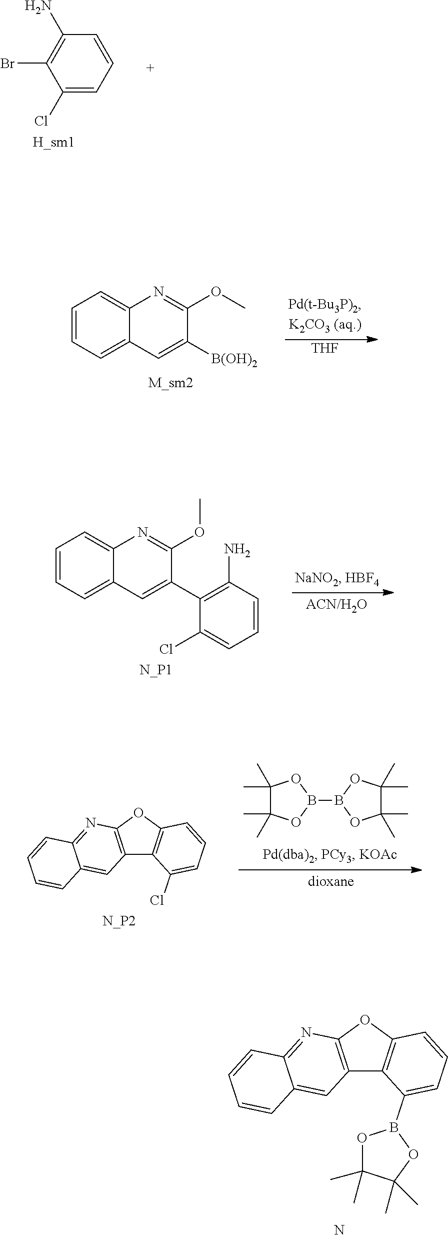

14.7 g of Compound N was prepared in the same manner as in Synthesis Example H, except that M-sm2 was used instead of H-sm2 as a starting material in Synthesis Example H. (Yield: 72%, MS: [M+H]⁺=346).

### SYNTHESIS EXAMPLE O: SYNTHESIS OF INTERMEDIATE COMPOUND O

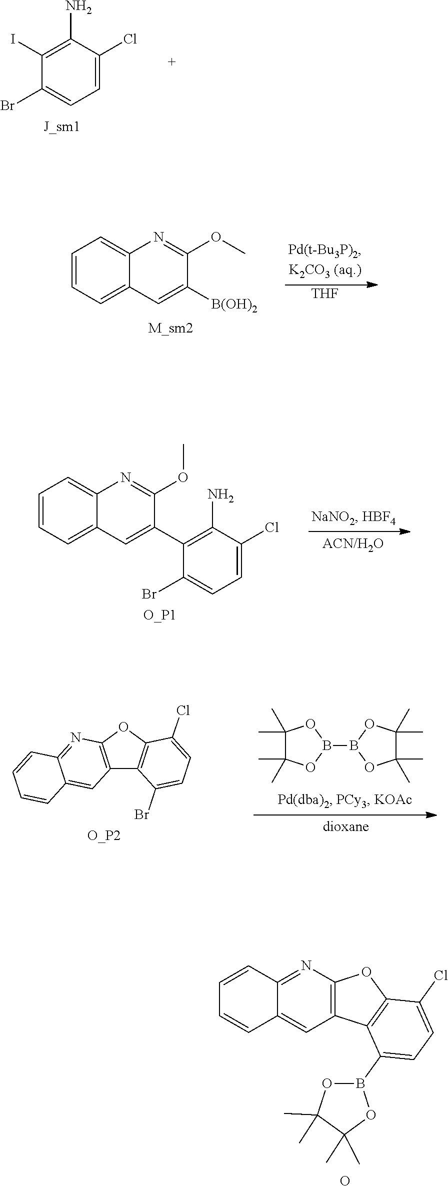

13.2 g of Compound O was prepared in the same manner as in Synthesis Example I, except that J-sm1 was used instead of I-sm1 and M-sm2 was used instead of H-sm2 as a starting material in Synthesis Example I. (Yield: 77%, MS: [M+H]⁺=380).

### SYNTHESIS EXAMPLE P: SYNTHESIS OF INTERMEDIATE COMPOUND P

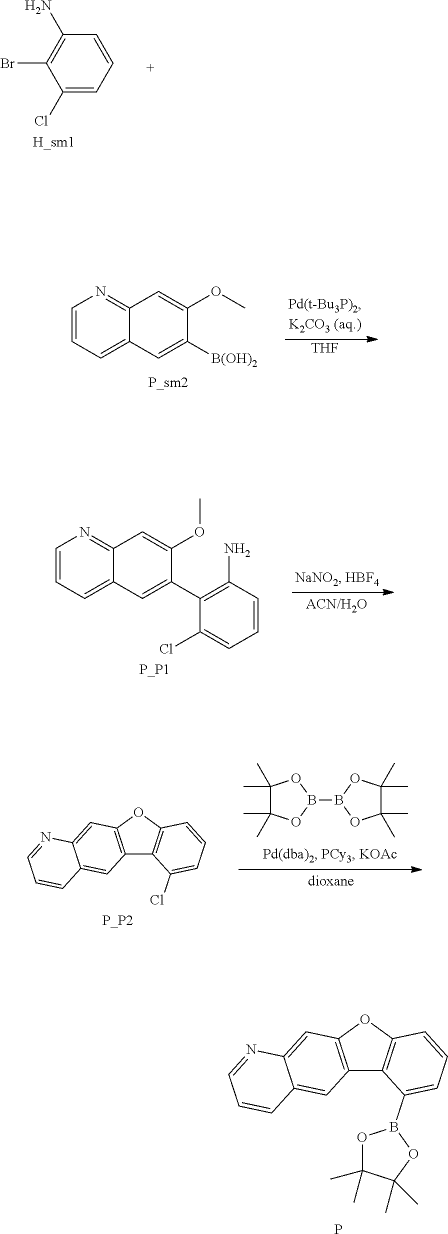

14.7 g of Compound P was prepared in the same manner as in Synthesis Example H, except that P-sm2 was used instead of H-sm2 as a starting material in Synthesis Example H. (Yield: 72%, MS: [M+H]⁺=346).

### SYNTHESIS EXAMPLE Q: SYNTHESIS OF INTERMEDIATE COMPOUND Q

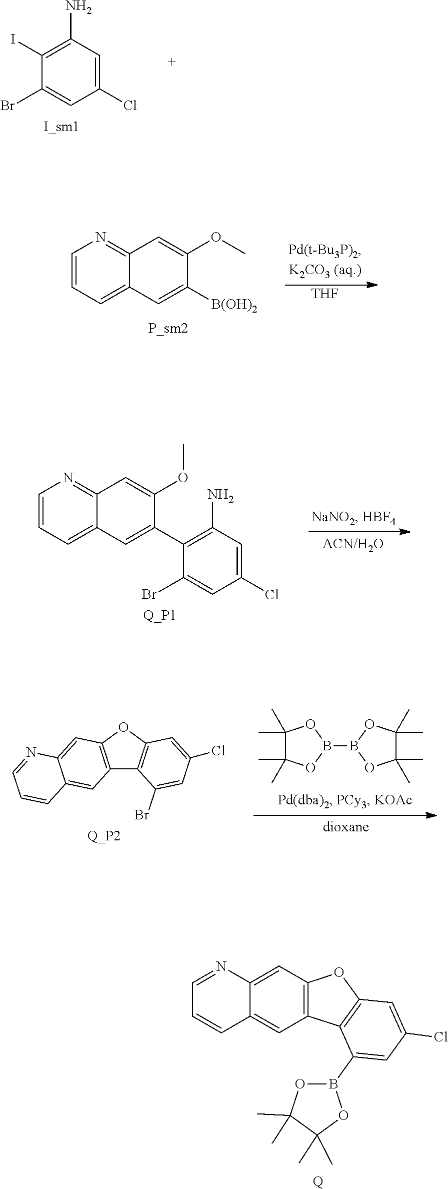

12.8 g of Compound Q was prepared in the same manner as in Synthesis Example I, except that P-sm2 was used instead of H-sm2 as a starting material in Synthesis Example I. (Yield: 75%, MS: [M+H]⁺=380).

### SYNTHESIS EXAMPLE R: SYNTHESIS OF INTERMEDIATE COMPOUND R

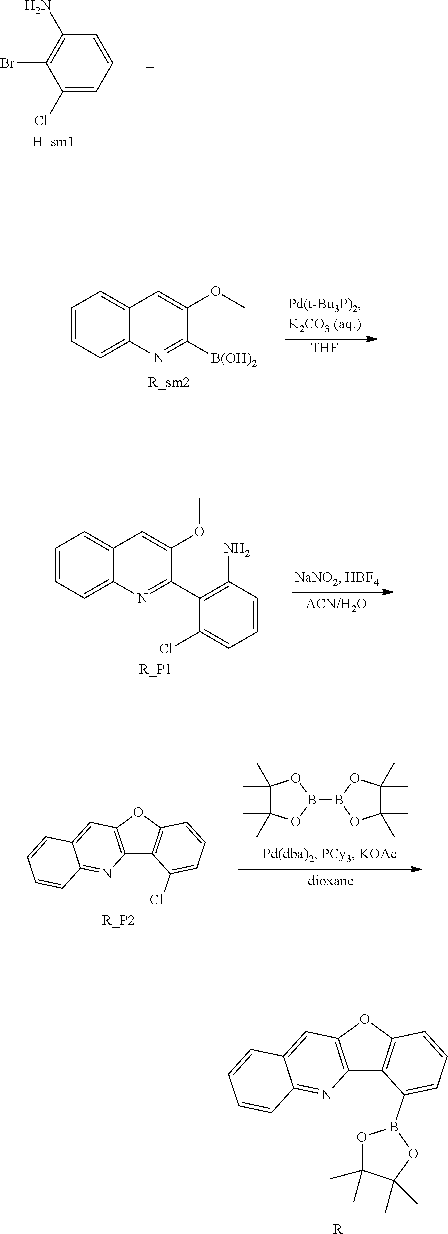

13.3 g of Compound R was prepared in the same manner as in Synthesis Example H, except that R-sm2 was used instead of H-sm2 as a starting material in Synthesis Example H. (Yield: 65%, MS: [M+H]⁺=346).

### SYNTHESIS EXAMPLE S: SYNTHESIS OF INTERMEDIATE COMPOUND S

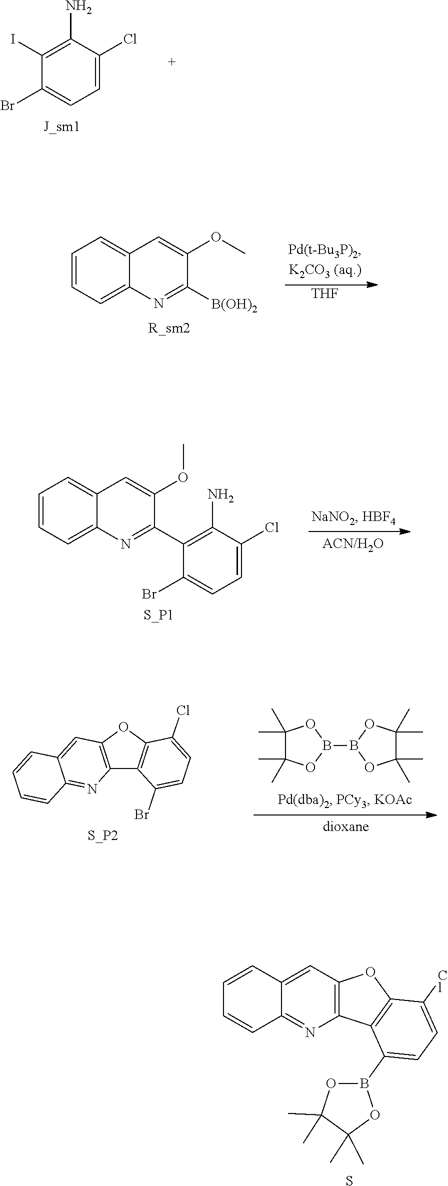

11.3 g of Compound S was prepared in the same manner as in Synthesis Example I, except that J-sm1 was used instead of I-sm1 and R-sm2 was used instead of H-sm2 as a starting material in Synthesis Example I. (Yield: 66%, MS: [M+H]⁺=380).

### SYNTHESIS EXAMPLE T: SYNTHESIS OF INTERMEDIATE COMPOUND T

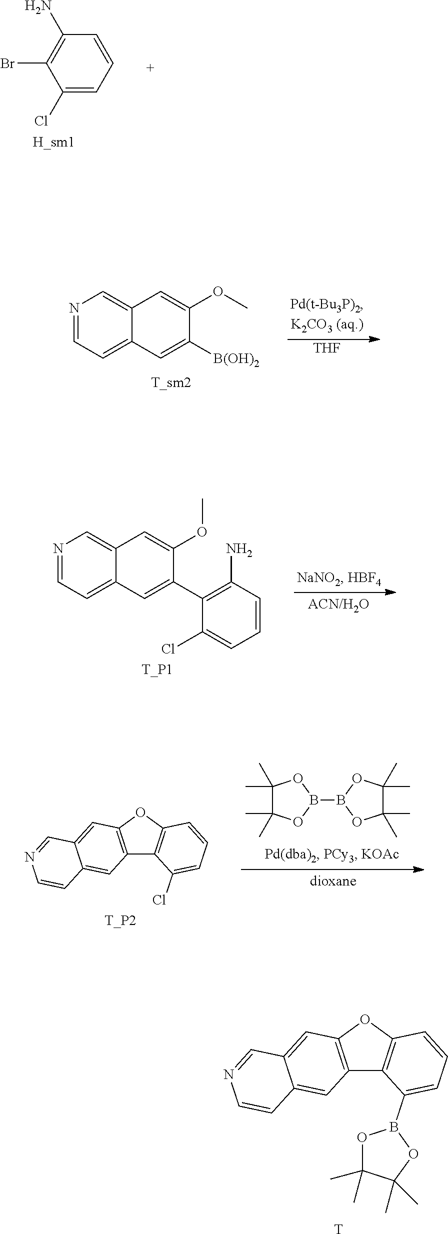

13.9 g of Compound T was prepared in the same manner as in Synthesis Example H, except that T-sm2 was used instead of H-sm2 as a starting material in Synthesis Example H. (Yield: 68%, MS: [M+H]⁺=346).

### SYNTHESIS EXAMPLE U: SYNTHESIS OF INTERMEDIATE COMPOUND U

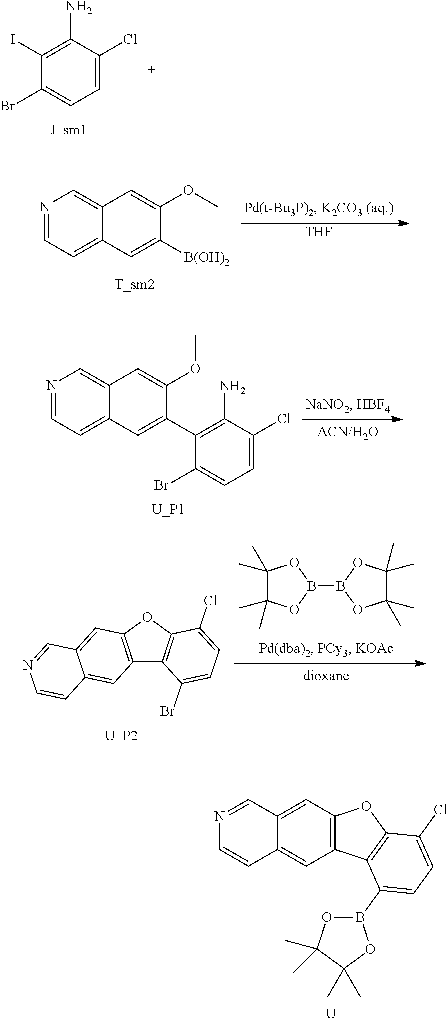

13 g of Compound U was prepared in the same manner as in Synthesis Example I, except that J-sm1 was used instead of I-sm1, and T-sm2 was used instead of H-sm2 as a starting material in Synthesis Example I. (Yield: 76%, MS: [M+H]⁺=380).

### SYNTHESIS EXAMPLE V: SYNTHESIS OF INTERMEDIATE COMPOUND V

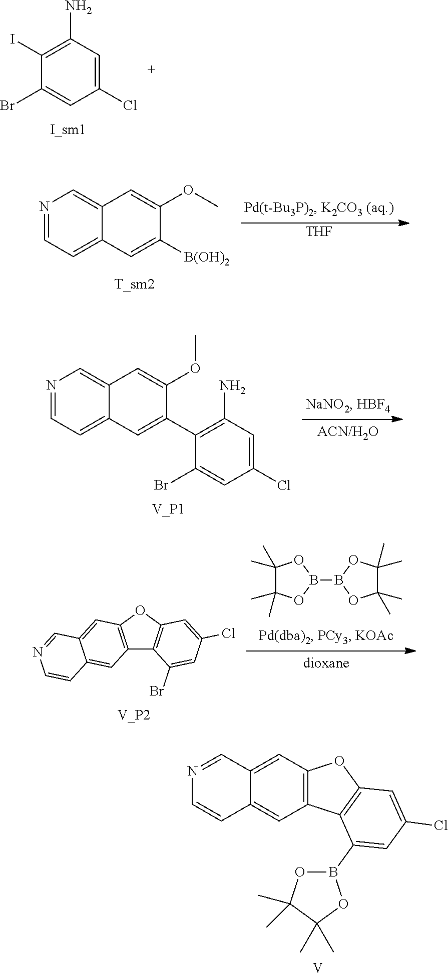

12.3 g of Compound V was prepared in the same manner as in Synthesis Example I, except that T-sm2 was used instead of H-sm2 as a starting material in Synthesis Example I. (Yield: 72%, MS: [M+H]⁺=380).

### SYNTHESIS EXAMPLE 1: PREPARATION OF COMPOUND 1

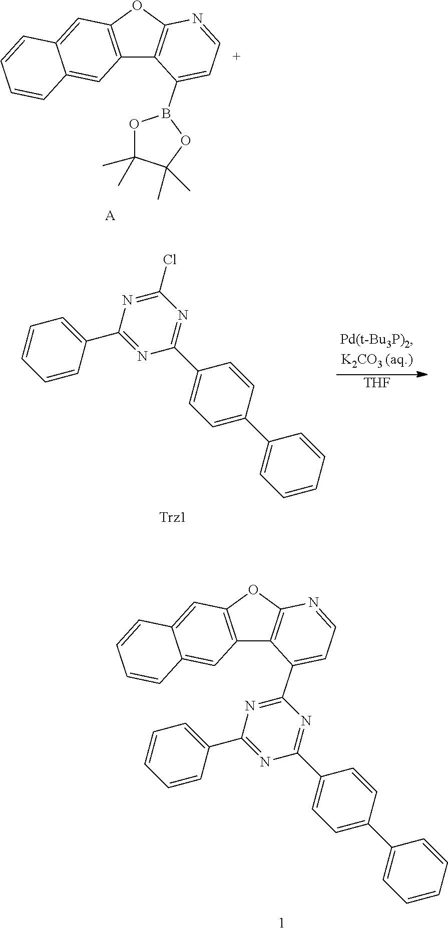

Compound A (15 g, 46.1 mmol) and Trz1 (16.7 g, 48.4 mmol) were added to 300 mL. of THF under a nitrogen atmosphere, and the mixture was stirred and refluxed. Then, potassium carbonate (12.7 g, 92.2 mmol) was dissolved in 38 ml of water, added thereto, sufficiently stirred, and then bis(tri-tert-butylphosphine)palladium(0) (0.2 g, 0.5 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 17.2 g of Compound 1. (Yield: 71%, MS: [M+H]⁺=527).

### SYNTHESIS EXAMPLE 2: PREPARATION OF COMPOUND 2

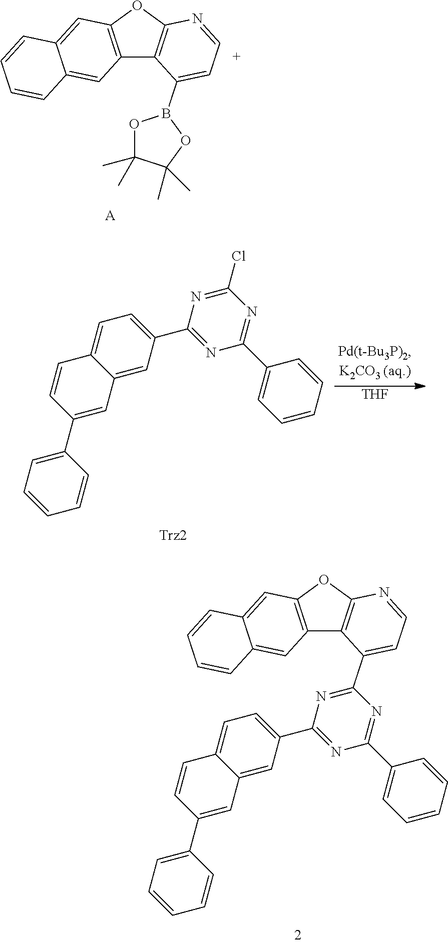

Compound A (15 g, 46.1 mmol) and Trz2 (19.1 g, 48.4 mmol) were added to 300 mL of THF under a nitrogen atmosphere, and the mixture was stirred and refluxed. Then, potassium carbonate (12.7 g, 92.2 mmol) was dissolved in 38 ml of water, added thereto, sufficiently stirred, and then bis(tri-tert-butyl-phosohine)palladium(0) (0.2 g, 0.5 mmol) was added. After reacting for 11 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 18.1 g of Compound 2. (Yield: 68%, MS: [M+H]⁺=577).

### SYNTHESIS EXAMPLE 3: PREPARATION OF COMPOUND 3

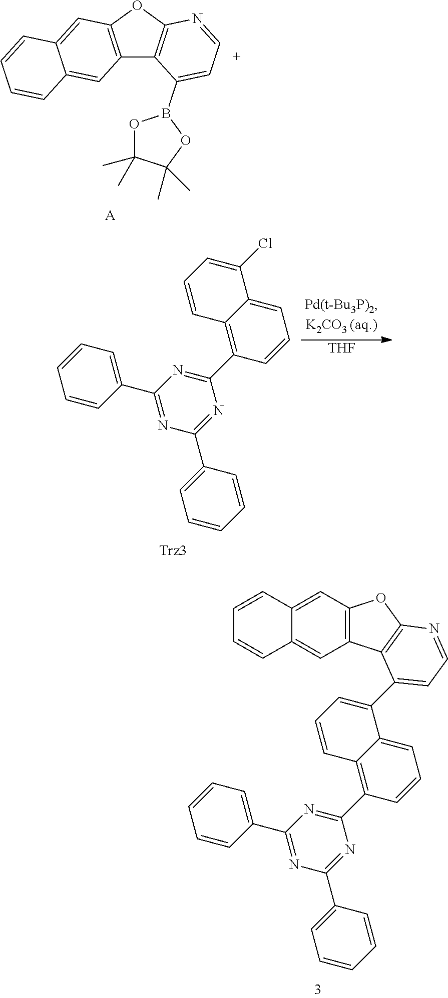

Compound A (15 g, 46.1 mmol) and Trz3 (19.1 g, 48.4 mmol) were added to 300 mL of THF under a nitrogen atmosphere, and the mixture was stirred and refluxed. Then, potassium carbonate (12.7 g, 92.2 mmol) was dissolved in 38 ml of water, added thereto, sufficiently stirred, and then bis(tri-tert-butylphosphine)palladium(0) (0.2 g, 0.5 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 20.7 g of Compound 3. (Yield: 78%, MS: [M+H]⁺=577).

### SYNTHESIS EXAMPLE 4: PREPARATION OF COMPOUND 4

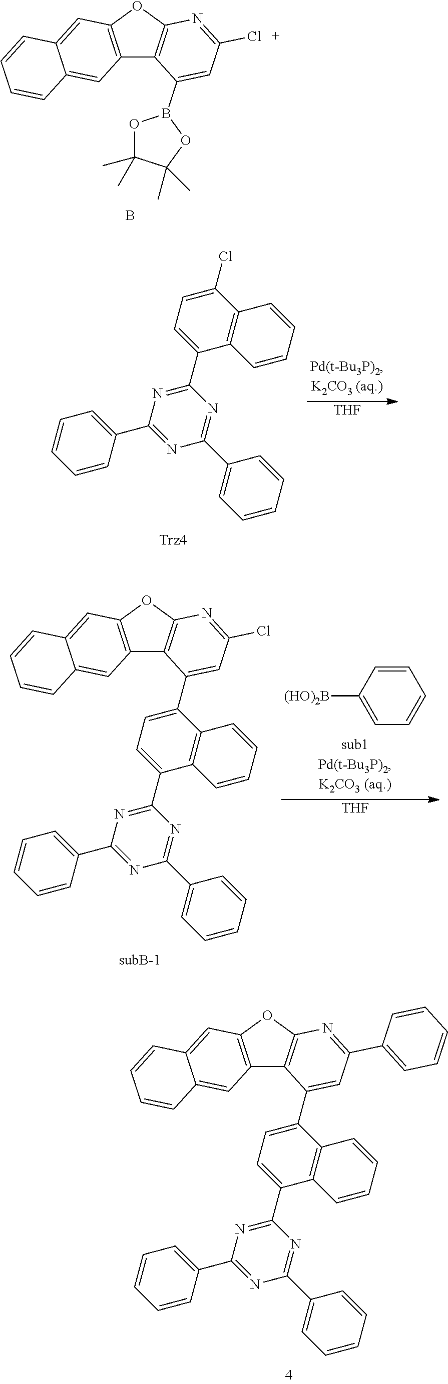

Compound B (15 g, 39.5 mmol) and Trz4 (15.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 17.6 g of subB-1. (Yield: 73%, MS: [M+H]⁺=611).

Next, subB-1 (15 g, 24.5 mmol) and sub1 (3.1 g, 25.8 mmol) were added to 300 mL of THF under a nitrogen atmosphere, and the mixture was stirred and refluxed. Then, potassium carbonate (6.8 g, 49.1 mmol) was dissolved in 20 ml of water, added thereto, sufficiently stirred, and then bis(tri-tert-butylphosphine)-palladium(0) (0.1 g, 0.2 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 11.2 g of Compound 4. (Yield: 70%, MS: [M+H]⁺=653).

### SYNTHESIS EXAMPLE 5: PREPARATION OF COMPOUND 5

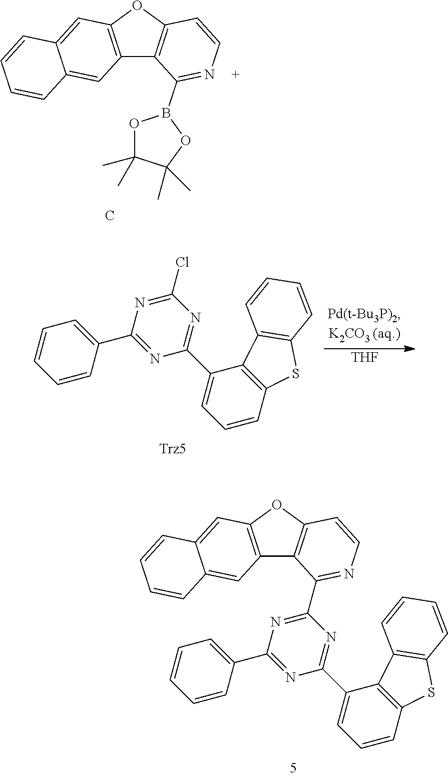

Compound C (15 g, 46.1 mmol) and Trz5 (18.1 g, 48.4 mmol) were added to 300 mL of THF under a nitrogen atmosphere, and the mixture was stirred and refluxed. Then, potassium carbonate (12.7 g, 92.2 mmol) was dissolved in 38 ml of water, added thereto, sufficiently stirred, and then bis(tri-tert-butylphosphine)-palladium(0) (0.2 g, 0.5 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 19.5 g of Compound 5. (Yield: 76%, MS: [M+H]⁺=557).

### SYNTHESIS EXAMPLE 6: PREPARATION OF COMPOUND 6

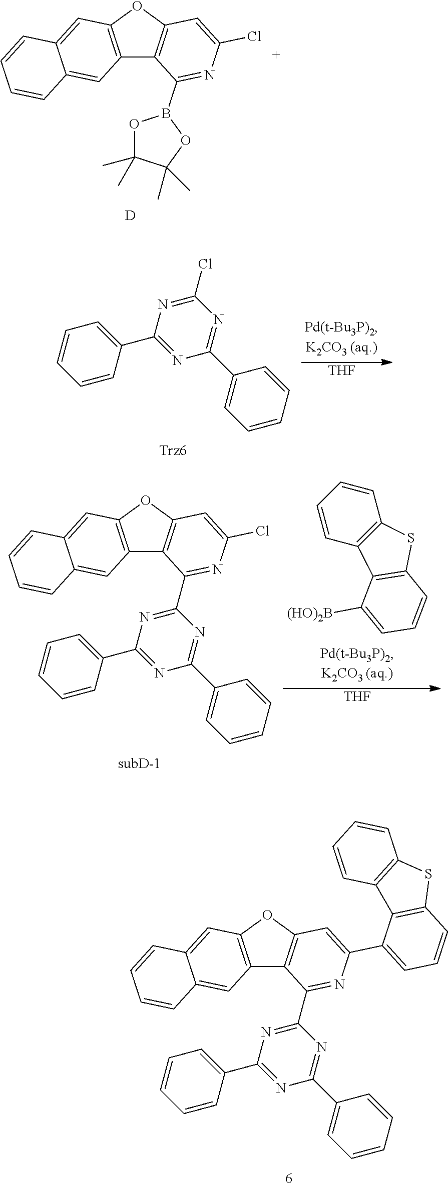

Compound D (15 g, 39.5 mmol) and Trz6 (10.4 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 12.5 g of subD-1. (Yield: 65%, MS: [M+H]⁺=487).

Next, subD-1 (15 g, 30.8 mmol) and sub2 (7.4 g, 32.3 mmol) were added to 300 mL of THF under a nitrogen atmosphere, and the mixture was stirred and refluxed. Then, potassium carbonate (8.5 g, 61.6 mmol) was dissolved in 26 mL of water, added thereto, sufficiently stirred, and then bis(tri-tert-butylphosphine)-palladium(0) (0.2 g, 0.3 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.3 g of Compound 6. (Yield: 68%, MS: [M+H]⁺=635).

### SYNTHESIS EXAMPLE 7: PREPARATION OF COMPOUND 7

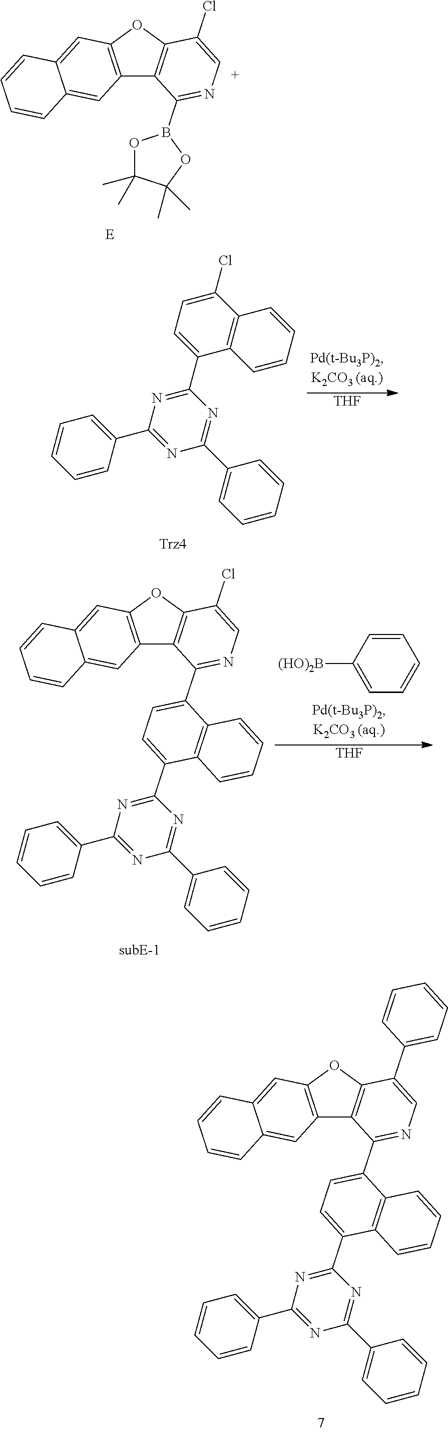

Compound E (15 g, 39.5 mmol) and Trz4 (15.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 19.3 g of subE-1. (Yield: 80%, MS: [M+H]⁺=611).

Next, subE-1 (15 g, 24.5 mmol) and sub1 (3.1 g, 25.8 mmol) were added to 300 mL of THF under a nitrogen atmosphere, and the mixture was stirred and refluxed. Then, potassium carbonate (6.8 g, 49.1 mmol) was dissolved in 20 ml of water, added thereto, sufficiently stirred, and then bis(tri-tert-butylphosphine)-palladium(0) (0.1 g, 0.2 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 11.2 g of Compound 7. (Yield: 70%, MS: [M+H]⁺=653).

### SYNTHESIS EXAMPLE 8: PREPARATION OF COMPOUND 8

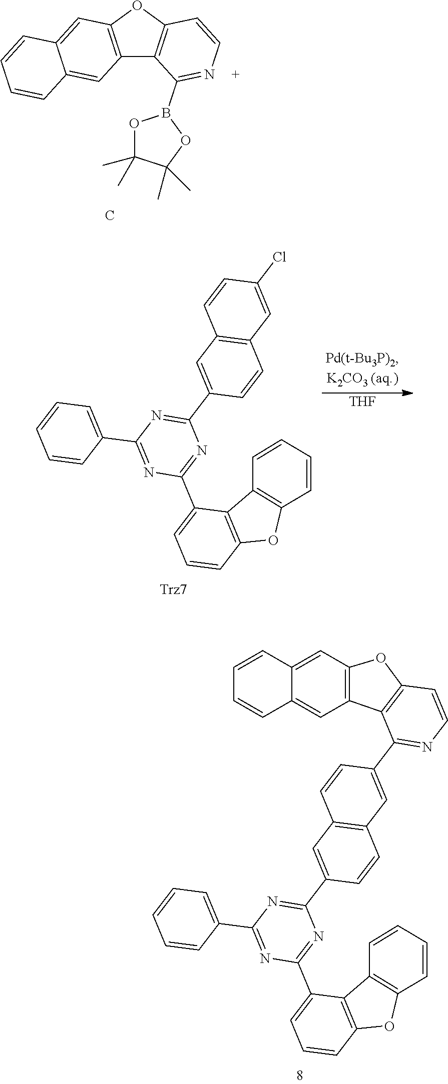

Compound C (15 g, 43.5 mmol) and Trz7 (22.1 g, 45.6 mmol) were added to 300 mL. of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 19.1 g of Compound 8. (Yield: 66%, MS: [M+H]⁺=667).

### SYNTHESIS EXAMPLE 9: PREPARATION OF COMPOUND 9

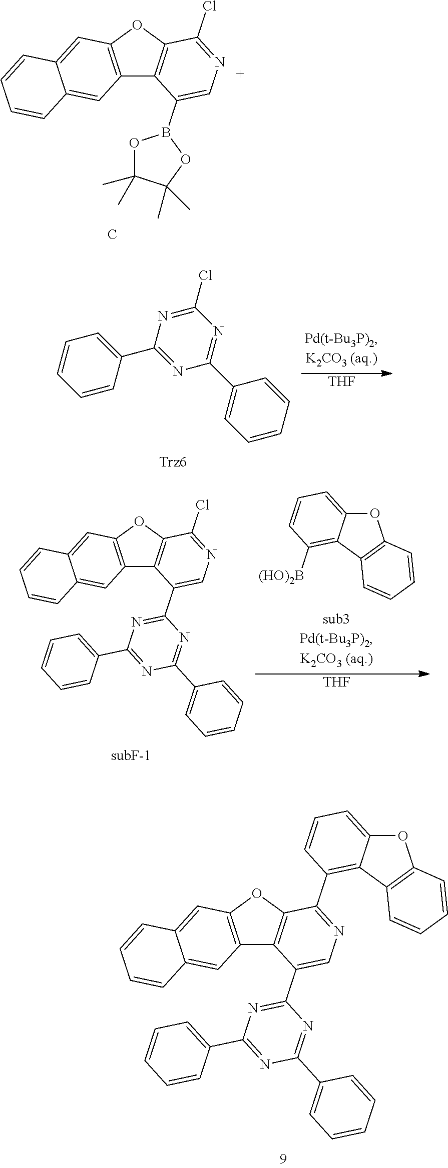

Compound F (15 g, 39.5 mmol) and Trz6 (10.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 14.7 g of subF-1. (Yield: 77%, MS: [M+H]⁺=485).

Next, subF-1 (15 g, 30.9 mmol) and sub3 (6.9 g, 32.5 mmol) were added to 300 mL of THF under a nitrogen atmosphere, and the mixture was stirred and refluxed. Then, potassium carbonate (8.6 g, 61.9 mmol) was dissolved in 26 ml of water, added thereto, sufficiently stirred, and then bis(tri-tert-butylphosphine)-palladium(0) (0.2 g, 0.3 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 11.8 g of Compound 9. (Yield: 62%, MS: [M+H]⁺=617).

### SYNTHESIS EXAMPLE 10: PREPARATION OF COMPOUND 10

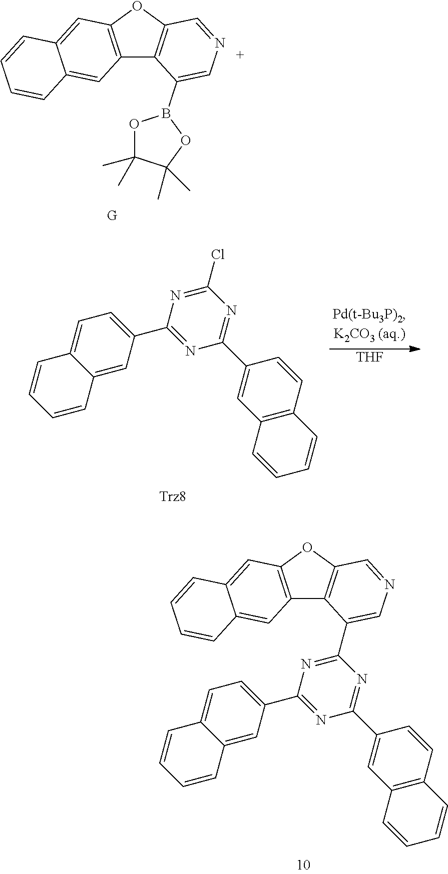

Compound G (15 g, 43.5 mmol) and Trz8 (16.8 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 18.9 g of Compound 10. (Yield: 79%, MS: [M+H]⁺=551).

### SYNTHESIS EXAMPLE 11: PREPARATION OF COMPOUND 11

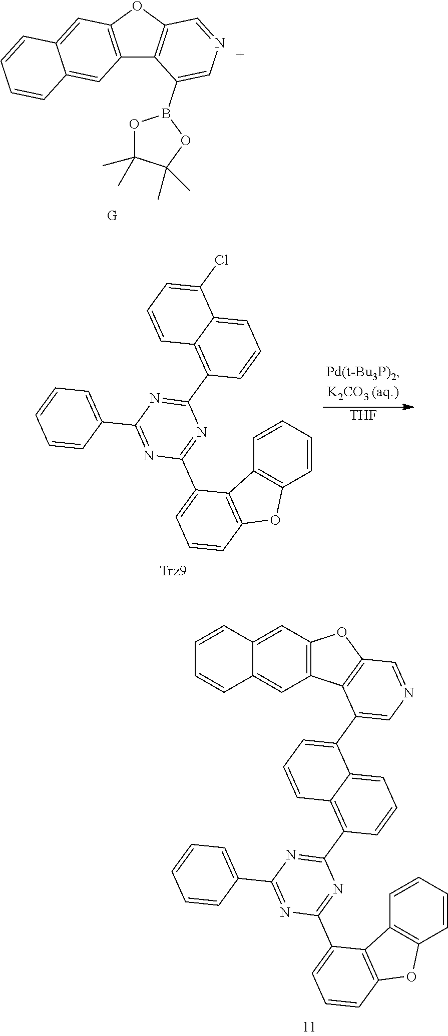

Compound G (15 g, 43.5 mmol) and Trz9 (22.1 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 17.4 g of Compound 11. (Yield: 60%, MS: [M+H]⁺=667).

### SYNTHESIS EXAMPLE 12: PREPARATION OF COMPOUND 12

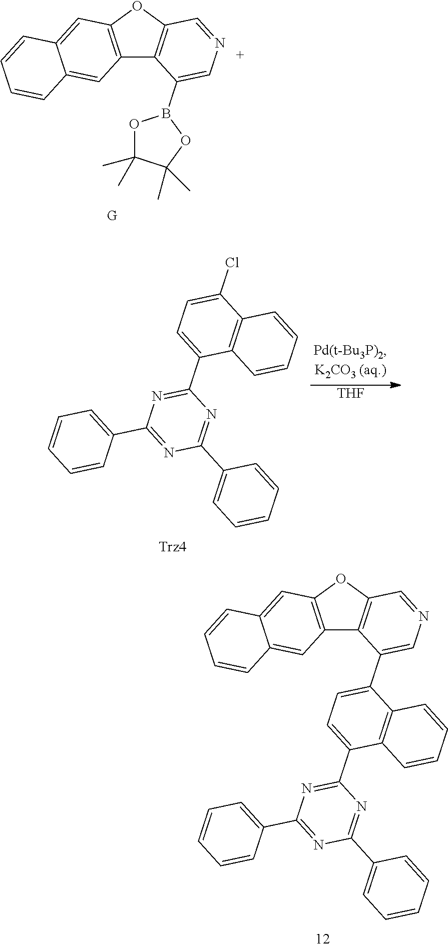

Compound G (15 g, 43.5 mmol) and Trz4 (18 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 16.5 g of Compound 12. (Yield: 66%, MS: [M+H]⁺=577).

### SYNTHESIS EXAMPLE 13: PREPARATION OF COMPOUND 13

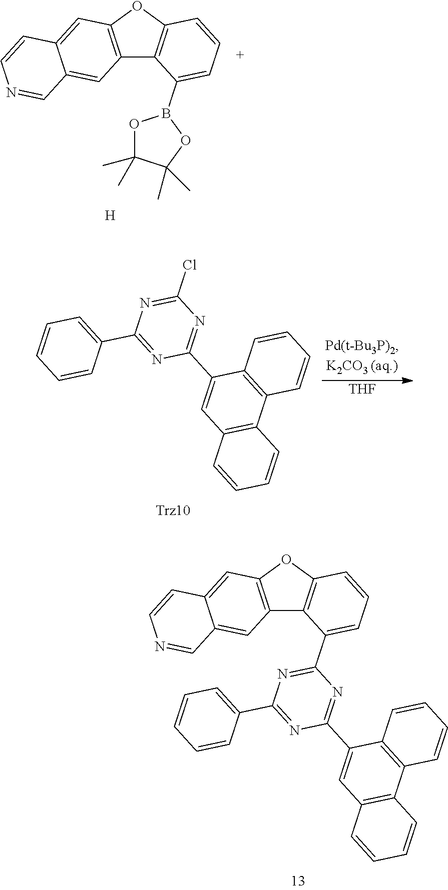

Compound H (15 g, 43.5 mmol) and Trz10 (16.8 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 17.7 g of Compound 13. (Yield: 74%, MS: [M+H]⁺=551).

### SYNTHESIS EXAMPLE 14: PREPARATION OF COMPOUND 14

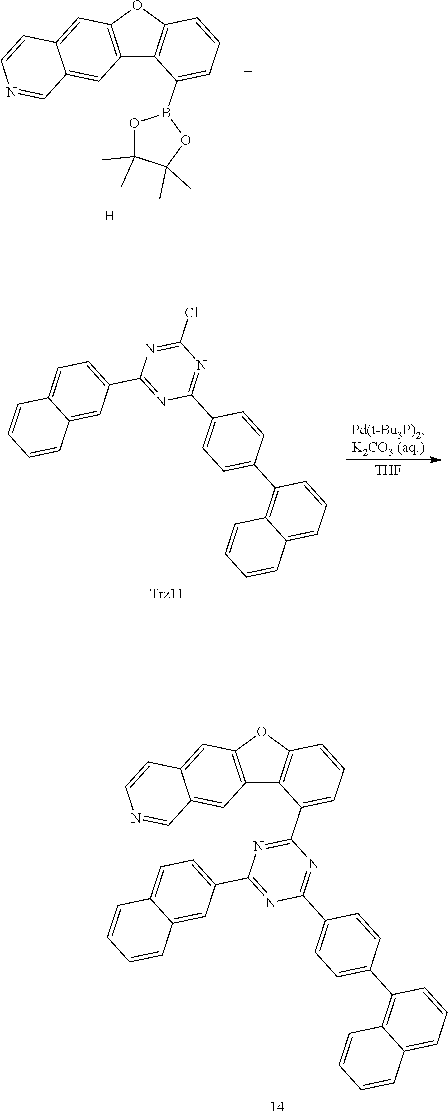

Compound H (15 g, 43.5 mmol) and Trz11 (20.3 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 21.5 g of Compound 14. (Yield: 79%, MS: [M+H]⁺=627).

### SYNTHESIS EXAMPLE 15: PREPARATION OF COMPOUND 15

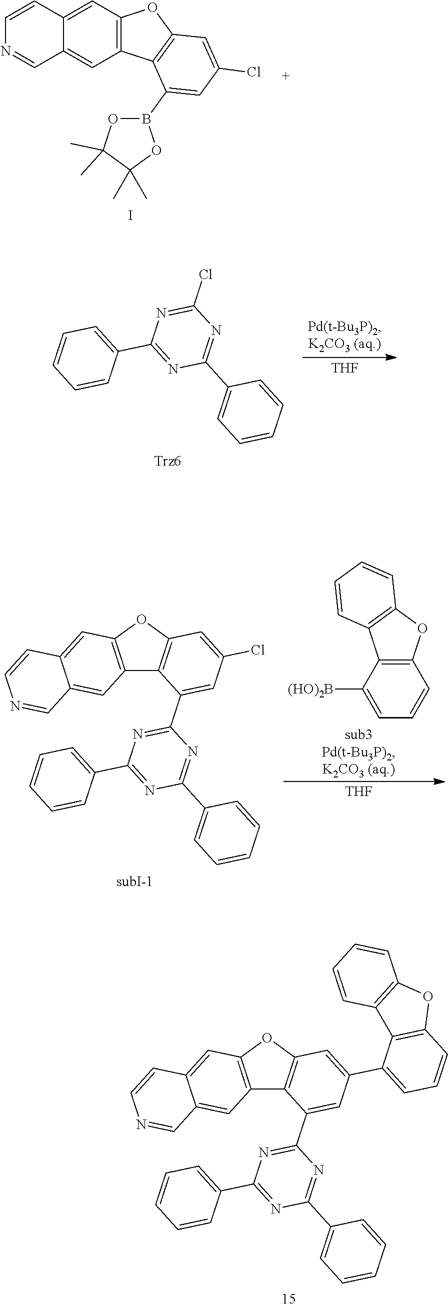

Compound 1 (15 g, 39.5 mmol) and Trz6 (10.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 12.6 g of subl-1. (Yield: 66%, MS: [M+H]⁺=485).

Next, subI-1 (15 g, 30.9 mmol) and sub3 (6.9 g, 32.5 mmol) were added to 300 mL of THF under a nitrogen atmosphere, and the mixture was stirred and refluxed. Then, potassium carbonate (8.6 g, 61.9 mmol) was dissolved in 26 ml of water, added thereto, sufficiently stirred, and then bis(tri-tert-butylphosphine)-palladium(0) (0.2 g, 0.3 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.2 g of Compound 15. (Yield: 69%, MS: [M+H]⁺=617).

### SYNTHESIS EXAMPLE 16: PREPARATION OF COMPOUND 16

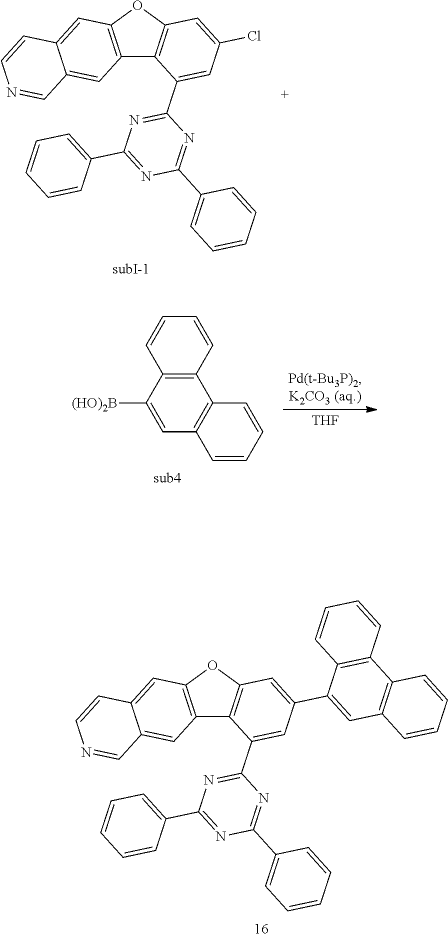 [SMILES: Clc1cc(-c2nc(-c3ccccc3)nc(-c3ccccc3)n2)c2c(c1)oc1cc3ccncc3cc12 ; OB(O)c1cc2ccccc2c2ccccc12 ; c1ccc(-c2nc(-c3ccccc3)nc(-c3cc(-c4cc5ccccc5c5ccccc45)cc4oc5cc6ccncc6cc5c34)n2)cc1 ; "THF"]

subI-1 (15 g, 30.9 mmol) and sub4 (7.2 g, 32.5 mmol) were added to 300 mL of THF under a nitrogen atmosphere, and the mixture was stirred and refluxed. Then, potassium carbonate (8.6 g, 61.9 mmol) was dissolved in 26 ml of water, added thereto, sufficiently stirred, and then bis(tri-tert-butylphosphine)-palladium(0) (0.2 g, 0.3 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 12.8 g of Compound 16. (Yield: 66%, MS: [M+H]⁺=627).

### SYNTHESIS EXAMPLE 17: PREPARATION OF COMPOUND 17

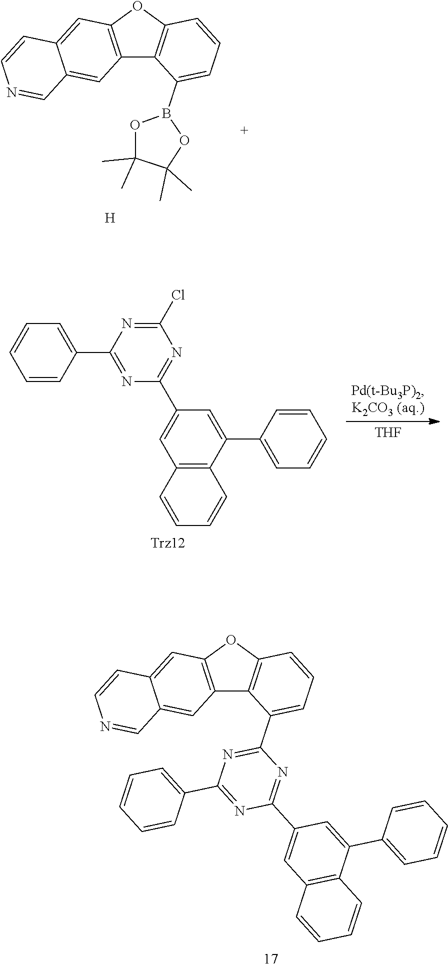

Compound H (15 g, 43.5 mmol) and Trz12 (18 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 15.3 g of Compound 17. (Yield: 61%, MS: [M+H]⁺=577).

### SYNTHESIS EXAMPLE 18: PREPARATION OF COMPOUND 18

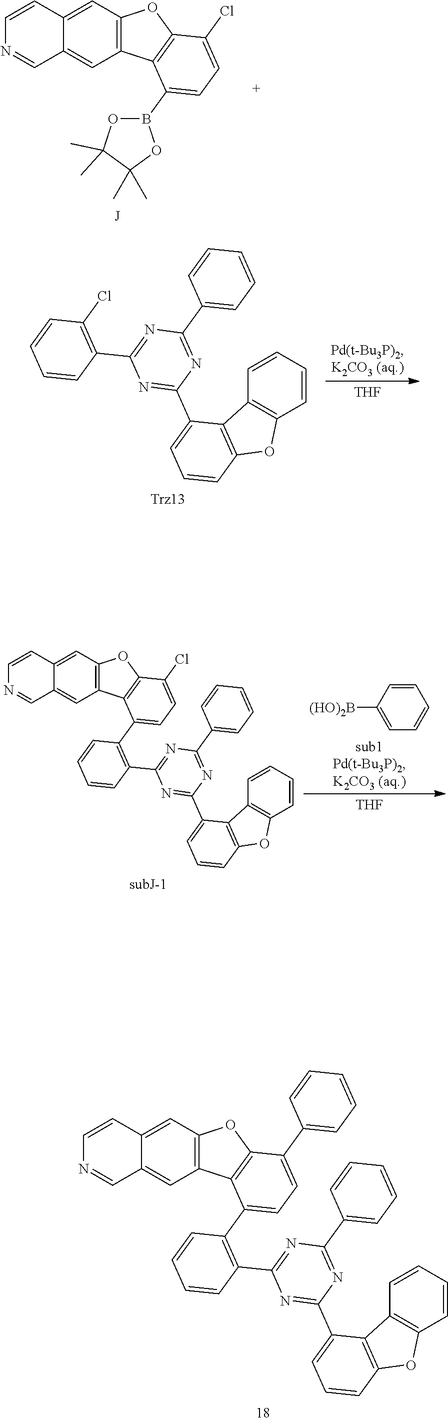

Compound J (15 g, 39.5 mmol) and Trz13 (17.1 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 19.5 g of subJ-1. (Yield: 76%, MS: [M+H]⁺=651).

Next, subJ-1 (15 g, 23 mmol) and sub1 (2.9 g, 24.2 mmol) were added to 300 mL of THF under a nitrogen atmosphere, and the mixture was stirred and refluxed. Then, potassium carbonate (6.4 g, 46.1 mmol) was dissolved in 19 ml of water, added thereto, sufficiently stirred, and then bis(tri-tert-butylphosphine)-palladium(0) (0.1 g, 0.2 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 11 g of Compound 18. (Yield: 69%, MS: [M+H]⁺=693).

### SYNTHESIS EXAMPLE 19: PREPARATION OF COMPOUND 19

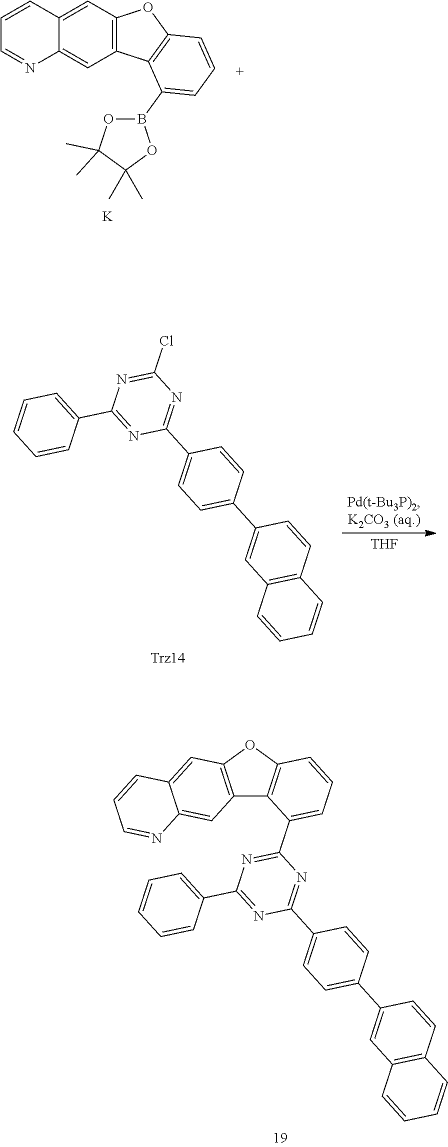

Compound K (15 g, 43.5 mmol) and Trz14 (18 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 19.8 g of Compound 19. (Yield: 79%, MS: [M+H]⁺=577).

### SYNTHESIS EXAMPLE 20: PREPARATION OF COMPOUND 20

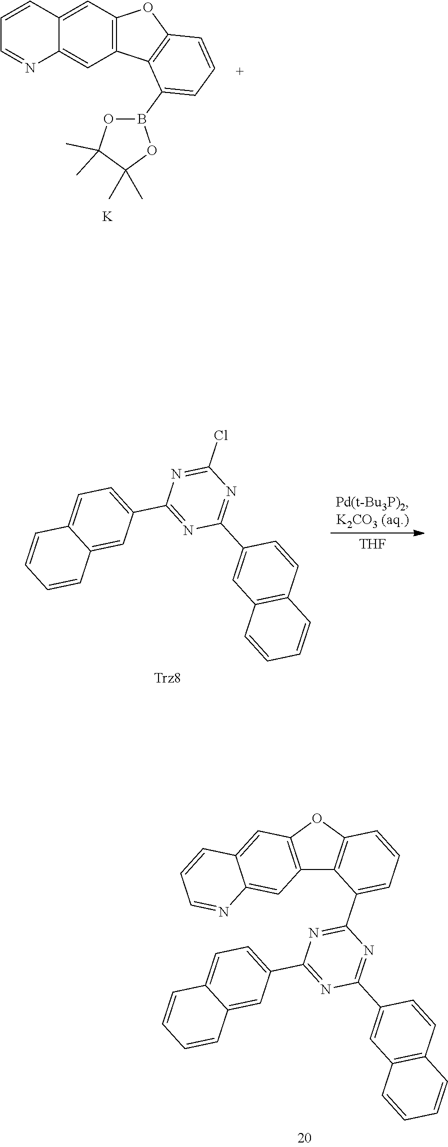

Compound K (15 g, 43.5 mmol) and Trz8 (16.8 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 18.6 g of Compound 20. (Yield: 78%, MS: [M+H]⁺=551).

### SYNTHESIS EXAMPLE 21: PREPARATION OF COMPOUND 21

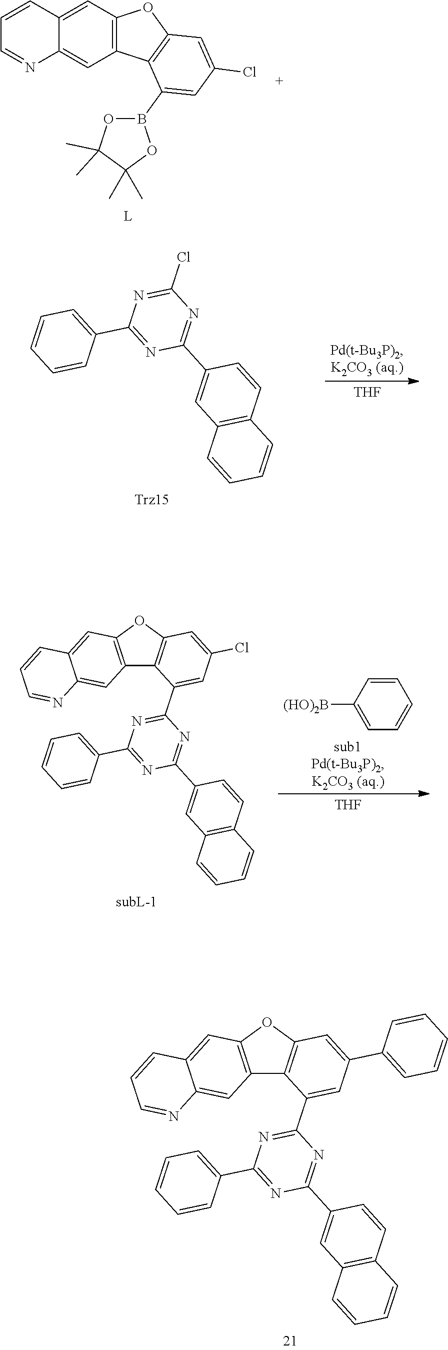

Compound L (15 g, 39.5 mmol) and Trz15 (12.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 14.4 g of subL-1. (Yield: 68%, MS: [M+H]⁺=535).

subL-1 (15 g, 28 mmol) and sub1 (3.6 g, 29.4 mmol) were added to 300 mL of THF under a nitrogen atmosphere, and the mixture was stirred and refluxed. Then, potassium carbonate (7.8 g, 56.1 mmol) was dissolved in 23 ml of water, added thereto, sufficiently stirred, and then bis(tri-tert-butylphosphine)-palladium(0) (0.1 g, 0.3 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 12.9 g of Compound 21. (Yield: 80%, MS: [M+H]⁺=577).

### SYNTHESIS EXAMPLE 22: PREPARATION OF COMPOUND 22

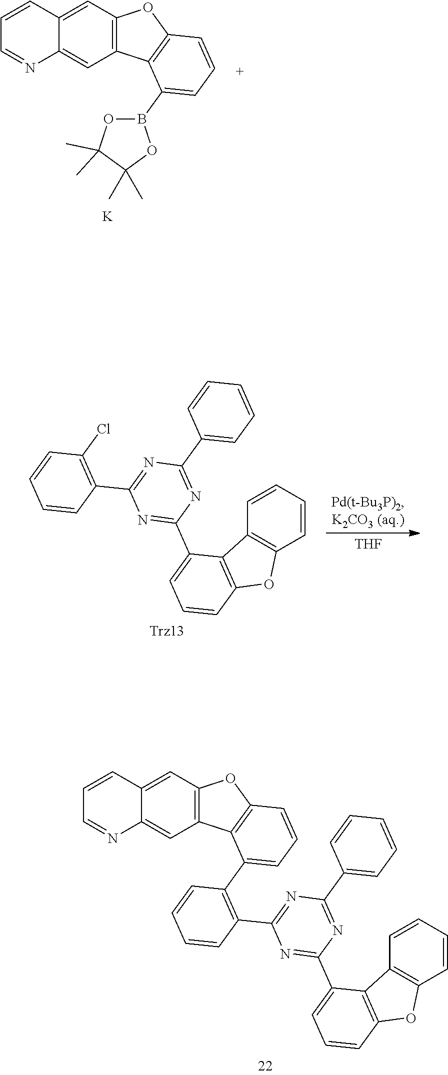

Compound K (15 g, 43.5 mmol) and Trz13 (19.8 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 17.9 g of Compound 22. (Yield: 67%, MS: [M+H]⁺=617).

### SYNTHESIS EXAMPLE 23: PREPARATION OF COMPOUND 23

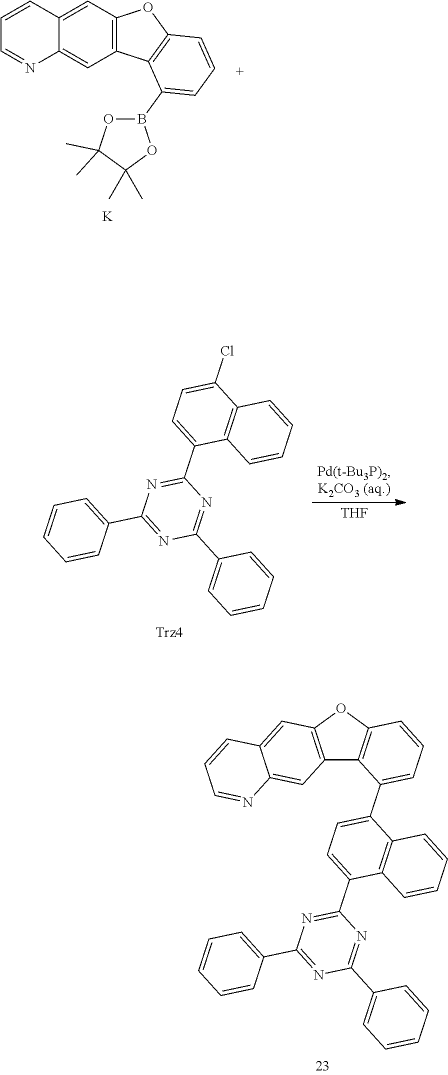

Compound K (15 g, 43.5 mmol) and Trz4 (18 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 17.8 g of Compound 23. (Yield: 71%, MS: [M+H]⁺=577).

### SYNTHESIS EXAMPLE 24: PREPARATION OF COMPOUND 24

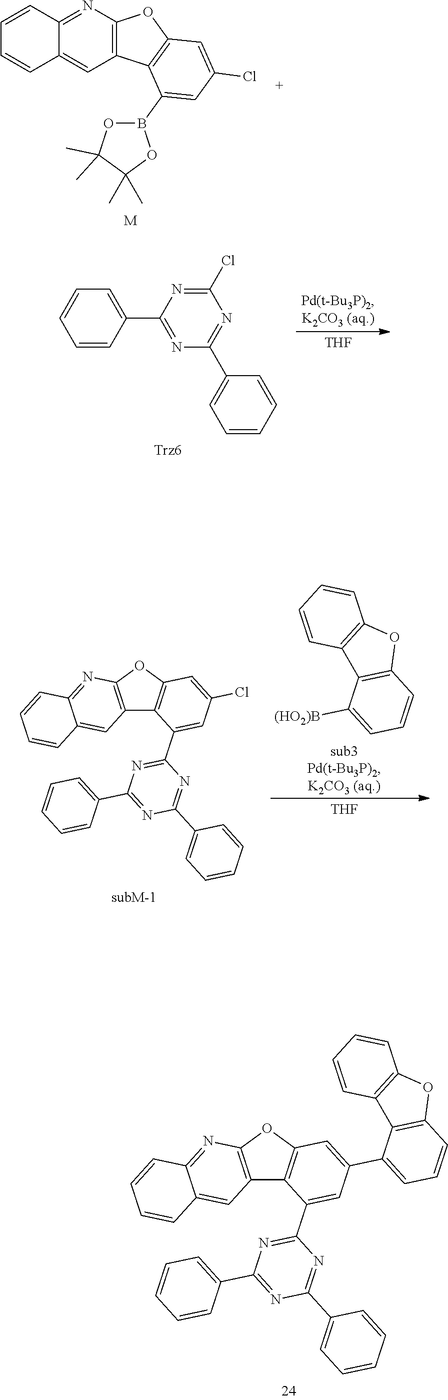

Compound M (15 g, 39.5 mmol) and Trz6 (10.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 14.9 g of subM-1. (Yield: 78%, MS: [M+H]⁺=485).

Next, subM-1 (15 g, 30.9 mmol) and sub3 (6.9 g, 32.5 mmol) were added to 300 mL of THF under a nitrogen atmosphere, and the mixture was stirred and refluxed. Then, potassium carbonate (8.6 g, 61.9 mmol) was dissolved in 26 ml of water, added thereto, sufficiently stirred, and then bis(tri-tert-butylphosphine)-palladium(0) (0.2 g, 0.3 mmol) was added. After reacting for 11 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 11.8 g of Compound 24. (Yield: 62%, MS: [M+H]⁺=617).

### SYNTHESIS EXAMPLE 25: PREPARATION OF COMPOUND 25

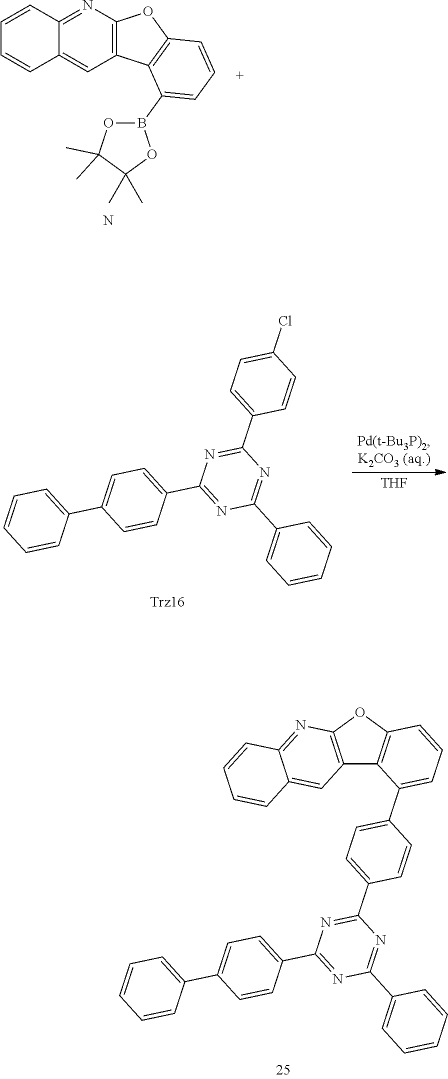

Compound N (15 g, 43.5 mmol) and Trz16 (19.2 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 19.6 g of Compound 25. (Yield: 75%, MS: [M+H]⁺=603).

### SYNTHESIS EXAMPLE 26: PREPARATION OF COMPOUND 26

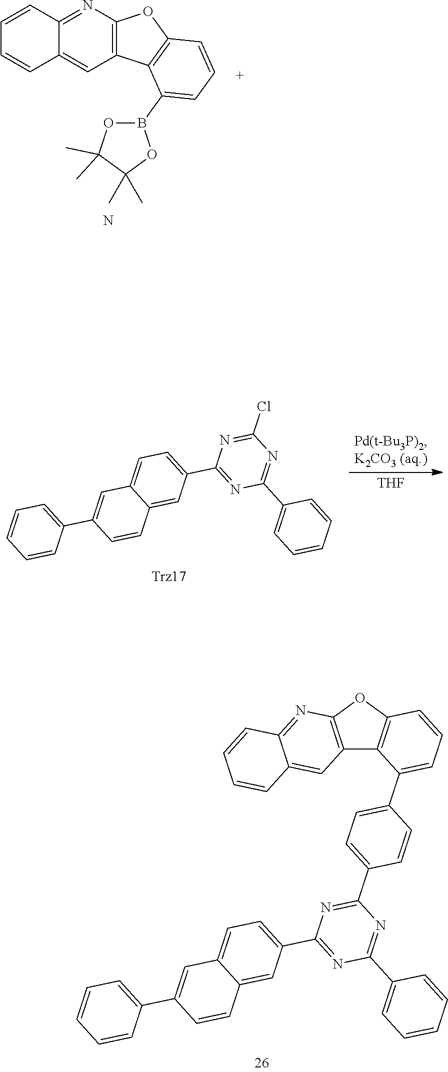

Compound N (15 g, 43.5 mmol) and Trz17 (18 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 16 g of Compound 26. (Yield: 64%, MS: [M+H]⁺=577).

### SYNTHESIS EXAMPLE 27: PREPARATION OF COMPOUND 27

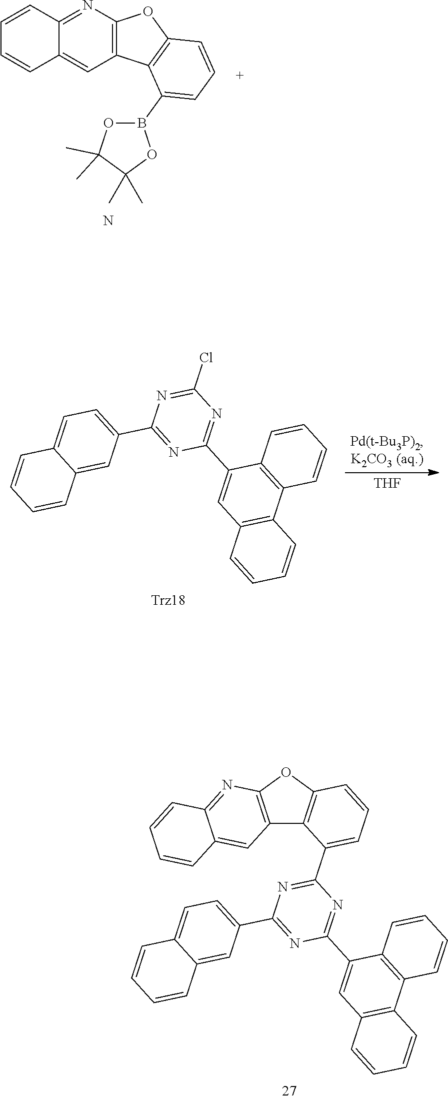

Compound N (15 g, 43.5 mmol) and Trz18 (19.1 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 17.2 g of Compound 27. (Yield: 66%, MS: [M+H]⁺=601).

### SYNTHESIS EXAMPLE 28: PREPARATION OF COMPOUND 28

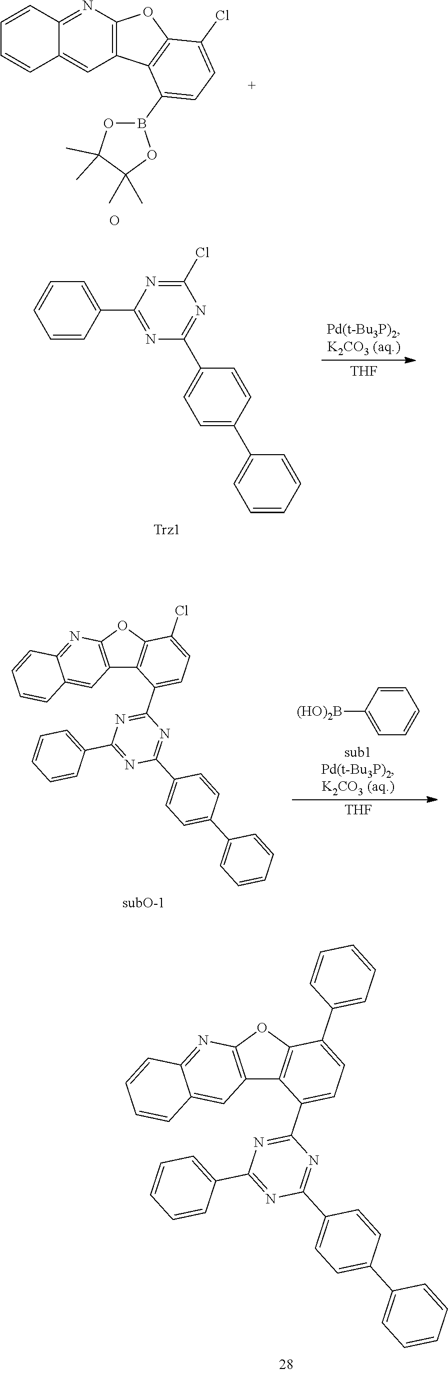

Compound O (15 g, 39.5 mmol) and Trz1 (13.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.5 g of subO-1. (Yield: 61%, MS: [M+H]⁺=561).

Next, subO-1 (15 g, 26.7 mmol) and sub1 (3.4 g, 28.1 mmol) were added to 300 mL of THF under a nitrogen atmosphere, and the mixture was stirred and refluxed. Then, potassium carbonate (7.4 g, 53.5 mmol) was dissolved in 22 ml of water, added thereto, sufficiently stirred, and then bis(tri-tert-butylphosphine)-palladium(0) (0.1 g, 0.3 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 12.2 g of Compound 28. (Yield: 76%, MS: [M+H]⁺=603).

### SYNTHESIS EXAMPLE 29: PREPARATION OF COMPOUND 29

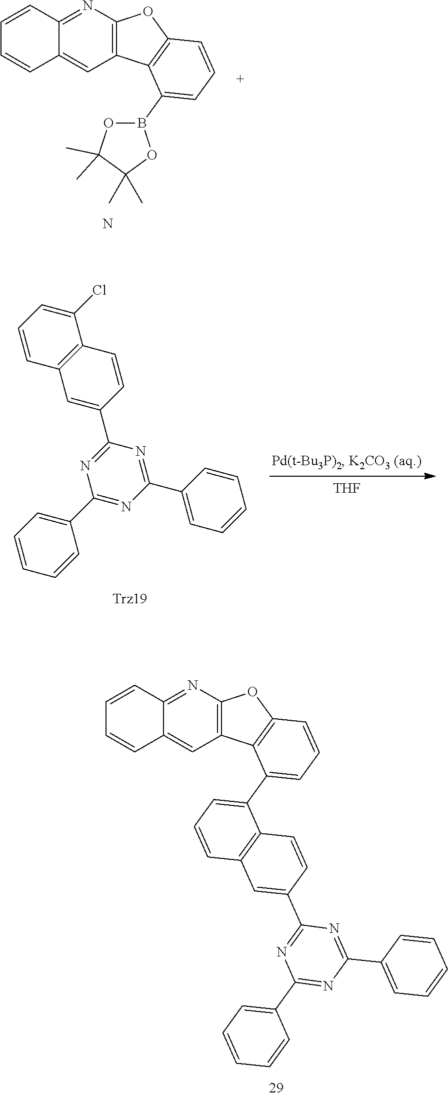

Compound N (15 g, 43.5 mmol) and Trz19 (18 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 18.8 g of Compound 29. (Yield: 75%, MS: [M+H]⁺=577).

### SYNTHESIS EXAMPLE 30: PREPARATION OF COMPOUND 30

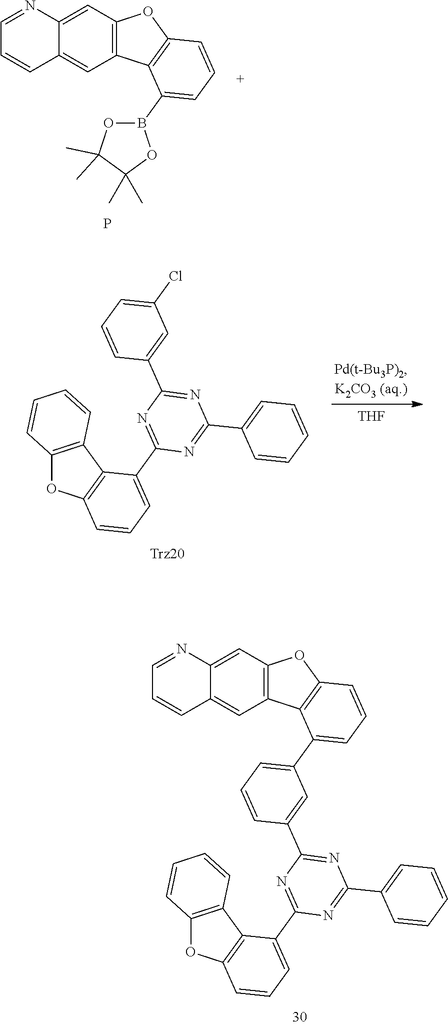

Compound P (15 g, 43.5 mmol) and Trz20 (19.8 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 11 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 16.3 g of Compound 30. (Yield: 61%, MS: [M+H]⁺=617).

### SYNTHESIS EXAMPLE 31: PREPARATION OF COMPOUND 31

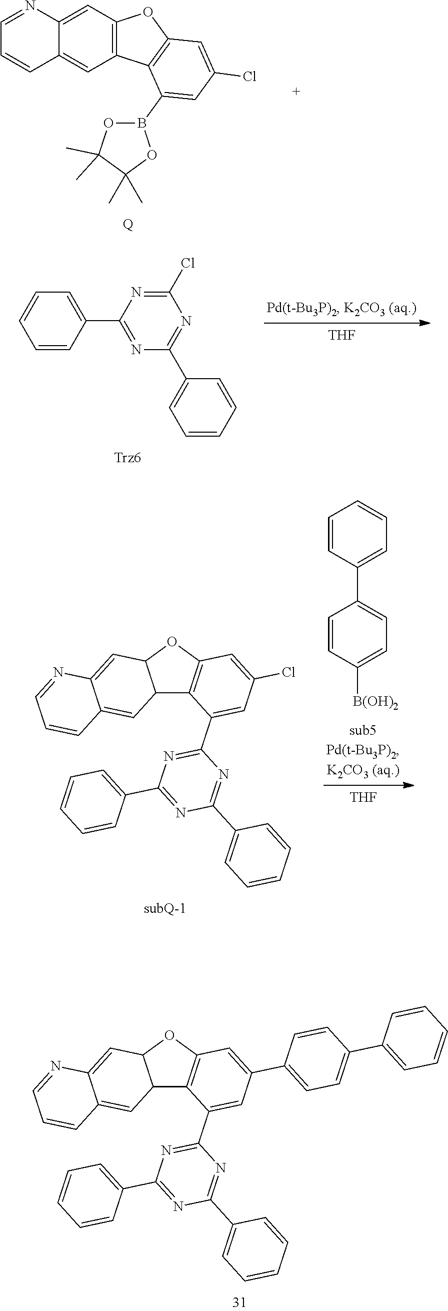

Compound Q (15 g, 39.5 mmol) and Trz6 (10.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0,2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 11.7 g of subQ-1. (Yield: 61%, MS: [M+H]⁺=487).

Next, subQ-1 (15 g, 30.8 mmol) and sub5 (6.4 g, 32.3 mmol) were added to 300 mL of THF under a nitrogen atmosphere, and the mixture was stirred and refluxed. Then, potassium carbonate (8.5 g, 61.6 mmol) was dissolved in 26 ml of water, added thereto, sufficiently stirred, and then bis(tri-tert-butylphosphine)-palladium(0) (0.2 g, 0.3 mmol) was added. After reacting for 11 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 11.5 g of Compound 31. (Yield: 62%, MS: [M+H]⁺=605).

### SYNTHESIS EXAMPLE 32: PREPARATION OF COMPOUND 32

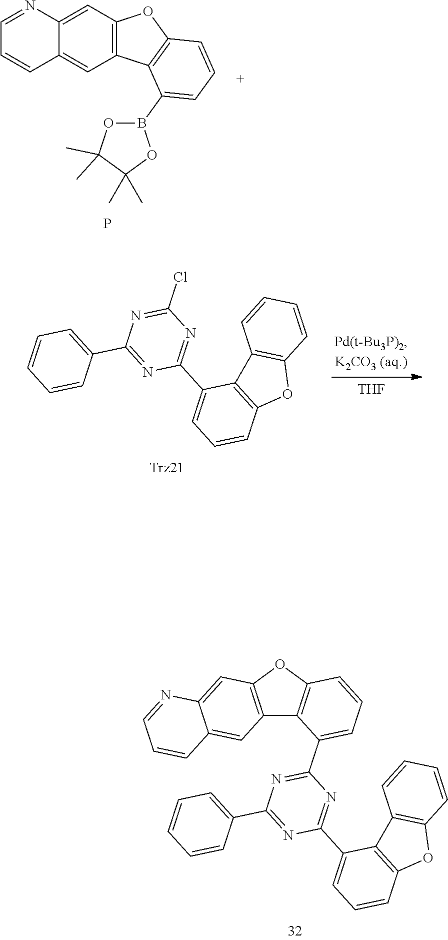

Compound P (15 g, 43.5 mmol) and Trz21 (16.3 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 11 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 16.7 g of Compound 32. (Yield: 71%, MS: [M+H]⁺=541).

### SYNTHESIS EXAMPLE 33: PREPARATION OF COMPOUND 33

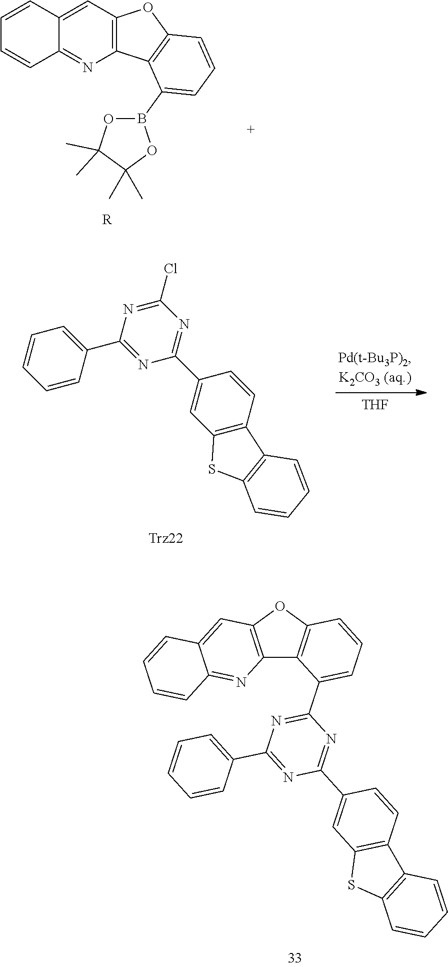

Compound R (15 g, 43.5 mmol) and Trz22 (17.1 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 14.5 g of Compound 33. (Yield: 60%, MS: [M+H]⁺=557).

### SYNTHESIS EXAMPLE 34: PREPARATION OF COMPOUND 34

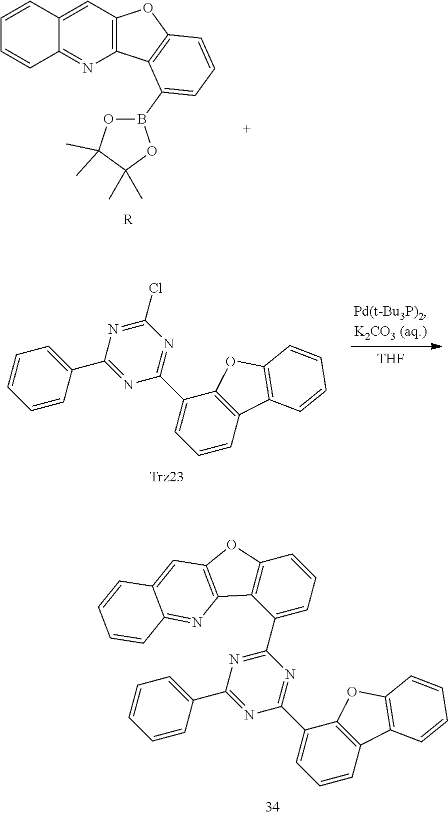

Compound R (15 g, 43.5 mmol) and Trz23 (16.3 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 15.7 g of Compound 34. (Yield: 67%, MS: [M+H]⁺=541).

### SYNTHESIS EXAMPLE 35: PREPARATION OF COMPOUND 35

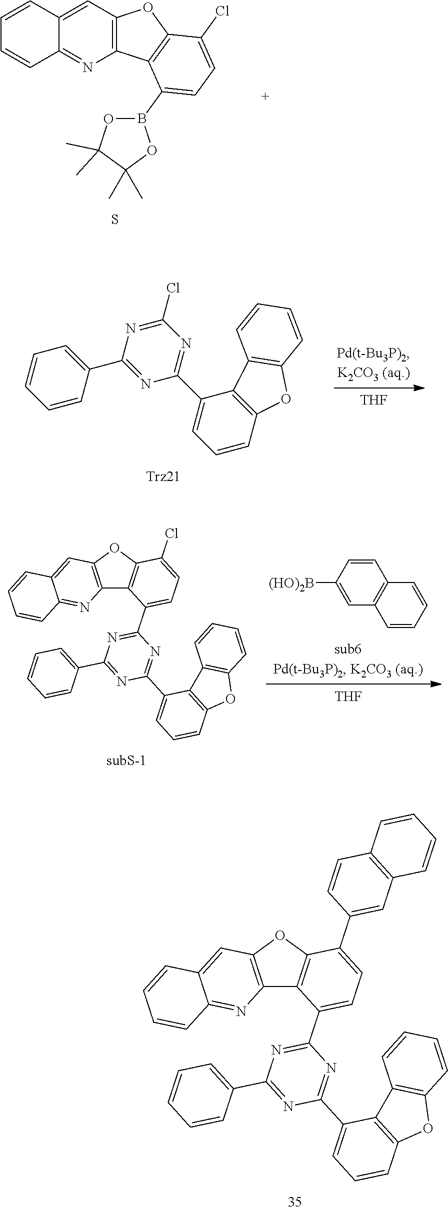

Compound S (15 g, 39.5 mmol) and Trz21 (14.1 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 11 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 16.1 g of subS-1. (Yield: 71%, MS: [M+H]⁺=575).

Next, subS-1 (15 g, 26.1 mmol) and sub6 (4.7 g, 27.4 mmol) were added to 300 mL of THF under a nitrogen atmosphere, and the mixture was stirred and refluxed. Then, potassium carbonate (7.2 g, 52.2 mmol) was dissolved in 22 ml of water, added thereto, sufficiently stirred, and then bis(tri-tert-butylphosphine)-palladium(0) (0.1 g, 0.3 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.4 g of Compound 35. (Yield: 77%, MS: [M+H]⁺=667).

### SYNTHESIS EXAMPLE 36: PREPARATION OF COMPOUND 36

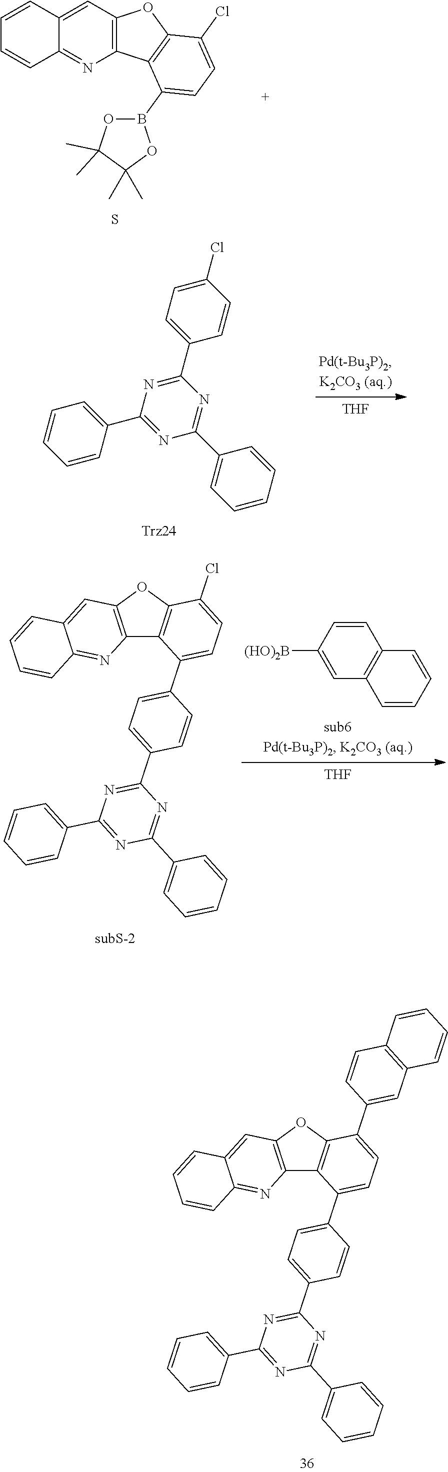

Compound S (15 g, 39.5 mmol) and Trz24 (13.6 g, 39.5 mmol) were added to 300 mL. of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 15.3 g of subS-2. (Yield: 69%, MS: [M+H]⁺=561).

subS-2 (15 g, 26.7 mmol) and sub6 (4.8 g, 28.1 mmol) were added to 300 mL of THF under a nitrogen atmosphere, and the mixture was stirred and refluxed. Then, potassium carbonate (7.4 g, 53.5 mmol) was dissolved in 22 ml of water, added thereto, sufficiently stirred, and then bis(tri-tert-butylphosphine)-palladium(0) (0.1 g, 0.3 mmol) was added. After reacting for 10 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.8 g of Compound 36. (Yield: 79%, MS: [M+H]⁺=653).

### SYNTHESIS EXAMPLE 37: PREPARATION OF COMPOUND 37

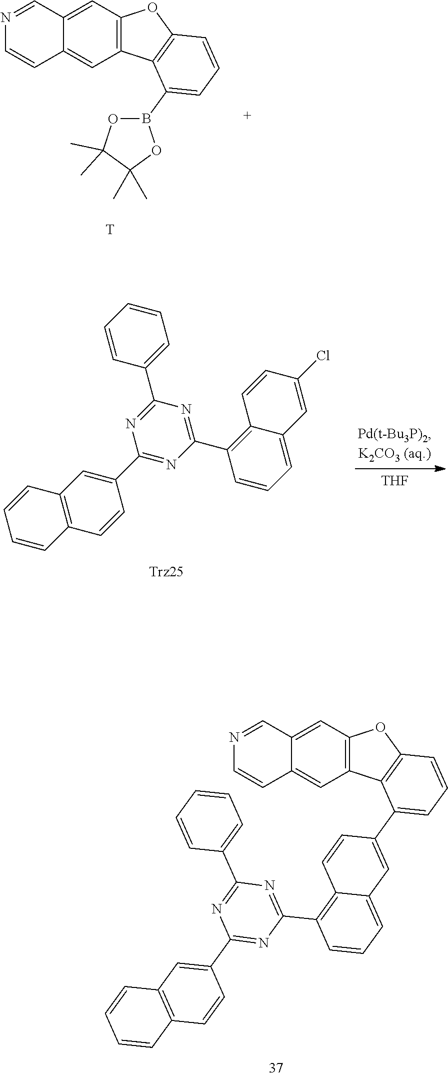

Compound T (15 g, 43.5 mmol) and Trz25 (20.3 g, 45.6 mmol) were added to 300 mL of THF under a nitrogen atmosphere, and the mixture was stirred and refluxed. Then, potassium carbonate (12 g, 86.9 mmol) was dissolved in 36 ml of water, added thereto, sufficiently stirred, and then bis(tri-tert-butylphosphine)-palladium(0) (0.2 g, 0.4 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 19 g of Compound 37. (Yield: 70%, MS: [M+H]⁺=627).

### SYNTHESIS EXAMPLE 38: PREPARATION OF COMPOUND 38

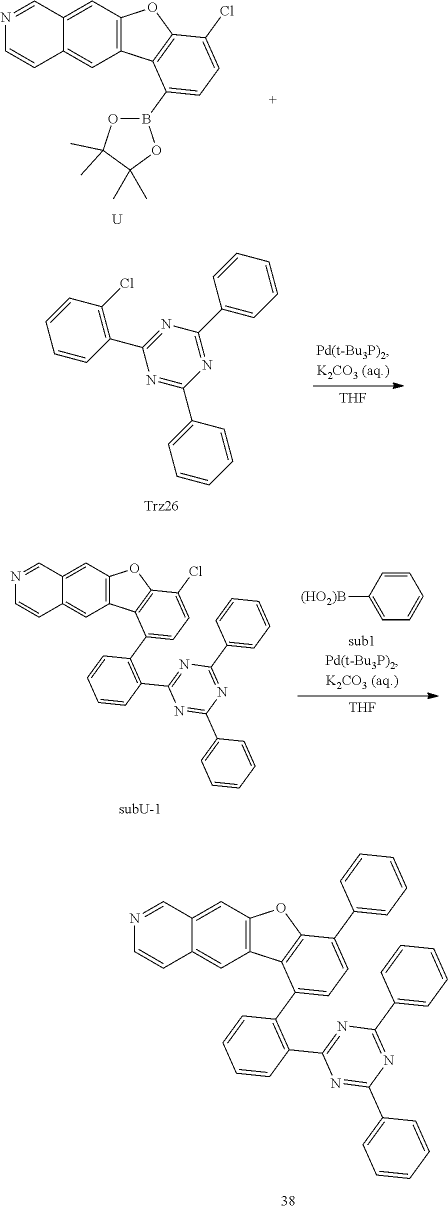

Compound U (15 g, 39.5 mmol) and Trz26 (13.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 17.5 g of subU-1. (Yield: 79%, MS: [M+H]⁺=561).

Next, subU-1 (15 g, 26.7 mmol) and sub1 (3.4 g, 28.1 mmol) were added to 300 mL of THF under a nitrogen atmosphere, and the mixture was stirred and refluxed. Then, potassium carbonate (7.4 g, 53.5 mmol) was dissolved in 22 ml of water, added thereto, sufficiently stirred, and then bis(tri-tert-butylphosphine)-palladium(0) (0.1 g, 0.3 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 12.2 g of Compound 38. (Yield: 76%, MS: [M+H]⁺=603).

### SYNTHESIS EXAMPLE 39: PREPARATION OF COMPOUND 39

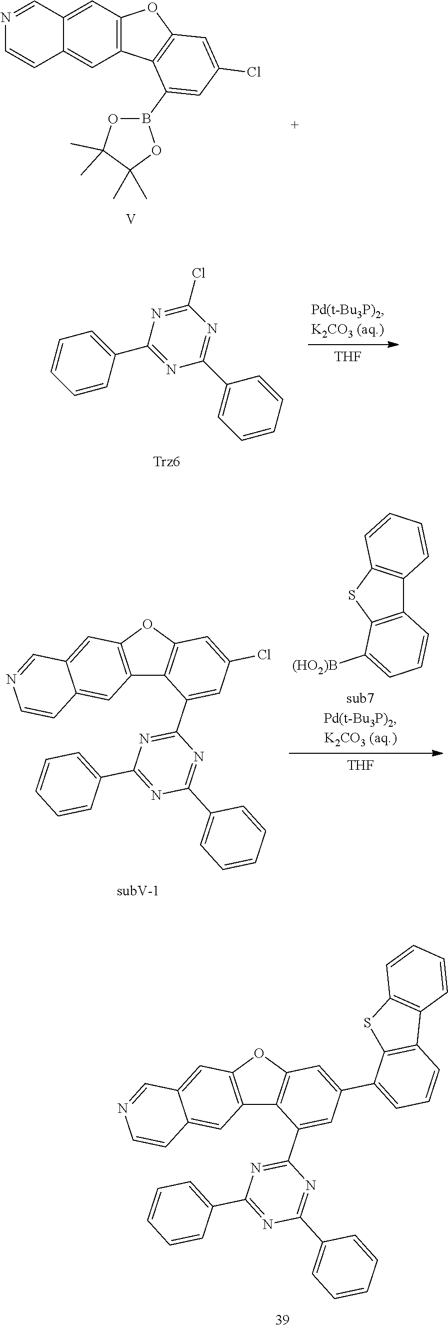

Compound V (15 g, 39.5 mmol) and Trz6 (10.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 13.2 g of subV-1. (Yield: 69%, MS: [M+H]⁺=485).

Next, subV-1 (15 g, 30.9 mmol) and sub7 (7.4 g, 32.5 mmol) were added to 300 mL of THF under a nitrogen atmosphere, and the mixture was stirred and refluxed. Then, potassium carbonate (8.6 g, 61.9 mmol) was dissolved in 26 ml of water, added thereto, sufficiently stirred, and then bis(tri-tert-butylphosphine)-palladium(0) (0.2 g, 0.3 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 12.7 g of Compound 39. (Yield: 65%, MS: [M+H]⁺=633).

### SYNTHESIS EXAMPLE 40: PREPARATION OF COMPOUND 40

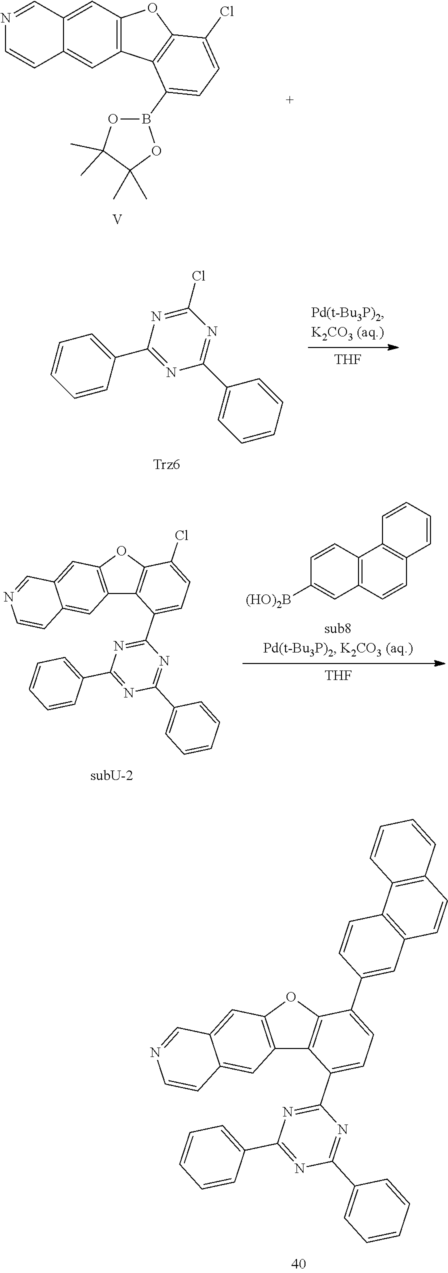

Compound U (15 g, 39.5 mmol) and Trz6 (10.6 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 9 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 14.3 g of subU-2. (Yield: 75%, MS: [M+H]⁺=485).

Next, subU-2 (15 g, 30.9 mmol) and sub8 (7.2 g, 32.5 mmol) were added to 300 mL of THF under a nitrogen atmosphere, and the mixture was stirred and refluxed. Then, potassium carbonate (8.6 g, 61.9 mmol) was dissolved in 26 ml of water, added thereto, sufficiently stirred, and then bis(tri-tert-butylphosphine)-palladium(0) (0.2 g, 0.3 mmol) was added. After reacting for 12 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 14.1 g of Compound 40. (Yield: 73%, MS: [M+H]⁺=627).

### SYNTHESIS EXAMPLE 41: PREPARATION OF COMPOUND 41

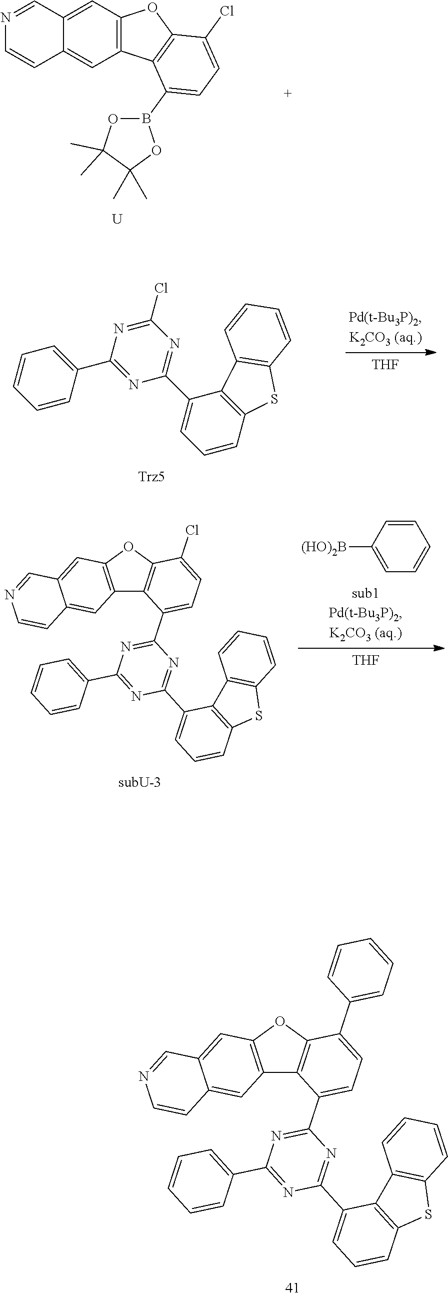

Compound U (15 g, 39.5 mmol) and Trz5 (14.8 g, 39.5 mmol) were added to 300 mL of THF under a nitrogen atmosphere, and the mixture was stirred and refluxed. Then, potassium carbonate (10.9 g, 79 mmol) was dissolved in 33 ml of water, added thereto, sufficiently stirred, and then bis(tri-tert-butylphosphine)-palladium(0) (0.2 g, 0.4 mmol) was added. After reacting for 11 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 15.2 g of subU-3. (Yield: 65%, MS: [M+H]⁺=591).

Next, subU-3 (15 g, 25.4 mmol) and sub1 (3.2 g, 26.6 mmol) were added to 300 mL of THF under a nitrogen atmosphere, and the mixture was stirred and refluxed. Then, potassium carbonate (7 g, 50.8 mmol) was dissolved in 21 ml of water, added thereto, sufficiently stirred, and then bis(tri-tert-butylphosphine)-palladium(0) (0.1 g, 0.3 mmol) was added. After reacting for 8 hours, the reaction mixture was cooled to room temperature, the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to give 12.2 g of Compound 41. (Yield: 76%, MS: [M+H]⁺=633).

### EXAMPLE 1

A glass substrate on which a thin film of ITO (indium tin oxide) was coated in a thickness of 1,000 Å was put into distilled water containing a detergent dissolved therein and ultrasonically washed. The detergent used was a product commercially available from Fisher Co., and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was formed to a thickness of 1150 Å A as a hole injection layer, but the following Compound A-1 was p-doped at a concentration of 1.5 wt. %. The following Compound HT-1 was vacuum deposited on the hole injection layer to form a hole transport layer with a film thickness of 800 ÅA. Then, the following Compound EB-1 was vacuum deposited to a film thickness of 150 Å on the hole transport layer to form an electron blocking layer.

Then, the Compound 1 prepared in Synthesis Example 1, and the following Compound Dp-7 were vacuum deposited in a weight ratio of 98:2 on the electron blocking layer to form a red light emitting layer with a thickness of 400 Å.

The following Compound HB-1 was vacuum deposited to a film thickness of 30 Å on the light emitting layer to form a hole blocking layer. The, the following Compound ET-1 and the following Compound LiQ were vacuum deposited in a weight ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

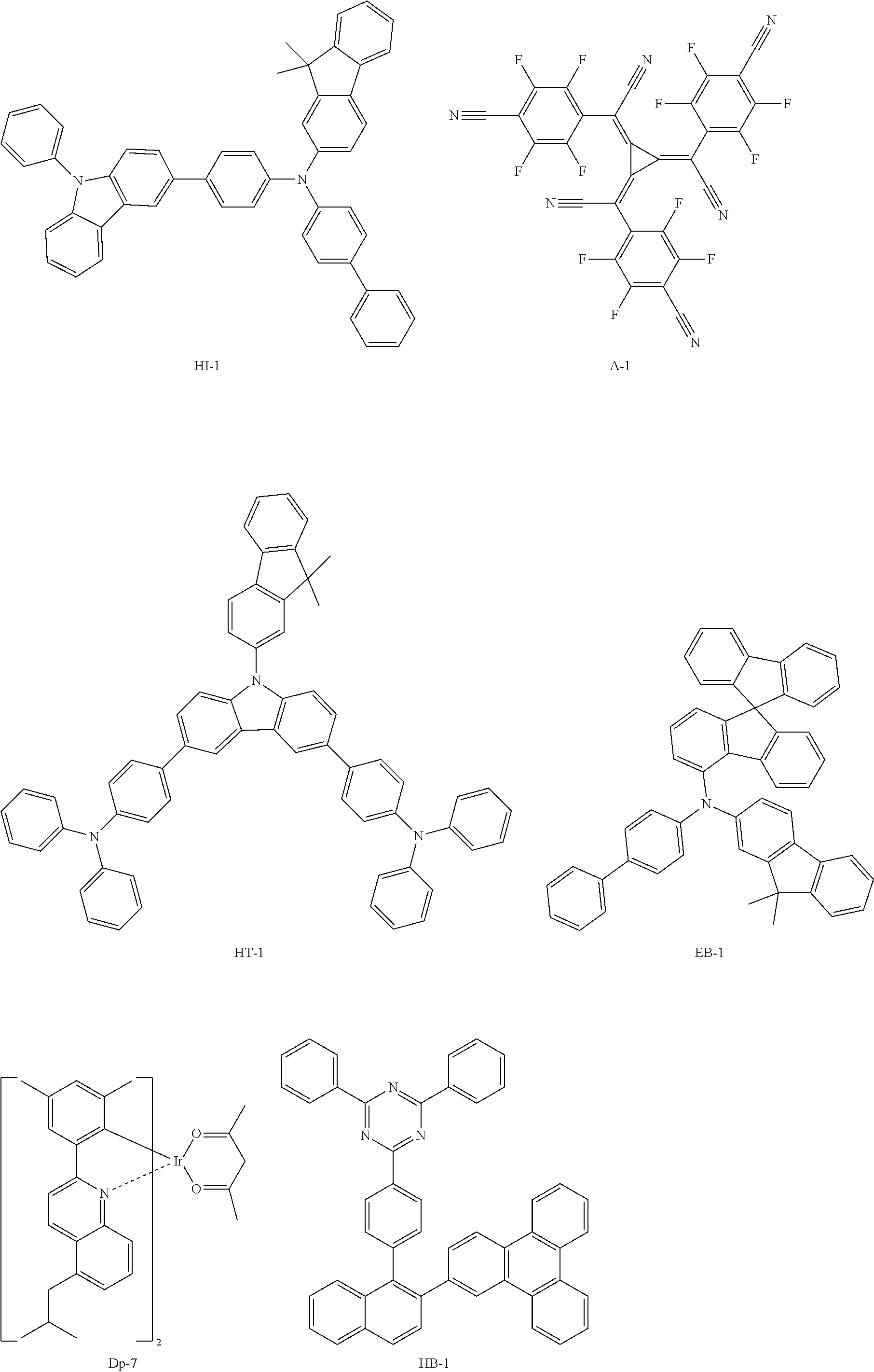

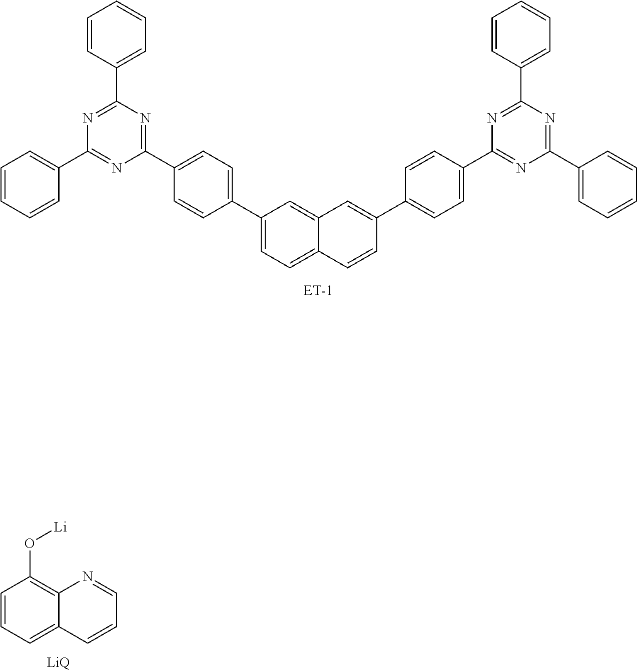

In the above-mentioned processes, the deposition rates of the organic materials were maintained at 0.4 to 0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷ to 5×10⁻⁶ torr, thereby manufacturing an organic light emitting device.

### EXAMPLES 2 to 24

The organic light emitting devices were manufactured in the same manner as in Example 1, except that the compounds shown in Table 1 below were used instead of Compound 1 in the organic light emitting device of Example 1.

### COMPARATIVE EXAMPLES 1 to 12

The organic light emitting devices were manufactured in the same manner as in Example 1, except that the compounds shown in Table 1 below were used instead of Compound 1 in the organic light emitting device of Example 1.

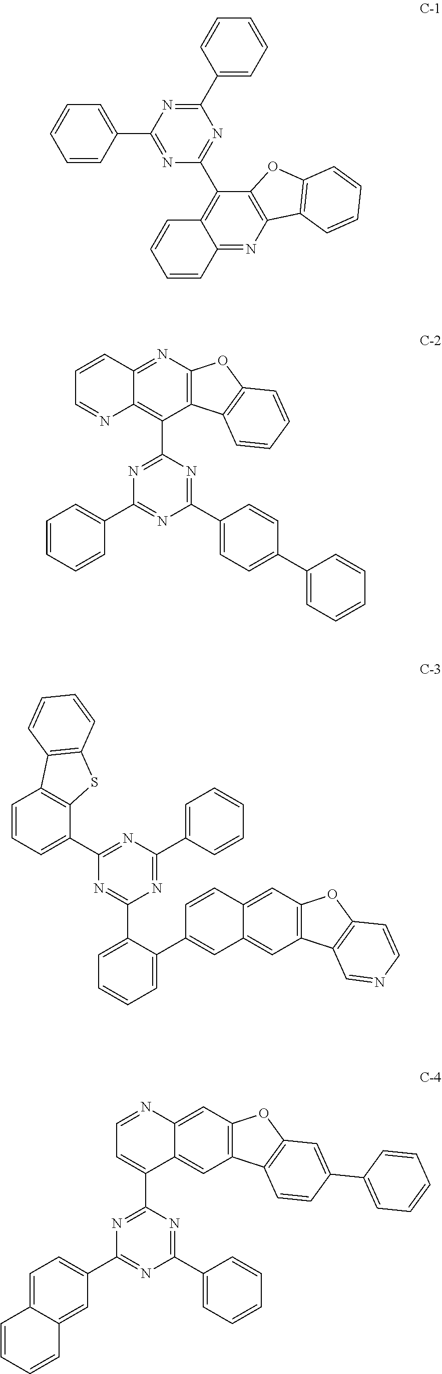 [SMILES: c1ccc(-c2nc(-c3ccccc3)nc(-c3c4ccccc4nc4c3oc3ccccc34)n2)cc1 ; c1ccc(-c2ccc(-c3nc(-c4ccccc4)nc(-c4c5ncccc5nc5oc6ccccc6c45)n3)cc2)cc1 ; c1ccc(-c2nc(-c3ccccc3-c3ccc4cc5oc6ccncc6c5cc4c3)nc(-c3cccc4c3sc3ccccc34)n2)cc1 ; c1ccc(-c2ccc3c(c2)oc2cc4nccc(-c5nc(-c6ccccc6)nc(-c6ccc7ccccc7c6)n5)c4cc23)cc1]

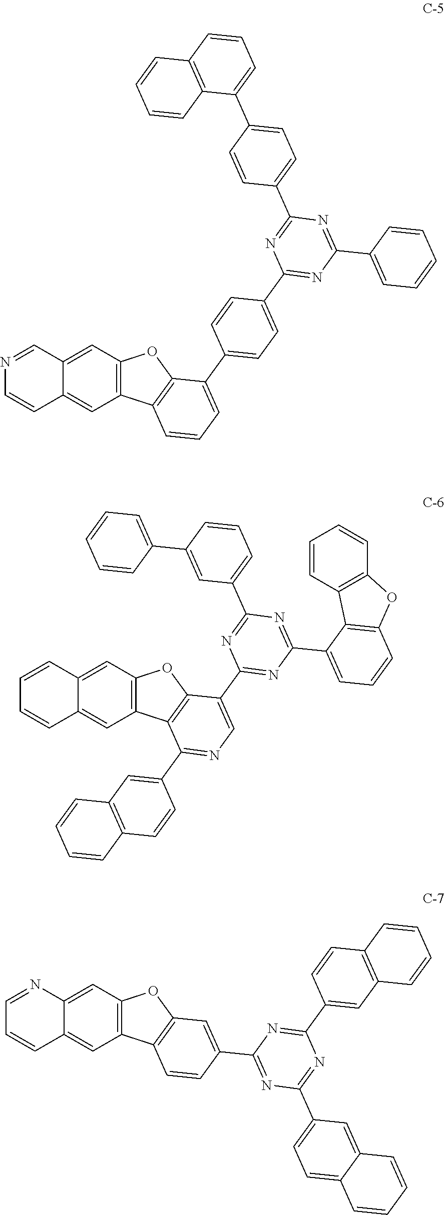 [SMILES: c1ccc(-c2nc(-c3ccc(-c4cccc5ccccc45)cc3)nc(-c3ccc(-c4cccc5c4oc4cc6cnccc6cc45)cc3)n2)cc1 ; c1ccc(-c2cccc(-c3nc(-c4cnc(-c5ccc6ccccc6c5)c5c4oc4cc6ccccc6cc45)nc(-c4cccc5oc6ccccc6c45)n3)c2)cc1 ; c1ccc2cc(-c3nc(-c4ccc5ccccc5c4)nc(-c4ccc5c(c4)oc4cc6ncccc6cc45)n3)ccc2c1]

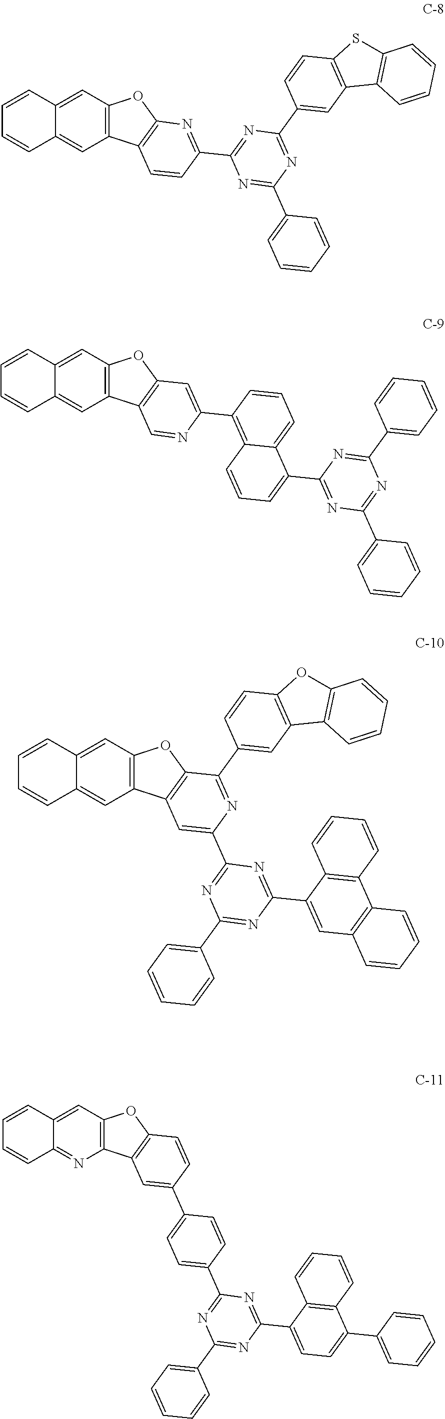 [SMILES: c1ccc(-c2nc(-c3ccc4sc5ccccc5c4c3)nc(-c3ccc4c(n3)oc3cc5ccccc5cc34)n2)cc1 ; c1ccc(-c2nc(-c3ccccc3)nc(-c3cccc4c(-c5cc6oc7cc8ccccc8cc7c6cn5)cccc34)n2)cc1 ; c1ccc(-c2nc(-c3cc4c(oc5cc6ccccc6cc54)c(-c4ccc5oc6ccccc6c5c4)n3)nc(-c3cc4ccccc4c4ccccc34)n2)cc1 ; c1ccc(-c2nc(-c3ccc(-c4ccc5oc6cc7ccccc7nc6c5c4)cc3)nc(-c3ccc(-c4ccccc4)c4ccccc34)n2)cc1]

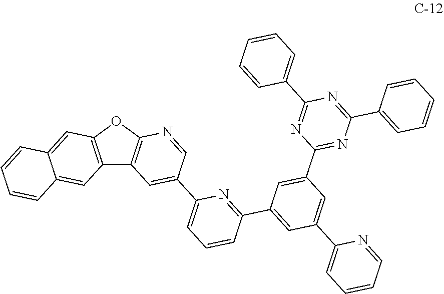 [SMILES: c1ccc(-c2nc(-c3ccccc3)nc(-c3cc(-c4ccccn4)cc(-c4cccc(-c5cnc6oc7cc8ccccc8cc7c6c5)n4)c3)n2)cc1]

### EXPERIMENTAL EXAMPLE

The driving voltage and efficiency were measured by applying a current (15 mA/cm²) to the organic light emitting devices manufactured in the Examples 1 to 24 and Comparative Examples 1 to 12, and the results are shown in Table 1 below. T95 means the time required for the luminance to be reduced to 95% of the initial luminance (6000 nit).

TABLE 1

| | Material | | | | |
|---|---|---|---|---|---|
| | (Light | Driving | | | |
| | emitting | voltage | Efficiency | Lifetime | Luminous |
| Category | layer) | (V) | (cd/A) | T95 (hr) | color |
| Example 1 | Compound | 3.85 | 20.3 | 143 | Red |
| | 1 | | | | |
| Example 2 | Compound | 3.83 | 20.9 | 139 | Red |
| | 2 | | | | |
| Example 3 | Compound | 3.92 | 18.3 | 147 | Red |
| | 4 | | | | |
| Example 4 | Compound | 3.77 | 19.7 | 143 | Red |
| | 6 | | | | |
| Example 5 | Compound | 3.83 | 18.0 | 150 | Red |
| | 8 | | | | |
| Example 6 | Compound | 3.72 | 20.3 | 165 | Red |
| | 9 | | | | |
| Example 7 | Compound | 3.78 | 21.0 | 163 | Red |
| | 10 | | | | |
| Example 8 | Compound | 3.84 | 18.9 | 150 | Red |
| | 11 | | | | |
| Example 9 | Compound | 3.91 | 20.5 | 161 | Red |
| | 16 | | | | |
| Example 10 | Compound | 3.82 | 20.4 | 164 | Red |
| | 17 | | | | |
| Example 11 | Compound | 3.86 | 18.8 | 157 | Red |
| | 18 | | | | |
| Example 12 | Compound | 3.71 | 21.1 | 165 | Red |
| | 19 | | | | |
| Example 13 | Compound | 3.76 | 19.3 | 144 | Red |
| | 23 | | | | |
| Example 14 | Compound | 3.70 | 21.5 | 171 | Red |
| | 24 | | | | |
| Example 15 | Compound | 3.73 | 20.8 | 168 | Red |
| | 27 | | | | |
| Example 16 | Compound | 3.80 | 18.6 | 165 | Red |
| | 29 | | | | |
| Example 17 | Compound | 3.90 | 17.8 | 148 | Red |
| | 30 | | | | |
| Example 18 | Compound | 3.84 | 19.2 | 135 | Red |
| | 31 | | | | |
| Example 19 | Compound | 3.92 | 19.5 | 158 | Red |
| | 33 | | | | |
| Example 20 | Compound | 3.75 | 20.4 | 152 | Red |
| | 35 | | | | |
| Example 21 | Compound | 3.87 | 18.5 | 161 | Red |
| | 36 | | | | |
| Example 22 | Compound | 3.84 | 19.1 | 153 | Red |
| | 37 | | | | |
| Example 23 | Compound | 3.77 | 20.0 | 187 | Red |
| | 39 | | | | |
| Example 24 | Compound | 3.71 | 19.3 | 179 | Red |
| | 40 | | | | |
| Comparative | C-1 | 4.24 | 17.0 | 104 | Red |
| Example 1 | | | | | |
| Comparative | C-2 | 4.53 | 15.1 | 52 | Red |
| Example 2 | | | | | |
| Comparative | C-3 | 4.28 | 16.2 | 89 | Red |
| Example 3 | | | | | |
| Comparative | C-4 | 4.06 | 17.4 | 117 | Red |
| Example 4 | | | | | |
| Comparative | C-5 | 4.35 | 16.3 | 91 | Red |
| Example 5 | | | | | |
| Comparative | C-6 | 4.05 | 17.1 | 108 | Red |
| Example 6 | | | | | |
| Comparative | C-7 | 4.09 | 16.6 | 94 | Red |
| Example 7 | | | | | |
| Comparative | C-8 | 4.17 | 16.0 | 75 | Red |
| Example 8 | | | | | |
| Comparative | C-9 | 4.08 | 17.1 | 98 | Red |
| Example 9 | | | | | |
| Comparative | C-10 | 4.17 | 16.3 | 79 | Red |
| Example 10 | | | | | |
| Comparative | C-11 | 4.22 | 15.8 | 54 | Red |
| Example 11 | | | | | |
| Comparative | C-12 | 4.35 | 13.2 | 17 | Red |
| Example 12 | | | | | |

As shown in Table 1, it was confirmed that the organic light emitting devices of Examples using the compound of Chemical Formula 1 as a host material of the light emitting layer exhibited excellent luminous efficiency and remarkably improved lifetime characteristics, as compared with the organic light emitting devices of Comparative Examples using the compounds not included in Chemical Formula 1.

Specifically, considering that the device according to Examples exhibited a remarkably lowered driving voltage and improved efficiency characteristics, as compared with Comparative Examples in which Comparative Example Compounds C-1 to C-12 were employed as the host material of the light emitting layer, it can be seen that energy transfer from the compound of Chemical Formula 1, which is the host material, to the red dopant was effectively performed. In addition, considering that the organic light emitting devices of Examples were improved in lifetime characteristics as well as the efficiency, it is judged that the compound of Chemical Formula 1 also has high stability to electrons and holes. Therefore, when the compound of Chemical Formula 1 is used as the host material of the organic light emitting device, it can be confirmed that the driving voltage, luminous efficiency and lifetime characteristics of the organic light emitting device can be improved. In general, considering that the luminous efficiency and lifetime characteristics of an organic light emitting devices have a trade-off relationship with each other, this can be considered that the organic light emitting devices of Examples exhibit remarkably improved device characteristics as compared with the devices of Comparative Examples.

<Description of Symbols>

| | |
|---|---|
| 1: substrate | 2: anode |
| 3: light emitting layer | 4: cathode |
| 5: hole injection layer | 6: hole transport layer |
| 7: electron blocking layer | 8: hole blocking layer |

9: electron injection and transport layer

1. A compound of Chemical Formula 1: 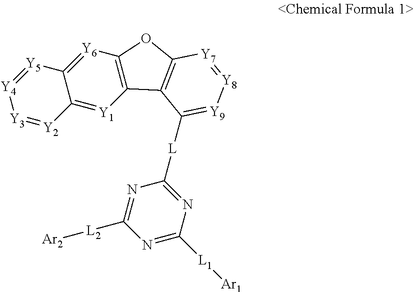
wherein, in Chemical Formula 1;
Y₁ to Y₉ are each independently N, C—H, C-D, or C-L′—R, with the proviso that at least one of Y₁ to Y₉ is N;
L′ is a single bond or a substituted or unsubstituted C₆₋₆₀ arylenel;
R is a substituted or unsubstituted C₆₋₆₀ or a substituted or unsubstituted C₂₋₆₀ heteroaryl containing any one or more heteroatoms selected from the group consisting of N, O and S;
L is a single bond, a substituted or unsubstituted C₆₋₆₀ arylene, or a substituted or unsubstituted C₂₋₆₀ heteroarylene containing any one or more heteroatoms selected from the group consisting of N, O and S;
L₁ and L₂ are each independently a single bond or a substituted or unsubstituted C₆₋₆₀ arylene; and
Ar₁ and Ar₂ are each independently a substituted or unsubstituted C₆₋₆₀ aryl or a substituted or unsubstituted C₂₋₆₀ heteroaryl containing any one or more heteroatoms selected from the group consisting of N, O and S.

2. The compound of claim 1, wherein:
one of Y₁ to Y₉ is N.

3. The compound of claim 2, wherein:
one of Y₁ to Y₉ is N, and the rest are each independently C—H, or C-D; or one of Y₁ to Y₉ is N, one of the rest is C-L′ R, and the remaining are each independently C—H, or C-D.

4. The compound of claim 1, wherein:
L′ is a single bond; phenylene that is unsubstituted or substituted with deuterium; or naphthylene that is unsubstituted or substituted with deuterium.

5. The compound of claim 1, wherein:
R is any one substituent selected from the group consisting of the following: 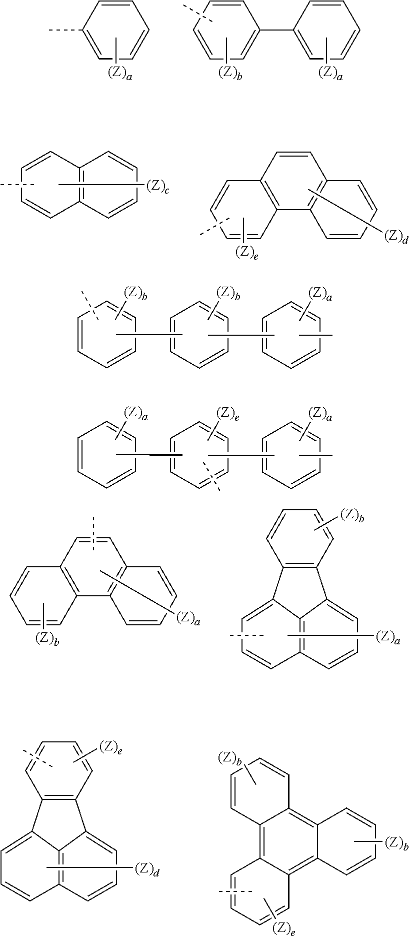
wherein;
X₁ and X₂ are each independently O, S, or N(phenyl);
each Z is independently deuterium (D), a C₁₋₁₀ alkyl, or a C₆₋₂₀ aryl;
each a is independently an integer of 0 to 5;
each b is independently an integer of 0 to 4;
each c is independently an integer of 0 to 7;
each d is independently an integer of 0 to 6; and
each e is independently an integer of 0 to 3.

6. The compound of claim 1, wherein:
L is a single bond, or any one selected from the group consisting of the following: 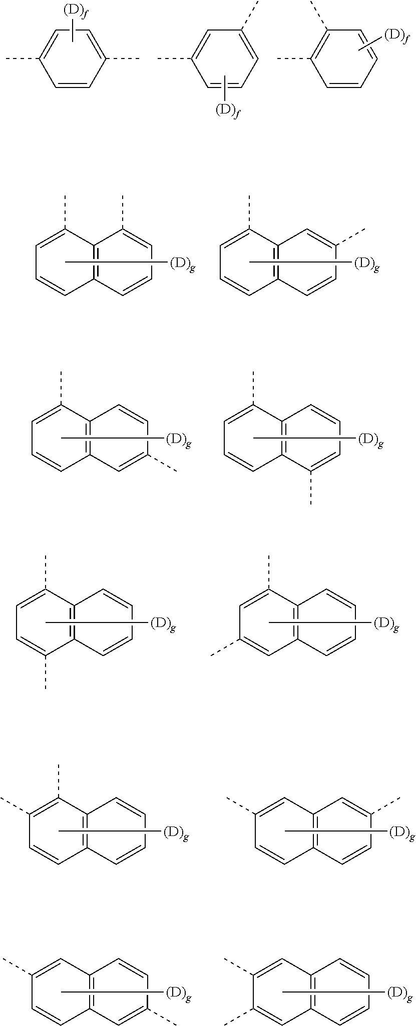
wherein;
D means deuterium;
each f is independently an integer of 0 to 4; and
each g is independently an integer of 0 to 6.

7. The compound of claim 1, wherein:
L₁ and L₂ are each independently a single bond; phenylene that is unsubstituted or substituted with deuterium; biphenyldiyl that is unsubstituted or substituted with deuterium; or naphthylene that is unsubstituted or substituted with deuterium.

8. The compound of claim 1, wherein:
Ar₁ and Ar₂ are each independently phenyl, biphenylyl, terphenylyl, naphthyl, phenanthryl, fluorenyl, dibenzofuranyl, dibenzothiophenyl, benzonaphthofuranyl, benzonaphthothiophenyl, carbazolyl, or benzocarbazolyl,
where Ar₁ and Ar₂ are unsubstituted or substituted with one or more substituents selected from the group consisting of deuterium, a C₁₋₁₀ alkyl and a C₆₋₂₀ aryl.

9. The compound of claim 1, wherein:
one of Ar₁ and Ar₂ is phenyl, biphenylyl, or naphthyl.

10. The compound of claim 1, wherein:
the compound is any one of the following Chemical Formulas 1-1 to 1-3: 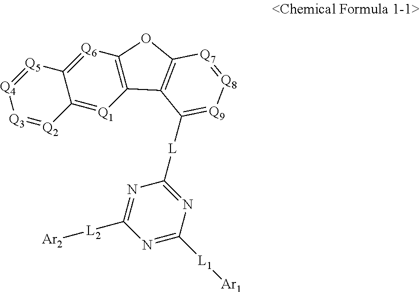
wherein, in Chemical Formula 1-1;
Q₁ to Q₉ are each independently N or C—H, with the proviso that one of Q₁ to Q₉ is N; and
L, L₁, L₂, Ar₁ and Ar₂ are as defined in claim 1; 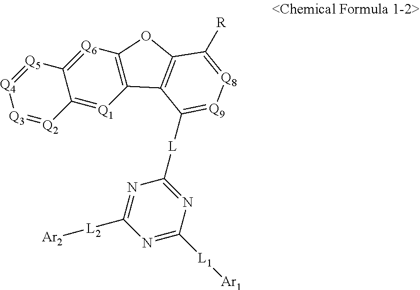
wherein, in Chemical Formula 1-2;
Q₁ to Q₆, Q₈ and Q₉ are each independently N or C—H, with the proviso that one of Q₁ to Q₆, Q₈ and Q₉ is N; and
R, L, L₁, L₂, Ar₁ and Ar₂ are as defined in claim 1; 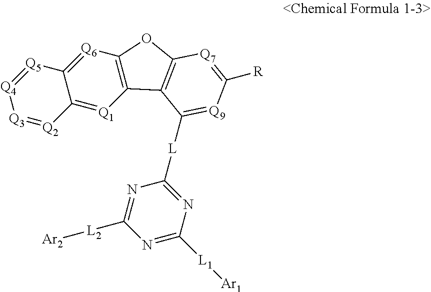
wherein, in Chemical Formula 1-3;
Q₁ to Q₇ and Q₉ are each independently N or C—H, with the proviso that one of Q₁ to Q₇ and Q₉ is N; and
R, L, L₁, L₂, Ar₁ and Ar₂ are as defined in claim 1.

11. The compound of claim 1, wherein:
the compound is any one selected from the group consisting of the following compounds:

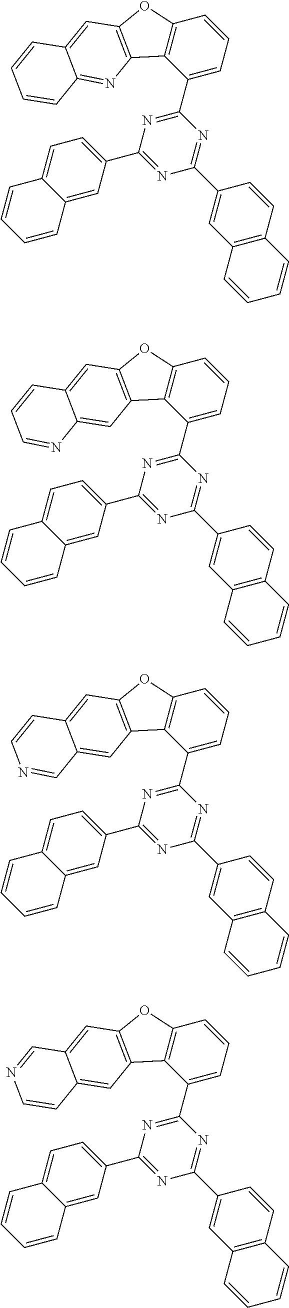 [SMILES: c1ccc2cc(-c3nc(-c4ccc5ccccc5c4)nc(-c4cccc5oc6cc7ccccc7nc6c45)n3)ccc2c1 ; c1ccc2cc(-c3nc(-c4ccc5ccccc5c4)nc(-c4cccc5oc6cc7cccnc7cc6c45)n3)ccc2c1 ; c1ccc2cc(-c3nc(-c4ccc5ccccc5c4)nc(-c4cccc5oc6cc7ccncc7cc6c45)n3)ccc2c1 ; c1ccc2cc(-c3nc(-c4ccc5ccccc5c4)nc(-c4cccc5oc6cc7cnccc7cc6c45)n3)ccc2c1]

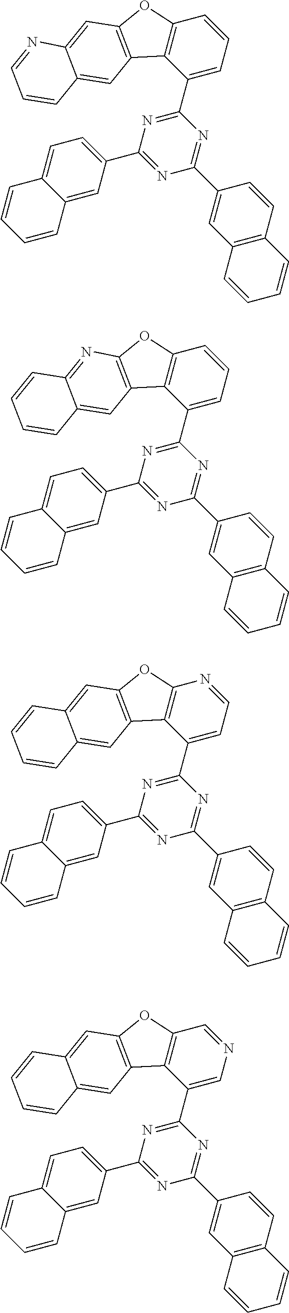 [SMILES: c1ccc2cc(-c3nc(-c4ccc5ccccc5c4)nc(-c4cccc5oc6cc7ncccc7cc6c45)n3)ccc2c1 ; c1ccc2cc(-c3nc(-c4ccc5ccccc5c4)nc(-c4cccc5oc6nc7ccccc7cc6c45)n3)ccc2c1 ; c1ccc2cc(-c3nc(-c4ccc5ccccc5c4)nc(-c4ccnc5oc6cc7ccccc7cc6c45)n3)ccc2c1 ; c1ccc2cc(-c3nc(-c4ccc5ccccc5c4)nc(-c4cncc5oc6cc7ccccc7cc6c45)n3)ccc2c1]

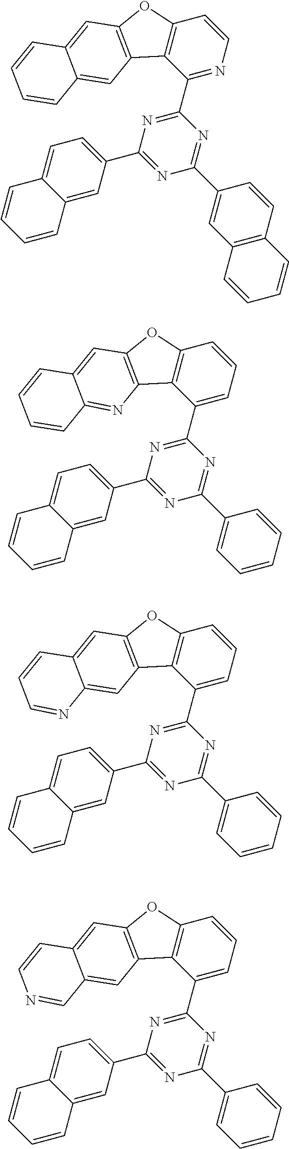 [SMILES: c1ccc2cc(-c3nc(-c4ccc5ccccc5c4)nc(-c4nccc5oc6cc7ccccc7cc6c45)n3)ccc2c1 ; c1ccc(-c2nc(-c3ccc4ccccc4c3)nc(-c3cccc4oc5cc6ccccc6nc5c34)n2)cc1 ; c1ccc(-c2nc(-c3ccc4ccccc4c3)nc(-c3cccc4oc5cc6cccnc6cc5c34)n2)cc1 ; c1ccc(-c2nc(-c3ccc4ccccc4c3)nc(-c3cccc4oc5cc6ccncc6cc5c34)n2)cc1]

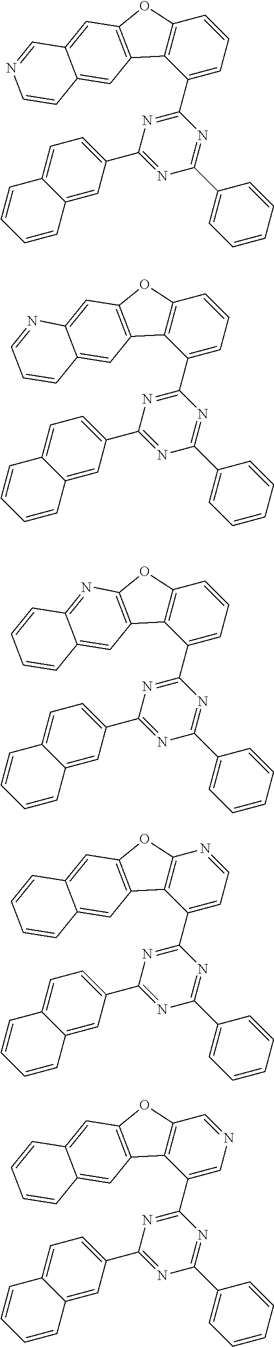

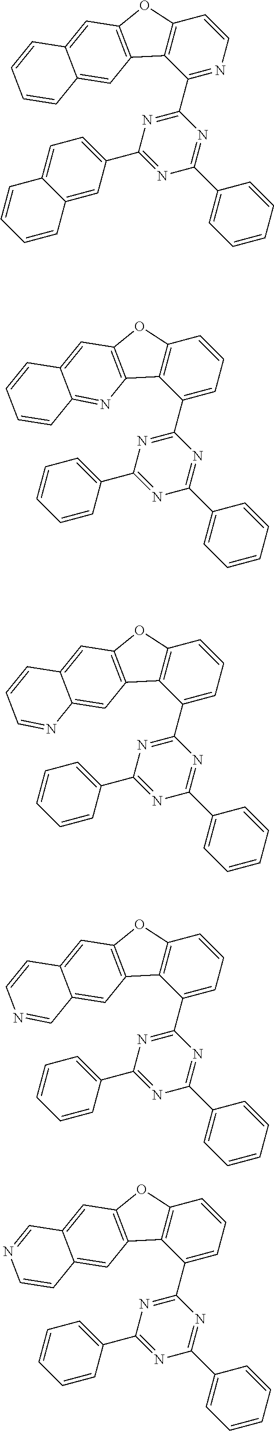

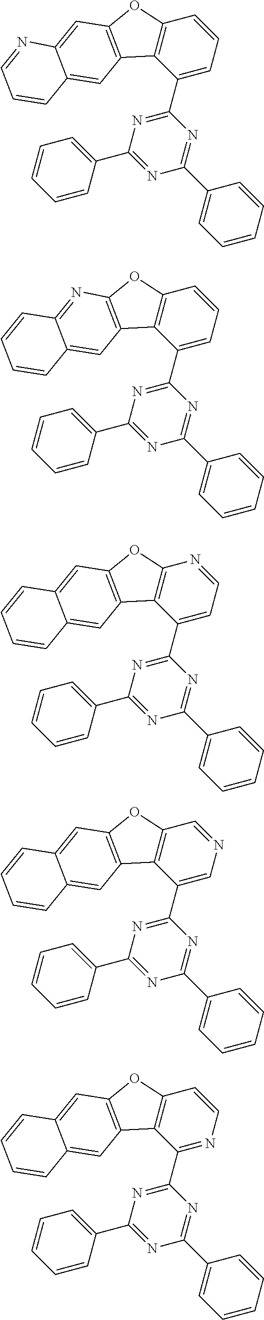

12. An organic light emitting device comprising:
a first electrode;
a second electrode that is provided opposite to the first electrode; and
one or more organic material layers that are provided between the first electrode and the second electrode, wherein one or more layers of the organic material layers comprise the compound of claim 1.

13. The organic light emitting device of claim 12, wherein
the organic material layer comprising the compound is a light emitting layer.
